(19) United States
(12) Patent Application Publication
(10) Pub. No.: US 2022/0364124 A1
(43) Pub. Date: Nov. 17, 2022

(54) EPIGENETIC MODULATION OF GENOMIC TARGETS TO CONTROL EXPRESSION OF PWS-ASSOCIATED GENES

(71) Applicant: Duke University, Durham, NC (US)

(72) Inventors: Charles A. Gersbach, Chapel Hill, NC (US); Joshua B. Black, Durham, NC (US)

(21) Appl. No.: 17/766,003

(22) PCT Filed: Oct. 2, 2020
(86) PCT No.: PCT/US2020/054160

§ 371 Date: Apr. 1, 2022

Related U.S. Application Data
(60) Provisional application No. 63/074,934, filed on Sep. 4, 2020.
(60) Provisional application No. 62/975,634, filed on Feb. 12, 2020.
(60) Provisional application No. 62/909,567, filed on Oct. 2, 2019.

(51) Int. Cl. C12N 15/90; C12N 15/86; C12N 15/10; C12N 15/113

(52) U.S. Cl. CPC C12N 15/907; C12N 15/86; C12N 15/102; C12N 15/113; C12N 2310/20

(57) ABSTRACT

Disclosed herein are DNA targeting systems that target a regulatory element of a gene within the 15q11-13 locus. Further provided are DNA targeting systems including at least one gRNA and a Cas9 protein, as well as compositions comprising the same. The compositions may be used in methods for treating Prader-Willi Syndrome (PWS) in a subject. The method may include administering to a subject the DNA targeting system.

### CROSS-REFERENCE TO RELATED APPLICATIONS

This application claims priority to U.S. Provisional Patent Application No. 62/909,587, filed Oct. 2, 2019, U.S. Provisional Patent Application No. 62/975,634, filed Feb. 12, 2020, and U.S. Provisional Patent Application No. 63/074,934, filed Sep. 4, 2020, each of which is incorporated herein by reference in its entirety.

### SEQUENCE LISTING

This application includes a Sequence Listing which has been submitted electronically in ASCII format and is hereby incorporated by reference in its entirety. Said ASCII copy created on Sep. 4, 2020, is named “SeqListing_ST25.txt” and is 484 Kbytes in size.

### FIELD

This disclosure relates to compositions and methods for the treatment of genetic and epigenetic disorders by modulating expression of genes in the region of the Prader-Willi Syndrome (PWS) imprinted locus.

### INTRODUCTION

Prader-Willi Syndrome (PWS) is a neuroendocrine and neurobehavioral disorder associated with genetic and epigenetic abnormalities within the 15q11-13 imprinted locus. Individuals with PWS display mild cognitive impairment and develop a false mental state of starvation that causes hyperphagia beginning in childhood, often resulting in extreme obesity unless strict environmental controls are enforced by caregivers to physically limit access to food. Other symptoms include neonatal hypotonia (weak muscles at birth), growth hormone deficiency, behavioral disturbances such as tantrums, outbursts and self-harm, anxiety, and compulsivity.

While the exact genetic basis of PWS remains unclear, patient mutation profiles have implicated a snoRNA cluster SNORD116 downstream of the SNURF-SNRPN open reading frame controlled by a CpG island imprinting center as a likely contributor to the disease etiology. The genes implicated in PWS are typically expressed only from the paternal copy of chromosome 15, while the PWS genes present on the maternal chromosome are epigenetically silenced. Thus, for example, a patient with paternal deletions or mutations within 15q11.2-13 can present with PWS while retaining functional copies of these genes on the maternal allele. Seventy percent of PWS cases are caused by a large 4-5 Mb deletion on the paternal allele. Twenty-five percent of PWS cases are caused by uniparental maternal disomy (UPD) 15, in which two copies of the maternal chromosome are inherited instead of one copy from each parent. Infrequently, PWS is caused by mutations or microdeletions of the PWS imprinting center. Exceedingly rare cases of PWS are caused by paternal microdeletions of PWS critical region genes, including SNORD116.

Thus, the vast majority of individuals with PWS have at least one “good” (unmutated from a DNA sequence perspective) copy of the PWS chromosomal region in every cell. The vast majority of individuals with UPD have two good copies. These genes, however, are silenced due to genomic imprinting. Attempts to activate the silenced PWS region maternal genes using small molecules have met challenges. These challenges have primarily been caused by the redundancy of epigenetic regulation, which makes it difficult for a single agent to remove the imprinting, or by undesirable genome-wide effects. For example, treatment of PWS cells with 5-Aza-DC, a compound that demethylates genomic DNA, activates maternal gene expression from the PWS locus but also causes DNA demethylation genome wide. The resulting altered expression of many other genes confers significant toxicity.

Currently there is no cure for PWS and no effective treatment for its symptoms of hyperphagia and anxiety. There remains a need for improved and/or additional therapies for treating PWS.

### SUMMARY

In an aspect, the disclosure relates to a method of treating a subject having Prader Willi Syndrome (PWS) or Prader-Willi-like disorder. The method may include administering to the subject a DNA Targeting System that targets the 15q11-13 PWS-associated locus.

In some embodiments, the DNA Targeting System is a Targeted Activator System that binds to a target region selected from the group consisting of nucleotide positions −127023 to −125023, nucleotide positions −93065 to −91065, and nucleotide positions −1104 to +896. Relative to position 1 being the start site of the SNRPN gene exon 1 of the PWS imprinting center on chromosome 15, additional target regions of interest for activation and/or demethylation may include regions at positions −126547 to −124695 [mat1]; −131937 to −130580 [mat1A]; −129415 to −127715 [mat1B]; −123798 to −122440 [mat1C]; −92568 to −91460 [mat2]; −797 to +1346 [mat3]; and/or +12858 to +14026 [mat4]. More specifically, within these regions, further subregions may include target regions at positions −126047 to −125195 [mat1]; −131437 to −131080 [mat1A]; −128915 to −128215 [mat1B]; −123298 to −122940 [mat1C]; −92068 to −91960 [mat2]; −297 to +846 [mat3]; and/or +13358 to +13526 [mat4].

In some embodiments, the nucleotide position is relative to position 1 being the start site of SNRPN exon 1 of the PWS imprinting center on chromosome 15. In some embodiments, the subject is administered a Targeted Activator System that binds to any one or more of the foregoing target regions. In some embodiments, the nucleotide position is relative to position 1 being the start site of SNRPN exon 1 of the PWS imprinting center on chromosome 15. In some embodiments, the Targeted Activator System targets a target region that results in increased expression of SNORD116 or its products. In some embodiments, the Targeted Activator System targets a target region that results in increased expression of MAGEL2. In some embodiments, at least a first Targeted Activator System targets a target region that results in increased expression of SNORD116 and at least a second Targeted Activator System targets a region that results in increased expression of MAGEL2.

In some embodiments, the DNA Targeting System is a Targeted Repressor System that binds to a target region selected from the group consisting of nucleotide positions +23022 to +25022 and nucleotide positions +34734 to +36734. Relative to position 1 being the start site of the SNRPN gene exon 1 of the PWS imprinting center on chromosome 15, additional target regions of interest for repression may include regions at positions −101358 to −94223 [pat1]; −58232 to −51914 [pat2]; −4847 to −3047 [pat3]; −1774 to +2421 [pat4]; +2446 to +24016 [pat5]; +23346 to +25082 [pat6]; +24340 to +35718 [pat7]; and/or +35206 to +36668 [pat8]. More specifically, within these regions, further subregions may include target regions at positions −100858 to −94723[pat1]; −57732 to −52414 [pat2]; −4347 to −3547 [pat3]; −1275 to +1921 [pat4]; +2946 to +23516 [pat5]; +23846 to +24582 [pat6]; +24840 to +35218 [pat7]; and/or +35706 to +36168 [pat8]. In some embodiments, the nucleotide position is relative to position 1 being the start site of SNRPN exon 1 of the PWS imprinting center on chromosome 15. In some embodiments, the subject is administered a Targeted Repressor System that binds to any one or more of the foregoing target regions. In some embodiments, the nucleotide position is relative to position 1 being the start site of SNRPN exon 1 of the PWS imprinting center on chromosome 15. In some embodiments, the administering to said subject results in increased expression of one or more of the following gene or gene products: MKRN3, MAGEL2, NDN, C15ORF2, SNURF-SNRPN, SNORD107, SNORD64, SNORD109A, SNORD116, SNORD116@, SPA1, SPA2, 116HG, SNORD116-1 to 30, Sno-Inc RNA 1 to 5, IPW, SNORD115, SNORD115@, 115HG, SNORD115-1 to 48, SNORD109B, and/or SNG14. In some embodiments, the subject has a PWS Type 1 large deletion, PWS Type 2 large deletion, PWS imprinting center mutation, or PWS uniparental disomy. In some embodiments, the subject has a PWS microdeletion encompassing SNORD116, but not MAGEL2. In some embodiments, the subject has a PWS or PWS-like atypical deletion encompassing MAGEL2, but not SNORD116. In some embodiments, the subject has heterozygous Schaaf-Yang syndrome or MAGEL2 disorder.

In some embodiments, the Targeted Activator System is a CRISPR-Cas Type II system. In some embodiments, the Targeted Activator System comprises a fusion protein comprising a Cas9 polypeptide with reduced nuclease activity and an activator; and one or more guide RNAs (gRNA) that bind to a target region in the 15q1-13 PWS-associated locus. In some embodiments, at least one gRNA comprises at least 10, 11, 12, 13, 14, 15, 16, 17, 18, 19, or 20 consecutive nucleotides of any of SEQ ID NOs: 1-12, or of any of SEQ ID NOs: 47-86 and 91-1122, or a corresponding allelic variant thereof. In some embodiments, the Cas9 is a S. pyogenes Cas9 (for example, the polypeptide sequence of SEQ ID NO: 24). In some embodiments, the subject is administered said fusion protein and said one or more gRNAs; or a nucleic acid sequence encoding said fusion protein, and said one or more gRNAs; or a nucleic acid sequence encoding said fusion protein, and nucleic acid sequence(s) encoding said one or more gRNAs. In some embodiments, the subject is administered a vector comprising a nucleic acid encoding said fusion protein and/or a vector comprising a nucleic acid encoding said gRNA. In some embodiments, the vector is a viral vector, optionally a retroviral, lentiviral, adenovirus, adeno-associated virus vector, synthetic vector, or is a vector within a lipid nanoparticle. In some embodiments, the Targeted Activator System comprises a Cas9 polypeptide and one or more dead gRNAs that bind to a target region in the 151q1-13 PWS-associated locus. In some embodiments, the Targeted Activator System is a CRISPR-Cas Type II system comprising one or more gRNAs. In some embodiments, at least one gRNA comprises a nucleotide sequence at least 80% complementary to at least 10-20 consecutive nucleotides of said target region. In some embodiments, the at least one gRNA comprises a nucleotide sequence at least 80% complementary to at least 18 consecutive nucleotides of said target region. In some embodiments, the Targeted Activator System is a ZF-based and/or TALE-based system. In some embodiments, the Targeted Activator System comprises one or more fusion proteins comprising a TALE that binds to the target region of the 15q11-13 PWS-associated locus and an activator. In some embodiments, the Targeted Activator System comprises one or more fusion proteins comprising a ZF that binds to the target region of the 15q11-13 PWS-associated locus and an activator. In some embodiments, the activator is VP64, VP16; GAL4; p65 subdomain (NFkB); KMT2 family transcriptional activators: hSET1A, hSET1B, MLL1 to 5, ASH1, and homologs (Trx, Trr, Ash1); KMT3 family: SYMD2, NSD1; KMT4 family: DOT1L and homologs; KDM1: LSD1/BHC110 and homologs (SpLsd1/Swm1/Saf110, Su(var)3-3); KDM3 family: JHDM2a/b; KDM4 family: JMJD2A/JHDM3A, JMJD2B, JMJD2C/GASC1, JMJD2D, and homologs (Rph1); KDM6 family: UTX, JMJD3, VP64-p65-Rta (VPR); synergistic action mediator (SAM); p300; VP160; VP64-dCas9-BFP-VP64; KAT2 family: hGCN5, PCAF, and homologs (dGCN5/PCAF, Gcn5; KAT3 family: CBP, p300 and homologs (dCBP/NEJ); KAT4: TAF1 and homologs (dTAF1); KAT5: TIP60/PLIP, and homologs; KAT6: MOZ/MYST3, MORF/MYST4, and homologs (Mst2, Sas3, CG1894); KAT7: HBO1/MYST2, and homologs (CHM, Mst2); KAT8: HMOF/MYST1, and homologs (dMOF, CG1894, Sas2, Mst2); KAT13 family: SRC1, ACTR, P160, CLOCK, and homologs; AID/Apobed deaminase family: AID; TET dioxygenase family: TET1; DEMETER glycosylase family: DME, DML1, DML2, or ROS1. In some embodiments, the Targeted Repressor System is a CRISPR-Cas Type II system. In some embodiments, the Targeted Repressor System comprises a fusion protein comprising a Cas9 polypeptide with reduced nuclease activity and a repressor, and one or more guide RNAs (gRNA) that bind to the target region of the 151q1-13 PWS-associated locus. In some embodiments, at least one gRNA comprises at least 10, 11, 12, 13, 14, 15, 16, 17, 18, 19, or 20 consecutive nucleotides of any of SEQ ID NOs: 1-12, or of any of SEQ ID NOs: 47-86 and 91-1122, or a corresponding allelic variant thereof. In some embodiments, the Cas9 is a S. pyogenes Cas9 (SEQ ID NO: 24). In some embodiments, the subject is administered said fusion protein and said one or more gRNAs; or a nucleic acid sequence encoding said fusion protein, and said one or more gRNAs; or a nucleic acid sequence encoding said fusion protein, and nucleic acid sequence(s) encoding said one or more gRNAs. In some embodiments, the subject is administered a vector comprising a nucleic acid encoding said fusion protein and/or a vector comprising a nucleic acid encoding said gRNA. In some embodiments, the vector is a viral vector, optionally a retroviral, lentiviral, adenovirus, adeno-associated virus vector, synthetic vector, or is a vector within a lipid nanoparticle. In some embodiments, the Targeted Repressor System comprises a Cas9 polypeptide with reduced nuclease activity and one or more gRNAs that bind to the target region of the 15q11-13 PWS-associated locus. In some embodiments, the Targeted Repressor System is a CRISPR-Cas Type II system comprising one or more gRNAs, wherein at least one gRNA comprises a nucleotide sequence at least 80% complementary to at least 10-20 consecutive nucleotides of said target region. In some embodiments, the at least one gRNA comprises a nucleotide sequence at least 80% complementary to at least 18 consecutive nucleotides of said target region. In some embodiments, the Targeted Repressor System is a ZF-based and/or TALE-based system.

In some embodiments, the Targeted Repressor System comprises one or more fusion proteins comprising a TALE that binds to the target region of the 15q11-13 PWS-associated locus and a repressor. In some embodiments, the Targeted Repressor System comprises one or more fusion proteins comprising a ZF that binds to the target region of the 15q11-13 PWS-associated locus and a repressor. In some embodiments, the repressor comprises KRAB, Mad mSIN3 interaction domain (SID); the ERF repressor domain (ERD); KMT1 family: SUV39H1, SUV39H2, G9A, ESET/SETBD1, and homologs (Cir4, Su(var)3-9); KMT5 family: Pr-SET7/8, SUV4-20H1, and homologs (PR-set7, Suv4-20, and Set9); KMT6: EZH2, KMT8: RIZ1, KDM4 family: JMJD2A/JHDM3A, JMJD2B, JMJ2D2C/GASC1, JMJD2D, and homologs (Rph1); KDM5 family JARID1A/RBP2, JARID1B/PLU-1, JARID1C/SMCX, JARID1D/SMCY, and homologs (Lid, Jhn2, Jmj2); HDAC1, HDAC2, HDAC3, HDAC8, and its homologs (Rpd3, Hos1, Cir6); HDAC4, HDAC5, HDAC7, HDAC9, and its homologs (Hda1, Cir3); SIRT1, SIRT2, and its homologs (Sir2, Hst1, Hst2, Hst3, and Hst4); HDAC11, DNMT1, DNMT3a/3b, MET1, DRM3, and homologs, ZMET2, CMT1, CMT2, Laminin A, Laminin B, or CTCF.

In a further aspect, the disclosure relates to a DNA Targeting System that binds to a target region. The target region may be selected from the group consisting of nucleotide positions −127023 to −125023, nucleotide positions −93065 to −91065, and nucleotide positions −1104 to +896. Alternatively, the target region may be within any of the following ranges: positions −126547 to −124695 [mat1]; −131937 to −130580 [mat1A]; −129415 to −127715 [mat1B]; −123798 to −122440 [mat1C]; −92568 to −91460 [mat2]; −797 to +1346 [mat3]; and/or +12858 to +14026 [mat4]. The nucleotide position is relative to position 1 being the start site of SNRPN exon 1 of the PWS imprinting center on chromosome 15.

Another aspect of the disclosure provides a DNA Targeting System that binds to a target region. The target region may be selected from the group consisting of nucleotide positions −126523 to −125523, nucleotide positions −92565 to −91565, and nucleotide positions −604 to +395. Alternatively, the target region may be within any one of the following ranges: positions −126047 to −125195 [mat1]; −131437 to −131080 [mat1A]; −128915 to −128215 [mat1B]; −123298 to −122940 [mat1C]; −92068 to −91960 [mat2]; −297 to +846 [mat3]; and/or +13358 to +13526 [mat4]. The nucleotide position may be relative to position 1 being the start site of SNRPN exon 1 of the PWS imprinting center on chromosome 15. In some embodiments, the DNA Targeting System is a Targeted Activator System.

Another aspect of the disclosure provides a DNA Targeting System that binds to a target region selected from the group consisting of nucleotide positions +23022 to +25022 and nucleotide positions +34734 to +36734. Alternatively, the target region may be within any one of the following ranges: positions +23346 to +25082 [pat6]; and/or +35206 to +36668 [pat8]. The nucleotide position may be relative to position 1 being the start site of SNRPN exon 1 of the PWS imprinting center on chromosome 15.

Another aspect of the disclosure provides a DNA Targeting System that binds to a target region selected from the group consisting of nucleotide positions +23522 to +24522 and nucleotide positions +35234 to +36234. Alternatively, the target region may be within any one of the following ranges: positions +23846 to +24582 [pat6]; and/or +35706 to +36168 [pat8]. The nucleotide position may be relative to position 1 being the start site of SNRPN exon 1 of the PWS imprinting center on chromosome 15.

In some embodiments, the DNA Targeting System is a Targeted Repressor System. In some embodiments, the DNA Targeting System is a CRISPR-Cas Type II system comprising one or more gRNAs. In some embodiments, at least one gRNA comprises a nucleotide sequence at least 80% complementary to at least 10-20 consecutive nucleotides of said target region.

In some embodiments, the DNA Targeting System has demethylase activity. In some embodiments, the DNA Targeting System binds to a target region selected from the group consisting of nucleotide positions −797 to +1346 [mat3]; and/or +12858 to +14026 [mat4]; or more specifically, 297 to +846 [mat3] and/or +13358 to +13526 [mat4], relative to position 1 being the start site of SNRPN exon 1 of the PWS imprinting center on chromosome 15.

Another aspect of the disclosure provides a nucleic acid encoding at least one component of said DNA Targeting System.

Another aspect of the disclosure provides a vector comprising a nucleic acid as disclosed herein.

Another aspect of the disclosure provides a first nucleic acid encoding at least one gRNA that may comprise a nucleotide sequence at least 80% complementary to at least 10-20 consecutive nucleotides of said target region, and a second nucleic acid encoding a Cas9 protein, or a Cas9 fusion protein. The first and second nucleic acids may be on the same or different vectors.

In some embodiments, the vector is a viral vector, optionally a retroviral, lentiviral, adenovirus, adeno-associated virus vector, synthetic vector, or is a vector within a lipid nanoparticle.

Another aspect of the disclosure provides a ribonucleoprotein. The ribonucleoprotein may comprise a Cas9 protein or a Cas9 fusion protein and at least one gRNA. The gRNA may comprise a nucleotide sequence at least 80% complementary to at least 10-20 consecutive nucleotides of said target region.

Another aspect of the disclosure provides a pharmaceutical composition. The pharmaceutical composition may comprise said DNA Targeting System as disclosed herein, said nucleic acids as disclosed herein, said vector as disclosed herein, or said ribonucleoprotein as disclosed herein.

Another aspect of the disclosure provides a guide RNA (gRNA). The gRNA may comprise a polynucleotide sequence corresponding to at least one of SEQ ID NOs: 1-12, or at least one of SEQ ID NOs: 47-86 and 91-1122, or a truncation thereof, a complement thereof, or an allelic variant thereof.

Another aspect of the disclosure provides a DNA Targeting System that binds to a regulatory element of a gene within the 15q11-13 locus. The DNA Targeting System may comprise at least one gRNA that binds and targets a polynucleotide sequence. The polynucleotide sequence may comprise a nucleotide sequence corresponding to a complement of at least one of SEQ ID NOs: 1-12, or at least one of SEQ ID NOs: 47-86 and 91-1122, or a truncation thereof, a complement thereof, or an allelic variant thereof.

In some embodiments, the at least one gRNA comprises a polynucleotide sequence corresponding to at least one of SEQ ID NOs: 1-12, or at least one of SEQ ID NOs: 47-86 and 91-1122, or a variant thereof. In some embodiments, the DNA Targeting System further comprises a Clustered Regularly Interspaced Short Palindromic Repeats associated (Cas) protein or a fusion protein. In some embodiments, the fusion protein comprises two heterologous polypeptide domains, wherein the first polypeptide domain comprises a Cas protein and the second polypeptide domain has an activity selected from the group consisting of transcription activation activity, transcription repression activity, transcription release factor activity, histone modification activity, nuclease activity, nucleic acid association activity, methylase activity, demethylase activity, acetylation activity, and deacetylation activity. In some embodiments, the Cas protein comprises a Streptococcus pyogenes Cas9 protein, or a variant thereof. In some embodiments, the Cas protein comprises a VQR variant of the S. pyogenes Cas9 protein. In some embodiments, the DNA targeting system comprises a fusion protein. In some embodiments, the second polypeptide domain has transcription activation activity, transcription repression activity, histone modification activity, or a combination thereof. In some embodiments, the fusion protein comprises dCas9-VP64, VP64-dCas9-VP64, dCas9-p300, dCas9-KRAB, or dCas9-Tet1c. In some embodiments, the Cas protein comprises a Cas9 that recognizes a Protospacer Adjacent Motif (PAM) of NGG (SEQ ID NO: 13), NGA (SEQ ID NO: 14), NGAN (SEQ ID NO:15), or NGNG (SEQ ID NO:16). In some embodiments, the gene within the 15q11-13 locus is selected from SNRPN, SNORD115, SNORD116, SPA1, SPA2, and MAGEL2.

Another aspect of the disclosure provides an isolated polynucleotide sequence comprising the gRNA as disclosed herein.

Another aspect of the disclosure provides an isolated polynucleotide sequence encoding the DNA targeting system as disclosed herein.

Another aspect of the disclosure provides a vector comprising the isolated polynucleotide sequence as disclosed herein.

Another aspect of the disclosure provides a vector encoding the gRNA as disclosed herein and a Clustered Regularly Interspaced Short Palindromic Repeats associated (Cas) protein. In some embodiments, the Cas protein comprises a Streptococcus pyogenes Cas9 protein, or variant thereof. In some embodiments, the Cas protein comprises a VQR variant of the S. pyogenes Cas9 protein.

Another aspect of the disclosure provides a cell comprising the gRNA as disclosed herein, the DNA targeting system as disclosed herein, the isolated polynucleotide sequence as disclosed herein, or the vector as disclosed herein, or a combination thereof.

Another aspect of the disclosure provides a pharmaceutical composition comprising the gRNA as disclosed herein, the DNA targeting system as disclosed herein, the isolated polynucleotide sequence as disclosed herein, or the vector as disclosed herein, or the cell of as disclosed herein, or a combination thereof.

Another aspect of the disclosure provides a method for treating Prader-Willi Syndrome (PWS) in a subject, the method comprising administering to the subject the DNA targeting system as disclosed herein, the isolated polynucleotide sequence as disclosed herein, the vector as disclosed herein, or the cell as disclosed herein, or a combination thereof.

In some embodiments, the expression of at least one gene within the 15q11-q13 locus is increased. In some embodiments, the gene within the 15q11-13 locus is selected from SNRPN, SNORD115, SNORD116, SNORD109A, IPW, SPA1, SPA2, and MAGEL2. In some embodiments, the expression of at least one RNA transcript selected from SNRPN, SNORD115, SNORD116, SPA1, SPA2, and MAGEL2, or a combination thereof, is increased. In some embodiments, the initiation of transcription from the SNRPN promoter, SNORD115 promoter, SNORD116 promoter, or a combination thereof, is increased.

The disclosure provides for other aspects and embodiments that will be apparent in light of the following detailed description and accompanying figures.

### BRIEF DESCRIPTION OF THE DRAWINGS

FIG. 1A-FIG. 1C. Generation of a SNRPN-2A-GFP reporter cell line. (FIG. 1A) Schematic representation of the knock-in of a P2A-GFP cassette into exon ten of SNRPN in a human pluripotent stem cell line using Cas9 nuclease and a donor template. (FIG. 1B) Imprinting at the 15q11-13 locus imparts monoallelic expression of SNRPN only from the paternal allele. Consequently, paternal SNRPN-2A-GFP cells are GFP-positive while maternal SNRPN-2A-GFP cells are GFP-negative. (FIG. 1C) Mean fluorescence intensity of human induced pluripotent stem cells that were transfected with the SNRPN-2A-GFP construct. Clone genotype was assessed by PCR. As expected, heterozygous clones that harbor one GFP-tagged allele and one wild-type allele displayed a bimodal distribution in GFP fluorescence, presumably due to a mixture of paternal and maternal insertions. In the heterozygous clones (middle column), the dashed line circle around the top set of dots indicates the paternal insertion, while the solid line circle around the bottom set of dots indicates the maternal insertion.

FIG. 2A-FIG. 2C. A CRISPRi screen in patSNRPN-2A-GFP cells identified genomic elements regulating paternal SNRPN expression. (FIG. 2A) Schematic representation of the gRNA library generated in a CRISPRi/a screen for putative regulatory elements controlling expression of the SNRPN host transcript at the 15q11-13 PWS-associated locus in human induced pluripotent stem cells. A dCas9^{KRAB} patSNPRN-2A-GFP reporter cell line was transduced with the PWS pooled lentiviral gRNA library at low MOI and sorted for GFP expression via FACS. gRNA abundance in each cell bin was measured by deep sequencing and depleted or enriched gRNAs were identified by differential expression analysis. (FIG. 2B) SNRPN-2A-GFP cells were sorted for the highest and lowest 10% expressing cells based on GFP signal. A bulk unsorted population of cells was also taken to establish the baseline gRNA distribution. (FIG. 2C) Differential expression analysis of normalized gRNA counts between the GFP-High and GFP-low cell populations. Open circle data points indicate FDR<0.01 by differential DESeq2 analysis (n=3 biological replicates).

FIG. 3A-3B. (FIG. 3A) Results of the CRISPRi screen where differentially enriched gRNAs map to defined regions within the 15q11-13 locus. A map of a sub-region of 15q11-13 containing a high density of gRNAs overlayed to genomic annotations of SNRPN transcripts. Light grey shaded regions (labeled 1, 2, 3, 4 and 6) contain gRNAs enriched in the GFP-low expressing cells, and white outlined regions (labeled 5 and 7) contain gRNAs enriched in the GFP-high expressing cells. Three elements upstream of the imprinting center, including two sites within SNRPN introns and one site proximal to an alternative SNRPN transcriptional start site, were enriched in GFP-low expressing cells. The strongest effects were localized to the imprinting center and throughout the gene body of SNRPN. Two regions downstream of the SNPRN gene body were enriched in GFP-high expressing cells. Several of the differentially enriched regions did not overlay with DNase hypersensitivity signal in human pluripotent stem cells. (FIG. 3B) DNA strand-specific effects of dCas9KRAB targeting of paternal SNRPN. Genome browser track depicting gRNA enrichment in the dCas9KRAB screen of the paternal allele separated by DNA strand. The positive DNA strand (+) consists of the sense strand of the SNRPN gene.

FIG. 4A-FIG. 4H. Independent validations of gRNAs from the CRISPRi patSNRPN-2A-GFP screen in human induced pluripotent stem cells. (FIG. 4A) Genomic track of the SNRPN locus with positions of the selected gRNAs annotated. The gRNA tracks indicate the direction of the predicted effect on SNRPN-2A-GFP expression. Open rectangle (gRNA target sites: 24022 and 35734) is increased, and solid rectangle (gRNA target sites: −98584, −3666, −104, 11531, and 26886) is decreased. gRNA position is relative to the SNRPN transcription start site at the imprinting center (PWS-IC). (FIG. 4B) Mean fluorescence intensity of dCas9^{KRAB} patSNRPN-2A-GFP cells five days after transduction of the indicated gRNAs. (FIG. 4C) Relative RNA expression of paternal or maternal SNRPN (a PWS region gene) five days after transduction of the indicated gRNAs. (FIG. 4D) Relative RNA expression of ncRNAs within the 15q11-13 locus including SNORD116, SPA1 and SPA2 five days after transduction of the indicated gRNAs. *p<0.05 by global one-way ANOVA with Dunnett's post hoc test comparing all groups to Empty Vector, n=2 biological replicates. Relative SNRPN expression with dCas9KRAB and gRNAs targeting the indicated regions, as assessed via (FIG. 4E) mean fluorescence intensity via flow cytometry and (FIG. 4F) quantification of total SNRPN RNA. (FIG. 4G) Quantification of polyA-enriched RNA. (FIG. 4H) Expression of non-coding RNAs SPA1, SPA2, and SNORD116 downstream of SNRPN coding exons with dCas9KRAB and gRNAs targeting the indicated regions (*p<0.05 global one-way ANOVA with Dunnett's post hoc test comparing all conditions to Empty gRNA vector, n=2 biological replicates).

FIG. 5A-FIG. 5E. A CRISPRa screen in matSNRPN-2A-GFP cells identifies genomic elements regulating maternal SNRPN expression. (FIG. 5A) Schematic representation of a CRISPRa screen for regulatory elements controlling expression of the SNRPN host transcript at the 15q11-13 PWS-associated locus in human pluripotent stem cells. A ^{VP64}dCas9^{VP64} matSNPRN-2A-GFP reporter cell line was transduced with the PWS pooled lentiviral gRNA library at low MOI and sorted for GFP expression via FACS. gRNA abundance in each cell bin was measured by deep sequencing and depleted or enriched gRNAs were identified by differential expression analysis. (FIG. 5B) Differential expression analysis of normalized gRNA counts between the GFP-High and GFP-low cell populations. Open circle data points indicate FDR<0.01 by differential DESeq2 analysis (n=3 biological replicates). (FIG. 5C) Comparison of the fold change in gRNA abundance for the 12 gRNAs enriched in the CRISPRa matSNPRN-2A-GFP versus the corresponding fold changes observed in the CRISPRi patSNPRN-2A-GFP. (FIG. 5D) Genomic track of the SNRPN locus with positions of the selected gRNAs annotated. The solid (paternal) or open (maternal) rectangular marks at the bottom of the gRNA tracks indicates which screen identified a given region as significant. gRNA position is relative to the SNRPN transcription start site at the imprinting center (PWS-IC). (FIG. 5E) Relative expression of maternal SNRPN with the indicated gRNAs and ^{VP64}dCas9^{VP64} after six days of transgene expression. *p<0.05 by global one-way ANOVA with Dunnett's post hoc test comparing all groups to No gRNA, n=2 biological replicates.

FIG. 6A-FIG. 6C. Genomic regulatory elements controlling allele-specific expression of the SNRPN host transcript. (FIG. 6A) Genome browser track depicting the screen results. Boxes labeled pat # and mat # indicate regions of enriched or depleted gRNAs in the CRISPRi or CRISPRa screens, respectively. (FIG. 6B) Individual gRNA validations for paternal and (FIG. 6C) Individual gRNA validations for maternal SNRPN-2A-GFP expression (*p<0.05 global one-way ANOVA with Dunnett's post hoc test comparing all groups to Empty gRNA vector). AS-IC indicates Angelman syndrome imprinting center; PWS-IC indicates Prader-Willi syndrome imprinting center.

FIG. 7A-FIG. 7F. gRNAs identified in the matSNRPN-GFP cells were tested in patient-derived iPSCs. (FIG. 7A) The patient-derived iPSCs harbored a 15q11-13 large deletion on the paternal allele. (FIG. 7B) Stable expression of VP64-dCas9-VP64 was established via lentiviral transduction, and gRNAs were delivered on a separate lentiviral vector. Cells were harvested seven days after transduction of the gRNAs, and qRT-PCR was used to assess expression of maternal genes within 151q1-13. Expression of imprinted genes was depicted for SNRPN (FIG. 7C), SNORD116 (FIG. 7D), SPA1 (FIG. 7E), and UBE3A (FIG. 7F) seven days after transduction of the indicated gRNAs (*p<0.05 global one-way ANOVA with Dunnett's post hoc test comparing all groups to Empty gRNA vector, n=3 biological replicates). See Example 5.

FIG. 8A-FIG. 8C. Identification of additional SNRPN regulatory elements with a CRISPRa screen using guide RNAs targeting two different regions. (FIG. 8A) Schematic representation of a CRISPRa pooled screen in maternal SNRPN-2A-GFP cells with a gRNA vector expressing a mat1-targeting gRNA along with each gRNA from the original library. Cells were transduced with the pooled lentiviral library at MOI=0.2 and sorted for GFP expression via FACS at Day 9. gRNA abundance in each cell bin was measured by deep sequencing and depleted or enriched gRNAs were identified by differential expression analysis. (FIG. 8B) Genome browser track depicting results from both the single and dual gRNA screens. (FIG. 8C) Comparison of the significant hits (FDR<0.05) between the single and dual gRNA CRISPRa screens. See Example 7.

FIG. 9A-FIG. 9E Activation of PWS-associated genes in iPSC-derived neurons. (FIG. 9A) Schematic representation of the neuronal differentiation of PWS patient iPSCs via NEUROG3 overexpression. Mat1 or mat2 gRNAs were delivered three days after the onset of differentiation, and cells were FACS sorted for NCAM expression and harvested for RNA on day six. Expression of PWS-associated genes including (FIG. 9B) SNRPN, (FIG. 9C) SPA1, (FIG. 9D) SNORD116, and (FIG. 9E) UBE3A, with delivery of an empty gRNA vector or gRNAs targeting mat1 or mat2 (*p<0.05 global one-way ANOVA with Dunnett's post hoc test comparing all conditions to Empty gRNA vector, n=3 biological replicates). See Examples 9 and 8.

FIG. 10A-FIG. 10E. A targeted DNA demethylation screen in matSNRPN-2A-GFP cells identifies target regions capable of re-activating maternal PWS-associated genes. (FIG. 10A) Schematic representation of a CRISPR/Cas9 pooled screen to identify target regions for demethylation to increase expression of the SNRPN host transcript at the 15q11-13 PWS-associated locus in human pluripotent stem cells. A matSNPRN-2A-GFP reporter cell line was transduced with the pooled lentiviral gRNA library at low MOI and sorted for GFP expression via FACS. gRNA abundance in each cell bin was measured by deep sequencing and depleted or enriched gRNAs were identified by differential expression analysis. (FIG. 10B) Differential expression analysis of normalized gRNA counts between the GFP-High and GFP-Low cell populations. Open circle data points indicate FDR<0.05 by differential DESeq2 analysis. (FIG. 10C) Comparison of the significant hits (FDR<0.05) between dCas9-VP84 and Tet1CD-dCas9 for reactivation of SNRPN-GFP. (FIG. 10D) Genome browser track depicting region with positions annotated of the gRNAs which, together with Tet1CD-dCas9 activity, re-activated SNRPN transcript expression. gRNA position is relative to the SNRPN transcription start site of the PWS Imprinting Center. Boxes labeled mat3 and mat4 indicate regions of enriched gRNAs in the GFP-high cell bin. (FIG. 10E) Genome browser track depicting the Tet1CD dCas9 screen results. Boxes labeled mat3 and mat4 indicate regions of enriched gRNAs in the GFP-high cell bin. See Examples 10 and 9.

FIG. 11A-FIG. 11B. Expression of PWS-associated genes, with delivery of an empty gRNA vector or a single gRNA targeting the PWS locus at the indicated position. (FIG. 11A) SNRPN-GFP transcript levels were quantified by RT-qPCR at nine days post-transduction, relative to non-targeting gRNA control. (FIG. 11B) Cells were harvested nine days post-transduction and analyzed by flow cytometry. GFP expression of the transduced cell population was quantified by mean fluorescence intensity (MFI) of the FITC channel. See Example 10.

### DETAILED DESCRIPTION

The present invention is directed to methods of treating Prader-Willi Syndrome (PWS), Prader-Willi-like syndrome, or disorders that would benefit from activation of the genes within the PWS locus, wherein activation of the genes within one allele of the 15q11-13 locus reintroduces lost functional gene expression. A tiled screen across the PWS locus employing either dCas9-VP64 (activator) or dCas9-KRAB (repressor) identified regions that if activated or repressed would result in increased gene expression from the maternal allele in the PWS region. Two independent screens were performed: (1) dCas9KRAB-based repression of the paternal allele and (2) VP64dCas9VP64-based activation of the maternal allele.

A further tiled screen across the PWS locus employing Tet1CD-dCas9 (a demethylase designated Ten-eleven translocation methylcytosine dioxygenase 1) identified target regions that if demethylated would result in increased gene expression from the maternal allele in the PWS region. While non-targeted therapy with small molecules that inhibit epigenetic-modifying enzymes, such as G9a or 5-Aza-dC has been attempted in the past, such therapy has an added risk of off-target activity. The more targeted approach disclosed herein is expected to achieve similar or superior epigenetic modification at the target region with minimal off-target activity.

These methods identified regions of regulatory elements within the 15q11-13 imprinted region that control expression of genes such as SNRPN and the transcript containing SNORD115 and SNORD116. Targeting some regions with CRISPRa technology resulted in activation of silenced imprinted genes, while targeting other regions with CRISPRi technology also resulted in activation of silenced imprinted genes. The identification of these regions may permit extremely targeted regulation of gene expression, without the substantial off-target toxicities encountered with less specific small molecule epigenome modification approaches.

Surprisingly, the use of TET1CD-dCas9 was sufficient for reactivation of SNRPN transcript expression, including in regions where targeting using VP64-dCas9-VP64 was insufficient to reactivate SNPRN transcript expression. Thus, the use of multiple types of effectors targeted through dCas9-based systems may obtain synergistic activity. In addition, targeting two different regions with the same effector (for example, using two guide RNAs that targeted two different regions, with VP64-dCas9-VP64) was shown to provide synergistic activity.

Results described herein showed that the expression of maternal genes, which are typically present but silenced by imprinting in PWS patients, was successfully activated using targeted epigenetic editing. Successful reactivation was demonstrated in iPSC cells from PWS patients, as well as in iPSC-derived neurons from PWS patients. iPSC cells and iPSC-derived neurons were used because neuron pathology may play a role in the neurological defects seen in PWS. The disclosure provides a new avenue for epigenetic therapy, by identifying target regions for DNA Targeting Systems, including Targeted Activator Systems and/or Targeted Repressor Systems. DNA Targeting Systems that bind to such target regions, compositions comprising such DNA Targeting Systems, and methods of using such DNA Targeting Systems, are disclosed herein.

The disclosure also provides novel in vitro disease models of PWS to better uncover the role of cell-type specificity and epigenetic perturbation in the mechanisms of gene regulation in PWS, such as by providing various cell types that have been modified by such DNA Targeting Systems or that comprise, for example, Targeted Activator Systems and/or Targeted Repressor Systems. Cells may be modified, such as, by insertion, deletion and/or substitution within such target regions, optionally within or near regulatory elements. Cells may also be modified, such as, by methylation, demethylation of DNA, or by acetylation or deacetylation of associated histones.

The disclosure further provides potential drug targets for next-generation epigenetic therapies, and methods of screening for compounds that target the target regions identified herein by (a) contacting (i) modified cells produced with such DNA Targeting Systems, or (ii) cells comprising such Targeted Activator Systems and/or Targeted Repressor Systems, with a candidate compound, (b) detecting expression of PWS genes, and (c) selecting compounds that increase or decrease expression of PWS genes.

In the studies described herein, the PWS region was targeted with 11,000 guide RNAs tiled across the PWS region. Guide RNA placement was designed to follow known regions of putative regulator element tracts utilizing data from the ENCODE project. These included H3K27Ac, a marker that is often found near regulatory elements, areas of DNase hypersensitivity, and CpG islands. Additionally, guide RNA density was further increased at regions of known epigenetic modulation of PWS region gene expression, such as around the PWS imprinting center. The screen was performed in human induced pluripotent stem cells (hiPSCs) in which GFP was inserted after the last exon of the SNRPN gene on either the maternal or paternal allele.

Hits from the screen were validated in independent experiments in hiPSCs carrying the maternal SNRPN-GFP by FACS as well as by qRT-PCR. Surprisingly, it has now been demonstrated that targeting a single region can activate maternal gene expression of multiple genes in the PWS region in iPSCs from an individual with PWS due to large paternal deletion at 15q11.2-13. Data indicate that remarkably large fold increases in activation of important genes such as SNORD116 can be achieved, without adversely impacting expression levels of other genes such as UBE3A (a gene in the PWS locus that is paternally imprinted and maternally expressed, and for which loss of function causes Angelman syndrome).

Specifically described herein are gRNAs that bind to the identified target regions for use with a CRISPR-Cas system, and DNA Targeting Systems, including Targeted Activator System and Targeted Repressor Systems, that include the gRNAs, that bind and target a target region within the 15q11-13 locus. The compositions and methods detailed herein may increase the expression of at least one gene within the 15q11-q13 locus. As such, the compositions and methods detailed herein may be used to treat Prader-Willi Syndrome (PWS) or other disorders described herein in a subject, including PWS-like syndrome, PWS Type 1 large deletion, PWS Type 2 large deletion, PWS imprinting center mutation or PWS uniparental disomy, PWS microdeletion, atypical deletion encompassing MAGEL2, Heterozygous Schaaf-Yang syndrome, Chitayat-Hall syndrome, MAGEL2 disorder, MAGEL2-related disorder. The gene within the 15q11-13 locus may be selected from, for example, SNRPN, SNORD115, SNORD116, SNORD109A, IPW and/or MAGEL2. The DNA Targeting System(s), Targeted Activator System(s) or Targeted Repressor System(s) may further include a Clustered Regularly Interspaced Short Palindromic Repeats associated (Cas) protein or a fusion protein comprising a Cas protein.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and,” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The term “about” as used herein as applied to one or more values of interest, refers to a value that is similar to a stated reference value. In certain aspects, the term “about” refers to a range of values that fall within 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

“Adeno-associated virus” or “AAV” as used interchangeably herein refers to a small virus belonging to the genus Dependovirus of the Parvoviridae family that infects humans and some other primate species, including variants thereof. AAV is not currently known to cause disease and consequently the virus causes a very mild immune response.

“Amino acid” as used herein refers to naturally occurring and non-natural synthetic amino acids, as well as amino acid analogs and amino acid mimetics that function in a manner similar to the naturally occurring amino acids. Naturally occurring amino acids are those encoded by the genetic code. Amino acids can be referred to herein by either their commonly known three-letter symbols or by the one-letter symbols recommended by the IUPAC-IUB Biochemical Nomenclature Commission. Amino acids include the side chain and polypeptide backbone portions.

“Binding region” as used herein refers to the region within a target region that is recognized and bound by the DNA binding portion of a DNA Targeting System including a Targeted Activator System or a Targeted Repressor System, such as a nuclease or DNA binding domain fused to an activator or repressor.

“Coding sequence” means a nucleotide sequence (RNA or DNA) which encodes a protein. The coding sequence can further include initiation and termination signals operably linked to regulatory elements including a promoter and polyadenylation signal capable of directing expression in the cells of an individual or mammal to which the nucleic acid is administered. The coding sequence may be codon optimized.

“Complement” or “complementary” as used herein with respect to a nucleic acid means Watson-Crick (such as, A-T/U and C-G) or Hoogsteen base pairing between nucleotides or nucleotide analogs of nucleic acids. “Complementarity” refers to a property shared between two nucleic acid sequences, such that when they are aligned antiparallel to each other, the nucleotide bases at each position will be complementary.

The terms “control,” “reference level,” and “reference” are used herein interchangeably. The reference level may be a predetermined value or range, which is employed as a benchmark against which to assess the measured result. “Control group” as used herein refers to a group of control subjects. The predetermined level may be a cutoff value from a control group. The predetermined level may be an average from a control group. Cutoff values (or predetermined cutoff values) may be determined by Adaptive Index Model (AIM) methodology. Cutoff values (or predetermined cutoff values) may be determined by a receiver operating curve (ROC) analysis from biological samples of the patient group. ROC analysis, as generally known in the biological arts, is a determination of the ability of a test to discriminate one condition from another, such as, to determine the performance of each marker in identifying a patient having CRC. A description of ROC analysis is provided in P. J. Heagerty et al. (Biometrics 2000, 56, 337-44), the disclosure of which is hereby incorporated by reference in its entirety. Alternatively, cutoff values may be determined by a quartile analysis of biological samples of a patient group. For example, a cutoff value may be determined by selecting a value that corresponds to any value in the 25th-75th percentile range, preferably a value that corresponds to the 25th percentile, the 50th percentile or the 75th percentile, and more preferably the 75th percentile. Such statistical analyses may be performed using any method known in the art and can be implemented through any number of commercially available software packages (such as, from Analyse-it Software Ltd., Leeds, UK; StataCorp LP, College Station, Tex.; SAS Institute Inc., Cary, N.C.). The healthy or normal levels or ranges for a target or for a protein activity may be defined in accordance with standard practice. A control may be an subject or cell without an agonist as detailed herein. A control may be a subject, or a sample therefrom, whose disease state is known. The subject, or sample therefrom, may be healthy, diseased, diseased prior to treatment, diseased during treatment, or diseased after treatment, or a combination thereof.

“Donor DNA”, “donor template,” and “repair template” as used interchangeably herein refers to a double-stranded DNA fragment that includes at least a portion of the gene of interest.

“Frameshift” or“frameshift mutation” as used interchangeably herein refers to a type of gene mutation wherein the addition or deletion of one or more nucleotides causes a shift in the reading frame of the codons in the mRNA. The shift in reading frame may lead to the alteration in the amino acid sequence at protein translation, such as a missense mutation or a premature stop codon.

“Fusion protein” as used herein refers to a chimeric protein created through the covalent or non-covalent joining of two or more separate proteins. In some embodiments, translation of a fusion gene created through joining of two or more genes that originally coded for separate proteins results in a single polypeptide with functional properties derived from each of the original proteins.

“Genetic construct” as used herein refers to the DNA or RNA that comprise a polynucleotide that encodes a protein or RNA. The coding sequence includes initiation and termination signals operably linked to regulatory elements including a promoter and polyadenylation signal capable of directing expression in the cells of the individual to whom the nucleic acid is administered. As used herein, the term “expressible form” refers to gene constructs that contain the necessary regulatory elements operable linked to a coding sequence that encodes a protein or RNA such that when present in the cell of the individual, the coding sequence will be expressed.

“Homology-directed repair” or “HDR” as used interchangeably herein refers to a mechanism in cells to repair double strand DNA lesions when a homologous piece of DNA is present in the nucleus, mostly in G2 and S phase of the cell cycle. HDR uses a donor DNA template to guide repair and may be used to create specific sequence changes to the genome, including the targeted addition of whole genes. If a donor template is provided along with the CRISPR/Cas9-based gene editing system, then the cellular machinery will repair the break by homologous recombination, which is enhanced several orders of magnitude in the presence of DNA cleavage. Men the homologous DNA piece is absent, non-homologous end joining may take place instead.

“Genome editing” as used herein refers to changing a gene. Genome editing may include correcting or restoring a mutant gene or adding additional mutations. Genome editing may include knocking out a gene, such as a mutant gene or a normal gene.

“Identical” or “identity” as used herein in the context of two or more nucleic acids or polypeptide sequences means that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0. Identity of related peptides can be readily calculated by known methods. Such methods include, but are not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; Sequence Analysis Primer, Gribskov, M. and Devereux, J., eds., M. Stockton Press, New York, 1991; and Carillo et al, SIAM J. Applied Math. 48, 1073 (1988), herein incorporated by reference in their entirety.

As used herein, the term “imprinting” refers to the differential expression of alleles of the same gene in a parent-of-origin-specific manner, or to the biological process by which such a pattern is established. An “imprinted gene” is a gene that is subject to imprinting. Mammalian somatic cells are normally diploid, i.e., they contain two homologous sets of autosomes (chromosomes that are not sex chromosomes)—one set inherited from each parent, and a pair of sex chromosomes. Thus, mammalian somatic cells normally contain two copies of each autosomal gene—a maternal copy and a paternal copy. The two copies (often referred to as “alleles”) may be identical or may differ at one or more nucleotide positions. For most genes, the alleles inherited from the mother and father exhibit similar expression levels. In contrast, imprinted genes are normally expressed in a parent-of-origin specific manner-either the maternal allele (the allele on the chromosome inherited from the mother) is expressed and the paternal allele (the allele present on the chromosome inherited from the father) is not, or the paternal allele is expressed and the maternal allele is not. The allele that is not expressed may be referred to as the “imprinted allele” or “imprinted copy”. Imprinted genes can occur in large, coordinately regulated clusters or small domains composed of only one or two genes. Imprinting has generally been found to be conserved between mice and humans, i.e., if a gene is imprinted in mice, the orthologous gene is typically imprinted in humans as well, and vice versa. Parental allele-specific expression of imprinted genes is generally due to an imprinting control region.

As used herein, an “imprinting center” is a DNA region that controls the imprinting of at least one gene (typically a cluster of genes). In other words, the imprinting center controls the mono-allelic expression of the at least one gene in a manner that depends on the parental origin of the alleles. An imprinting center must be on the same chromosome as the imprinted gene(s) whose expression it affects but can be located a considerable distance away (such as, up to several megabases away).

The term “imprinting disorder” refers to any disorder caused by alterations in the normal imprinting pattern, any disorder caused by changes in expression or gene dosage of an imprinted gene, and/or any disorder caused by the mutation or deletion of an imprinted gene. Non-limiting examples of imprinting disorders include Angelman syndrome, Prader-Willi syndrome.

“Mutant gene” or “mutated gene” as used interchangeably herein refers to a gene that has undergone a detectable mutation. A mutant gene has undergone a change, such as the loss, gain, or exchange of genetic material, which affects the normal transmission and expression of the gene. A “disrupted gene” may refer to a mutant gene that has a mutation that causes a premature stop codon. The disrupted gene product is truncated relative to a full-length undisrupted gene product.

“Non-homologous end joining (NHEJ) pathway” as used herein refers to a pathway that repairs double-strand breaks in DNA by directly ligating the break ends without the need for a homologous template. The template-independent re-ligation of DNA ends by NHEJ is a stochastic, error-prone repair process that introduces random micro-insertions and micro-deletions (indels) at the DNA breakpoint. This method may be used to intentionally disrupt, delete, or alter the reading frame of targeted gene sequences. NHEJ typically uses short homologous DNA sequences called microhomologies to guide repair.

These microhomologies are often present in single-stranded overhangs on the end of double-strand breaks. When the overhangs are perfectly compatible, NHEJ usually repairs the break accurately, yet imprecise repair leading to loss of nucleotides may also occur but is much more common when the overhangs are not compatible.

“Normal gene” as used herein refers to a gene that has not undergone a change, such as a loss, gain, or exchange of genetic material. The normal gene undergoes normal gene transmission and gene expression. For example, a normal gene may be a wild-type gene.

“Nuclease mediated NHEJ” as used herein refers to NHEJ that is initiated after a nuclease cuts double stranded DNA.

“Nucleic acid” or “oligonucleotide” or “polynucleotide” as used herein means at least two nucleotides covalently linked together. The depiction of a single strand also defines the sequence of the complementary strand. Thus, a polynucleotide also encompasses the complementary strand of a depicted single strand. Many variants of a polynucleotide may be used for the same purpose as a given polynucleotide.

Polynucleotides may be single stranded or double stranded, or may contain portions of both double stranded and single stranded sequence. The polynucleotide can be nucleic acid, natural or synthetic, DNA, genomic DNA, cDNA, RNA, or a hybrid, where the polynucleotide can contain combinations of deoxyribo- and ribo-nucleotides, and combinations of bases including uracil, adenine, thymine, cytosine, guanine, inosine, xanthine hypoxanthine, isocytosine, and isoguanine. Polynucleotides can be obtained by chemical synthesis methods or by recombinant methods.

“Open reading frame” refers to a stretch of codons that begins with a start codon and ends at a stop codon. In eukaryotic genes with multiple exons, introns are removed, and exons are then joined together after transcription to yield the final mRNA for protein translation. An open reading frame may be a continuous stretch of codons. In some embodiments, the open reading frame only applies to spliced mRNAs, not genomic DNA, for expression of a protein.

“Operably linked” as used herein means that expression of a gene is under the functional control of a regulatory element, such as a promoter. For instance, a promoter or enhancer is operably linked to a coding sequence if it regulates, or contributes to the modulation of, the transcription of the coding sequence. A promoter may be positioned 5′ (upstream) or 3′ (downstream) of a gene under its control. As is known in the art, variation in the distance between the promoter and the gene it controls may be accommodated without loss of promoter function. Enhancers may function when separated from the promoter by up to several kilobases or more. Thus, regulatory elements may be operably linked without being contiguous.

“Partially-functional” as used herein describes a protein that is encoded by a mutant gene and has less biological activity than a functional protein but more than a non-functional protein.

A “peptide” or “polypeptide” is a linked sequence of two or more amino acids linked by peptide bonds. The polypeptide can be natural, synthetic, or a modification or combination of natural and synthetic. The terms “polypeptide”, “protein,” and “peptide” are used interchangeably herein. “Primary structure” refers to the amino acid sequence of a particular peptide. “Secondary structure” refers to locally ordered, three dimensional structures within a polypeptide. These structures are commonly known as domains, such as, enzymatic domains, extracellular domains, transmembrane domains, pore domains, and cytoplasmic tail domains. “Domains” are portions of a polypeptide that form a compact unit of the polypeptide and are typically 15 to 350 amino acids long. Exemplary domains include domains with enzymatic activity or ligand binding activity. Typical domains are made up of sections of lesser organization such as stretches of beta-sheet and alpha-helices. “Tertiary structure” refers to the complete three dimensional structure of a polypeptide monomer. “Quaternary structure” refers to the three dimensional structure formed by the noncovalent association of independent tertiary units. A “motif” is a portion of a polypeptide sequence and includes at least two amino acids. A motif may be 2 to 20, 2 to 15, or 2 to 10 amino acids in length. In some embodiments, a motif includes 3, 4, 5, 6, or 7 sequential amino acids. A domain may be comprised of a series of the same type of motif.

“Premature stop codon” or “out-of-frame stop codon” as used interchangeably herein refers to nonsense mutation in a sequence of DNA, which results in a stop codon at location not normally found in the wild-type gene. A premature stop codon may cause a protein to be truncated or shorter compared to the full-length version of the protein.

The term “recombinant” when used with reference to, for example, a cell, nucleic acid, protein, or vector, indicates that the cell, nucleic acid, protein, or vector, has been modified by the introduction of a heterologous nucleic acid or protein or the alteration of a native nucleic acid or protein, or that the cell is derived from a cell so modified. Thus, for example, recombinant cells express genes that are not found within the native (naturally occurring) form of the cell or express a second copy of a native gene that is otherwise normally or abnormally expressed, under expressed, or not expressed at all.

“Transcriptional regulatory elements” or “regulatory elements” refers to a genetic element which can control the expression of nucleic acid sequences, such as activate, enhancer, or decrease expression, or after the spatial and/or temporal expression of a nucleic acid sequence. Examples of regulatory elements include promoters, enhancers, splicing signals, polyadenylation signals, and termination signals. “Promoter” as used herein means a synthetic or naturally-derived nucleotide sequence which is capable of conferring, activating or enhancing expression of a nucleic acid in a cell. A promoter may also comprise distal enhancer or repressor elements, which may be located as much as several thousand base pairs from the start site of transcription. A promoter or other regulatory element may be derived from sources including viral, bacterial, fungal, plants, insects, and animals. A promoter may regulate the expression of a gene component constitutively, or differentially with respect to cell, the tissue or organ in which expression occurs or, with respect to the developmental stage at which expression occurs, or in response to external stimuli such as physiological stresses, pathogens, metal ions, or inducing agents. The selection of a particular promoter and enhancer depends on the recipient cell type. Some eukaryotic promoters and enhancers have a broad host range while others are functional in a limited subset of cell types Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, SV40 early promoter or SV40 late promoter, human U6 (hU6) promoter, and CMV IE promoter. A promoter and/or enhancer can be “endogenous,” “exogenous,” or “heterologous” with respect to the gene to which it is operably linked. An “endogenous” promoter/enhancer is one which is naturally linked with a given gene in the genome. An “exogenous” or “heterologous” enhancer or promoter is one which is not normally linked with a given gene but is placed in operable linkage with a gene by genetic manipulation.

As used herein, the term “heterologous” refers to a nucleic acid or polypeptide comprising two or more subsequences that are not found in the same relationship to each other in nature. For instance, a nucleic acid that is recombinantly produced typically has two or more sequences from unrelated genes synthetically arranged to make a new functional nucleic acid, such as, a promoter from one source and a coding region from another source. The two nucleic acids are thus heterologous to each other in this context. When added to a cell, the recombinant nucleic acids would also be heterologous to the endogenous genes of the cell.

“Sample” or “test sample” as used herein can mean any sample in which the presence and/or level of a target is to be detected or determined or any sample comprising a DNA targeting system or component thereof as detailed herein. Samples may include liquids, solutions, emulsions, or suspensions. Samples may include a medical sample. Samples may include any biological fluid or tissue, such as blood, whole blood, fractions of blood such as plasma and serum, muscle, interstitial fluid, sweat, saliva, urine, tears, synovial fluid, bone marrow, cerebrospinal fluid, nasal secretions, sputum, amniotic fluid, bronchoalveolar lavage fluid, gastric lavage, emesis, fecal matter, lung tissue, peripheral blood mononuclear cells, total white blood cells, lymph node cells, spleen cells, tonsil cells, cancer cells, tumor cells, bile, digestive fluid, skin, or combinations thereof. In some embodiments, the sample comprises an aliquot. In other embodiments, the sample comprises a biological fluid. Samples can be obtained by any means known in the art. The sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art.

“Subject” as used herein can mean a mammal that is in need of the herein described compositions or methods. The subject may be a patient. The subject may be a human or a non-human. The subject may be any vertebrate. The subject may be a mammal. The mammal may be a primate or a non-primate. The mammal can be a non-primate such as, for example, dog, cat, horse, cow, pig, mouse, rat, mouse, camel, llama, goat, rabbit, sheep, hamster, and guinea pig. The mammal can be a primate such as a human. The mammal can be a non-human primate such as, for example, monkey, cynomolgous monkey, rhesus monkey, chimpanzee, gorilla, orangutan, and gibbon. The subject may be of any age or stage of development, such as, for example, an adult, an adolescent, a child, such as age 0-2, 2-4, 2-6, or 6-12, or an infant, such as age 0-1. The subject may be male. The subject may be female. In some embodiments, the subject has a specific genetic marker. The subject may be undergoing other forms of treatment.

“Substantially identical” can mean that a first and second amino acid or polynucleotide sequence are at least 60%, 65%, 70%, 75%, 80%, 85%, 90%, 95%, 96%, 97%, 98%, or 99% over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 amino acids or nucleotides, respectively.

“Target gene” as used herein refers to any nucleotide sequence encoding a known or putative gene product that is intended to be corrected or for which its expression is intended to be modulated. The target gene may be a mutated gene involved in a genetic disease. In certain embodiments, the target gene is within or near the 15q11-q13 locus.

“Target region” as used herein refers to the region of the chromosome to which the DNA Targeting System, Targeted Activator System or Targeted Repressor System is designed to bind and modulate.

“Transgene” as used herein refers to a gene or genetic material containing a gene sequence that has been isolated from one organism and is introduced into a different organism. This non-native segment of DNA may retain the ability to produce RNA or protein in the transgenic organism, or it may alter the normal function of the transgenic organism's genetic code. The introduction of a transgene has the potential to change the phenotype of an organism.

“Treatment” or “therapy” or “treating,” when referring to protection of a subject from a disease, means suppressing, repressing, ameliorating, or completely eliminating the disease. Preventing the disease involves administering a composition of the present invention to a subject at risk of having the disease, prior to onset of the disease. Suppressing the disease involves administering a composition of the present invention to a subject after induction of the disease but before its clinical appearance. Repressing or ameliorating the disease involves administering a composition of the present invention to a subject after clinical appearance of the disease. Such treatment will result in a reduction in the incidence, frequency, severity or duration of symptoms of the disease.

As used herein, the term “gene therapy” refers to a method of treating a patient wherein polypeptides or nucleic acid sequences are transferred into cells of a patient such that activity and/or the expression of a particular gene is modulated. In certain embodiments, the expression of the gene is suppressed. In certain embodiments, the expression of the gene is enhanced. In certain embodiments, the temporal or spatial pattern of the expression of the gene is modulated.

“Variant” used herein with respect to a polynucleotide means (i) a portion or fragment of a referenced nucleotide sequence; (ii) the complement of a referenced nucleotide sequence or portion thereof; (iii) a nucleic acid that is at least 60%, 65%, 70%, 75%, 80%, 85%, 90%, 95%, 96%, 97%, 98%, or 99% identical to a referenced nucleic acid or the complement thereof over its full length or over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 nucleotides; or (iv) a nucleic acid that hybridizes under stringent conditions to the referenced nucleic acid, or complement thereof.

“Variant” with respect to a peptide or polypeptide means a polypeptide that differs in amino acid sequence from a referenced amino acid sequence by the insertion, deletion, and/or conservative substitution of amino acids, such as, at least 60%, 65%, 70%, 75%, 80%, 85%, 90%, 95%, 96%, 97%, 98%, or 99% identical over its full length or over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 amino acids, but which retains at least one biological activity. Representative examples of “biological activity” include the ability to be bound by a specific antibody or polypeptide or to promote a physiological response. Variant can mean a functional fragment thereof. Variant can also mean multiple copies of a polypeptide. The multiple copies can be in tandem or separated by a linker. A conservative substitution of an amino acid, i.e., replacing an amino acid with a different amino acid of similar properties (such as, hydrophilicity, degree and distribution of charged regions) is recognized in the art as typically involving a minor change. These minor changes may be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. It is known in the art that amino acids of similar hydropathic indexes may be substituted and still retain protein function. In one aspect, amino acids having hydropathic indexes of ±2 are substituted. The hydrophilicity of amino acids may also be used to reveal substitutions that would result in proteins retaining biological function. A consideration of the hydrophilicity of amino acids in the context of a peptide permits calculation of the greatest local average hydrophilicity of that peptide. Substitutions may be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

“Vector” as used herein means a nucleic acid construct capable of directing the delivery or transfer of a polynucleotide sequence to target cells, where it can be replicated or expressed. A vector may contain an origin of replication, one or more regulatory elements, and/or one or more coding sequences. A vector can be integrating or non-integrating. Major types of vectors include, but are not limited to, a plasmids, episomal vectors, viral vectors, cosmids, and artificial chromosomes. A vector may be a DNA or RNA vector. A vector may be a self-replicating extrachromosomal vector, and preferably, is a DNA plasmid. Viral vectors include, but are not limited to, adenovirus vector, adeno-associated virus vector, retrovirus vector, or lentivirus vector. A DNA plasmid vector may be delivered within a lipid nanoparticle.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics and protein and nucleic acid chemistry and hybridization described herein are those that are well known and commonly used in the art. The meaning and scope of the terms should be clear; in the event however of any latent ambiguity, definitions provided herein take precedent over any dictionary or extrinsic definition. Further, unless otherwise required by context, singular terms shall include pluralities and plural terms shall include the singular.

### 2. Prader-Willi Syndrome (PWS)

Prader-Willi Syndrome (PWS) is a rare genetic disease with a prevalence ranging from approximately one in 8,000 to one in 25,000 patients in the U.S. It is believed that the genetics underlying PWS involve a loss of function of one or more genes on chromosome 15 in humans, in particular, within the PWS region 15q11-13 (Schaaf et al. Nat. Genet. 2013, 45, 1405-09). Loss of function in individuals with PWS can be caused by a de novo deletion in the paternally inherited chromosome (˜70-75%), maternal uniparental disomy (UPD) (˜20-30%), and/or microdeletions or epimutations of the imprinting center (i.e., imprinting defects) (˜2-5%) (Bittel and Butler, Expert Rev. Mol. Med. 2005, 7, 1-20; Cassidy and Driscoll, Eur. J. Hum. Genet. 2009, 17, 3-13).

There are several imprinted genes within the 15q11-13 locus (the PWS-associated locus), including the paternally-expressed coding genes MAGEL2, NDN and SNURF-SNRPN, and MKRN3, along with numerous noncoding RNAs (ncRNAs), including the snoRNA clusters SNORD115 and SNORD116. As noted above, PWS patient genotypes most commonly consist of deletions within 15q11-13 that encompass both coding and noncoding genes, although a rare subset of genotypes emphasize the snoRNA clusters as having particular influence in the etiology of PWS (Bieth et al., Eur. J. Hum. Genet. 2015, 23, 252-255; de Smith et al., Hum. Mol. Genet. 2009, 18, 3257-3265; Duker et al., Eur. J. Hum. Genet. 2010, 18, 1196-1201; Sahoo et al., Nat. Genet. 2008, 40, 719-721). Further evidence suggests that SNURF-SNRPN and downstream ncRNAs, including SPA RNAs and snoRNAs, are processed from a single host transcript that initiates at the imprinting center located in upstream exon 1 of SNRPN (Wu et al., Mol. Cell 2016, 64, 534-548).

Further known genes and gene products of the PWS-associated locus are as follows: NPAP1 (NCBI gene ID: 23742), SNORD107 (snoRNA) (NCBI gene ID: 91380), SNORD64 (snoRNA cluster) (NCBI gene ID: 347686), SNORD109A (snoRNA) (NCBI gene ID: 338428), SNORD116 or SNORD116@ (snoRNA gene cluster) (NCBI gene ID: 692236), SPA1 (long noncoding RNA transcribed from the SNORD116 gene cluster), SPA2 (long noncoding RNA transcribed from the SNORD116 gene cluster) (for SPA1 and SPA2, see Wu et al., Mol. Cell 64(3): 534-48 (2016)), 116HG (long non-coding RNA transcribed from SNORD116 gene cluster) (Kocher et al., Genes, 8(12): 358 (2017)), SNORD116-1 to 30 (snoRNAs or processed snoRNA derivatives transcribed from the SNORD116 cluster) (SNORD116 1-30 NCBI gene ID Nos: 100033413, 100033414, 100033415, 00033416, 100033417, 100033418, 100033419, 100033420, 100033421, 100033422, 100033423, 100033424, 100033425, 100033426, 100033427, 100033428, 100033429, 100033430, 727708, 100033431, 100033432, 100033433, 100033434, 100033435, 100033436, 100033438, 100033439, 100033820, and 100033821, respectively), SNORD116-30: 100873856, Sno-Inc RNA 1 to 5 (long non coding RNA with snoRNA ends transcribed from the SNORD116 cluster) (Yin et al., Mol. Cell, 48(2): 219-30 (2012)), IPW (long noncoding RNA) (NCBI gene ID: 3653), SNORD115 or SNORD115@ (noncoding snoRNA cluster) (NCBI gene ID: 493919), 115HG (long noncoding snoRNA transcribed from SNORD115 cluster) (Powell et al., Hum. Molec. Genet. 22: 4318-28 (2013)), SNORD115-1 to 48 (snoRNAs or processed snoRNA derivates transcribed from SNORD115 cluster) (SNORD115 1-48 NCBI gene ID Nos: 338433, 100033437, 100033440, 100033441, 100033442, 100033443, 100033444, 100033445, 100033446, 100033447, 100033448, 100033449, 100033450, 100033451, 100033453, 100033454, 100033455, 100033456, 100033458, 100033460, 100033603, 100033799, 100033800, 100036563, 100033801, 100033802, 100036564, 100036565, 100033803, 100033804, 100033805, 100033806, 100033807, 100033808, 100033809, 100033810, 100033811, 100033812, 100033813, 100033814, 100033815, 100033816, 100033817, 100033818, 100036566, 100873857, 100036567, 100033822, or SNORD109B (snoRNA) (NCBI gene ID: 338429), SNHG14 (PWS region long transcript) (NCBI gene ID: 104472715).

Prader-Willi-like syndromes and disorders may include but are not limited to Scaaf Yang Syndrome (SYS), Chitayat-Hall Syndrome, Magel2 related disorders, and deletions encompassing Magel2, but not SNORD116. Schaaf-Yang Syndrome (SYS) or MAGEL2-related disorder is a disorder caused by paternally inherited truncating mutations in the MAGEL2 gene (McCarthy et al. Am J Med Genet A; 176(12):2564-2574 (2018)). Chitayat-Hall syndrome can also be cause by paternally inherited truncating mutations in the MAGEL2 gene (Jobling et al. J. Med. Genet; 55:316-321 (2018)).

MAGEL2 is a maternally imprinted gene in the PWS region. Patients with Schaaf-Yang syndrome (SYS) display many overlapping symptoms as patients with PWS, including neonatal hypotonia, feeding difficulties during infancy, global developmental delay, and intellectual disabilities (McCarthy et al. Am J Med Genet A; 176(12):2564-2574 (2018)). However, there are several features that do not overlap with PWS. Individuals with SYS very commonly present with arthrogryposis or joint contractures which have never been reported in PWS. Additionally, people with SYS have a higher prevalence of Autism spectrum disorder than is observed in people with PWS. MAGEL2 is a monoexonic gene and therefore missense mutations are not subject to nonsense mediated decay. This, along with the additional phenotypes observed in SYS that are not seen in PWS, or in paternal deletions encompassing MAGEL2 but not SNORD116, suggests that the truncated forms of MAGEL2 present in SYS may have dominant negative activity. Although SYS does not completely overlap with PWS, it demonstrates the importance of the loss of function of the MAGEL2 gene to the PWS phenotype.

The disclosure contemplates that the methods of using one or more DNA Targeting Systems, such as one or more Targeted Activator Systems or one or more Targeted Repressor Systems as described herein, will treat a subject with any of the following disorders: PWS, PWS-like syndrome, PWS Type 1 large deletion, PWS Type 2 large deletion, PWS imprinting center mutation or PWS uniparental disomy; PWS microdeletion, atypical deletion encompassing MAGEL2, Heterozygous Schaaf-Yang syndrome, Chitayat-Hall syndrome, MAGEL2 disorder, MAGEL2-related disorder.

DNA Targeting System(s), including one or more Targeted Activator Systems or one or more Targeted Repressor Systems, can be delivered, such as via gene therapy, to cells of the patients to be treated. The Targeted Activator Systems are designed to target the target regions identified herein as amenable to increasing PWS gene expression through administration of activators. The Targeted Repressor Systems are designed to target the target regions identified herein as amenable to increasing PWS gene expression through administration of repressors. Alternatively, the gene therapy methods of the disclosure can be accomplished by CRISPR/Cas9 based gene editing to incorporate an insertion, deletion and/or substitution in any of the target regions identified herein that eliminates the imprinting (silencing) of the PWS region genes.

The disclosure also contemplates that expression of one or more of the following genes or gene products (including noncoding RNAs) or clusters is upregulated, i.e., increased, in the subject by administration of the DNA Targeting System(s), such as Targeted Activator System(s) or Targeted Repressor System(s), described herein: MKRN3 (gene), MAGEL2 (gene), NDN (gene), C15ORF2, SNURF-SNRPN (gene), SNORD107 (snoRNA), SNORD64 (snoRNA duster), SNORD109A (snoRNA), SNORD116 or SNORD116@ (snoRNA gene cluster), SPA1 (long noncoding RNA transcribed from the SNORD116 gene cluster), SPA2 (long noncoding RNA transcribed from the SNORD116 gene cluster), 116HG (long non-coding RNA transcribed from SNORD116 gene cluster), SNORD116-1 to 30 (snoRNAs transcribed from the SNORD116 cluster), Sno-Inc RNA 1 to 5 (long non coding RNA with snoRNA ends transcribed from the SNORD116 cluster), IPW (long noncoding RNA), SNORD115 or SNORD115@ (noncoding snoRNA cluster), 115HG (long noncoding snoRNA transcribed from SNORD115 cluster), SNORD115-1 to 48 (snoRNAs transcribed from SNORD115 cluster), or SNORD109B (snoRNA), SNHG14 (PWS region long transcript).

In some embodiments, the DNA Targeting System, such as Targeted Activator System or Targeted Repressor System, described herein targets a region that results in increased expression of SNORD116, or increased expression of MAGEL2, or both. In some embodiments, a first Targeted Activator System targets a region that results in increased expression of SNORD116 and a second Targeted Activator System targets a region that results in increased expression of MAGEL2. The disclosure contemplates that additional Targeted Activator Systems may be utilized concurrently. In some embodiments, a first Targeted Repressor System targets a region that results in increased expression of SNORD116 and a second Targeted Repressor System targets a region that results in increased expression of MAGEL2. The disclosure contemplates that additional Targeted Repressor Systems may be utilized concurrently. Multiple DNA Targeting Systems may be utilized concurrently.

The treatment methods as described herein, for PWS, may result in amelioration/reduction of symptoms including, for example, hypotonia, growth hormone deficiency, infantile failure to thrive, global developmental delay, neonatal hypophagia, anxiety, obsessive compulsive disorder, obsessive compulsive-like disorder, intellectual impairment, intellectual disability, hyperphagia, obesity due to hyperphagia, metabolic syndrome secondary to obesity, type 2 diabetes in PWS, behavioral disturbances such as tantrums, outbursts and self-harm, anxiety and compulsivity, and/or skin picking. Other characteristics or symptoms may include small hands, small feet, straight ulnar borders on hands, characteristic facial features: almond shaped eyes, thin upper lip, temperature instability, chronic constipation, decreased gut/intestinal motility, scoliosis, hyperghrelinemia, and/or hypoinsulinemia.

The treatment methods as described herein, for SYS, may result in amelioration of symptoms including neonatal hypotonia, growth hormone deficiency, infantile failure to thrive, global developmental delay, hyperghrelinemia, autism spectrum disorder, infantile respiratory distress, gastroesophageal reflux, chronic constipation, skeletal abnormalities, sleep apnea, temperature instability, and/or arthrogryposis.

### 3. DNA Targeting Systems

A “DNA Targeting System” as used herein is a system capable of specifically targeting a particular region of DNA and activating gene expression by binding to that region. Non-limiting examples of these systems are CRISPR-Cas-based systems, zinc finger (ZF)-based systems, and/or transcription activator-like effector (TALE)-based systems. The DNA Targeting System may be a nuclease system that acts through mutating or editing the target region (such as by insertion, deletion or substitution) or it may be a system that delivers a functional second polypeptide domain, such as an activator or repressor, to the target region.

A “DNA Targeting System” may be a Targeted Activator System, or a Targeted Repressor System, or a combination thereof. A “Targeted Activator System” as used herein is a system capable of specifically targeting a particular region of DNA and activating gene expression by binding to that target region. A “Targeted Repressor System” as used herein is a system capable of specifically targeting a particular region of DNA and repressing gene expression by binding to that target region.

Each of these systems comprises a DNA-binding portion or domain, such as a guide RNA, a ZF, or a TALE, that specifically recognizes and binds to a particular target region of a target DNA. The DNA-binding portion (Cas9 protein, ZF or TALE) can be linked to a second protein domain, such as a polypeptide with transcription activation activity, transcription repression activity, transcription release factor activity, histone modification activity, nuclease activity, nucleic acid association activity, methylase activity, demethylase activity, acetylation activity, or deacetylation activity. For example, the DNA-binding portion can be linked to an activator and thus guide the activator to a specific target region of the target DNA. Similarly, the DNA-binding portion can be linked to a repressor and thus guide the repressor to a specific target region of the target DNA.

Some CRISPR-Cas-based systems can operate to activate or repress expression using the Cas protein alone, not linked to an activator or repressor. For example, a nuclease-null Cas9 can act as a repressor on its own, or a nuclease-active Cas9 can act as an activator when paired with an inactive (dead) guide RNA. In addition, RNA or DNA that hybridizes to a particular target region of the target DNA can be directly linked (covalently or non-covalently) to an activator or repressor.

### 4. CRISPR/Cas9-Based Gene Editing System

The gene therapy methods of the disclosure can be accomplished by administering a DNA Targeting System, such as Targeted Activator System or Targeted Repressor System, that comprises a CRISPR-Cas-based system, which comprises (a) one or more guide RNAs and (b) one or more Cas polypeptides. In some embodiments, the Cas polypeptides are fusion proteins comprising a Cas protein or fragment or variant thereof, and a second heterologous polypeptide domain, and administration of the DNA Targeting System upregulates expression of one or more genes within the 15q11-13 locus. Alternatively, the gene therapy methods of the disclosure can be accomplished by CRISPR/Cas9 based gene editing to incorporate an insertion, deletion, and/or substitution that eliminates the imprinting (silencing) of the PWS region genes.

CRISPR (Clustered Regularly Interspaced Short Palindromic Repeats)-Cas, refers to RNA-guided endonuclease systems that comprise (a) an RNA portion that guides the endonuclease system to target DNA by hybridizing to a DNA sequence within the target region of the target DNA, and (b) a nuclease portion that binds to and cleaves the target DNA at or near that location. The most commonly used CRISPR-Cas systems are the Type II CRISPR systems, such as CRISPR-Cas9 or CRISPR-Cpf1, in which the nuclease portion is a single enzyme. However, multi-protein nuclease systems, such as the Type I system, can be harnessed for the same purpose. One example of such a Type I multi-protein nuclease complex is described in U.S. Patent Appl. Pub. No. 2018/0334688 (Gersbach), incorporated by reference herein in its entirety. Another example of a Cpf1-based complex is described in U.S. Patent Appl. Pub. No. 2019/0151476 (Gersbach), incorporated by reference herein in its entirety

Provided herein are genetic constructs for genome editing, genomic alteration, or altering gene expression of a gene, for example, on chromosome 15, for the treatment of PWS, PWS-like syndrome, PWS Type 1 large deletion, PWS Type 2 large deletion, PWS imprinting center mutation or PWS uniparental disomy; PWS microdeletion, atypical deletion encompassing MAGEL2, Heterozygous Schaaf-Yang syndrome, Chitayat-Hall syndrome, MAGEL2 disorder, or MAGEL2-related disorder. The genetic constructs include at least one gRNA that targets a target region, such as a gene sequence or a regulatory element thereof. The disclosed gRNAs can be included in a CRISPR/Cas9-based gene editing system to target regions in the 15q11-13 imprinted locus, or a regulatory element of a gene within the 15q11-13 locus, causing activation of imprinted genes within the 15q11-13 locus in cells from patients such as PWS patients.

In some embodiments, the at least one gRNA targets an activating regulatory element of a gene within the 15q11-13 locus. In some embodiments, the gRNA may be combined with a Cas9 protein that introduces a mutation in the regulatory element such as an insertion, deletion, and/or substitution, as detailed below, such that the activity of the activating regulatory element is increased, thereby activating expression of the maternal gene within the 15q11-13 locus for the treatment of PWS. In other embodiments, the at least one gRNA targets an inhibitory regulatory element of a gene within the 15q11-13 locus. In some embodiments, the gRNA may be combined with a Cas9 protein that introduces a mutation in the regulatory element such as an insertion, deletion, and/or substitution, as detailed below, such that the activity of the inhibitory regulatory element is decreased, thereby activating expression of the maternal gene within the 15q11-13 locus for the treatment of PWS.

In some embodiments, the gRNA may be combined with a fusion protein that activates transcription, as detailed below, such that the activity of the activating regulatory element is increased, thereby activating expression of an imprinted gene within the 15q11-13 locus for the treatment of PWS.

In other embodiments, the gRNA may be combined with a fusion protein that represses transcription, as detailed below, such that the activity of the inhibitory regulatory element is decreased, thereby activating expression of the maternal gene within the 151q1-13 locus for the treatment of PWS.

A CRISPR/Cas9-based system may be specific fora gene within the 15q11-13 locus or a regulatory element thereof.

“Clustered Regularly Interspaced Short Palindromic Repeats” and “CRISPRs”, as used interchangeably herein, refers to loci containing multiple short direct repeats that are found in the genomes of approximately 40% of sequenced bacteria and 90% of sequenced archaea. The CRISPR system in nature is a microbial nuclease system involved in defense against invading phages and plasmids that provides a form of acquired immunity. The CRISPR loci in microbial hosts contain a combination of CRISPR-associated (Cas) genes as well as non-coding RNA elements capable of programming the specificity of the CRISPR-mediated nucleic acid cleavage. Short segments of foreign DNA, called spacers, are incorporated into the genome between CRISPR repeats, and serve as a “memory” of past exposures.

CRISPR (Clustered Regularly Interspaced Short Palindromic Repeats)-Cas, refers to RNA-guided endonuclease systems that comprise (a) an RNA portion that guides the endonuclease system to target DNA by hybridizing to a DNA sequence within the target region of the target DNA, and (b) a nuclease portion that binds to and cleaves the target DNA at or near that location.

Three classes of CRISPR systems (Types I, II, and III effector systems) are known. The natural Type II effector system carries out targeted DNA double-strand breaks using a complex comprising a single effector enzyme, Cas9, together with a duplex of two RNAs, a crRNA and a tracrRNA. Collectively, the duplex of two RNAs is called the “guide RNA.” A predefined 20 bp portion at the 5′ end of the natural crRNA recognizes its target by complementary base pairing to a DNA sequence of the target DNA. This 20 bp portion may be swapped for a different portion of similar nucleotide length to change the target recognition of the Type II effector system. The CRISPR-Cas systems can target multiple distinct genomic loci by co-expressing a single Cas9 or Cpf1 protein with two or more guide RNA.

An engineered improvement to this system links the two RNAs together, either via chemical covalent linkage, or with a nucleotide linker (such as GAAA), to form a single guide RNA (sgRNA). As explained below, the RNA(s) can be chemically modified to improve stability and reduce degradation in the cellular environment. Type II Cas9 systems and their use in Targeted Activator Systems are described, such as in Perez-Pinera et al., Nat Methods. 2013 October; 10(10): 973-976. Type II Cpf1 systems and their use in Targeted Activator Systems are described, such as in Zhang et al., Protein Cell 9, 380-383 (2018).

For DNA cleavage, the DNA sequence recognized by the crRNA must also be immediately followed by the protospacer-adjacent motif (PAM), a short sequence recognized by the Cas9. Different Cas9 from different bacteria have differing PAM requirements. For example, the PAM for S. pyogenes Cas9 (SpCas9) as 5′-NRG-3′, where R is either A or G. Thus, the DNA-targeting mechanism of the type II CRISPR-Cas9 system involves a guide RNA which directs the Cas9 endonuclease to cleave the target DNA in a sequence-specific manner, dependent on the presence of a Protospacer Adjacent Motif (PAM) on the target DNA.

For example, the S. pyogenes Type II system naturally prefers to use an “NGG” sequence, where “N” can be any nucleotide, but also accepts other PAM sequences, such as “NAG” in engineered systems (Hsu et al., Nature Biotechnology 2013 doi:10.1038/nbt.2647). Similarly, the Cas9 derived from Neisseria meningitidis (NmCas9) normally has a native PAM of NNNNGATT, but has activity across a variety of PAMs, including a highly degenerate NNNNGNNN PAM (Esveft et al. Nature Methods 2013 doi:10.1038/nmeth.2681).

A Cas9 protein of S. aureus recognizes the sequence motif NNGRR (R=A or G) (SEQ ID NO: 19) and directs cleavage of a target nucleic acid sequence 1 to 10, such as, 3 to 5, bp upstream from that sequence. In certain embodiments, a Cas9 protein of S. aureus recognizes the sequence motif NNGRRN (R=A or G) (SEQ ID NO: 20) and directs cleavage of a target nucleic acid sequence 1 to 10, such as, 3 to 5, bp upstream from that sequence. In certain embodiments, a Cas9 protein of S. aureus recognizes the sequence motif NNGRRT (R=A or G) (SEQ ID NO: 21) and directs cleavage of a target nucleic acid sequence 1 to 10, such as, 3 to 5, bp upstream from that sequence. In certain embodiments, a Cas9 protein of S. aureus recognizes the sequence motif NNGRRV (R=A or G) (SEQ ID NO: 22) and directs cleavage of a target nucleic acid sequence 1 to 10, such as, 3 to 5, bp upstream from that sequence. In the aforementioned embodiments, N can be any nucleotide residue, such as, any of A, G, C, or T. Cas9 proteins can be engineered to alter the PAM specificity of the Cas9 protein.

a. Cas Protein

The CRISPR/Cas9-based gene editing system can include a Cas protein or a Cas fusion protein. The Cas9 protein can be from any bacterial or archaea species, including, but not limited to, Streptococcus pyogenes (also in U.S. Patent Appl. Pub. No. 2019/0127713 (Gersbach), incorporated by reference herein in its entirety), Staphylococcus aureus (S. aureus) (also in U.S. 2019/0127713 (Gersbach), incorporated by reference herein in its entirety), Streptococcus thermophilus (LMD-9, YP_820832.1), L. innocua (Clip11262, NP_472073.1), Acidovorax avenae, Actinobacillus pleuropneumoniae, Actinobacillus succinogenes, Actinobacillus suis, Actinomyces sp., Cycliphilus denitrificans, Aminomonas paucivorans, Azospirillum, Bacillus cereus, Bacillus smithii, Bacillus thuringiensis, Bacteroides sp., Blastopirellula manna, Bradyrhizobium sp., Brevibacillus laterosporus, Campylobacter coli, Campylobacter jejuni (subsp. jejuni NCTC 11168, YP_002344900.1), Campylobacter lari, Candidatus puniceispirillum, Clostridium cellulolyticum, Clostridium perfringens, Corynebacterium accolens, Corynebacterium diphtheria, Corynebacterium matruchotii, Dinoroseobacter shibae, Eubacterium dolichum, Eubacterium ventriosum, gamma proteobacterium, Gluconacetobacter diazotrophicus, Haemophilus parainfluenzae, Haemophilus sputorum, Helicobacter canadensis, Helicobacter cinaedi, Helicobacter mustelae, Ilyobacter polytropus, Kingella kingae, Lactobacillus crispatus, Lactobacillus farciminis, Listena ivanovii, Listera monocytogenes, Listeraceae bacterium, Methylocystis sp., Methylosinus trichosporium, Mobiluncus mulieris, Neisseria bacilliformis, Neisseria cinerea, Neisseria flavescens, Neisseria lactamica, N. meningitidis (Z2491, YP_002342100.1), Neisseria sp., Neisseria wadsworthii, Nitratifractor salsuginis, Nitrosomonas sp., Parvibaculum lavamentivorans, Pasteurella multocida, Phascolarctobacterium succinatutens, Ralstonia syzygii, Rhodopseudomonas palustris, Rhodovulum sp., Roseburia intestinalis, Simonsiella muelleri, Sphaerochaeta, Sphingomonas sp., Sporolactobacillus vineae, Staphylococcus lugdunensis, Streptococcus sp., Streptococcus pasteurianus, Subdoligranulum sp., Tistrella mobilis, Treponema sp., or Verminephrobacter eiseniae. CRISPR loci have been identified in more than 40 prokaryotes (See such as, Jansen et al., Mol. Microbiol., 43:1565-1575, 2002; and Mojica et al., J. Molec. Evolution, 60(2), 174-82, 2005) including, but not limited to Aeropyrum, Pyrobaculum, Sulfolobus, Archaeoglobus, Halocarcula, Methanobacterium, Methanococcus, Methanosarcina, Methanopyrus. Pyrococcus, Picrophilus, Thermoplasma, Corynebacterium, Mycobacterium, Streptomyces, Aquifex, Porphyromonas, Chlorobium, Thermus, Bacillus, Listeria, Staphylococcus, Clostridium, Thermoanaerobacter, Mycoplasma, Fusobacterium, Azarcus, Chromobacterium, Neisseria, Nitrosomonas, Desulfovibrio, Geobacter, Myxococcus, Campylobacter, Wolinella, Acinetobacter, Erwinia, Escherichia, Legionella, Methylococcus, Pasteurella, Photobacterium, Salmonella, Xanthomonas. Yersinia, Treponema, and Thermotoga.

In certain embodiments, the Cas9 protein is a Streptococcus pyogenes Cas9 protein (also referred herein as “SpCas9”) or a variant thereof. In certain embodiments, the Cas9 protein is a Staphylococcus aureus Cas9 protein (also referred herein as “SaCas9”) or a variant thereof.

The Cas polypeptide can also comprise a modified form of the Cas polypeptide that retains DNA-targeting activity and is at least 65% identical, preferably at least 70%, 75%, 80%, 85%, 90%, 95%, 96%, 97%, 98%, or at least 99% identical to a naturally occurring Cas protein amino acid sequence. The modified form of the Cas polypeptide can include an amino acid change (such as, deletion, insertion, and/or substitution) that reduces the naturally occurring nuclease activity of the Cas protein. For example, in some instances, the modified form of the Cas protein has less than 50%, less than 40%, less than 30%, less than 20%, less than 10%, less than 5%, or less than 1% of the nuclease activity of the corresponding wild-type Cas polypeptide (US20140068797 published 6 Mar. 2014). In some cases, the modified form of the Cas polypeptide has no substantial nuclease activity and is referred to as “nuclease null” or “deactivated” Cas (dCas).” For Cas9, this can be accomplished, such as, by mutating one or both of the nuclease domains of Cas9, the HNH domain or the RuvC domain. Each of these nuclease domains is responsible for cleaving one of the two strands of the target DNA. For S. pyogenes cas9, mutations at positions 10 and 840 (such as D10A, H840A) render it nuclease null. For S. aureus Cas9, mutations at positions 10 and 580 (such as D10A, N580A) render it nuclease null.

The ability of a Cas9 protein or a Cas9 fusion protein to recognize a PAM sequence can be determined, such as, using a transformation assay as known in the art. In certain embodiments, the ability of a Cas9 protein or a Cas9 fusion protein to interact with and cleave a target nucleic acid is PAM sequence dependent. A PAM sequence is a sequence in the target nucleic acid. In certain embodiments, cleavage of the target nucleic acid occurs upstream from the PAM sequence. Cas9 proteins from different bacterial species can recognize different sequence motifs (such as, PAM sequences). In certain embodiments, a Cas9 protein of S. pyogenes recognizes the sequence motif NGG and directs cleavage of a target nucleic acid sequence 1 to 10, such as, 3 to 5, bp upstream from that sequence. In certain embodiments, a Cas9 protein of S. thermophilus recognizes the sequence motif NGGNG (SEQ ID NO: 33) and/or NNAGAAW (W=A or T) (SEQ ID NO: 17) and directs cleavage of a target nucleic acid sequence 1 to 10, such as, 3 to 5, bp upstream from these sequences. In certain embodiments, a Cas9 protein of S. mutans recognizes the sequence motif NGG and/or NAAR (R=A or G) (SEQ ID NO: 18) and directs cleavage of a target nucleic acid sequence 1 to 10, such as, 3 to 5 bp, upstream from this sequence. In certain embodiments, a Cas9 protein of S. aureus recognizes the sequence motif NNGRR (R=A or G) (SEQ ID NO: 19) and directs cleavage of a target nucleic acid sequence 1 to 10, such as, 3 to 5, bp upstream from that sequence. In certain embodiments, a Cas9 protein of S. aureus recognizes the sequence motif NNGRRN (R=A or G) (SEQ ID NO: 20) and directs cleavage of a target nucleic acid sequence 1 to 10, such as, 3 to 5, bp upstream from that sequence. In certain embodiments, a Cas9 protein of S. aureus recognizes the sequence motif NNGRRT (R=A or G) (SEQ ID NO: 21) and directs cleavage of a target nucleic acid sequence 1 to 10, such as, 3 to 5, bp upstream from that sequence. In certain embodiments, a Cas9 protein of S. aureus recognizes the sequence motif NNGRRV (R=A or G; V=A or C or G) (SEQ ID NO: 22) and directs cleavage of a target nucleic acid sequence 1 to 10, such as, 3 to 5, bp upstream from that sequence. In the aforementioned embodiments, N can be any nucleotide residue, such as, any of A, G, C, or T. Cas9 proteins can be engineered to alter the PAM specificity of the Cas9 protein.

In certain embodiments, the vector encodes at least one Cas9 protein that recognizes a Protospacer Adjacent Motif (PAM) of either NNGRRT (SEQ ID NO: 21) or NNGRRV (SEQ ID NO: 22). In certain embodiments, the at least one Cas9 protein is an S. aureus Cas9 protein. In certain embodiments, the at least one Cas9 protein is a mutant S. aureus Cas9 protein.

The Cas9 protein can be mutated so that the nuclease activity is inactivated. An inactivated Cas9 protein (“iCas9”, also referred to as “dCas9”) with no endonuclease activity has been targeted to genes in bacteria, yeast, and human cells by gRNAs to silence gene expression through steric hindrance. Exemplary mutations with reference to the S. pyogenes Cas9 sequence to inactivate the nuclease activity include: D10A, E762A, H840A, N854A, N863A, and/or D986A. Exemplary mutations with reference to the S. aureus Cas9 sequence to inactivate the nuclease activity include D10A and N580A. In certain embodiments, the Cas9 protein is a mutant S. aureus Cas9 protein.

In certain embodiments, the mutant S. aureus Cas9 protein comprises a D10A mutation. The nucleotide sequence encoding this mutant S. aureus Cas9 protein is set forth in SEQ ID NO: 31.

In certain embodiments, the mutant S. aureus Cas9 protein comprises a N580A mutation. The nucleotide sequence encoding this mutant S. aureus Cas9 protein is set forth in SEQ ID NO: 32.

In some embodiments, the Cas9 protein is a VQR variant. The VQR variant of Cas9 is a mutant with a different PAM recognition, as detailed in Kleinstiver, et al. (Nature 2015, 523, 481-485, incorporated herein by reference).

A polynucleotide encoding a Cas9 protein can be a synthetic polynucleotide. For example, the synthetic polynucleotide can be chemically modified. The synthetic polynucleotide can be codon optimized, such as, at least one non-common codon or less-common codon has been replaced by a common codon. For example, the synthetic polynucleotide can direct the synthesis of an optimized messenger mRNA, such as, optimized for expression in a mammalian expression system, such as, described herein.

Additionally or alternatively, a nucleic acid encoding a Cas9 polypeptide may comprise a nuclear localization sequence (NLS). Nuclear localization sequences are known in the art. An exemplary codon optimized nucleic acid sequence encoding a Cas9 protein of S. pyogenes is set forth in SEQ ID NO: 23. The corresponding amino acid sequence of an S. pyogenes Cas9 protein is set forth in SEQ ID NO: 24.

Exemplary codon optimized nucleic acid sequences encoding a Cas9 protein of S. aureus, and optionally containing nuclear localization sequences (NLSs), are set forth in SEQ ID NOs: 25-29, 34, and 35. Another exemplary codon optimized nucleic acid sequence encoding a Cas9 protein of S. aureus comprises the nucleotides 1293-4451 of SEQ ID NO: 37. An amino acid sequence of an S. aureus Cas9 protein is set forth in SEQ ID NO: 30. An amino acid sequence of an S. aureus Cas9 protein is set forth in SEQ ID NO: 36.

Alternatively or additionally, the CRISPR/Cas9-based gene editing system can include a fusion protein as described herein.

b. Guide RNA (gRNA)

The CRISPR/Cas-based gene editing system includes at least one gRNA molecule. The at least one gRNA molecule can bind and recognize a target region. The guide RNA is the part of the CRISPR-Cas system that provides DNA targeting specificity to the system. The guide RNA comprises at its 5′ end a DNA-targeting domain that is sufficiently complementary to the target region to be able to hybridize to 10 to 20 nucleotides of the target region of the target DNA, when it is followed by an appropriate Protospacer Adjacent Motif (PAM). It has been reported that the specificity of the CRISPR system can be improved by reducing the length of the DNA-targeting domain in the guide RNA to 17-18 nucleotides (Fu et al. Nat. Biotechnol. 2014, 32, 279-284). The “target region” or “target sequence” or “protospacer” refers to the region of the target gene to which the CRISPR/Cas9-based gene editing system targets and binds. The portion of the gRNA that targets the target sequence in the genome may be referred to as the “targeting sequence” or “targeting portion” or “targeting domain.” “Protospacer” or “gRNA spacer” may refer to the region of the target gene to which the CRISPR/Cas9-based gene editing system targets and binds; “protospacer” or “gRNA spacer” may also refer to the portion of the gRNA that is complementary to the targeted sequence in the genome. The gRNA may include a gRNA scaffold. A gRNA scaffold facilitates Cas9 binding to the gRNA and may facilitate endonuclease activity. The gRNA scaffold is a polynucleotide sequence that follows the portion of the gRNA corresponding to sequence that the gRNA targets. Together, the gRNA targeting portion and gRNA scaffold form one polynucleotide. The constant region of the gRNA may include the sequence of SEQ ID NO: 1141 (RNA), which is encoded by a sequence comprising SEQ ID NO: 1140 (DNA). For example, the sequence of the full gRNA corresponding to SEQ ID NO: 588 (defined below) may be SEQ ID NO: 1142.

The DNA-targeting domain of the guide RNA does not need to be perfectly complementary to the target region of the target DNA. In example embodiments, the DNA-targeting domain of the guide RNA sequence is at least 80% complementary, preferably at least 85%, 90%, 95%, 96%, 97%, 98%, or at least 99% complementary to (or has 1, 2 or 3 mismatches compared to) the target region over a length of, such as, 10, 11, 12, 13, 14, 15, 16, 17, 18, 19 or 20 nucleotides. For example, the DNA-targeting domain of the guide RNA sequence is at least 80% complementary over at least 18 nucleotides of the target region. The target region may be on either strand of the target DNA.

The portion of the guide RNA that corresponds to the tracrRNA can be variably truncated and a range of lengths has been shown to function in both a system comprising separate RNAs and a system comprising a single-guide RNA. For example, in some embodiments, tracrRNA may be truncated from its 3′ end by at least 1, 2, 3, 4, 5, 6, 7, 8, 9, 10, 15, 20, 25, 30, 35, or 40 nucleotides. In some embodiments, the tracrRNA may be truncated from its 5′ end by at least 1, 2, 3, 4, 5, 6, 7, 8, 9, 10, 15, 20, 21, 22, 23, 24, or 25 nucleotides. Alternatively, the tracrRNA may be truncated from both the 5′ and 3′ end, such as, by at least 1, 2, 3, 4, 5, 6, 7, 8, 9, 10, 15, or 20 nucleotides on the 5′ end and at least 1, 2, 3, 4, 5, 6, 7, 8, 9, 10, 15, 20, 25, 30, 35, or 40 nucleotides on the 3′ end. See, such as, Jinek et al., Science 2012; 337:816-821; Mali et al., Science. 2013 Feb. 15; 339(6121):823-6; Cong et al., Science. 2013 Feb. 15; 339(6121):819-23; and Hwang and Fu et al., Nat Biotechnol. 2013 March; 31(3):227-9; Jinek et al., Elife 2, e00471 (2013)).

The guide RNAs are complementary to a target region of the genomic target DNA identified herein by the tiled screen of Targeted Activator Systems and Targeted Repressor Systems. For example, they are complementary to a target region within about 100-1000, about 100-900, about 100-800, about 100-700, about 100-600, about 100-500, about 100-400, about 100-300 or about 100-200 bp upstream, or downstream, of the target region identified herein.

The guide RNA can be designed to target known transcription response elements (such as, promoters, enhancers, etc.), known upstream activating sequences (UAS), sequences of unknown or known function that are suspected of being able to control expression of the target DNA, etc. In some such cases, the CRISPR-Cas-based DNA Targeting System, including Targeted Activator System or Targeted Repressor System, is targeted by the guide RNA to a specific location (i.e., sequence) in the target region of the DNA and exerts locus-specific regulation such as blocking RNA polymerase binding to a promoter (which selectively inhibits transcription activator function), and/or modifying the local chromatin status or epigenetic status (such as modifying the target DNA or proteins associated with the target DNA, such as, nucleosomal histones. In some cases, the changes are transient (such as, transcription repression or activation). In some cases, the changes are inheritable by daughter cells.

The CRISPR/Cas9-based gene editing system includes at least one gRNA. In any of the embodiments, more than one target region can be targeted with 2, 3, 4, 5, or more gRNAs directed to different sites in the same locus of the target DNA. The at least one gRNA may target an activating regulatory element of a gene within the 15q11-13 locus. The at least one gRNA may target an inhibitory regulatory element of a gene within the 15q11-13 locus. The number of gRNAs encoded by a genetic construct (such as, an AAV vector) can be at least 1 gRNA, at least 2 different gRNA, at least 3 different gRNA at least 4 different gRNA, at least 5 different gRNA, at least 6 different gRNA, at least 7 different gRNA, at least 8 different gRNA, at least 9 different gRNA, at least 10 different gRNAs, at least 11 different gRNAs, at least 12 different gRNAs, at least 13 different gRNAs, at least 14 different gRNAs, at least 15 different gRNAs, at least 16 different gRNAs, at least 17 different gRNAs, at least 18 different gRNAs, at least 18 different gRNAs, at least 20 different gRNAs, at least 25 different gRNAs, at least 30 different gRNAs, at least 35 different gRNAs, at least 40 different gRNAs, at least 45 different gRNAs, or at least 50 different gRNAs. The number of gRNAs encoded by a presently disclosed vector can be between at least 1 gRNA to at least 50 different gRNAs, at least 1 gRNA to at least 45 different gRNAs, at least 1 gRNA to at least 40 different gRNAs, at least 1 gRNA to at least 35 different gRNAs, at least 1 gRNA to at least 30 different gRNAs, at least 1 gRNA to at least 25 different gRNAs, at least 1 gRNA to at least 20 different gRNAs, at least 1 gRNA to at least 16 different gRNAs, at least 1 gRNA to at least 12 different gRNAs, at least 1 gRNA to at least 8 different gRNAs, at least 1 gRNA to at least 4 different gRNAs, at least 4 gRNAs to at least 50 different gRNAs, at least 4 different gRNAs to at least 45 different gRNAs, at least 4 different gRNAs to at least 40 different gRNAs, at least 4 different gRNAs to at least 35 different gRNAs, at least 4 different gRNAs to at least 30 different gRNAs, at least 4 different gRNAs to at least 25 different gRNAs, at least 4 different gRNAs to at least 20 different gRNAs, at least 4 different gRNAs to at least 16 different gRNAs, at least 4 different gRNAs to at least 12 different gRNAs, at least 4 different gRNAs to at least 8 different gRNAs, at least 8 different gRNAs to at least 50 different gRNAs, at least 8 different gRNAs to at least 45 different gRNAs, at least 8 different gRNAs to at least 40 different gRNAs, at least 8 different gRNAs to at least 35 different gRNAs, 8 different gRNAs to at least 30 different gRNAs, at least 8 different gRNAs to at least 25 different gRNAs, 8 different gRNAs to at least 20 different gRNAs, at least 8 different gRNAs to at least 16 different gRNAs, or 8 different gRNAs to at least 12 different gRNAs. In certain embodiments, the genetic construct (such as, an AAV vector) encodes one gRNA, i.e., a first gRNA, and optionally a Cas9 protein. In certain embodiments, a first genetic construct (such as, a first AAV vector) encodes one gRNA, i.e., a first gRNA, and optionally a Cas9 protein, and a second genetic construct (such as, a second AAV vector) encodes one gRNA, i.e., a second gRNA, and optionally a Cas9 protein.

The gRNA may comprise a “G” at the 5′ end of the targeting domain or complementary polynucleotide sequence, such as a result of in vitro transcription by a T7 RNA polymerase. The DNA-targeting domain of a gRNA may comprise at least a 10 base pair, at least a 11 base pair, at least a 12 base pair, at least a 13 base pair, at least a 14 base pair, at least a 15 base pair, at least a 16 base pair, at least a 17 base pair, at least a 18 base pair, at least a 19 base pair, at least a 20 base pair, at least a 21 base pair, at least a 22 base pair, at least a 23 base pair, at least a 24 base pair, at least a 25 base pair, at least a 30 base pair, or at least a 35 base pair polynucleotide sequence complementary to the target region DNA sequence followed by a PAM sequence. In certain embodiments, the targeting domain of a gRNA has 19-25 nucleotides in length. In certain embodiments, the targeting domain of a gRNA is 20 nucleotides in length. In certain embodiments, the targeting domain of a gRNA is 21 nucleotides in length. In certain embodiments, the targeting domain of a gRNA is 22 nucleotides in length. In certain embodiments, the targeting domain of a gRNA is 23 nucleotides in length.

The gRNA may target a region within or near the 15q11-q13 locus or a regulatory element thereof. In certain embodiments, the gRNA can target at least one of exons, introns, the promoter region, the enhancer region, or the transcribed region of the gene. In some embodiments, the gRNA targets a gene selected from SNRPN, SNORD115, SNORD116, SPA1, SPA2, and MAGEL2, or a combination thereof. In some embodiments, the gRNA targets a SNRPN activating regulatory element, SNORD115 activating regulatory element, SNORD116 activating regulatory element, or a combination thereof. In some embodiments, the gRNA targets a SNRPN promoter, SNORD115 promoter, SNORD116 promoter, or a combination thereof. The gRNA may include a targeting domain that comprises a polynucleotide sequence corresponding to at least 10, 11, 12, 13, 14, 15, 16, 17, 18, 19, or 20 consecutive bases of any one of SEQ ID NOs: 1-12, or of any one of SEQ ID NOs: 47-86, 91-1122, 591, 585, 685, 697, 750, 752, 763, 771, 196, 812, 861, and 1069 (TABLE 1, TABLE 2, TABLE 3, TABLE 4) or a complement thereof or an allelic variant thereof. The protospacers and guides represented in TABLE 1 may be useful for targeted delivery of polypeptides that have activator activity. The protospacers and guides represented in TABLE 2 and TABLE 4 may be useful for targeted delivery of polypeptides that have demethylase activity. SEQ ID NOs: 96-516 may be especially useful for targeted delivery of polypeptides that have repressor activity. SEQ ID NOs: 519-580 may be especially useful for targeted delivery of polypeptides that have activator activity when targeted with a pair of gRNAs. The DNA Targeting system may include at least one gRNA corresponding to any one of SEQ ID NOs: 1-12, 47-86, 91-1122, 96-516, 519-580, 591, 585, 685, 697, 750, 752, 763, 771, 196, 812, 861, and 1069, or a truncation, complement, and/or variation thereof. The DNA Targeting system may include at least one gRNA that includes a targeting domain that comprises a polynucleotide sequence corresponding to at least 10, 11, 12, 13, 14, 15, 16, 17, 18, 19, or 20 consecutive bases of any one of SEQ ID NOs: 1-12, 47-86, 91-1122, 591, 585, 685, 697, 750, 752, 763, 771, 196, 812, 861, and 1069. In some embodiments, the at least one gRNA includes a targeting domain that comprises a polynucleotide sequence corresponding to at least 10, 11, 12, 13, 14, 15, 16, 17, 18, 19, or 20 consecutive bases of any one of SEQ ID NOs: 685, 697, 750, 752, 763, 771, 196, 812, and 861, or a truncation, complement, and/or variation thereof. In some embodiments, the at least one gRNA is complementary to a polynucleotide sequence corresponding to at least 10, 11, 12, 13, 14, 15, 16, 17, 18, 19, or 20 consecutive bases of any one of SEQ ID NOs: 53, 55, 64, 65, 68, 70, 196, 291, or 335, or a truncation, complement, and/or variation thereof.

The protospacer sequence (also referred to as target sequence of the gRNA) and the gRNAs in TABLE 1 and TABLE 2 and TABLE 4 bind to (are complementary to) the same sequence (on the opposite strand) of the target DNA. For example, a guide RNA that corresponds to the target region identified by AAAGCATGCGCTACAATAAC (SEQ ID NO: 47) may comprise at least 18 consecutive bases of the sequence

(SEQ ID NO: 591)
AAAGCAUGCGCUACAAUAAC.

TABLE 1
Examples of gRNAS targeting a gene within or
near the 15q11-q13 locus.

| | SEQ | | SEQ | |
|---|---|---|---|---|
| gRNA name | ID NO | protospacer sequence | ID NO | gRNA sequence |
| PWS_G.3511 | 1 | CTAAGCAGTGGACTAAGGAT | 1052 | CUAAGCAGUGGACUAAGGAU |
| PWS_G.3549 | 2 | CTACTTTAGATTCTATTGTA | 1122 | CUACUUUAGAUUCUAUUGUA |
| PWS_G.4662 | 3 | GCAGCTCAGAAAGTGATGCA | 1054 | GCAGCUGAGAAAGUGAUGGA |
| PWS_G.4738 | 4 | GCCAGGTTACTGATTCATAG | 1058 | GCCAGGUUACUGAUUCAUAG |
| PWS_G.4862 | 5 | GCGCACTGCAGCGCAGACCA | 1057 | GCGCACUGCAGCGCAGACCA |
| PWS_G.5117 | 6 | GGAACCAGTCAGAACAGGTG | 588 | GGAACCAGUCAGAACAGGUG |
| PWS_G.5706 | 7 | GGTCGGCTTCAGGAGGGAAG | 1051 | GGUCGGCUUCAGGAGGGAAG |
| PWS_G.6131 | 8 | GTGGATAGGTGCGTTCAAGG | 1059 | GUGGAUAGGUGCGUUCAAGG |
| PWS_G.6318 | 9 | GTTTCAGGCCTGGACTGGGT | 1053 | GUUUCAGGCCUGGACUGGGU |
| PWS_G.8738 | 10 | ACAGTTCAGGGCATGAGATA | 1055 | ACAGUUCAGGGCAUGAGAUA |
| PWS_G.9414 | 11 | CAGTTCAGGGCATGAGATAA | 586 | CAGUUCAGGGCAUGAGAUAA |
| PWS G.10218 | 12 | GGGCATGAGATAAGGGCAGT | 1056 | GGGCAUGAGAUAAGGGCAGU |
| PWS_G.717 | 91 | ACCCACCCAGCAACCACCAG | 581 | ACCCACCCAGCAACCACCAG |
| (pat1) | | | | |
| PWS_G.9876 | 92 | GACAATGACAAGGGTTGTGG | 582 | GACAAUGACAAGGGUUGUGG |
| (pat3) | | | | |
| PWS_G.7517 | 93 | TGCTTGTTTGCCGCAGTGCA | 584 | GGUUUUGAGCAAGCCCUCCU |
| (pat5) | | | | |
| PWS_G.7097 | 94 | TCGTTGTGACACTACTGAGT | 586 | UGGUUGUGACACUACUGAGU |
| (pat7) | | | | |
| PWS_G.3780 | 95 | CTGCCACGCTGTGACTCAGA | 587 | CUGCCACGCUGUGACUCAGA |
| (pat8) | | | | |

TABLE 2
Examples of target regions within or near the 15q11-q13 locus.

| | SEQ | | SEQ | |
|---|---|---|---|---|
| gRNA name | ID NO | Protospacer sequence | ID NO | gRNA sequence |
| PWS_G.81 | 47 | AAAGCATGCGCTACAATAAC | 591 | AAAGCAUGCGCUACAAUAAC |
| PWS_G.595 | 48 | ACAGATGCGTCAGGCATCTC | 606 | ACAGAUGCGUCAGGCAUCUC |
| PWS_G.648 | 49 | ACATCCTCTATTCTGATCAT | 585 | ACAUCCUCUAUUCUGAUCAU |
| PWS_G.1288 | 50 | AGGAGCGGTCAGTGACGCGA | 634 | AGGAGCGGUCAGUGACGCGA |
| PWS_G.1867 | 51 | ATGCCTGACGCATCTGTCTG | 657 | AUGCCUGACGCAUCUGUCUG |
| PWS_G.2532 | 52 | GATAAGCAACCTGGGATCAA | 679 | CAUAAGCAACCUGGGAUCAA |
| PWS_G.2815 | 53 | CCAGGTCATTCCGGTGAGGG | 685 | CCAGGUCAUUCCGGUGAGGG |
| PWS_G.2977 | 54 | CCCTCACCGGAATGACCTGG | 696 | CCCUCACCGGAAUGACCUGG |
| PWS_G.2979 | 55 | CCCTCAGGTCTTCCTATGTG | 697 | CCCUCAGGUCUUCCUAUGUG |
| PWS_G.3026 | 56 | CCGCACATAGGAAGACCTGA | 698 | CCGCACAUAGGAAGACCUGA |
| PWS_G.3107 | 57 | CCTATTGCGGGTGTCTGCGG | 704 | CCUAUUGCGGGUGUCUGCGG |
| PWS_G.3193 | 58 | CCTGTCGCGACACCACAGTT | 709 | CCUGUCGCGACACCACAGUU |
| PWS_G.3268 | 59 | CGAGCGGACAGGATACCATC | 712 | CGAGCGGACAGGAUACCAUC |
| PWS_G.3437 | 60 | CGGTCAGTGACGCGATGGAG | 717 | CGGUCAGUGACGCGAUGGAG |
| PWS_G.3477 | 61 | CGTGGGGGGACCAGTGCATA | 719 | CGUGGGGGGACCAGUGCAUA |
| PWS_G.3482 | 62 | CGTGTGGCGAGGGTACGTGG | 720 | CGUGUGGCGAGGGUACGUGG |
| PWS_G.3858 | 63 | CTGTCGCGACACCACAGTTG | 734 | CUGUCGCGACACCACAGUUG |
| PWS_G.4363 | 64 | GAGCGGACAGGATACCATCG | 750 | GAGCGGACAGGAUACCAUCG |
| PWS_G.4521 | 65 | GATGCGTCAGGCATCTCCGG | 752 | GAUGCGUCAGGCAUCUCCGG |
| PWS_G.4627 | 66 | GCACGCCTGCGCGGCCGCAG | 759 | GCACGCCUGCGCGGCCGCAG |
| PWS_G.4658 | 67 | GCAGCGAGTCTGGCGCAGAG | 761 | GCAGCGAGUCUGGCGCAGAG |
| PWS_G.4670 | 68 | GCAGGCTGGCGCGCATGCTC | 763 | GCAGGCUGGCGCGCAUGCUC |
| PWS_G.4871 | 69 | GCGCCCCAATGCGAGCGGAC | 770 | GCGCCCCAAUGCGAGCGGAG |
| PWS_G.4903 | 70 | GCGGCGACAGTGGGTATTGG | 771 | GCGGCGACAGUGGGUAUUGG |
| PWS_G.5274 | 71 | GGCAAACAAGCACGCCTGCG | 785 | GGCAAACAAGCACGCCUGCG |
| PWS_G.5478 | 72 | GGGACGCGCCCCAATGCGAG | 792 | GGGACGCGCCCCAAUGCGAG |
| PWS_G.5691 | 73 | GGTCAGTGACGCGATGGAGC | 798 | GGUCAGUGACGCGAUGGAGC |
| PWS_G.6001 | 74 | GTCGCGACAGGTCCTATTGG | 808 | GUCGCGACAGGUCCUAUUGC |
| PWS_G.6065 | 75 | GTGACGCGATGGAGCGGGCA | 813 | GUGACGCGAUGGAGCGGGCA |
| PWS_G.6298 | 76 | GTTGTGCCGTTCTGCCCCGA | 819 | GUUGUGCCGUUCUGCCCCGA |
| PWS_G.7011 | 77 | TCCGTGTGGCGAGGGTACGT | 847 | UCCGUGUGGCGAGGGUACGU |
| PWS_G.7296 | 78 | TGACCGAGGCGAGGAGGCTA | 858 | UGACCGAGGCGAGGAGGCUA |
| PWS_G.7302 | 79 | TGACGCATCTGTCTGAGGAG | 859 | UGACGCAUCUGUCUGAGGAG |
| PWS_G.7440 | 80 | TGCCGCTGCTGCAGCGAGTC | 862 | UGCCGCUGCUGCAGCGAGUC |
| PWS_G.7517 | 81 | TGCTTGTTTGCCGCAGTGCA | 583 | UGCUUGUUUGCCGCAGUGCA |
| PWS_G.7751 | 82 | TGTCGCGAGACGACAGTTGG | 870 | UGUCGCGACACCACAGUUGG |
| PWS_G.7924 | 83 | TTAGGCGGAGGTAGGTATAT | 876 | UUAGGCGGAGGUAGGUAUAU |
| PWS_G.9680 | 84 | CTCAAATCAATCTCCCTGTA | 941 | CUCAAAUCAAUCUCCCUGUA |
| PWS_G.9721 | 85 | CTGCAATTCGTTCCTTCCAG | 943 | CUGCAAUUCGUUCCUUCCAG |
| PWS_G.10360 | 86 | GTGAGTGGAGGTTGACTAAA | 973 | GUGAGUGGAGGUUGACUAAA |

TABLE 3 identifies the regions of the DNA targeted in the paternal screens (CRISPRi screens) with a repressor in which gRNA hits were enriched relative to other targeted DNA regions (pat1-pat8), and the regions of DNA targeted in the maternal screens (CRISPRa screens) with an activator or demnethylase in which gRNA hits were enriched relative to other targeted DNA regions (mat1-mat2 or mat3-mat4, respectively). Guide RNAs within two regions, pat6 and pat8, increased observed levels of SNRPN expression when paired with a Cas9-repressor protein. Guide RNAs within four regions, mat1-mat4, increased observed levels of SNRPN expression when paired with an activator or epigenetic modifier protein.

TABLE 3
Regions of DNA targeted in the paternal and maternal screens.

| | | | | | Chr. coordinate | SNRPN coordinate |
|---|---|---|---|---|---|---|
| | | SEQ | Starting | Ending | of example | of example |
| | Chromosomal | ID | SNRPN | SNRPN | validated | validated |
| Region | coordinates | NO: | coordinate | coordinate | single gRNA | single gRNA |
| pat1 | chr15: 25099211- | 1123 | −100858 | −94723 | 25101568 | −98501 |
| | 25105346 | | | | | |
| pat2 | chr15: 25142337- | 1124 | −57732 | −52414 | | |
| | 25147655 | | | | | |
| pat3 | chr15: 25195722- | 1125 | −4347 | −3547 | 25196467 | −3602 |
| | 25196522 | | | | | |
| pat4 | chr15: 25198794- | 1126 | −1275 | +1921 | 25200029 | −40 |
| | 25201990 | | | | | |
| pat5 | chr15: 25203015- | 1127 | +2720 | +23516 | 25211664 | 11595 |
| | 25223585 | | | | | |
| pat6 | chr15: 25223912- | 1128 | +23843 | +24582 | 25224155 | 24086 |
| | 25224651 | | | | | |
| pat7 | chr15: 25224909- | 1129 | +24840 | +35218 | 25227019 | 26950 |
| | 25235287 | | | | | |
| pat8 | chr15: 25235775- | 1130 | +35706 | +36168 | 25235867 | 35798 |
| | 25236237 | | | | | |
| mat1 | chr15: 25074022- | 1131 | −126047 | −125228 | 25074809 | −125260 |
| | 25074874 | | | | | |
| mat1-A | chr15: 25068632- | 1132 | −131437 | −131080 | | |
| | 25068989 | | | | | |
| mat1-B | chr15: 25071154- | 1133 | −128915 | −128215 | | |
| | 25071854 | | | | | |
| mat1-C | chr15: 25076771- | 1134 | −124298 | −122940 | | |
| | 25077129 | | | | | |
| mat2 | chr15: 25108001- | 1135 | −92068 | −91960 | 25108002 | −92067 |
| | 25108109 | | | | | |
| mat3 | chr15: 25199722- | 1136 | −347 | +876 | | |
| | 25200915 | | | | | |
| mat4 | chr15: 25213427- | 1137 | +13358 | +13526 | | |
| | 25213595 | | | | | |

Single or multiplexed gRNAs can be designed to restore expression of imprinted genes within the 15q11-13 locus. Following treatment with a construct or system as detailed herein, expression of imprinted genes within the 15q11-13 locus can be restored in PWS patient cells ex vivo. Genetically corrected patient cells may be transplanted into a subject.

i) Dead gRNAs

It has been reported that “dead” guide RNA can be used to guide catalytically active Cas9 to activate transcription without cleaving DNA. Dead guide RNA can be prepared by reducing the length of the DNA-targeting domain to 14-15 nucleotides (nt), and by adding MS2 binding loops into the sgRNA backbone. Guides having a DNA-targeting domain of from 20 bases to 16 bases resulted in indel formation (indicating DNA cleavage), whereas shorter guides (11 bases to 15 bases) did not show detectable levels of indel formation and were able to increase gene expression by as much as 10,000 fold. Dahlman et al., “Orthogonal gene knockout and activation with a catalytically active Cas9 nuclease,” Nat. Biotechnol. 2015, 33, 1159-1161; correction in Nat. Biotechnol. 2016, 34, 441.

Thus, the disclosure contemplates administering a Cas polypeptide with dead guide RNA comprising a DNA-targeting domain about 11-15, or 14-15 bases in length, or a DNA-targeting domain complementary to about 11-15 or 14-15 bases of the target region of the target DNA. The guide RNA may comprise mismatches at the 5′ end of the DNA-targeting domain.

One example system utilizes dead guide RNAs (comprising 14 or 15 bases of DNA-targeting domain) in conjunction with an engineered hairpin aptamer that contains two MS2 domains, which can recruit the MS2:P65:HSF1 (MPH) transcriptional activation complex to the target locus. Liao et al., Cell 171, 1495-1507, 2017.

ii) Modifications to gRNA

The activity, stability, or other characteristics of gRNAs can be altered through the incorporation of certain modifications. As one example, transiently expressed or delivered nucleic acids can be prone to degradation by, such as, cellular nucleases. Accordingly, the gRNAs described herein can contain one or more modified nucleosides or nucleotides which introduce stability toward nucleases. In addition, certain modified gRNAs described herein can exhibit a reduced innate immune response when introduced into cells. Such modifications include, without limitation, (a) alteration of the backbone linkage, such as, replacement, of one or both of the non-linking phosphate oxygens and/or of one or more of the linking phosphate oxygens in the phosphodiester backbone linkage; (b) alteration, such as, replacement, of the sugar, or a constituent of the ribose sugar, such as, of the 2′ hydroxyl on the ribose sugar; (c) replacement of the phosphate moiety with “dephospho” linkers; (d) modification or replacement of a naturally occurring nucleobase; (e) replacement or modification of the ribose-phosphate backbone; (f) modification of the 3′ end or 5′ end of the oligonucleotide, such as, removal, modification or replacement of a terminal phosphate group, capping, or conjugation of a moiety, and any combinations thereof. For example, a modified guide RNA may comprise one or more modified sugars and one or more modified backbone linkage. In other embodiments, a modified guide RNA may comprise one or more modified sugars and one or more modified nucleobases. Modifications, such as base, sugar or backbone linkages discussed in this section, can be included at every position or just some positions within a gRNA sequence including, without limitation at or near the 5′ end (such as, within 1-10, 1-5, 1-4, 1-3, or 1-2 nucleotides of the 5 end) and/or at or near the 3′ end (such as, within 1-10, 1-5, 1-4, 1-3, or 1-2 nucleotides of the 3′ end). For example, three positions at the 5′ end and three positions at the 3′ end may be modified. In some cases, modifications are positioned within functional motifs, such as the repeat-anti-repeat duplex of a Cas9 gRNA, a stem loop structure of a Cas9 or Cpf1 gRNA, and/or a targeting domain of a gRNA.

As one example, the 5′ end of a gRNA can include a eukaryotic mRNA cap structure or cap analog (such as, a G(5′)ppp(5′)G cap analog, a m7G(5)ppp(5′)G cap analog, or a 3′-O-Me-m7G(5)ppp(5′)G anti reverse cap analog (ARCA)). The 5′ end of the gRNA can lack a 5′ triphosphate. The 3′ terminal U ribose can be modified by oxidizing the two terminal hydroxyl groups of the U ribose to aldehyde groups with a concomitant opening of the ribose ring to afford a modified nucleoside. The 3′ terminal U ribose can be modified with a 2′3′ cyclic phosphate.

Guide RNAs can contain modified nucleotides such as modified uridines, such as, 5-(2-amino)propyl uridine, and 5-bromo uridine, modified adenosines and guanosines, such as, with modifications at the 8-position, such as, 8-bromo guanosine, a deaza nucleotide, such as, 7-deaza-adenosine, or O- and N-alkylated nucleotides, such as, N6-methyl adenosine, or a modified nucleotide which is multicyclic (such as, tricyclo; and “unlocked” forms, such as glycol nucleic acid (GNA) (such as, R-GNA or S-GNA, where ribose is replaced by glycol units attached to phosphodiester bonds), or threose nucleic acid (TNA, where ribose is replaced with α-L-threofuranosyl-(3′→2′)). In example embodiments, one or more or all of the nucleotides in a gRNA are deoxynucleotides. In such embodiments, guide RNAs that comprise RNA-DNA-combinations are still referred to as guide RNA.

Sugar-modified ribonucleotides can be incorporated into the gRNA, such as, wherein the 2′ OH-group is replaced by another group. Example groups include H, —OR, —R (wherein R can be, such as, alkyl, cycloalkyl, aryl, aralkyl, heteroaryl or sugar), halo, —F, —Br, —Cl or —I, —SH, —SR (wherein R can be, such as, alkyl, cycloalkyl, aryl, aralkyl, heteroaryl or sugar), -arabino, F-arabino, amino (wherein amino can be, such as, NH2; alkylamino, dialkylamino, heterocyclyl, arylamino, diarylamino, heteroarylamino, diheteroarylamino, or amino acid); or cyano (—CN). One or more of the nucleotides of the gRNA can each independently be a modified or unmodified nucleotide including, but not limited to 2′-sugar modified, such as, 2′-O-methyl, 2′-O-methoxyethyl, or 2′-Fluoro modified including, such as, 2′-F or 2-O-methyl, adenosine (A), 2′-F or 2′-O-methyl, cytidine (C), 2′-F or 2′-O-methyl, uridine (U), 2′-F or 2′-O-methyl, thymidine (T), 2′-F or 2′-O-methyl, guanosine (G), 2′-O-methoxyethyl-5-methyluridine (Teo), 2′-O-methoxyethyladenosine (Aeo), 2-O-methoxyethyl-5-methylcytidine (m5Ceo), and any combinations thereof. Although the majority of sugar analog alterations are localized to the 2′ position, other sites are amenable to modification, including the 4′ position. In certain embodiments, a gRNA comprises a 4′-S, 4′-Se or a 4′-C-aminomethyl-2′-O-Me modification.

The phosphate backbone can be modified, such as, with a phosphothioate (PhTx) group or phosphonoacetate, thiophosphonoacetate, methylphosphonate, boranophosphate, or phosphorodithioate.

Guide RNAs can also include “locked” nucleic acids (LNA) in which the 2′ OH-group can be connected, such as, by a C1-6 alkylene or C1-6 heteroalkylene bridge, to the 4′ carbon of the same ribose sugar. Any suitable moiety can be used to provide such bridges, include without limitation methylene, propylene, ether, or amino bridges; O-amino (wherein amino can be, such as, NH2; alkylamino, dialkylamino, heterocyclyl, arylamino, diarylamino, heteroarylamino, or diheteroarylamino, ethylenediamine, or polyamino) and aminoalkoxy or O(CH2)n-amino (wherein amino can be, such as, NH2; alkylamino, dialkylamino, heterocyclyl, arylamino, diarylamino, heteroarylamino, or diheteroarylamino, ethylenediamine, or polyamino).

As one example, gRNA having 2′-O-methyl (M) incorporated at three terminal nucleotides at both the 5′ and 3′ ends, generally exhibits improved stability against nucleases and also improved base pairing thermostability. As another example, gRNA having 2′-O-methyl-3′-phosphorothioate (MS) or 2′-O-methyl-3′-thioPACE (MSP) incorporated at three terminal nucleotides at both the 5′ and 3′ ends exhibits improved stability against nucleases.

### 5. Zinc Finger (ZF)-Based Systems

A DNA Targeting System, such as a Targeted Activator System or Targeted Repressor System, can comprise a zinc finger (ZF)-based system.

By a “zinc finger DNA binding domain” or “ZFBD” it is meant a polypeptide domain that binds DNA in a sequence-specific manner through one or more zinc fingers. A zinc finger is a domain of about 30 amino acids within the zinc finger binding domain whose structure is stabilized through coordination of a zinc ion. Examples of zinc fingers include, but are not limited to, C2H2 zinc fingers, C3H zinc fingers, and C4 zinc fingers. Each finger typically contacts and selectively binds to three base pairs of DNA. Combining different zinc fingers together allows production of sequence-specific ZFBD. A “designed” zinc finger domain is a domain not occurring in nature whose design/composition results principally from rational criteria, such as, application of substitution rules and computerized algorithms for processing information in a database storing information of existing ZFP designs and binding data. See, for example, Kim and Kini, “Engineering and Application of Zinc Finger Proteins and TALEs for Biomedical Research,” Mol Cells; 40(8):533-541, 2017; U.S. Pat. Nos. 6,140,081; 6,453,242; and 6,534,261; see also WO 98/53058; WO 98/53059; WO 98/53060; WO 02/016536 and WO03/016496. A “selected” zinc finger domain is a domain not found in nature whose production results primarily from an empirical process such as phage display, interaction trap or hybrid selection. ZFBD can be fused to a nuclease, such as the FokI nuclease, to form a zinc finger nuclease (ZFN). ZFBD can also be fused to an activator or repressor.

Thus, a ZF-Based System may comprise a ZFN or a fusion protein comprising (a) a ZF targeting a target region, or a variant thereof, and (b) an activator, or a variant thereof. A ZF-Based System may comprise a fusion protein comprising (a) a ZF targeting a target region, or a variant thereof and (b) a repressor, or a variant thereof. Alternatively, the ZF can be linked to the activator or repressor through reversible or irreversible covalent linkage or through a non-covalent linkage.

### 6. Transcription Activator-Like Effector (TALE)-Based Systems

A DNA Targeting System, such as a Targeted Activator System or Targeted Repressor System, can comprise a transcription activator-like effector-(TALE)-based system.

A TALE is a “transcription activator-like effector DNA binding domain”, “TAL effector DNA binding domain”, or “TALE DNA binding domain” that is the polypeptide domain of TAL effector proteins that is responsible for binding of the TAL effector protein to DNA. TAL effector proteins are secreted by plant pathogens of the genus Xanthomonas during infection. These proteins enter the nucleus of the plant cell, bind effector-specific DNA sequences via their DNA binding domain, and activate gene transcription at these sequences via their transactivation domains. TAL effector DNA binding domain specificity depends on modules consisting of repetitive sequences of 33-35 amino acid repeats, which comprise polymorphisms at select repeat positions, usually the 12th and 13th position, called repeat variable-diresidues (RVD). These RVD determine the nucleotide specificity of each module. Combining these modules allows production of sequence-specific TALEs. TALEs are described in greater detail, for example, in US Patent Application No. 2011/0145940, Kim and Kini, supra, and Moore et al., ACS Synth Biol.; 3(10):708-716, 2014. A TALE can be fused to a nuclease, such as the FokI nuclease, to form a TALE nuclease (TALEN). A TALE can also be fused to an activator or repressor.

Thus, a TALE-Based System may comprise a TALEN or a fusion protein comprising (a) a TALE targeting a target region, or a variant thereof and (b) an activator, or a variant thereof. A TALE-Based System may comprise a fusion protein comprising (a) a TALE targeting a target region, or a variant thereof and (b) a repressor, or a variant thereof. Alternatively, the TALE can be linked to the activator or repressor through reversible or irreversible covalent linkage or through a non-covalent linkage.

### 7. Fusion Protein

Polypeptides may be linked to a second polypeptide domain such as an activator or repressor to form a fusion protein. The fusion protein can comprise two heterologous polypeptide domains. The first polypeptide domain comprises a DNA binding protein such as a ZFBD, TALE, or Cas polypeptide. The first polypeptide domain is fused to at least one second polypeptide domain. The second heterologous polypeptide domain has an activity such as transcription activation activity, transcription repression activity, transcription release factor activity, histone modification activity, nuclease activity, nucleic acid association activity, methylase activity, demethylase activity, acetylation activity, or deacetylation activity.

The linkage to the second polypeptide domain can be through reversible or irreversible covalent linkage or through a non-covalent linkage, as long as the linker does not interfere with the function of the second polypeptide domain. For example, a ZFBD, TALE or Cas polypeptide can be linked to a second polypeptide domain as part of a fusion protein. As another example, they can be linked through reversible non-covalent interactions such as avidin (or streptavidin)-biotin interaction, histidine-divalent metal ion interaction (such as, Ni, Co, Cu, Fe), interactions between multimerization (such as, dimerization) domains, or glutathione S-transferase (GST)-glutathione interaction. As yet another example, they can be linked covalently but reversibly with linkers such as dibromomaleimide (DBM) or amino-thiol conjugation.

The second polypeptide domain may be at the C-terminal end of the first polypeptide domain, or at the N-terminal end of the first polypeptide domain, or a combination thereof. The fusion protein may include one second polypeptide domain, or two of the second polypeptide domains. For example, the fusion protein may include a second polypeptide domain at the N-terminal end as well as a second polypeptide domain at the C-terminal end of the first polypeptide domain. The two second polypeptide domains may be the same or different. In other embodiments, the fusion protein may include more than one (for example, two or three) second polypeptide domains in tandem.

In example embodiments, a fusion protein comprising (a) nuclease-active Cas9, a nuclease null Cas9 (dCas9), or a ZFBD, or a TALE linked to (b) an activator or repressor can be used to modulate gene expression (see examples of activators or repressors below). The fusion proteins optionally include a linker between the dCas9 (or ZF DNA-binding domain or TALE DNA-binding domain) and the activator or repressor. Suitable linkers include short stretches of amino acids (such as 2-20 amino acids) and are typically flexible (i.e. comprising amino acids with high degree of freedom such as glycine, alanine and serine). In example embodiments, the linker comprises one or more units consisting of GGGS or GGGGS, such as two, three, four or more repeats of the GGGS or GGGGS unit. Other linker sequences known in the art can also be used.

In some embodiments, the fusion protein includes a Cas9 protein or a mutated Cas9 protein, fused to a second polypeptide domain that has an activity such as transcription activation activity, transcription repression activity, transcription release factor activity, histone modification activity, nuclease activity, nucleic acid association activity, methylase activity, demethylase activity, acetylation activity, or deacetylation activity.

a. Transcription Activation Activity

The second polypeptide domain can have transcription activation activity, i.e., a transactivation domain. For example, gene expression of endogenous mammalian genes, such as human genes, can be achieved by targeting a fusion protein of dCas9 and a transactivation domain to mammalian promoters via combinations of gRNAs. The transactivation domain can include a VP 16 protein, multiple VP 16 proteins, such as a VP48 domain or VP64 domain, or p65 domain of NF kappa B transcription activator activity. For example, the fusion protein may be dCas9-VP64. In some embodiments, the fusion protein may be VP64-dCas9-VP64 (SEQ ID NO: 43, encoded by the polynucleotide of SEQ ID NO: 44). A transcription activation domain may include p300, such as p300-core. A fusion protein that activates transcription may also include dCas9-p300, such as the polypeptide of SEQ ID NO: 45 or SEQ ID NO: 48.

Non-limiting examples of activators may include: VP64, VP16; GAL4; p65 subdomain (NFkB); KMT2 family transcriptional activators: hSET1A, hSET1B, MLL1 to 5, ASH1, and homologs (Trx, Trr, Ash1); KMT3 family: SYMD2, NSD1; KMT4 family: DOT1L and homologs; KDM1: LSD1/BHC110 and homologs (SpLsd1/Swm1/Saf110, Su(var)3-3); KDM3 family: JHDM2a/b; KDM4 family: JMJD2A/JHDM3A, JMJD2B, JMJD2C/GASC1, JMJD2D, and homologs (Rph1); KDM6 family: UTX, JMJD3, VP64-p65-Rta (VPR); synergistic action mediator (SAM); p300; VP160; VP64-dCas9-BFP-VP64; KAT2 family: hGCN5, PCAF, and homologs (dGCN5/PCAF, Gcn5; KAT3 family: CBP, p300 and homologs (dCBP/NEJ); KAT4: TAF1 and homologs (dTAF1); KAT5: TIP60/PLIP, and homologs; KAT6: MOZ/MYST3, MORF/MYST4, and homologs (Mst2, Sas3, CG1894); KAT7: HBO1/MYST2, and homologs (CHM, Mst2); KAT8: HMOF/MYST1, and homologs (dMOF, CG1894, Sas2, Mst2); KAT13 family: SRC1, ACTR, P160, CLOCK, and homologs; AID/Apobed deaminase family: AID; TET dioxygenase family: TET1; DEMETER glycosylase family: DME, DML1, DML2, or ROS1.

b. Transcription Repression Activity

The second polypeptide domain can have transcription repression activity. The second polypeptide domain can have a Kruppel associated box activity, such as a KRAB domain, ERF repressor domain activity, Mxil repressor domain activity, SID4X repressor domain activity, Mad-SID repressor domain activity, or TATA box binding protein activity. For example, the fusion protein may be dCas9-KRAB (polynucleotide sequence SEQ ID NO: 87; protein sequence SEQ ID NO: 88).

Non-limiting examples of repressors may include: KRAB, Mad mSIN3 interaction domain (SID); the ERF repressor domain (ERD); KMT1 family: SUV39H1, SUV39H2, G9A, ESET/SETBD1, and homologs (Cir4, Su(var)3-9); KMT5 family: Pr-SET7/8, SUV4-20H1, and homologs (PR-set7, Suv4-20, and Set9); KMT6: EZH2, KMT8: RIZ1, KDM4 family: JMJD2A/JHDM3A, JMJD2B, JMJ2D2C/GASC1, JMJD2D, and homologs (Rph1); KDM5 family JARID1A/RBP2, JARIDIB/PLU-1, JARIDIC/SMCX, JARIDID/SMCY, and homologs (Lid, Jhn2, Jmj2); HDAC1, HDAC2, HDAC3, HDAC8, and its homologs (Rpd3, Hos1, Cir6); HDAC4, HDAC5, HDAC7, HDAC9, and its homologs (Hda1, Cir3); SIRT1, SIRT2, and its homologs (Sir2, Hst1, Hst2, Hst3, and Hst4); HDAC11, DNMT1, DNMT3a/3b, MET1, DRM3, and homologs, ZMET2, CMT1, CMT2, Laminin A, Laminin B, or CTCF.

c. Transcription Release Factor Activity

The second polypeptide domain can have transcription release factor activity. The second polypeptide domain can have eukaryotic release factor 1 (ERF1) activity or eukaryotic release factor 3 (ERF3) activity.

d. Histone Modification Activity

The second polypeptide domain can have histone modification activity. The second polypeptide domain can have histone deacetylase, histone acetyltransferase, histone demethylase, or histone methyltransferase activity. The histone acetyltransferase may be p300 or CREB-binding protein (CBP) protein, or fragments thereof, such as p300-core. For example, the fusion protein may be dCas9-p300.

e. Nuclease Activity

The second polypeptide domain can have nuclease activity that is different from the nuclease activity of the Cas9 protein. A nuclease, or a protein having nuclease activity, is an enzyme capable of cleaving the phosphodiester bonds between the nucleotide subunits of nucleic acids. Nucleases are usually further divided into endonucleases and exonucleases, although some of the enzymes may fall in both categories. Well known nucleases include deoxyribonuclease and ribonuclease.

f. Nucleic Acid Association Activity

The second polypeptide domain can have nucleic acid association activity or nucleic acid binding protein-DNA-binding domain (DBD). A DBD is an independently folded protein domain that contains at least one motif that recognizes double- or single-stranded DNA. A DBD can recognize a specific DNA sequence (a recognition sequence) or have a general affinity to DNA. A nucleic acid association region may be selected from helix-turn-helix region, leucine zipper region, winged helix region, winged helix-turn-helix region, helix-loop-helix region, immunoglobulin fold, B3 domain, Zinc finger, HMG-box, Wor3 domain, TAL effector DNA-binding domain.

g. Methylase Activity

The second polypeptide domain can have methylase activity, which involves transferring a methyl group to DNA, RNA, protein, small molecule, cytosine or adenine. In some embodiments, the second polypeptide domain includes a DNA methyltransferase.

h. Demethylase Activity

The second polypeptide domain can have demethylase activity. The second polypeptide domain can include an enzyme that removes methyl (CH3-) groups from nucleic acids, proteins (in particular histones), and other molecules. Alternatively, the second polypeptide can convert the methyl group to hydroxymethylcytosine in a mechanism for demethylating DNA. The second polypeptide can catalyze this reaction. For example, the second polypeptide that catalyzes this reaction can be Tet1, also known as Tet1CD (Ten-eleven translocation methylcytosine dioxygenase 1; polynucleotide sequence SEQ ID NO: 1138; amino acid sequence SEQ ID NO: 1139).

i. Regulation of Gene Expression Using Cas Polypeptides Alone

A Targeted Activator System or Targeted Repressor System can comprise a Cas polypeptide alone, not linked to an activator or repressor. Catalytically active Cas polypeptides or nuclease null Cas polypeptides can be administered with a dead guide RNA to activate gene expression as described above. Cas polypeptides with reduced nuclease activity can also be administered with normal guide RNA to repress gene expression as described in Qi et al., Cell. February 28; 152(5): 1173-1183, 2013.

### 8. Repair Pathways

The DNA Targeting System may target a regulatory element of a gene in the PWS-associated locus and alter its activity by introducing a mutation in the regulatory element. In some embodiments, the at least one gRNA targets an inhibitory regulatory element of a gene within the 15q1-13 locus. In such embodiments, the gRNA may be combined with a Cas9 protein that introduces a mutation in the regulatory element such as an insertion, deletion and/or substitution, such that activity of the inhibitory regulatory element is decreased, thereby activating expression of the gene within the 15q11-13 locus for the treatment of PWS. In other embodiments, the at least one gRNA targets an activating regulatory element of a gene within the 15q11-13 locus. In such embodiments, the gRNA may be combined with a Cas9 protein that introduces a mutation in the regulatory element such as an insertion, deletion and/or substitution, such that activity of the activating regulatory element is increased, thereby activating expression of the gene within the 15q11-13 locus for the treatment of PWS.

A nuclease system, such as a CRISPR/Cas9-based gene editing system, may be used to introduce site-specific single or double strand breaks at targeted regions of genomic loci, such as a regulatory element of a gene within the 15q11-13 locus. Site-specific breaks are created when any of the nuclease-based gene editing systems described herein binds to a target DNA sequences, thereby permitting cleavage of the target DNA. This DNA cleavage may stimulate the natural DNA-repair machinery, leading to one of two possible repair pathways: homology-directed repair (HDR) or the non-homologous end joining (NHEJ) pathway.

a. Homology-Directed Repair (HDR)

Restoration of protein expression from a gene within the 15q11-13 locus may involve homology-directed repair. A donor template may be administered to a cell that has been treated with a nuclease system to induce a single- or double-stranded DNA break. The donor template may include a nucleotide sequence encoding a mutated version of a regulatory element (an inhibitory regulatory element or an activating regulatory element) of a gene within the 15q11-13 locus. Mutations may include, for example, nucleotide substitutions, insertions, deletions, or a combination thereof. For example, introduced mutation(s) into the inhibitory regulatory element of a gene within the 15q11-13 locus may reduce the transcription of or binding to the inhibitory regulatory element, thereby activating expression of the gene within the 15q11-13 locus for the treatment of PWS.

b. Non-Homologous End Joining (NHEJ)

Restoration of protein expression from a gene within the 15q11-13 locus may be through template-free NHEJ-mediated DNA repair. In certain embodiments, NHEJ is a nuclease mediated NHEJ, which in certain embodiments, refers to NHEJ that is initiated by a Cas9 protein that cuts double stranded DNA. The method comprises administering a nuclease system disclosed herein, such as a CRISPR/Cas9-based gene editing system, or a composition comprising thereof to a subject for genome editing.

Nuclease mediated NHEJ may mutate an regulatory element (an inhibitory regulatory element or an activating regulatory element) of a gene within the 15q11-13 locus. Nuclease mediated NHEJ may offer several potential advantages over the HDR pathway. For example, NHEJ does not require a donor template, which may cause nonspecific insertional mutagenesis. In contrast to HDR, NHEJ operates efficiently in all stages of the cell cycle and therefore may be effectively exploited in both cycling and post-mitotic cells, such as muscle fibers. This provides a robust, permanent gene restoration alternative to oligonucleotide-based exon skipping or pharmacologic forced read-through of stop codons and could theoretically require as few as one drug treatment. NHEJ-based mutation of a regulatory element using a CRISPR/Cas9-based gene editing system, as well as other engineered nucleases including meganucleases and zinc finger nucleases, may be combined with other existing ex vivo and in vivo platforms for cell- and gene-based therapies, in addition to the plasmid electroporation approach described here. For example, delivery of a CRISPR/Cas9-based gene editing system by mRNA-based gene transfer or as purified cell permeable proteins could enable a DNA-free genome editing approach that would circumvent any possibility of insertional mutagenesis.

### 9. CRISPR-Cas DNA Targeting Systems

Further provided herein are CRISPR-Cas DNA targeting systems or compositions that comprise such genetic constructs. The DNA targeting compositions include at least one gRNA (such as, two gRNAs) that targets a target region. The at least one gRNA can bind and recognize a target region as described herein. The target region may be a gene (such as, a gene within the 15q11-13 locus) or a regulatory element thereof, as described above. In some embodiments, the gRNAs target the sense strand. In some embodiments, the gRNAs target the antisense strand.

In some embodiments, the DNA targeting composition includes a first gRNA and a second gRNA. In some embodiments, the first gRNA and the second gRNA comprise different targeting domains that bind different target regions.

The DNA targeting composition may further include at least one Cas9 protein or a Cas9 fusion protein. In some embodiments, the Cas9 protein or a Cas9 fusion protein recognizes a PAM of either NNGRRT (SEQ ID NO: 21) or NNGRRV (SEQ ID NO: 22). In some embodiments, the DNA targeting composition includes a nucleotide sequence set forth in SEQ ID NO: 37. In certain embodiments, the vector is configured to form a first and a second double strand break in a first and a second location of a gene within the 15q11-13 locus, respectively, thereby deleting a segment of the gene within the 15q11-13 locus or a regulatory element thereof.

The DNA targeting composition may further comprise a donor DNA or a transgene. The deletion efficiency of the presently disclosed vectors can be related to the deletion size, i.e., the size of the segment deleted by the vectors. In certain embodiments, the length or size of specific deletions is determined by the distance between the PAM sequences in the gene being targeted. In certain embodiments, a specific deletion of a segment of the gene, which is defined in terms of its length and a sequence it comprises, is the result of breaks made adjacent to specific PAM sequences within the target gene. In some embodiments, the deletion size is about 10 to about 2,000 base pairs (bp).

### 10. Pharmaceutical Compositions

Further provided herein are pharmaceutical compositions comprising the above-described genetic constructs or DNA Targeting systems, including Targeted Activator System(s), Targeted Repressor System(s) or nuclease systems. Such systems, or at least one component thereof, as detailed herein may be formulated into pharmaceutical compositions in accordance with standard techniques well known to those skilled in the pharmaceutical art. The pharmaceutical compositions can be formulated according to the mode of administration to be used. In cases where pharmaceutical compositions are injectable pharmaceutical compositions, they are sterile, pyrogen free, and particulate free. An isotonic formulation is preferably used. Generally, additives for isotonicity may include sodium chloride, dextrose, mannitol, sorbitol and lactose. In some cases, isotonic solutions such as phosphate buffered saline are preferred. Stabilizers include gelatin and albumin. In some embodiments, a vasoconstriction agent is added to the formulation.

The pharmaceutical composition may comprise about 1 ng to about 10 mg of DNA encoding the DNA Targeting System(s), including the Targeted Activator System(s), Targeted Repressor System(s) or nuclease systems described herein, such as in the form of a DNA construct, an AAV vector or a lentivector. The pharmaceutical composition may comprise about 1 ng to about 10 mg of the gRNA described herein.

The composition may further comprise a pharmaceutically acceptable excipient. The pharmaceutically acceptable excipient may be functional molecules as vehicles, adjuvants, carriers, or diluents. The pharmaceutically acceptable excipient may be a transfection facilitating agent, which may include surface active agents, quinone analogs, vesicles such as squalene and squalene, hyaluronic acid, lipids, liposomes, calcium ions, viral proteins, polyanions, polycations, including poly-L-glutamate, or nanoparticles, or other known transfection facilitating agents. The carrier may be a non-toxic, inert solid, semi-solid or liquid filler, diluent, encapsulating material or formulation auxiliary of any type. Pharmaceutically acceptable carriers include, for example, diluents, antioxidants, preservatives, solvents, suspending agents, wetting agents, surfactants, propellants, humectants, powders, pH adjusting agents, and combinations thereof. The pharmaceutically acceptable excipient may be a transfection facilitating agent, which may include surface active agents, such as immune-stimulating complexes (ISCOMS), Freunds incomplete adjuvant, LPS analog including monophosphoryl lipid A, muramyl peptides, quinone analogs, vesicles such as squalene and squalene, hyaluronic acid, lipids, liposomes, calcium ions, viral proteins, polyanions, polycations, including poly-L-glutamate, or nanoparticles, or other known transfection facilitating agents.

The transfection facilitating agent may be a polyanion, polycation, including poly-L-glutamate (LGS), or lipid. The transfection facilitating agent is poly-L-glutamate, and more preferably, the poly-L-glutamate is present in the composition for genome editing in skeletal muscle or cardiac muscle at a concentration less than 6 mg/mL. The transfection facilitating agent may also include surface active agents such as immune-stimulating complexes (ISCOMS), Freunds incomplete adjuvant, LPS analog including monophosphoryl lipid A, muramyl peptides, quinone analogs and vesicles such as squalene and squalene, and hyaluronic acid may also be used administered in conjunction with the genetic construct. In some embodiments, the DNA vector encoding the composition may also include a transfection facilitating agent such as lipids, liposomes, including lecithin liposomes or other liposomes known in the art, as a DNA-liposome mixture (see for example International Patent Publication No. WO9324640), calcium ions, viral proteins, polyanions, polycations, or nanoparticles, or other known transfection facilitating agents. In some embodiments, the transfection facilitating agent is a polyanion, polycation, including poly-L-glutamate (LGS), or lipid.

### 11. Administration

The DNA Targeting Systems, or at least one component thereof, as detailed herein, or the pharmaceutical compositions comprising the same, may be administered to a subject. Such compositions can be administered in dosages and by techniques well known to those skilled in the medical arts taking into consideration such factors as the age, sex, weight, and condition of the particular subject, and the route of administration. The presently disclosed DNA Targeting Systems, or at least one component thereof, genetic constructs, or compositions comprising the same, may be administered to a subject by different routes including orally, parenterally, sublingually, transdermally, rectally, transmucosally, topically, intranasal, intravaginal, via inhalation, via buccal administration, intrapleurally, intravenous, intraarterial, intraperitoneal, subcutaneous, intradermally, epidermally, intramuscular, intranasal, intrathecal, intracranial, and intraarticular or combinations thereof. In certain embodiments, the DNA Targeting System, genetic construct, or composition comprising the same, is administered to a subject intramuscularly, intravenously, or a combination thereof. For veterinary use, the DNA Targeting Systems, genetic constructs, or compositions comprising the same may be administered as a suitably acceptable formulation in accordance with normal veterinary practice. The veterinarian may readily determine the dosing regimen and route of administration that is most appropriate for a particular animal. The DNA Targeting Systems, genetic constructs, or compositions comprising the same may be administered by traditional syringes, needleless injection devices, “microprojectile bombardment gone guns,” or other physical methods such as electroporation (“EP”), “hydrodynamic method”, or ultrasound.

The DNA Targeting Systems, genetic constructs, or compositions comprising the same may be delivered to a subject by several technologies including DNA injection (also referred to as DNA vaccination) with and without in vivo electroporation, liposome mediated, nanoparticle facilitated, recombinant vectors such as recombinant lentivirus, recombinant adenovirus, and recombinant adenovirus associated virus. The composition may be injected into the skeletal muscle or cardiac muscle. For example, the composition may be injected into the tibialis anterior muscle or tail.

a. Delivery of Protein and Nucleic Acids

Guide RNA and Cas polypeptides (including Cas fusion proteins) can be delivered to cells as DNA, RNA, or as pre-formed ribonucleoprotein complexes (RNPs) formats. The DNA format for the guide RNA will be transcribed into RNA. The DNA and RNA formats for Cas polypeptides or Cas fusion proteins require transcription and/or translation after being introduced into the cell, and the Cas polypeptide preferably also includes one, two or more nuclear localization sequences (NLS) to enhance entry into nucleus. Transfection methods include transfection into the cytoplasm (electroporation, lipofection) or the nucleus (nucleofection, microinjection), all well known in the art.

The pre-formed RNP format does not require any transcription or translation. If using RNPs, then delivery to the nucleus (nucleofection, microinjection) requires fewer steps. Other techniques to deliver RNPs into cells include induction of transmembrane internalization assisted by membrane filtration (TRIAMF) and induced transduction by osmocytosis and propane betaine (iTOP).

Similarly, ZF fusion proteins or TALE fusion proteins, comprising a nuclease, an activator or a repressor, can be delivered in a DNA or RNA format. These proteins preferably also include one, two or more NLS to enhance entry into nucleus.

DNA constructs comprising DNA encoding the guide RNA and/or Cas polypeptides and/or ZF fusion protein and/or TALE fusion protein described herein may comprise, such as, heterologous regulatory elements or transcriptional control signals as described herein for expression of the coding sequences of the nucleic acid. The regulatory elements may include, for example, a promoter, an enhancer, an initiation codon, a stop codon, or a polyadenylation signal.

The DNA Targeting System, such as, nuclease system or Targeted Activator System or Targeted Repressor System, or one or more components thereof, may be encoded by or comprised within a genetic construct. Genetic constructs may include polynucleotides such as vectors and plasmids. The construct may be recombinant. In some embodiments, the genetic construct may comprise a promoter that is operably linked to the polynucleotide encoding at least one gRNA and/or a Cas9 protein. In some embodiments, the promoter is operably linked to the polynucleotide encoding a first gRNA, a second gRNA, and/or a Cas9 protein. The genetic construct may be present in the cell as a functioning extrachromosomal molecule that is not integrated into the chromosome. The genetic construct may be integrated into the chromosome. The genetic construct may be a linear minichromosome including centromere, telomeres, or plasmids or cosmids. The genetic construct may be transformed or transduced into a cell. Further provided herein is a cell transformed or transduced with a DNA targeting system or component thereof as detailed herein. In some embodiments, the cell is a stem cell. The stem cell may be a human stem cell. The stem cell may be a human pluripotent stem cell (iPSCs). Further provided are stem cell-derived neurons, such as neurons derived from iPSCs transformed or transduced with a DNA targeting system or component thereof as detailed herein.

i) Viral Vectors

Viral vectors can also be used to transfer DNA or RNA into cells via transduction. Further provided herein is a viral delivery system. The nucleic acid encoding the DNA Targeting system, such as, Targeted Activator System, Targeted Repressor System or nuclease system, is packaged into viral particles which are then introduced into cells. For example, nucleic acid encoding the gRNA and/or Cas sequence is packaged into viral particles. To make the viral particles, generally the plasmid containing the gRNA or Cas9 encoding sequence and plasmids containing viral genes are introduced into a packaging cell line, and viral particles are harvested. Suitable viral vectors include lentivirus, adenovirus, adeno-associated virus (AAV), and herpes viruses. Platforms such as adeno-associated viral vectors (AAVs) are commonly used and can provide sustained expression without integration into the genome. AAV vectors possess significantly lower packaging capability than LVs (<5 kb). Lentivirus are effective in a variety of cells including non-dividing cells and can integrate into the genome or can be non-integrating. Viral vectors can be used to deliver DNA and RNA in vivo to subjects or ex vivo to their cells.

In some embodiments, the vector is an adeno-associated virus (AAV) vector. The AAV vector is a small virus belonging to the genus Dependovirus of the Parvoviridae family that infects humans and some other primate species. As used herein, the term “adenoviral associated virus (AAV) vector” refers to a vector having functional or partly functional ITR sequences and transgenes. As used herein, the term “ITR” refers to inverted terminal repeats (ITR). The ITR sequences may be derived from an adeno-associated virus serotype, including without limitation, AAV-1, AAV-2, AAV-3, AAV-4, AAV-5, AAV-6, AAV-7, AAV-8, AAV-9, AAV-10, AAV-11, AAV-12. The ITRs, however, need not be the wild-type nucleotide sequences, and may be altered (such as, by the insertion, deletion and/or substitution of nucleotides), so long as the sequences retain function to provide for functional rescue, replication and packaging. AAV vectors may have one or more of the AAV wild-type genes deleted in whole or part, preferably the rep and/or cap genes but retain functional flanking ITR sequences. Functional ITR sequences function to, for example, rescue, replicate and package the AAV virion or particle. Thus, an “AAV vector” is defined herein to include at least those sequences required for insertion of the transgene into a subject's cells. Optionally included are those sequences necessary in cis for replication and packaging (such as, functional ITRs) of the virus.

AAV vectors may be used to deliver CRISPR/Cas9-based gene editing systems using various construct configurations. For example, AAV vectors may deliver Cas9 and gRNA expression cassettes on separate vectors or on the same vector. Alternatively, if the small Cas9 proteins, derived from species such as Staphylococcus aureus or Neissena meningitidis, are used then both the Cas9 and up to two gRNA expression cassettes may be combined in a single AAV vector within the 4.7 kb packaging limit.

In some embodiments, the AAV vector is a modified AAV vector. The modified AAV vector may have enhanced cardiac and/or skeletal muscle tissue tropism. The modified AAV vector may be capable of delivering and expressing the CRISPR/Cas9-based gene editing system in the cell of a mammal. For example, the modified AAV vector may be an AAV-SASTG vector (Piacentino et al. Human Gene Therapy 2012, 23, 635-846). The modified AAV vector may be based on one or more of several capsid types, including AAV1, AAV2, AAV5, AAV6, AAV8, and AAV9. The modified AAV vector may be based on AAV2 pseudotype with alternative muscle-tropic AAV capsids, such as AAV2/1, AAV2/6, AAV2/7, AAV2/8, AAV2/9, AAV2.5, and AAV/SASTG vectors that efficiently transduce skeletal muscle or cardiac muscle by systemic and local delivery (Seto et al. Current Gene Therapy 2012, 12, 139-151). The modified AAV vector may be AAV2i8G9 (Shen et al. J. Biol. Chem. 2013, 288, 28814-28823).

As used herein, the term “lentiviral vector” refers to a vector derived from lentivirus, a family of retroviruses characterized by long incubation periods. To be used safely as a vector, the lentivirus has been modified extensively to delete virulence and replication genes. In addition, the integrase of lentivirus can be deleted or mutated, resulting in a non-replicating and non-integrating lentivector. Integrase-deficient lentiviral vectors (IDLV) can be used to deliver CRISPR-cas systems. Lentivectors carrying Cas polypeptides and guide RNAs are described in U.S. Pub. App. No. 20180201951, incorporated herein by reference in its entirety.

ii) Nanoparticles

Lipid materials have been used to create lipid nanoparticles (LNPs) based on ionizable cationic lipids, which exhibit a cationic charge in the lowered pH of late endosomes to induce endosomal escape, because of the tertiary amines in their structure. These LNPs have been used, for example, to deliver RNA interference (RNAi) components, as well as genetic constructs or CRISPR-Cas systems. See, such as, Wilbie et al., Acc Chem Res.; 52(6):1555-1564, 2019. Wang et al., Proc Natl Acad Sci USA; 113(11):2868-2873, 2016 describe use of biodegradable cationic LNPs. Chang et al., Acc. Chem. Res., 52, 665-675, 2019 describe use of ionizable lipid along with cholesterol, DSPC, and a PEGylated lipid to create LNPs.

Polymer based particles can be used for genetic construct delivery in a similar manner as lipids. A number of materials have been used for delivery of nucleic acids. For example, cationic polymers such as polyethylenimine (PEI) can be complexed to nucleic acids and can induce endosomal uptake and release, similarly to cationic lipids. Dendrimeric structures of poly(amido-amine) (PAMAM) can also be used for transfection. These particles consist of a core from which the polymer branches. They exhibit cationic primary amines on their surface, which can complex to nucleic acids. Networks based on zinc to aid cross-linking of imidazole have been used as delivery methods, relying on the low pH of late endosomes which, upon uptake, results in cationic charges due to dissolution of the zeolitic imidazole frameworks (ZIF), after which the components are released into the cytosol. Colloidal gold nanoparticles have also been used. See Wilbie et al., supra.

### 12. Methods

a. Methods For Treating Prader-Willi Syndrome (PWS)

Provided herein are methods for treating Prader-Willi Syndrome (PWS) in a subject in need thereof. The method may include administering to the subject a DNA Targeting System as detailed herein, an isolated polynucleotide sequence as detailed herein, a vector or genetic construct as detailed herein, a modified cell as detailed herein, or a combination thereof. In some embodiments, the expression of at least one gene within the 15q11-q13 locus is increased in the subject or in a sample therefrom. In some embodiments, the expression of at least one gene within the 15q11-q13 locus is increased in the subject or in a sample therefrom, such as from activation of the maternal copy of the gene. In some embodiments, the expression of at least one RNA transcript selected from SNRPN, SNORD115, and SNORD116, or a combination thereof, is increased in the subject or in a sample therefrom. In some embodiments, the initiation of transcription from the SNRPN promoter, SNORD115 promoter, SNORD116 promoter, or a combination thereof, is increased in the subject or in a sample therefrom.

The disclosure herein identifies target regions within the PWS associated locus of chromosome 15q11-13 that can be targeted by the DNA Targeting Systems, such as, Targeted Activator Systems, for activation, to increase expression of genes and gene products to treat PWS. Relative to position 1 being the start sire of the SNRPN gene exon 1 of the PWS imprinting center on chromosome 15, the target regions of interest for activation are at nucleotide positions −127023 to −125023; nucleotide positions −93065 to −91065; and/or nucleotide positions −1104 to +896. More specifically, the target regions are at nucleotide positions −126523 to 125523; nucleotide positions −92565 to −91565; and/or nucleotide positions −604 to +396. Even more specifically, the target region encompasses nucleotide positions −126023, −92065, and/or −104, and may be within about 100-1000, about 100-900, about 100-800, about 100-700, about 100-600, about 100-500, about 100-400, about 100-300 or about 100-200 bp upstream, or downstream, of the target region of the DNA identified herein.

The disclosure herein also identifies target regions within the PWS associated locus of chromosome 15q11-13 that can be targeted by the DNA Targeting Systems, such as Targeted Repressor Systems, for repression, to increase expression of genes and gene products to treat PWS. Relative to position 1 being the start site of the SNRPN gene exon 1 of the PWS imprinting center on chromosome 15, the target regions of interest for activation are at nucleotide positions +23022 to +25022 and/or +34734 to +36734. More specifically, the target regions are at nucleotide positions +23522 to +24522 and/or +35234 to +36234. Even more specifically, the target region encompasses nucleotide positions +24022 and/or +35734, and may be within about 100-1000, about 100-900, about 100-800, about 100-700, about 100-600, about 100-500, about 100-400, about 100-300 or about 100-200 bp upstream, or downstream, of the target region of the DNA identified herein.

Relative to position 1 being the start site of the SNRPN gene exon 1 of the PWS imprinting center on chromosome 15, additional target regions of interest for activation and/or demethylation include regions at positions −126547 to −124695 [mat1]; −131937 to −130580 [mat1A]; −129415 to −127715 [mat1B]; −123798 to −122440 [mat1C]; −92568 to −91460 [mat2]; −797 to +1346 [mat3]; and/or +12858 to +14026 [mat4]. More specifically, within these regions, further subregions include target regions at positions: −126047 to −125195 [mat1]; −131437 to −131080 [mat1A]; −128915 to −128215 [mat1B]; −123298 to −122940 [mat1C]; −92068 to −91960 [mat2]; −297 to +846 [mat3]; and/or +13358 to +13526 [mat4]. Even more specifically, within these subregions, further subregions include target regions at positions: −126047 to −125947; −125997 to −125897; −125947 to −125847; −125897 to −125797; −125847 to −125747; −125797 to −125697; −125747 to −125647; −125697 to −125597; −125647 to −125547; −125597 to −125497; −125547 to −125447; −125497 to −125397; −125447 to −125347: −125397 to −125297; −125347 to −125247; −125297 to −125195; and/or −125247 to −125195 [mat1]; −131437 to −131337: −131387 to −131287; −131337 to −131237; −131287 to −131187; −131237 to −131137; −131187 to −131087; −131137 to −131037; and/or −131087 to −131080 [mat1A]; −128915 to −128815; −128865 to −128765; −128815 to −128715; −128765 to −128665; −128715 to −128615; −128665 to −128656; −128615 to −128515; −128565 to −128465; −128515 to −128415; −128465 to −128365; −128415 to −128315; −128385 to −128265; and/or −128315 to −128215 [mat1B]; −123298 to −123198; −123248 to −123148; −123198 to −123098; −123148 to −123048; −123098 to −122998; −123048 to −122948; and/or −122998 to −122940 [mat1C]-92068 to −91968; −92018 to −91918; and/or −91968 to −91960 [mat2]; −297 to −187; −247 to −147; −197 to −97; −147 to −47; −97 to +3; −47 to +53; +3 to +100; +53 to +153; +103 to +203; +153 to +253; +203 to +303; +253 to +353; +303 to +403; +353 to +453; +403 to +503; +453 to +553; +503 to +603; +553 to +653; +603 to +703; +653 to +753; +703 to +803; +753 to +846; and/or +803 to +846 [mat3] and/or +13358 to +13458; +13408 to +13508; +13458 to +13526; and/or +13508 to +13526 [mat4].

Relative to position 1 being the start site of the SNRPN gene exon 1 of the PWS imprinting center on chromosome 15, additional target regions of interest for repression include regions at positions −101358 to −94223 [pat1]; −58232 to −51914 [pat2]; −4847 to −3047 [pat3]; −1774 to +2421 [pat4]; +2446 to +24016 [pat5]; +23346 to +25082 [pat6]; +24340 to +35718 [pat7]; and/or +35206 to +36668 [pat8]. More specifically, within these regions, further subregions include target regions at positions: −100858 to −94723[pat1]; −57732 to −52414 [pat2]; −4347 to −3547 [pat3]; −1275 to +1921 [pat4]; +2948 to +23516 [pat5]; +23846 to +24582 [pat6]; +24840 to +35218 [pat7]; and/or +35706 to +36168 [pat8]. Even more specifically, within these subregions, further subregions include target regions at positions: +23846 to +23946; +23896 to +23996; +23946 to +24046; +23996 to +24096; +24046 to +24146; +24096 to +24196; +24146 to +24246; +24196 to +24296; +24246 to +24346; +24296 to +24396; +24346 to +24446; +24396 to +24496; +24446 to +24546; and/or +24496 to +24582; [pat6]; +35706 to +35806; +35756 to +35856; +35806 to +35906; +35856 to +35956; +35906 to +36006; +35956 to +36056; +36006 to +36106; +36056 to +36156; and/or +36106 to +36168 [pat8];

### 13. Examples

### Example 1

Initial screening of the PWS region was performed in human induced pluriplotent stem cells (iPSCs).

There are several imprinted genes within the 15q11-13 locus, including the paternally-expressed coding genes MAGEL2, NDN and SNURF-SNRPN, along with numerous noncoding RNAs (ncRNAs), including the snoRNA clusters SNORD115 and SNORD116 (Buiting, Am J Med Genet C Semin Med Genet 2010, 154C, 365-376). PWS patient genotypes commonly consist of deletions within 15q11-13 that encompass several of the coding and noncoding genes, although a subset of genotypes emphasize the snoRNA clusters as having particular influence in the etiology of PWS (Bieth et al., Eur J Hum Genet 2015, 23, 252-255; de Smith, Hum Mol Genet 2009, 18, 3257-3265; Duker et al., Eur J Hum Genet 2010, 18, 1196-1201; Sahoo et al., Nat Genet 2008, 40, 719-721). Further evidence suggests that SNURF-SNRPN and downstream ncRNAs, including SPA RNAs and snoRNAs, are processed from a single host transcript that initiates at the imprinting center located in exon 1 of SNRPN (Wu et al., Mol Cell 2016, 64, 534-548). Given that imprinting within 15q11-13 is thought to be orchestrated in part by the imprinting center in exon 1 of SNRPN, which also serves as the initiation of a host transcript that processes several paternally expressed genes highly implicated in the disease, SNRPN expression was selected as a proxy for the imprinting status of the 15q11-13 locus.

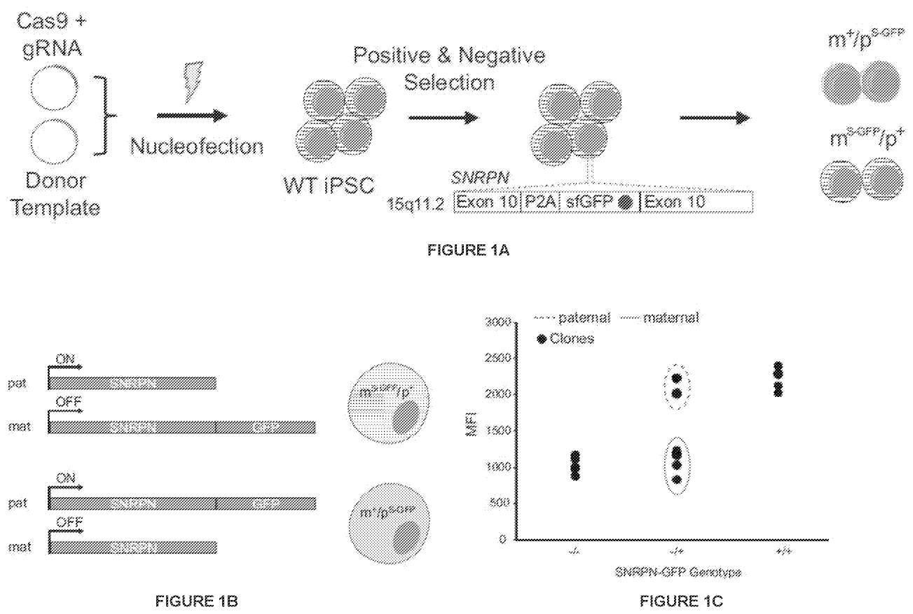

Superfolder GFP (sfGFP) was inserted into exon 10 of SNURF-SNRPN in frame with the SNRPN ORF in a wild-type human pluripotent stem cell line with two intact copies of 151q1-13 (FIG. 1A). A P2A skipping peptide was included between SNPRN and sfGFP to link the expression of the two proteins while avoiding disrupting SNPRN function, localization or stability with a direct fusion to sfGFP. Heterozygous clones were created with GFP-tagged SNRPN on either the maternal or paternal allele. Only the paternally-tagged cells were GFP-positive, indicating that imprinting is accurately maintained in this cell line (FIG. 1B). As expected, clonal lines were derived that had either the maternal or paternal allele tagged (GFP-tagged SNRPN on), with the heterozygous clones uniquely displaying a bimodal distribution in GFP fluorescence (FIG. 1C). These two SNRPN-2A-GFP lines independently report on SNRPN expression from the paternal or maternal allele, respectively. These cell lines were used in all subsequent CRISPR screens described in this study.

Construction of SNRPN-2A-GFP pluripotent stem cell lines. A human iPS cell line (RVR-iPSCs) was used to construct the maternal and paternal SNRPN-2A-GFP reporter lines. RVR-iPSCs were retrovirally reprogrammed from BJ fibroblasts and characterized previously (Lee et al., Cell 2012, 151, 547-558). To generate the SNRPN-2A-GFP reporter lines, 3×10⁶ cells were dissociated with Accutase (Stemcell Tech, 7920) and electroporated with 6 μg of gRNA-Cas9 expression vector and 3 μg of SNRPN targeting vector using the P3 Primary Cell 4D-Nucleofector Kit (Lonza, V4XP-3032). The SNRPN targeting vector contained ˜700 bp homology arms (surrounding the SNRPN stop codon), flanking a P2A-GFP sequence with a LoxP-puromycin resistance cassette. Transfected cells were plated into a 10 cm dish coated with Matrigel (Corning, 354230) in complete mTesR (Stemcell Tech, 85850) supplemented with 10 μM Rock Inhibitor (Y-27632, Stemcell Tech, 72304). 24 h after transfection, positive selection began with 1 μg/mL puromycin for 7 d. Following selection, cells were transfected with a CMV-CRE recombinase expression vector to remove the floxed puromycin selection cassette. Transfected cells were expanded and plated at low density for clonal isolation (180 cells/cm²). Resulting clones were mechanically picked and expanded and gDNA was extracted using QuickExtract DNA Extraction Solution (Lucigen, QE09050) for PCR screening of targeting vector integration. A polyclonal cell line expressing dCas9^{KRAB} or ^{VP64}dCas9^{VP64} was used for the CRISPR screens.

Lentiviral production and titration. HEK293T cells were acquired from the American Tissue Collection Center (ATCC) and purchased through the Duke University Cell Culture Facility. The cells were maintained in DMEM High Glucose supplemented with 10% FBS and 1% penicillin-streptomycin and cultured at 37° C. with 5% CO₂. For lentiviral production of the gRNA libraries, ^{VP64}dCas9^{VP64} and dCas9^{KRAB}, 4.5×10⁸ cells were transfected using the calcium phosphate precipitation method (Salmon and Trono, Curr Protoc Hum Genet 2007, Chapter 12, Unit 12 10) with 6 μg pMD2.G (Addgene #12259), 15 μg psPAX2 (Addgene #12260) and 20 μg of the transfer vector. The medium was exchanged 12-14 h after transfection, and the viral supernatant was harvested 24 and 48 h after this medium change. The viral supernatant was pooled and centrifuged at 600 g for 10 min, passed through a PVDF 0.45 μm filter (Millipore, SLHV033RB), and concentrated to 50× in 1×PBS using Lenti-X Concentrator (Clontech, 631232) in accordance with the manufacturer's protocol.

To produce lentivirus for gRNA validations, 0.4×10⁶ cells were transfected using Lipofectamine 3000 (Invitrogen, L3000008) according to the manufacturer's instructions with 200 ng pMD2.G, 600 ng psPAX2 (Addgene #12260) and 200 ng of the transfer vector. The medium was exchanged 12-14 h after transfection, and the viral supernatant was harvested 24 and 48 h after this medium change. The viral supernatant was pooled and centrifuged at 600 g for 10 min and concentrated to 50× in 1×PBS using Lenti-X Concentrator (Clontech, 631232) in accordance with the manufacturer's protocol.

The titer of the lentiviral gRNA library pool was determined by transducing 3×10⁴ cells with serial dilutions of lentivirus and measuring the percent mCherry expression 4 d after transduction with a SH800 FACS Cell Sorter (Sony Biotechnology). All lentiviral titrations were performed in the cell lines used in the CRISPR screens.

Quantitative RT-PCR. Cells were dissociated with Accutase (StemCell Tech, 7920) and centrifuged at 300 g for 5 min. Total RNA was isolated using RNeasy Plus (Qiagen, 74136) and QIAshredder kits (Qiagen, 79656). Reverse transcription was carried out on 0.1 μg total RNA per sample in a 10 μl reaction using the SuperScript VILO Reverse Transcription Kit (Invitrogen, 11754). 1.0 μl of cDNA was used per PCR reaction with Perfecta SYBR Green Fastmix (Quanta BioSciences, 95072) using the CFX96 Real-Time PCR Detection System (Bio-Rad). The amplification efficiencies over the appropriate dynamic range of all primers were optimized using dilutions of purified amplicon. All amplicon products were verified by gel electrophoresis and melting curve analysis. All qRT-PCR results are presented as fold change in RNA normalized to GAPDH expression.

Plasmid construction. The lentiviral ^{VP64}dCas9^{VP64} plasmid was generated by modifying Addgene #59791 to replace GFP with BSD blasticidin resistance. The gRNA expression plasmid for the single CRa-TF screen was generated by modifying Addgene #83925 to contain an optimized gRNA scaffold (Chen et al., Cell 2013, 155, 1479-1491), a puromycin resistance gene in place of Bsr and a mCherry transgene in place of GFP. Individual gRNAs were ordered as oligonucleotides (Integrated DNA Technologies) and cloned into the gRNA expression plasmids using BsmBI sites. The SNRPN targeting vector was cloned by inserting ˜700 bp homology arms (surrounding the SNRPN stop codon), amplified by PCR from genomic DNA of RVR-iPS cells, flanking a P2A-GFP sequence with a LoxP-puromycin resistance cassette.

Genomic DNA sequencing. The gRNA libraries were amplified from each gDNA sample across 100 μL PCR reactions using Q5 hot start polymerase (NEB, M0493) with 1 μg of gDNA per reaction. The PCR amplification was done according to the manufacturer's instructions, using 25 cycles at an annealing temperature of 60° C. with the following primers:

Fwd:
(SEQ ID NO: 38)
5′-AATGATACGGCGACCACCGAGATCTACACAATTTCTTGGG TAGTTTG
CAGTT
Rev:
(SEQ ID NO: 39)
5′-CAAGCAGAAGACGGCATACGAGAT-(6-bp index sequence)-
GACTCGGTGCCACTTTTTCAA

The amplified libraries were purified with Agencourt AMPure XP beads (Beckman Coulter, A63881) using double size selection of 0.65× and then 1× the original volume.

Each sample was quantified after purification with the Qubit dsDNA High Sensitivity assay kit (Thermo Fisher, Q32854). Samples were pooled and sequenced on a MiSeq (Illumina) with 21-bp paired-end sequencing using the following custom read and index primers:

Read1:
(SEQ ID NO: 40)
5′-GATTTCTTGGCTTTATATATCTTGTGGAAAGGACGAAACACCG
Index:
(SEQ ID NO: 41)
5′-GCTAGTCCGTTATCAACTTGAAAAAGTGGCACCGAGTC
Read2:
(SEQ ID NO: 42)
5′-GTTGATAACGGACTAGCCTTATTTTAACTTGCTATTTCTAGCTCTAAA
AC

Data processing and enrichment analysis. FASTQ files were aligned to custom indexes (generated from the bowtie2-build function) using Bowtie 2 (Langmead and Salzberg, Nat Methods 2012, 9, 357-359) with the options—p 32—end-to-end—very-sensitive −3 1 −1 0 −X 200. Counts for each gRNA were extracted and used for further analysis. All enrichment analysis was done with R. Individual gRNA enrichment was determined using the DESeq2 (Love et al., Genome Biol 2014, 15, 550) package to compare between high and low, unsorted and low, or unsorted and high conditions for each screen.

gRNA validations. The top enriched gRNAs from the screens were individually cloned into the appropriate gRNA expression vector as described previously. The gRNA validations were performed similarly as done with the screens using maternal or paternal SNRPN-2A-GFP lines stably expressing either dCas9^{KRAB} or ^{VP64}dCasd9^{VP64}, except the transductions were performed in 24-well plates and the virus was delivered at high MOI. For the validations of the dCas9^{KRAB} paternal screen, single gRNAs were tested per region. For the validations of the ^{VP64}dCas9^{VP64} maternal screen, pools of 3-4 gRNAs were tested per region. Cells were cultured on matrigel-coated 24-well plates in mTesR and harvested for flow cytometry or qRT-PCR 5-7 d after gRNA transduction.

Paternal and maternal SNRPN-2A-GFP screens. Each screen was performed in triplicate with independent transductions. For each replicate, 24×10⁶ SNRPN-2A-GFP ^{VP64}dCas9^{VP64} (maternal) or dCas9^{KRAB} (paternal) iPSCs were dissociated using Accutase (Stemcell Tech, 7920) and transduced in suspension across five matrigel-coated 15-cm dishes in mTesR (Stemcell Tech 85850) supplemented with 10 μM Rock Inhibitor (Y-27632, Stemcell Tech, 72304). Cells were transduced at a MOI of 0.2 to obtain one gRNA per cell and ˜430-fold coverage of the PWS gRNA library. The medium was changed to fresh mTesR without Rock Inhibitor 18-20 h after transduction. Antibiotic selection was started 30 h after transduction by adding 1 μg/mL puromycin (Sigma, P8833) directly to the plates without changing the medium. The cells were fed daily and passaged as necessary maintaining library coverage until harvest.

Cells were harvested for sorting 9 d after transduction of the gRNA library for both screens. Cells were washed once with 1×PBS, dissociated using Accutase, filtered through a 30 μm CellTrics filter (Sysmex, 04-004-2326) and resuspended in FACS Buffer (0.5% BSA (Sigma, A7906), 2 mM EDTA (Sigma, E7889) in PBS). Before sorting, an aliquot of 4.8×10⁶ cells were taken to represent a bulk unsorted population. The highest and lowest 10% of cells were sorted based on GFP expression and 4.8×10⁶ cells were sorted into each bin. Sorting was done with a SH800 FACS Cell Sorter (Sony Biotechnology). After sorting, genomic DNA was harvested with the DNeasy Blood and Tissue Kit (Qiagen, 69506).

### Example 2

### Screen of Paternal SNRPN-2A-GFP Cell Line with a Targeted Repressor System

Reactivation of PWS-associated genes on the maternal allele via G9a inhibition supports the role of histone modifications in the maintenance of imprinting within this locus (Kim et al., Nat Med 2017, 23, 213-222; Fulmer-Smentek and Francke, Hum Mol Genet 2001, 10, 645-852). However, G9a inhibition is likely to influence chromatin modifications genome-wide, and thus does not enable the identification of particular regulatory elements that govern gene reactivation. We hypothesized that reactivation of maternal PWS-associated genes is mediated by genomic elements where regulatory factors dock and influence chromatin state and gene expression. Previous studies using CRISPR-based screening approaches to uncover noncoding regulatory elements often found that these elements were located in the proximity (within a megabase) of their target genes and often were annotated with canonical markers of regulatory activity, such as DNase hypersensitivity (Klann et al., Nat Biotechnol 2017; Klann et al., Curr Opin Biotechnol 2018, 52, 32-41).

The PWS region of the 151q1-13 locus was targeted with a gRNA library of 11,221 gRNAs tiled across the region to screen for regulatory elements controlling expression of PWS-associated genes. The gRNA library consisted of a high density region covering ˜300 kilobases (kb) centered at the PWS-IC in exon 1 of SNRPN and extending upstream to alternative SNPRN exons and downstream to the SNORD116 clusters. High density saturation of this region surrounding the PWS imprinting center was targeted to uncover elements that establish imprinting at early stages of differentiation but which no longer harbor canonical signatures of regulatory elements. Additional gRNAs were designed to target putative regulatory elements throughout the remaining imprinted region based on DNase hypersensitivity signal in human embryonic stem cells (Consortium, Nature 2012, 489, 57-74), H3K27Ac, a marker that is often found near regulatory elements, and CpG islands. DNase-sequencing tracks have been used previously to uncover regulatory elements with CRISPR-based screens (Klann et al., Nat Biotechnol 2017).

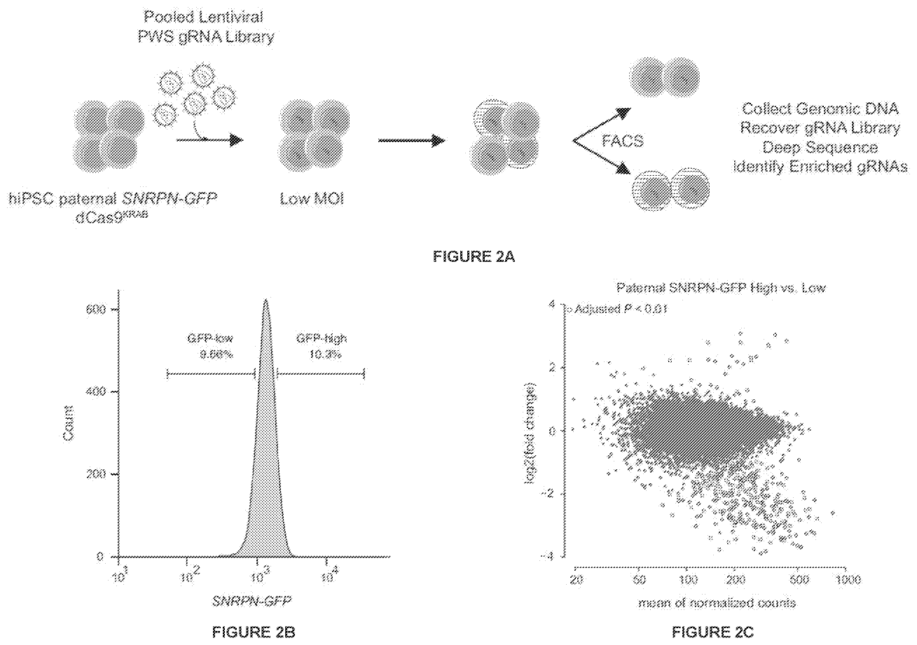

To confirm the functionality of the screen and to better understand mechanisms governing gene expression in the locus, a screen designed to repress gene expression with dCas9^{KRAB} was conducted in paternally-tagged SNPRN-GFP cells (patSNRPN-GFP) (FIG. 2A). Targeting of dCas9^{KRAB} with a set of four gRNAs to the PWS-IC in exon 1 of SNPRN had been demonstrated to be sufficient to knockdown SNRPNmRNA expression and reduce GFP signal in patSNRPN-GFP cells. A paternal screen could inform targets for the maternal allele. A polyclonal dCas9^{KRAB} patSNRPN-GFP line was transduced at low MOI with the PWS gRNA library, cultured for 9 days and sorted via FACS for the 10% highest and lowest expressing cells based on GFP fluorescence (FIG. 2A and FIG. 2B).

The screen identified many regulatory elements causing a downregulation in paternal SNPRPN expression but identified approximately 16 select guides in two regions that caused an increase in paternal SNRPN GFP expression.

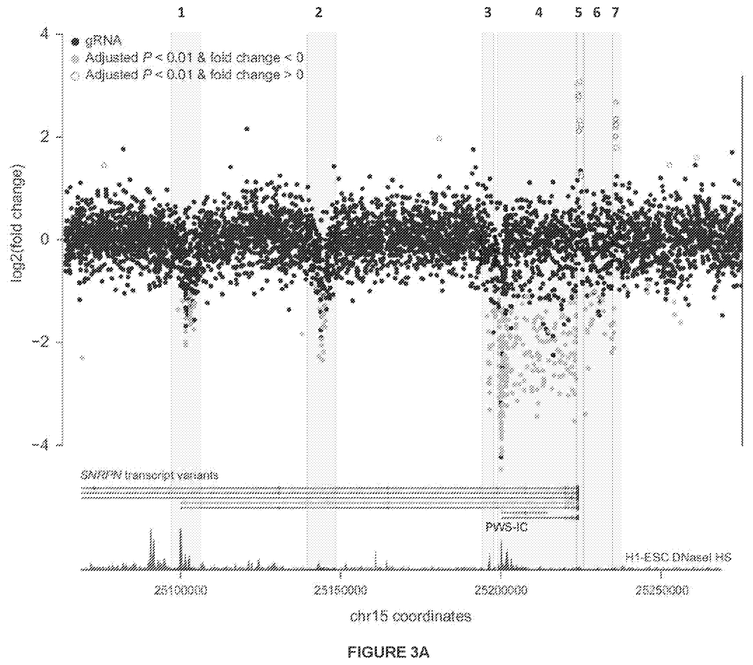

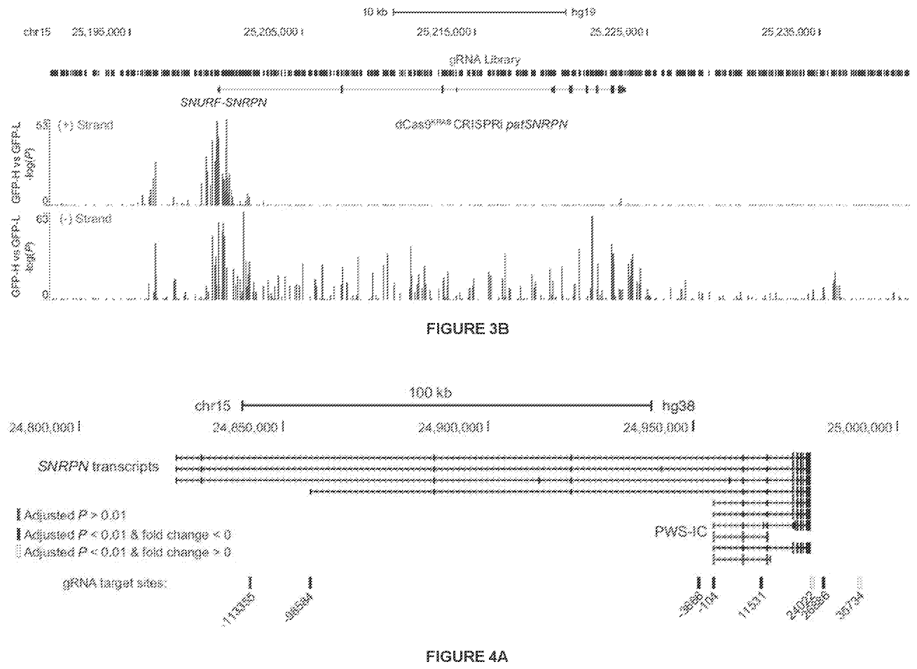

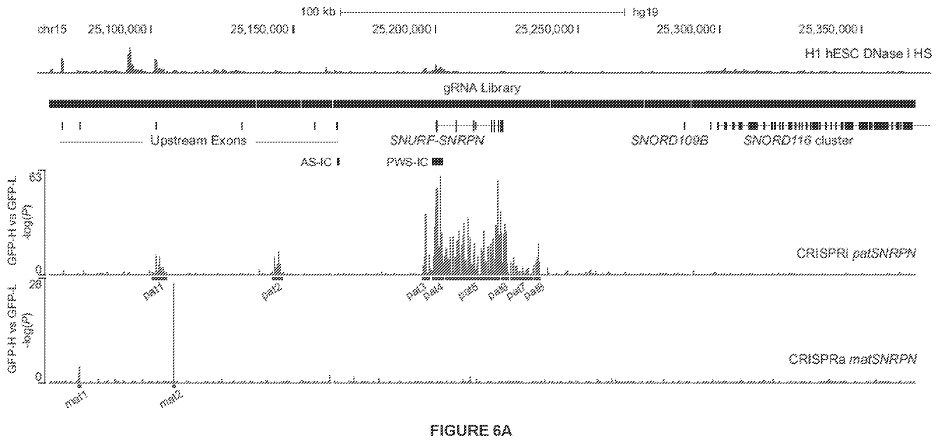

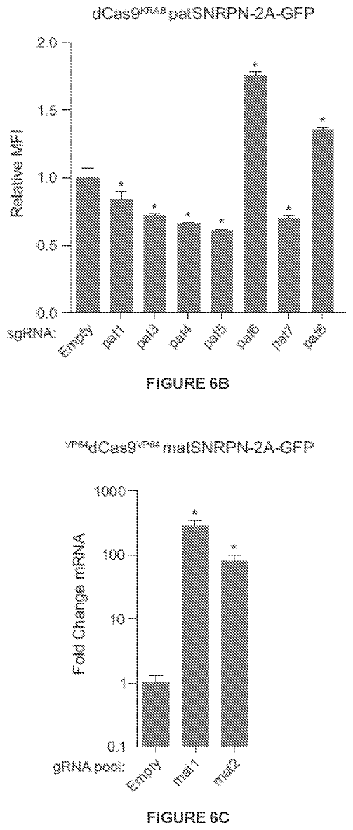

The majority of the differentially abundant gRNAs hits were enriched in the GFP-low population, in line with the expected repressive effect of dCas9^{KRAB} (FIG. 2C). However, several gRNAs were also enriched in the GFP-high cell bin (FIG. 2C). When mapped to their genomic loci, many of these differentially enriched gRNAs were located at sites outside of the PWS-IC (FIG. 3A and FIG. 3B). The majority of significantly enriched or depleted gRNAs (gRNA “hits”) targeted the ˜300 kb high-density region. See FIG. 6A. The CRISPRi screen of the paternal allele identified sites upstream, within and downstream of the PWS-IC, including throughout the gene body of SNRPN (pat1-pat8). These sites included three regions upstream of the PWS-IC, including two enhancers within SNRPN introns and one upstream alternative SNRPN exon. Surprisingly, the entire SNRPN gene body downstream of the PWS-IC contained gRNAs enriched in GFP-low expressing cells. This result contradicts previous findings that targeted repression with dCas9^{KRAB} is most effective proximal to the transcription start site, and it differs from results of another dCas9^{KRAB} screen within the gene body of MYC16. Unexpectedly, repression of two distinct sites downstream of SNRPN exons produced an increase in GFP signal indicative of upregulation of gene expression (FIG. 3A and FIG. 3B). FIG. 3A shows a map of a sub-region of 15q11-13 containing a high density of gRNAs overlayed to genomic annotations of SNRPN transcripts. Grey shaded regions (labeled 1, 2, 3, 4, and 6) contain gRNAs enriched in the GFP-low expressing cells, and white outlined regions (labeled 5 and 7) contain gRNAs enriched in the GFP-high expressing cells. Some of the identified regions align with DNase hypersensitivity sites in ESCs, while many of the sites do not harbor canonical signatures of regulatory elements. The hits within and downstream of the SNRPN gene body had a strong DNA strand bias, with almost all the hits located on the minus DNA strand and targeting the sense strand of the SNRPN gene (FIG. 3B).

Several gRNAs within two regions, pat6 and pat8, increased levels of measured GFP expression. These regions are both downstream of SNRPN exon 10, with pat6 directly adjacent to exon 10 and pat8 approximately 10 kb further downstream.

### Example 3

### Validations of gRNAs from patSNRPN-GFP Screen

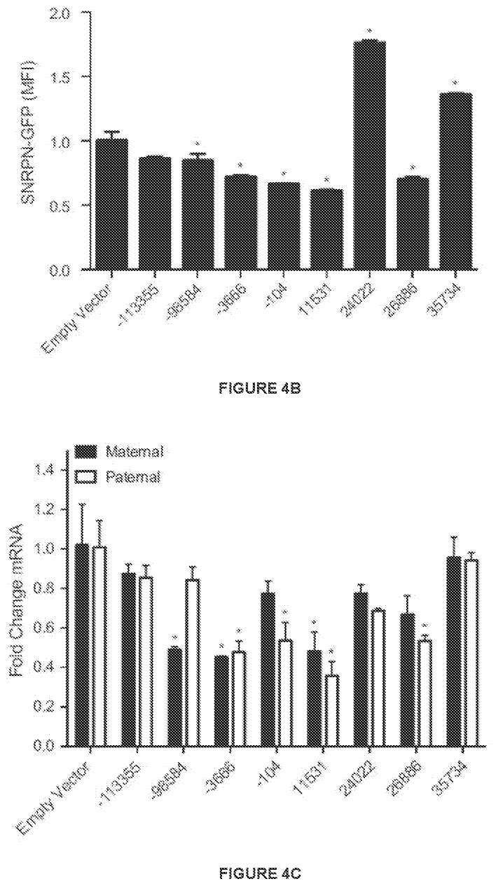

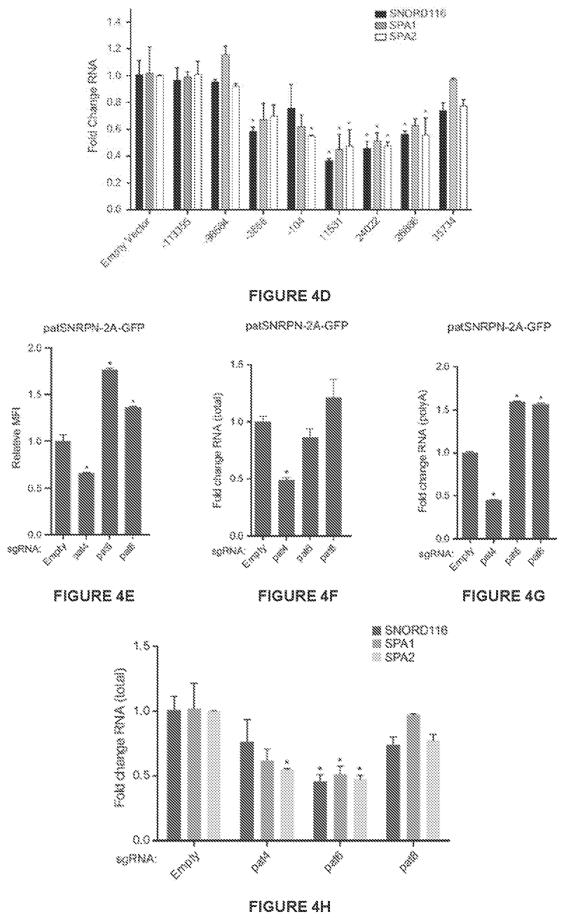

The most highly enriched gRNAs within the regions defined in FIG. 3A and FIG. 3B were selected for further evaluation (FIG. 4A). These guides targeted genomic regions around positions −113355, −98584, −3666, −104, +11531, +24022, +26886, and +35734. Of these, regions around positions +24022 and +35734 were the genomic regions that increased GFP signal (SNRPN expression). The gRNA was cloned into the appropriate expression vector described above and validations were performed similar to Example 1, except the transductions were performed in 24-well plates and the virus was delivered at high MOI. Cells were harvested for flow cytometry or qRT-PCR 5-7 d after gRNA transduction.

All of the gRNAs tested modulated the GFP reporter signal as expected, ranging from a 2-fold reduction in signal to a 2-fold increase in signal (FIG. 4B). See also FIG. 6B and FIG. 4E. However, the observed change in GFP fluorescence, reporting on the translation rate of SNRPN, did not always correlate with changes in the mRNA content as evaluated with qRT-PCR (FIG. 4C). See also FIG. 4F. Additionally, using primers that distinguish between the GFP-tagged paternal allele and the wild-type maternal allele, many of the gRNAs were observed to have an effect on both alleles (FIG. 4C). While maternal SNRPN expression is significantly dampened relative to that derived from the paternal allele due to the imprint, there is a measurable degree of leaky expression from the maternal allele that can be further repressed by several of the tested gRNAs (FIG. 4C). Expression of SPA1, SPA2 and SNORD116 was analyzed in response to the same gRNAs evaluated previously from the patSNRPN-GFP screen (FIG. 4D). See also FIG. 4H. Many of these gRNAs also repressed expression of these noncoding RNAs (FIG. 4D), indicating that expression of the noncoding SPA RNAs and snoRNAs within 151q1-13 is derived from a host transcript initiated at the PWS imprinting center and including SNRPN. Interestingly, the degree of repression of each ncRNA was similar across the three RNAs for a single gRNA. Of interest, a gRNA that increased the GFP reporter signal decreased expression of the downstream ncRNAs (FIG. 4D). This gRNA targets a region downstream of and adjacent to the last SNRPN exon.

Overall, an up-regulation in polyadenylated SNRPN with pat6 and pat8 targeting was observed (FIG. 4G) was observed as well as a down-regulation of some of the ncRNAs downstream of SNRPN, including SPA1, SPA2 and SNORD116, when targeting dCas9KRAB to either the PWS-IC, pat6 or pat8 regions (FIG. 4D and FIG. 4H).

### Example 4

### Screen of a Maternal SNRPN-GFP Cell Line with a Targeted Activator System

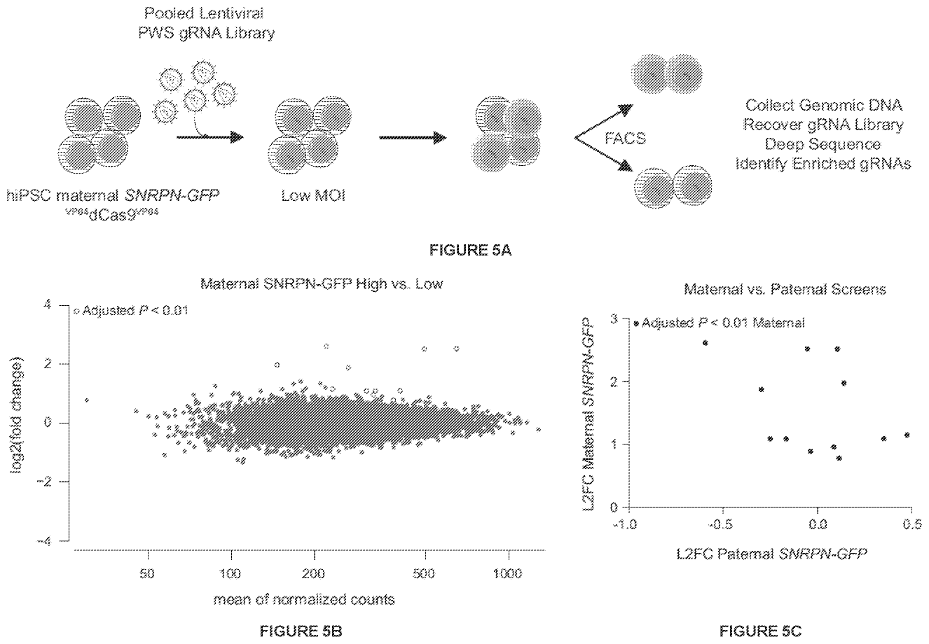

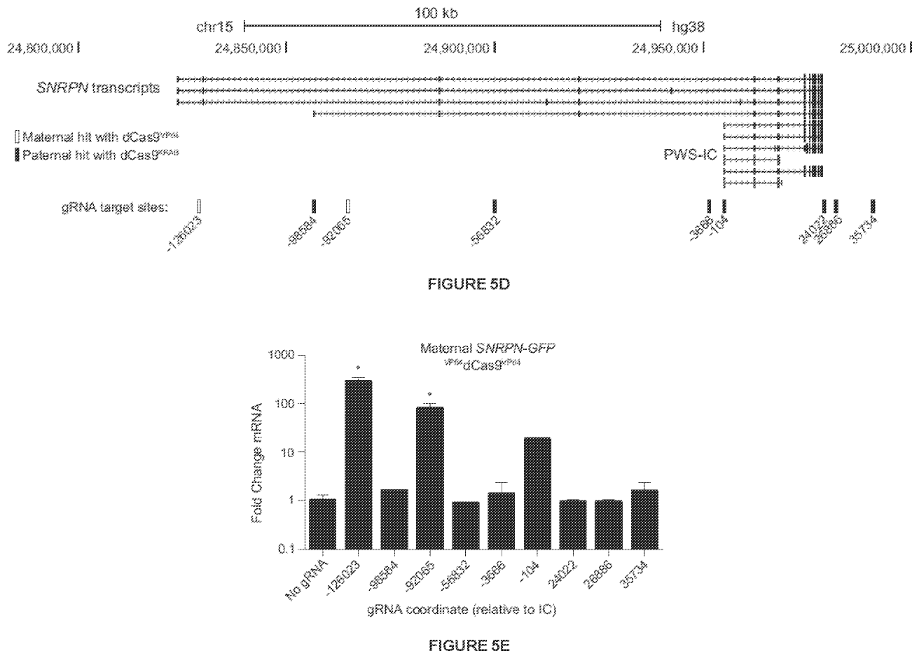

To identify regulatory elements controlling maternal SNRPN expression, the 11,221 guide RNAs described above were also used to performe a screen designed to activate gene expression in a maternally-tagged GFP cell line (matSNRPN-GFP) (FIG. 5A).

The matSNRPN-GFP cells are GFP-negative due to the maternal silencing of SNPRN. Thus, for this screen a CRISPR-based transcriptional activator (^{VP64}dCas9^{VP64}) was used to identify elements to reactivate the maternal imprint (FIG. 5A). The screen was performed in the same way as done with the CRIPSRi patSNRPN-GFP screen using the same gRNA library. A VP64dCas9VP64 matSNPRN-2A-GFP reporter cell line was transduced with the PWS pooled lentiviral gRNA library at low MOI and sorted for GFP expression via FACS. gRNA abundance in each cell bin was measured by deep sequencing and depleted or enriched gRNAs were identified by differential expression analysis. Cells were sorted via FACS for the 10% highest and lowest expressing cells based on GFP fluorescence.

The screen identified 12 gRNAs enriched in the GFP-high expressing cells, and no gRNAs were enriched in GFP-low expressing cells (FIG. 5B). Interestingly, there was no overlap in the gRNA hits between the maternal and paternal screen; the 12 gRNAs enriched in GFP-high expressing cells were not enriched in any cell bin from the paternal screen (FIG. 5C and FIG. 5D). The gRNAs from the CRISPRa screen clustered within two distinct regions (mat1 and mat2) ˜100 kb upstream of the imprinting center in the general region of annotated upstream SNRPN exons, at positions −126023 and −92065 (see FIG. 6A). Surprisingly, the imprinting center itself was not identified in this screen. When gRNAs within regions identified in either CRISPRi or CRISPRa screen were tested with ^{VP64}dCas9^{VP64} in matSNRPN-GFP cells, gRNAs targeting the imprinting center and the two regions identified from the activator screen were sufficient to up-regulate maternal SNRPN (FIG. 5E).

### Example 5

### Validations of gRNAs from matSNRPN-GFP and patSNRPN-GFP Screen in Maternal SNRPN-GFP Cell Line

The top enriched gRNAs within regions identified in either the repressor screen of paternal-tagged cell lines or the activator screen of maternal-tagged cell lines were tested individually with VP64dCas9VP64 in matSNRPN-GFP cells (FIG. 5E). Changes in the mRNA content were evaluated with qRT-PCR, and showed that gRNAs targeting the imprinting center and the two regions identified from the CRISPRa screen were sufficient to up-regulate maternal SNRPN (FIG. 5E), i.e. genomic regions around positions −126023, −92065 and −104.

Pools of three or four gRNAs were used due to the reported synergistic activity of multiple gRNAs (Maeder et al., 2013; Perez-Pinera et al., 2013). Both regions mat1 and mat2 led to significant upregulation of matSNRPN-2A-GFP as assessed by qRT-PCR (FIG. 6C).

To ensure that the lack of overlap between the maternal and paternal screens was not due to differences in sensitivity of the allele-specific GFP reporters, gRNA hits on the opposing alleles were individually tested. The majority of gRNAs tested did not influence expression of the other allele. Region pat5, which overlaps the PWS-IC, did have a minor influence (˜10-fold) on maternal SNRPN expression with VP64dCas9VP64. dCas9KRAB targeting to the mat1 region led to a slight up-regulation of paternal SNRPN.

### Example 6

### Validations of gRNAs from matSNRPN-GFP and patSNRPN-GFP Screen in PWS Patient-Derived Cells

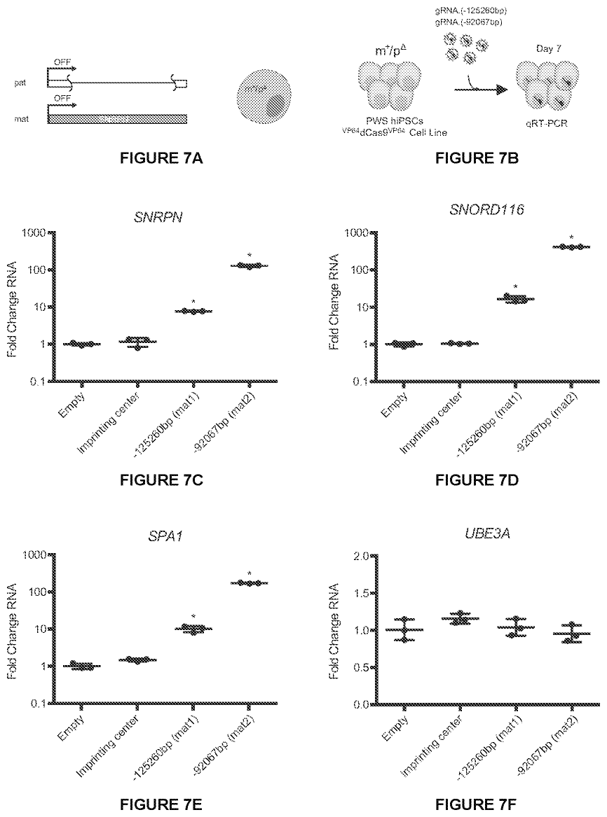

The gRNAs identified in the CRISPRa screen in matSNRPN-GFP cells (FIG. 5A-FIG. 5E), from the mat1 and mat2 regions, were tested in patient-derived iPSCs with a 15q11-13 large deletion on the paternal allele (Chamberlain et al., 2010) (FIG. 7A). The mat1 and mat2 gRNAs were first screened in matSNRPN-2A-GFP cells to find the most active single gRNA for continued analysis (FIG. 7C-7F).

Stable expression of VP64-dCas9-VP64 was established in the patient-derived cell line via lentiviral transduction, and single gRNAs targeting either mat1 or mat2 were delivered on a separate lentiviral vector. Specifically, the patient-derived cell line was cultured on matrigel-coated dishes in mTesR. A ^{VP64}dCas9^{VP64} polyclonal line was established by lentiviral transduction followed by antibiotic selection with 2 μg/mL blasticidin for five days. For testing mat1 and mat2 gRNAs in iPSCs, the ^{VP64}dCas9^{VP64} cells were transduced with the gRNA in suspension in matrigel-coated 24-well plates. The cells were fed daily with mTesR.

Cells were harvested for total RNA seven days after transduction of the gRNAs, and qRT-PCR was used to assess expression of maternal genes within 15q11-13 (FIG. 7B). Successful up-regulation of PWS-associated imprinted genes was detected, including SNRPN, SNORD116 and SPA1 in PWS patient-derived iPSCs (FIG. 7C, FIG. 7D, FIG. 7E). This activation did not cause a change in expression of UBE3A (FIG. 7F).

mRNA-sequencing of day seven cells treated with a mat2 gRNA revealed a highly-specific up-regulation of SNRPN and polyadenylated IncRNA components of the host transcript.

### Example 7

### Synergistic Activity of gRNAs Targeting Two Regions

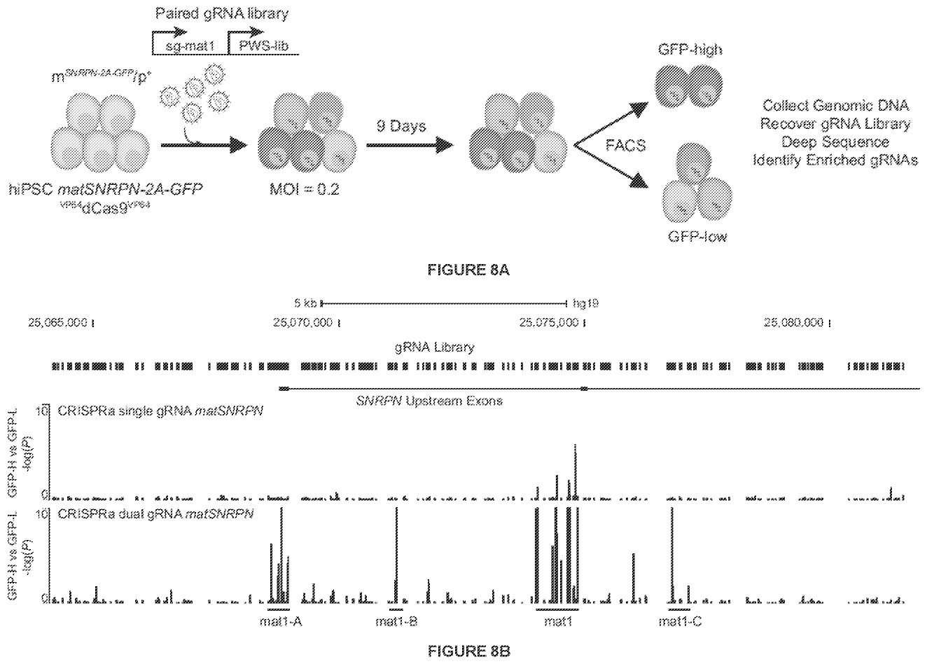

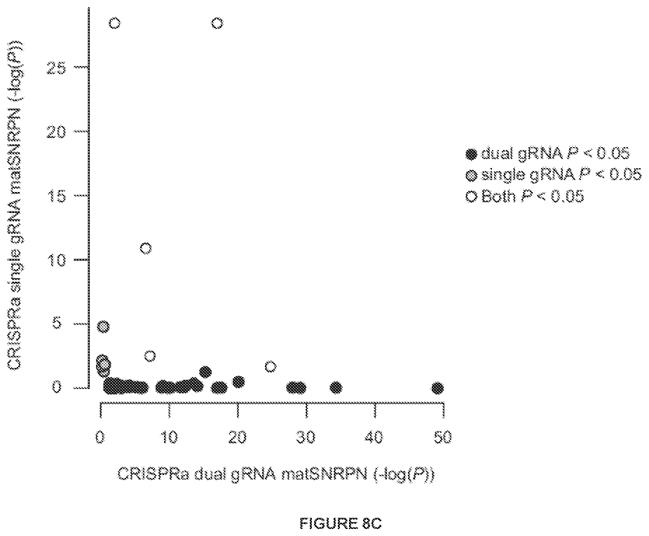

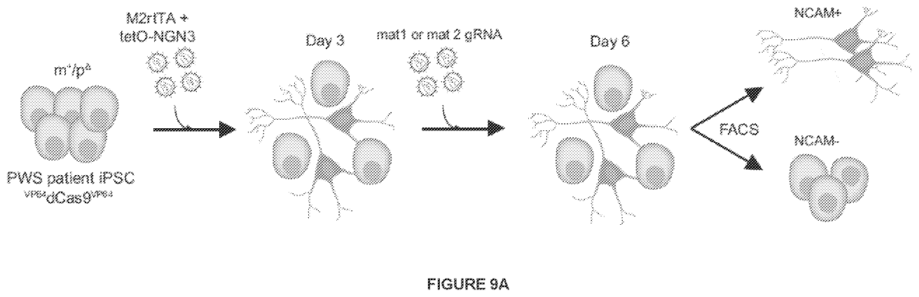

Several studies have demonstrated enhanced CRISPRa activity with the delivery of multiple gRNAs functioning synergistically (Maeder et al., 2013; Perez-Pinera et al., 2013). Furthermore, regulatory elements can function cooperatively to regulate gene expression (Li et al., 2002), and thus simultaneous targeting of these sites might be necessary to reveal their activity. An additional CRISPRa screen with VP64dCas9VP64 in matSNRPN-2A-GFP cells was performed with a dual gRNA vector, such that each cell now received a pair of gRNAs (FIG. 8A). Of the pair, one gRNA was fixed within the mat1 region (sg-mat1) and paired with a second gRNA from the remaining library. Specifically, the gRNA expression plasmid for the dual gRNA screen was generated by further modification of the single gRNA expression plasmid to contain an additional gRNA cassette expressing single-guide RNA “mat1” (comprising SEQ ID NO: 588) under control of the mU6 pollII promoter as described above. The constant region of the gRNA included the sequence of SEQ ID NO: 1141 (RNA), which was encoded by a sequence comprising SEQ ID NO: 1140 (DNA). The sequence of the full gRNA corresponding to SEQ ID NO: 588 is SEQ ID NO: 1142.

The screening protocol was as described above in Examples 1 and 2. As noted above, both mat1 and mat2 regions led to significant up-regulation of matSNRPN-2A-GFP assessed by qRT-PCR (FIG. 6C). The dual gRNA screen revealed several additional gRNAs and regulatory regions that were not identified in the single gRNA screen, including a region ˜5 kb upstream from mat1 and adjacent to an annotated SNRPN exon (FIG. 8B). The most significantly enriched gRNAs in the single gRNA screen were also identified in the dual screen, with the addition of several more strongly enriched gRNAs unique to the dual screen (FIG. 8C).

### Example 8

### Activation of PWS-Associated Genes in PWS Patient Neurons

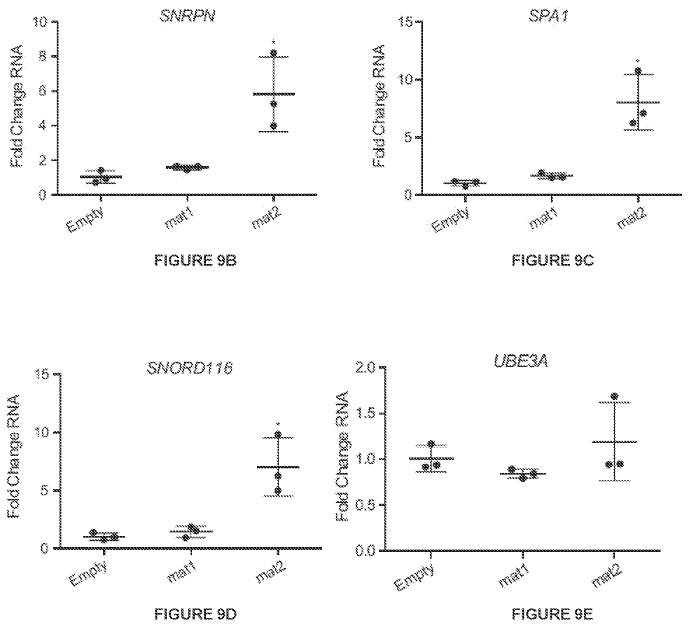

Since the brain is a primary organ involved in the etiology of PWS, CRISPRa activity at mat1 and mat2 sites was assessed in neurons derived from patient iPSCs. The iPSCs in Example 7 that stably expressed ^{VP64}dCas9^{VP64} were differentiated into a neuronal lineage through overexpression of the cDNA encoding NEUROG3 (FIG. 9A).

Specifically, the ^{VP64}dCas9^{VP64} iPSCs were co-transduced with lentiviral vectors encoding TetO-NEUROG3 and M2rtTA (Addgene #20342) in mTesR supplemented with 10 μM Rock Inhibitor. 18-20 hours after transduction, the medium was changed to N3 neurogenic medium (DMEM/F-12 Nutrient Mix (Gibco, 11320), 1× B-27 serum-free supplement (Gibco, 17504), 1×N-2 supplement (Gibco, 17502), and 25 μg/mL gentamicin (Sigma, G1397)) supplemented with 0.1 μg/mL doxycycline (Sigma, D9891). Three days after doxycycline addition, lentivirus encoding either sg-mat1 (comprising SEQ ID NO: 588) or sg-mat2 (comprising SEQ ID NO: 589) was added directly into the media with passaging the cells. The constant region of the gRNA included the sequence of SEQ ID NO: 1141 (RNA), which was encoded by a sequence comprising SEQ ID NO: 1140 (DNA). The sequence of the full gRNA corresponding to SEQ ID NO: 588 is SEQ ID NO: 1142. Three days after the addition of the gRNA virus, cells were dissociated and sorted for NCAM expression and harvested for total RNA.

The gRNAs were delivered on a separate lentiviral vector three days after the onset of differentiation to ensure that the CRISPRa activity initiated in the neurons and not in the iPSCs. Neurons were purified from undifferentiated cells via NCAM staining on day six of differentiation and harvested for RNA. For NCAM staining, cells were dissociated with Accutase (Stemcell Tech, 7920), centrifuged at 300 g for 5 min, and resuspended in staining buffer (0.5% BSA (Sigma, A7906) and 2 mM EDTA (Sigma, E7889) in PBS). Mouse anti-CD56 (NCAM, Invitrogen, 12-0567) was added at 0.6 μg per 1×10⁶ cells and incubated for 30 min at 4° C. Cells were washed with 1 mL staining buffer, centrifuged at 300 g for 5 min and resuspended in staining buffer for sorting on SH800 FACS Cell Sorter (Sony Biotechnology).

An up-regulation of SNRPN and downstream ncRNAs was detected only with the mat2 gRNA (FIG. 9B, FIG. 9C, and FIG. 9D). UBE3A expression was not changed with either gRNA (FIG. 9E). Two imprinted genes within 15q11-13 expressed primarily in neurons, MAGEL2 and NDN, were not detected in any condition.

### Example 9

### Targeted Epigenetic Editing with a DNA Targeting System that Demethylates DNA

Further screening of the 15q11-13 PWS-associated locus to identify genomic regulatory elements controlling expression of PWS-associated genes was performed with a CRISPR/dCas9-targeted demethylase. A dCas9-based epigenetic modifier fused to the catalytic domain of Ten-eleven translocation methylcytosine dioxygenase 1 (Tet1CDdCas9) was used to catalyze DNA demethylation at the target locus and assess the role of DNA methylation in the maintenance of the maternal imprint. This fusion (SEQ ID NO: 89) comprises Tet1CD at its N-terminus and dCas9 at its C-terminus and has been shown to demethylate DNA in a targeted manner and induce changes in gene expression (Josipovic et al., 2019; Liu et al., 2016).

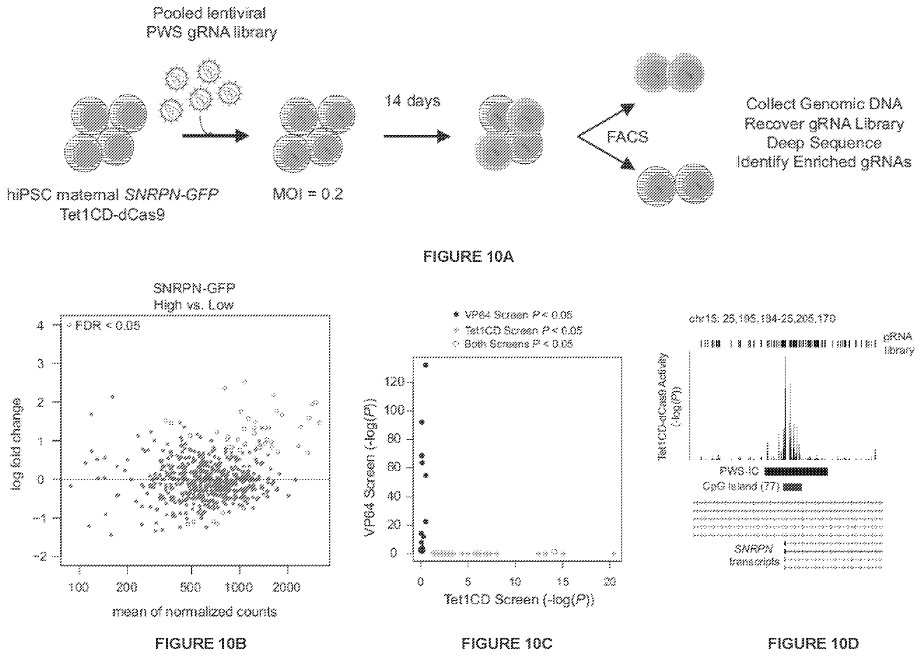

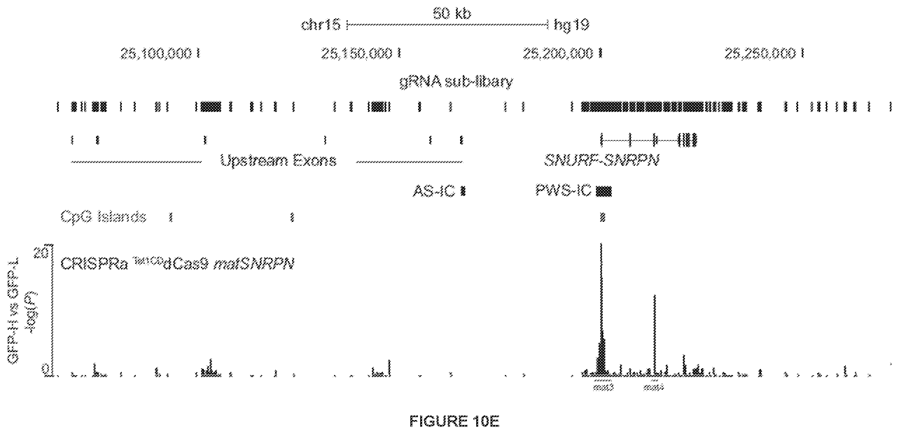

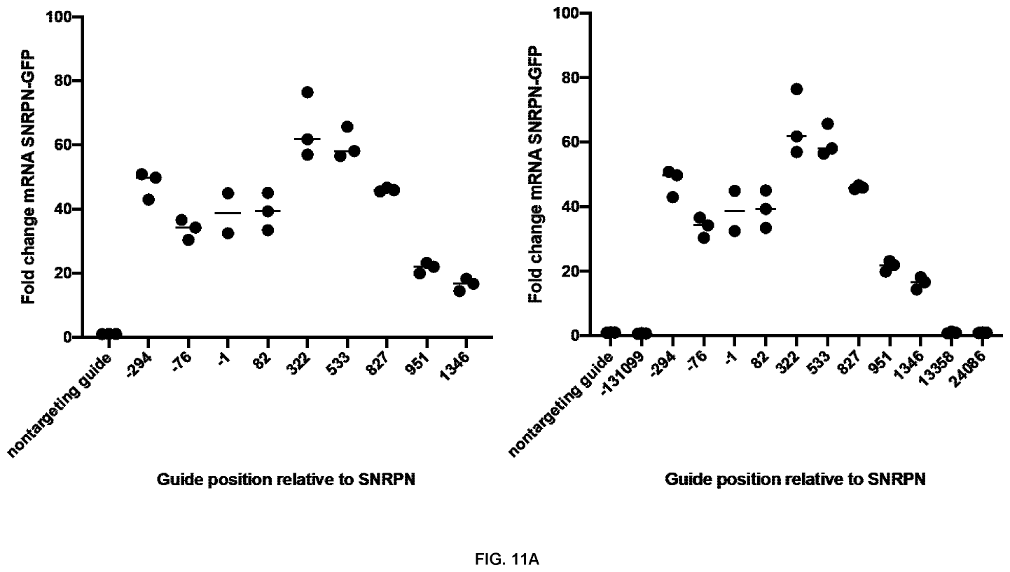

A gRNA sub-library was designed consisting of all of the hits from the three screens performed previously, totaling 583 gRNAs including 50 scrambled non-targeting controls. The guides were used in a pooled screen and expression of matSNRPN-2A-GFP was measured as described in Examples 1 and 2 to identify target regions within the PWS Imprinting Center that reactivate the SNRPN host transcript upon DNA demethylation (FIG. 10A).

The matSNPRN-2A-GFP reporter cell line described above in Example 1 was transduced with the pooled lentiviral gRNA library at low MOI (MOI=0.2) and sorted for GFP expression via FACS at Day 14. Specifically, the screen was performed in triplicate with independent transductions. For each replicate, 1.7×10⁶ matSNRPN-2A-GFP ^{Tet1CD}dCas9 iPSCs were dissociated using Accutase (Stemcell Tech, 7920) and transduced in suspension in a matrigel-coated 10-cm dish in mTesR (Stemcell Tech 85850) supplemented with 10 μM Rock Inhibitor (Y-27632, Stemcell Tech, 72304). Cells were transduced at a MOI of 0.2 to obtain one gRNA per cell and ˜580-fold coverage of the gRNA sub-library. The medium was changed to fresh mTesR without Rock Inhibitor 18-20 h after transduction. Antibiotic selection was started 30 h after transduction by adding 1 μg/mL puromycin (Sigma, P8833) directly to the plates without changing the medium. The cells were fed daily and passaged as necessary maintaining library coverage until harvest.

Cells were harvested for sorting 14 d after transduction of the gRNA sub-library. Cells were washed once with 1×PBS, dissociated using Accutase, filtered through a 30 μm CellTrics filter (Sysmex, 04-004-2326) and resuspended in FACS Buffer (0.5% BSA (Sigma, A7906), 2 mM EDTA (Sigma, E7889) in PBS). Before sorting, an aliquot of 0.4×10⁰ cells were taken to represent a bulk unsorted population. The highest 10% and lowest 10% of cells were sorted by FACS into GFP-High and GFP-Low bins, respectively based on GFP expression. About 0.4×10⁸ cells were sorted into each bin. Sorting was done with a SH800 FACS Cell Sorter (Sony Biotechnology). After sorting, genomic DNA was harvested with the DNeasy Blood and Tissue Kit (Qiagen, 69506).

gRNA abundance in each cell bin was measured by deep sequencing and depleted or enriched gRNAs were identified by differential expression analysis (FIG. 10B). The differential expression of normalized gRNA counts between the GFP-High and GFP-Low cell populations were analyzed. Open circle data points in FIG. 10B indicated FDR<0.05 by differential DESeq2 analysis. The reactivation of SNRPN-GFP by utilization of Tet1CD-dCas9 in comparison to dCas9-VP64 was also analyzed on regions within the PWS Imprinting Center with Tet1CD-dCas9 activity. The screen with Tet1CDdCas9 identified new gRNAs and putative regulatory regions not detected with VP64dCas9VP64 (FIG. 10C). The target sites identified in FIG. 10D and FIG. 10E provide a unique set of genomic targets that are independent of sites found previously to activate SNRPN with the dCas9-VP64 transactivator. New clusters of significantly enriched gRNAs were identified at the PWS-IC overlapping a CpG island within SNRPN exon one (mat3), as well as within a region ˜13 kb downstream of the PWS-IC and adjacent to SNRPN exon three (mat4) (FIG. 10E).

The data show that use of TET1CD-dCas9 was sufficient for reactivation of SNRPN transcript expression, including in regions where targeting using VP64-dCas9-VP64 was insufficient to reactivate SNPRN transcript expression. This targeted DNA demethylation through activation of maternal gene expression allows for a new therapeutic strategy in the treatment of PWS patients.

### Example 10

### Validation of Individual Enriched gRNAs from the dCas9Tet1c matSNRPN-2A-GFP CRISPRa Screen

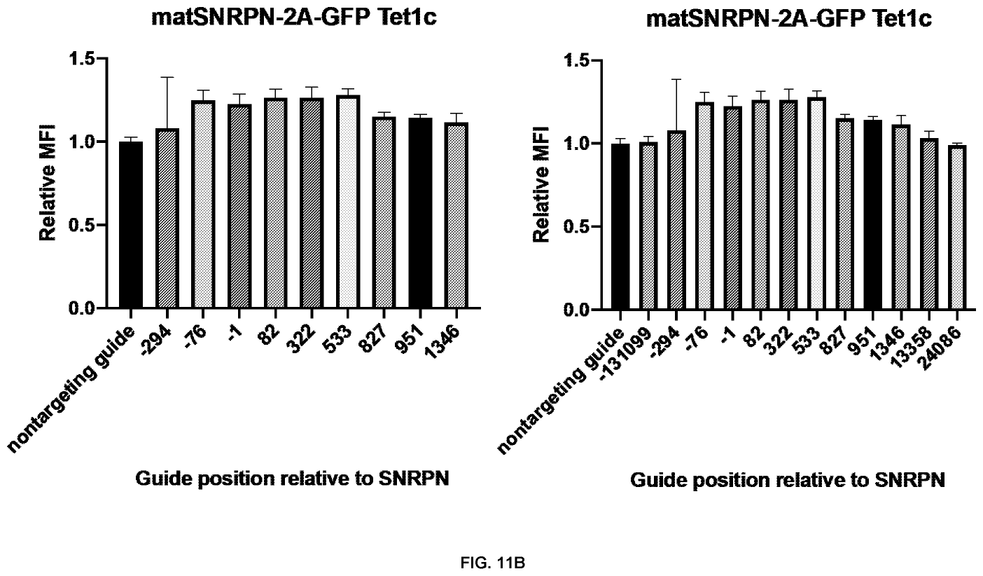

gRNAs identified from the demethylase screen described in Example 9 were further examined. A human induced pluripotent stem cell line, modified with a GFP reporter gene downstream of the SNRPN coding sequence on the maternal allele (matSNRPN-2A-GFP) as described in Example 9, was engineered to stably express dCas9-Tet1c and then transduced with lentiviral vectors encoding a single gRNA targeting the PWS locus at the indicated position. SNRPN-GFP transcript levels were quantified by RT-qPCR at nine days post-transduction, relative to non-targeting gRNA control. Results are shown in FIG. 11A. Cells were also harvested nine days post-transduction and analyzed by flow cytometry. GFP expression of the transduced cell population was quantified by mean fluorescence intensity (MFI) of the FITC channel. Results are shown in FIG. 11B. The gRNAs tested corresponded to SEQ ID NOs: 591, 585, 685, 697, 750, 752, 763, 771, 196, 812, 861, and 1069, with protospacers corresponding to SEQ ID NOs: 47, 49, 53, 55, 64, 65, 68, 70, 196, 291, 335, and 528. The gRNAs showing greatest activity corresponded to SEQ ID NOs: 685, 697, 750, 752, 763, 771, 196, 812, and 861, with protospacers corresponding to SEQ ID NOs: 53, 55, 64, 65, 68, 70, 196, 291, and 335. The gRNAs examined correspond to the protospacers and positions shown in TABLE 4.

TABLE 4
Examples of regions of DNA targeted in the demethylase screen.

| | | | relative | |
|---|---|---|---|---|
| | | | position to | |
| | | | SNRPN | |
| | | | exon 1 | |
| | | | start: | |
| gRNA name | Protospacer sequence | chr15 | (25200069) | gRNA sequence |
| PWS_G.2979 | CCCTCAGGTCTTCCTATGTG | 25199775 | −294 | CCCUCAGGUCUUCCUAUGUG |
| | (SEQ ID NO: 55) | | | (SEQ ID NO: 697) |
| PWS_G.4670 | GCAGGCTGGCGCGCATGCTC | 25200068 | −1 | GCAGGCUGGCGCGCAUGCUC |
| | (SEQ ID NO: 68) | | | (SEQ ID NO: 763) |
| PWS_G.4521 | GATGCGTCAGGCATCTCCGG | 25200151 | 82 | GAUGCGUCAGGCAUCUCCGG |
| | (SEQ ID NO: 65) | | | (SEQ ID NO: 752) |
| PWS_G.4363 | GAGCGGACAGGATACCATCG | 25200391 | 322 | GAGCGGACAGGAUACCAUCG |
| | (SEQ ID NO: 64) | | | (SEQ ID NO: 750) |
| PWS_G.4903 | GCGGCGACAGTGGGTATTGG | 25200896 | 827 | GCGGCGACAGUGGGUAUUGG |
| | (SEQ ID NO: 70) | | | (SEQ ID NO: 771) |
| PWS_G.81 | AAAGCATGCGCTACAATAAC | 25213427 | 13358 | AAAGCAUGCGCUACAAUAAC |
| | (SEQ ID NO: 47) | | | (SEQ ID NO: 591) |
| PWS_G.648 | ACATCCTCTATTCTGATCAT | 25224155 | 24086 | ACAUCCUCUAUUCUGAUCAU |
| | (SEQ ID NO: 49) | | | (SEQ ID NO: 585) |
| PWS_G.306 | AAGCAATATGAAATGTTACC | 25068970 | −131099 | AAGCAAUAUGAAAUGUUACC |
| | (SEQ ID NO: 528) | | | (SEQ ID NO: 1069) |
| PWS_G.6064 | GTGACGCAACACAGACCCCC | 25201415 | 1346 | GUGACGCAACACAGACCCCC |
| | (SEQ ID NO: 291) | | | (SEQ ID NO: 812) |
| PWS_G.2815 | CCAGGTCATTCCGGTGAGGG | 25199993 | −76 | CCAGGUCAUUCCGGUGAGGG |
| | (SEQ ID NO: 53) | | | (SEQ ID NO: 685) |
| PWS_G.7410 | TGCATAGGGATTTTAGGCGG | 25200602 | 533 | UGCAUAGGGAUUUUAGGCGG |
| | (SEQ ID NO: 335) | | | (SEQ ID NO: 861) |
| PWS_G.3052 | CCGGACAGCGACAGGCCCCG | 25201020 | 951 | CCGGACAGCGACAGGCCCCG |
| | (SEQ ID NO: 196) | | | (SEQ ID NO: 196) |

The foregoing description of the specific aspects will so fully reveal the general nature of the invention that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects but should be defined only in accordance with the following claims and their equivalents.

All publications, patents, patent applications, and/or other documents cited in this application are incorporated by reference in their entirety for all purposes to the same extent as if each individual publication, patent, patent application, and/or other document were individually indicated to be incorporated by reference for all purposes.

For reasons of completeness, various aspects of the invention are set out in the following numbered clauses:

Clause 1. A method of treating a subject having Prader Willi Syndrome (PWS) or Prader-Willi-like disorder comprising administering to the subject a DNA Targeting System that targets the 15q11-13 PWS-associated locus.

Clause 2. The method of clause 1, wherein the DNA Targeting System is a Targeted Activator System that binds to a target region selected from the group consisting of: nucleotide positions −127023 to −125023; nucleotide positions −93065 to −91065; and nucleotide positions −1104 to +896, wherein the nucleotide position is relative to position 1 being the start site of SNRPN exon 1 of the PWS imprinting center on chromosome 15.

Clause 3. The method of clause 1, wherein the subject is administered a Targeted Activator System that binds to a target region selected from the group consisting of: nucleotide positions −126523 to −125523; nucleotide positions −92565 to −91565; and nucleotide positions −604 to +395; wherein the nucleotide position is relative to position 1 being the start site of SNRPN exon 1 of the PWS imprinting center on chromosome 15.

Clause 4. The method of any of clauses 1-3, wherein the Targeted Activator System targets a target region that results in increased expression of SNORD116 or its products.

Clause 5. The method of any of clauses 1-3, wherein the Targeted Activator System targets a target region that results in increased expression of MAGEL2.

Clause 6. The method of any of clauses 1-3, wherein at least a first Targeted Activator System targets a target region that results in increased expression of SNORD116 and at least a second Targeted Activator System targets a region that results in increased expression of MAGEL2.

Clause 7. The method of clause 1, wherein DNA Targeting System is a Targeted Repressor System that binds to a target region selected from the group consisting of: nucleotide positions +23022 to +25022; and nucleotide positions +34734 to +36734, wherein the nucleotide position is relative to position 1 being the start site of SNRPN exon 1 of the PWS imprinting center on chromosome 15.

Clause 8. The method of clause 1, wherein the subject is administered a Targeted Repressor System that binds to a target region selected from the group consisting of: nucleotide positions +23522 to +24522; and nucleotide positions +35234 to +36234, wherein the nucleotide position is relative to position 1 being the start site of SNRPN exon 1 of the PWS imprinting center on chromosome 15.

Clause 9. The method of any of clauses 1-8, wherein the administering to said subject results in increased expression of one or more of the following gene or gene products: MKRN3, MAGEL2, NDN, C15ORF2, SNURF-SNRPN, SNORD107, SNORD64, SNORD109A, SNORD116, SNORD116@, SPA1, SPA2, 116HG, SNORD116-1 to 30, Sno-Inc RNA 1 to 5, IPW, SNORD115, SNORD115@, 115HG, SNORD115-1 to 48, SNORD109B, and/or SNG14.

Clause 10. The method of any of clauses 1-9, wherein the subject has a PWS Type 1 large deletion, PWS Type 2 large deletion, PWS imprinting center mutation or PWS uniparental disomy.

Clause 11. The method of any of clauses 1-9, wherein the subject has a PWS microdeletion encompassing SNORD116, but not MAGEL2.

Clause 12. The method of any of clauses 1-9, wherein the subject has a PWS or PWS-like atypical deletion encompassing MAGEL2, but not SNORD116.

Clause 13. The method of any of clauses 1-9, wherein the subject has heterozygous Schaaf-Yang syndrome or MAGEL2 disorder.

Clause 14. The method of any of clauses 1−13, wherein the Targeted Activator System is a CRISPR-Cas Type II system.

Clause 15. The method of clause 14, wherein the Targeted Activator System comprises: (a) a fusion protein comprising: (i) a Cas9 polypeptide with reduced nuclease activity, and (ii) an activator; and (b) one or more guide RNAs (gRNA) that bind to a target region in the 15q11-13 PWS-associated locus.

Clause 16. The method of clause 15, wherein at least one gRNA comprises at least 10, 11, 12, 13, 14, 15, 16, 17, 18, 19 or 20 consecutive nucleotides of SEQ ID NOs: 1-12, or a corresponding allelic variant thereof.

Clause 17. The method of clause 15 wherein the Cas9 is a S. pyogenes Cas9 (SEQ ID NO: 24).

Clause 18. The method of clause 15, wherein the subject is administered: (a) said fusion protein and said one or more gRNAs; or (b) a nucleic acid sequence encoding said fusion protein, and said one or more gRNAs; or (c) a nucleic acid sequence encoding said fusion protein, and nucleic acid sequence(s) encoding said one or more gRNAs.

Clause 19. The method of clause 18, wherein the subject is administered a vector comprising a nucleic acid encoding said fusion protein and/or a vector comprising a nucleic acid encoding said gRNA.

Clause 20. The method of clause 19 wherein the vector is a viral vector, optionally a retroviral, lentiviral, adenovirus, adeno-associated virus vector, synthetic vector, or is a vector within a lipid nanoparticle.

Clause 21. The method of clause 14, wherein the Targeted Activator System comprises (a) a Cas9 polypeptide, and (b) one or more dead gRNAs that bind to a target region in the 15q1-13 PWS-associated locus.

Clause 22. The method of any of clauses 15-20, wherein the Targeted Activator System is a CRISPR-Cas Type II system comprising one or more gRNAs, wherein at least one gRNA comprises a nucleotide sequence at least 80% complementary to at least 10-20 consecutive nucleotides of said target region.

Clause 23. The method of clause 22, wherein the at least one gRNA comprises a nucleotide sequence at least 80% complementary to at least 18 consecutive nucleotides of said target region.

Clause 24. The method any of clauses 1-13, wherein the Targeted Activator System is a ZF-based and/or TALE-based system.

Clause 25. The method of clause 24, wherein the Targeted Activator System comprises one or more fusion proteins comprising (a) a TALE that binds to the target region of the 15q11-13 PWS-associated locus and (b) an activator.

Clause 26. The method of clause 24, wherein the Targeted Activator System comprises one or more fusion proteins comprising (a) a ZF that binds to the target region of the 15q11-13 PWS-associated locus and (b) an activator.

Clause 27. The method of any of clauses 15-26 wherein the activator is VP64, VP16; GAL4; p65 subdomain (NFkB); KMT2 family transcriptional activators: hSET1A, hSET1B, MLL1 to 5, ASH1, and homologs (Trx, Trr, Ash1); KMT3 family: SYMD2, NSD1; KMT4 family: DOT1L and homologs; KDM1: LSD1/BHC110 and homologs (SpLsd1/Swm1/Saf110, Su(var)3-3); KDM3 family: JHDM2a/b; KDM4 family: JMJD2A/JHDM3A, JMJD2B, JMJD2C/GASC1, JMJD2D, and homologs (Rph1); KDM6 family: UTX, JMJD3, VP64-p65-Rta (VPR); synergistic action mediator (SAM); p300; VP160; VP64-dCas9-BFP-VP64; KAT2 family: hGCN5, PCAF, and homologs (dGCN5/PCAF, Gcn5; KAT3 family: CBP, p300 and homologs (dCBP/NEJ); KAT4: TAF1 and homologs (dTAF1); KAT5: TIP60/PLIP, and homologs; KAT6: MOZ/MYST3, MORF/MYST4, and homologs (Mst2, Sas3, CG1894); KAT7: HBO1/MYST2, and homologs (CHM, Mst2); KAT8: HMOF/MYST1, and homologs (dMOF, CG1894, Sas2, Mst2); KAT13 family: SRC1, ACTR, P160, CLOCK, and homologs; AID/Apobed deaminase family: AID; TET dioxygenase family: TET1; DEMETER glycosylase family: DME, DML1, DML2, or ROS1.

Clause 28. The method of any of clauses 1−13, wherein the Targeted Repressor System is a CRISPR-Cas Type II system.

Clause 29. The method of clause 28, wherein the Targeted Repressor System comprises (a) a fusion protein comprising: (i) a Cas9 polypeptide with reduced nuclease activity, and (ii) a repressor; and (b) one or more guide RNAs (gRNA) that bind to the target region of the 15q11-13 PWS-associated locus.

Clause 30. The method of clause 29 wherein the Cas9 is a S. pyogenes Cas9 (SEQ ID NO: 24).

Clause 31. The method of clause 29, wherein the subject is administered: (a) said fusion protein and said one or more gRNAs; or (b) a nucleic acid sequence encoding said fusion protein, and said one or more gRNAs; or (c) a nucleic acid sequence encoding said fusion protein, and nucleic acid sequence(s) encoding said one or more gRNAs.

Clause 32. The method of clause 31, wherein the subject is administered a vector comprising a nucleic acid encoding said fusion protein and/or a vector comprising a nucleic acid encoding said gRNA.

Clause 33. The method of clause 32 wherein the vector is a viral vector, optionally a retroviral, lentiviral, adenovirus, adeno-associated virus vector, synthetic vector, or is a vector within a lipid nanoparticle.

Clause 34. The method of clause 28, wherein the Targeted Repressor System comprises (a) a Cas9 polypeptide with reduced nuclease activity, and (b) one or more gRNAs that bind to the target region of the 15q11-13 PWS-associated locus.

Clause 35. The method of any of clauses 28-34, wherein the Targeted Repressor System is a CRISPR-Cas Type II system comprising one or more gRNAs, wherein at least one gRNA comprises a nucleotide sequence at least 80% complementary to at least 10-20 consecutive nucleotides of said target region.

Clause 36. The method of clause 35, wherein the at least one gRNA comprises a nucleotide sequence at least 80% complementary to at least 18 consecutive nucleotides of said target region.

Clause 37. The method any of clauses 1-13, wherein the Targeted Repressor System is a ZF-based and/or TALE-based system.

Clause 38. The method of clause 37, wherein the Targeted Repressor System comprises one or more fusion proteins comprising (a) a TALE that binds to the target region of the 15q11-13 PWS-associated locus and (b) a repressor.

Clause 39. The method of clause 37, wherein the Targeted Repressor System comprises one or more fusion proteins comprising (a) a ZF that binds to the target region of the 15q11-13 PWS-associated locus and (b) a repressor.

Clause 40. The method of any of clauses 29-39, wherein the repressor is KRAB, Mad mSIN3 interaction domain (SID); the ERF repressor domain (ERD); KMT1 family: SUV39H1, SUV39H2, G9A, ESET/SETBD1, and homologs (Cir4, Su(var)3-9); KMT5 family: Pr-SET7/8, SUV4-20H1, and homologs (PR-set7, Suv4-20, and Set9); KMT6: EZH2, KMT8: RIZ1, KDM4 family: JMJD2A/JHDM3A, JMJD2B, JMJ2D2C/GASC1, JMJD2D, and homologs (Rph1); KDM5 family JARID1A/RBP2, JARID1B/PLU-1, JARID1C/SMCX, JARID1D/SMCY, and homologs (Lid, Jhn2, Jmj2); HDAC1, HDAC2, HDAC3, HDAC8, and its homologs (Rpd3, Hos1, Cir6); HDAC4, HDAC5, HDAC7, HDAC9, and its homologs (Hda1, Cir3); SIRT1, SIRT2, and its homologs (Sir2, Hst1, Hst2, Hst3, and Hst4); HDAC11, DNMT1, DNMT3a/3b, MET1, DRM3, and homologs, ZMET2, CMT1, CMT2, Laminin A, Laminin B, or CTCF.

Clause 41. A DNA Targeting System that binds to a target region selected from the group consisting of: nucleotide positions −127023 to −125023; nucleotide positions −93065 to −91065; and nucleotide positions −1104 to +896; wherein the nucleotide position is relative to position 1 being the start site of SNRPN exon 1 of the PWS imprinting center on chromosome 15.

Clause 42. A DNA Targeting System that binds to a target region selected from the group consisting of: nucleotide positions −126523 to −125523; nucleotide positions −92565 to −91565: and nucleotide positions −604 to +395; wherein the nucleotide position is relative to position 1 being the start site of SNRPN exon 1 of the PWS imprinting center on chromosome 15.

Clause 43. The DNA Targeting System of clause 41 or 42 that is a Targeted Activator System.

Clause 44. A DNA Targeting System that binds to a target region selected from the group consisting of: nucleotide positions +23022 to +25022; and nucleotide positions +34734 to +36734, wherein the nucleotide position is relative to position 1 being the start site of SNRPN exon 1 of the PWS imprinting center on chromosome 15.

Clause 45. A DNA Targeting System that binds to a target region selected from the group consisting of: nucleotide positions +23522 to +24522; and nucleotide positions +35234 to +36234 wherein the nucleotide position is relative to position 1 being the start site of SNRPN exon 1 of the PWS imprinting center on chromosome 15.

Clause 46. The DNA Targeting System of clauses 44 or 45 that is a Targeted Repressor System.

Clause 47. The DNA Targeting System of any of clauses 41-46 that is a CRISPR-Cas Type II system comprising one or more gRNAs, wherein at least one gRNA comprises a nucleotide sequence at least 80% complementary to at least 10-20 consecutive nucleotides of said target region.

Clause 48. A nucleic acid encoding at least one component of said DNA Targeting System.

Clause 49. A vector comprising the nucleic acid of clause 48.

Clause 50. A first nucleic acid encoding at least one gRNA comprises a nucleotide sequence at least 80% complementary to at least 10-20 consecutive nucleotides of said target region, and a second nucleic acid encoding a Cas9 protein, or a Cas9 fusion protein, wherein the first and second nucleic acids are on the same or different vectors.

Clause 51. The vector of clause 49 wherein the vector is a viral vector, optionally a retroviral, lentiviral, adenovirus, adeno-associated virus vector, synthetic vector, or is a vector within a lipid nanoparticle.

Clause 52. A ribonucleoprotein comprising a Cas9 protein, or a Cas9 fusion protein, and at least one gRNA comprises a nucleotide sequence at least 80% complementary to at least 10-20 consecutive nucleotides of said target region.

Clause 53. A pharmaceutical composition comprising said DNA Targeting System of any of clauses 41-47, said nucleic acids of clause 48 or 50, said vector of clause 49 or 51, or said ribonucleoprotein of clause 52.

Clause 54. A guide RNA (gRNA) comprising a polynucleotide sequence corresponding to at least one of SEQ ID NOs: 1-12, or an allelic variant thereof.

Clause 55. A DNA Targeting System that binds to a regulatory element of a gene within the 15q11-13 locus, the DNA Targeting System comprising at least one gRNA that binds and targets a polynucleotide sequence comprising a nucleotide sequence corresponding to a complement of at least one of SEQ ID NOs: 1-12, or an allelic variant thereof.

Clause 56. The DNA Targeting System of clause 55, wherein the at least one gRNA comprises a polynucleotide sequence corresponding to at least one of SEQ ID NOs: 1-12, or a variant thereof.

Clause 57. The DNA Targeting System of any one of clauses 55-56, further comprising a Clustered Regularly Interspaced Short Palindromic Repeats associated (Cas) protein or a fusion protein, wherein the fusion protein comprises two heterologous polypeptide domains, wherein the first polypeptide domain comprises a Cas protein and the second polypeptide domain has an activity selected from the group consisting of transcription activation activity, transcription repression activity, transcription release factor activity, histone modification activity, nuclease activity, nucleic acid association activity, methylase activity, demethylase activity, acetylation activity, and deacetylation activity.

Clause 58. The DNA targeting system of clause 57, wherein the Cas protein comprises a Streptococcus pyogenes Cas9 protein, or a variant thereof.

Clause 59. The DNA targeting system of clause 58, wherein the Cas protein comprises a VQR variant of the S. pyogenes Cas9 protein.

Clause 60. The DNA targeting system of clause 59, wherein the DNA targeting system comprises a fusion protein.

Clause 61. The DNA targeting system of clause 60, wherein the second polypeptide domain has transcription activation activity, transcription repression activity, histone modification activity, or a combination thereof.

Clause 62. The DNA targeting system of clause 60, wherein the fusion protein comprises dCas9-VP64, VP64-dCas9-VP64, dCas9-p300, or dCas9-KRAB.

Clause 63. The DNA targeting system of any one of clauses 57-62, wherein the Cas protein comprises a Cas9 that recognizes a Protospacer Adjacent Motif (PAM) of NGG (SEQ ID NO: 13), NGA (SEQ ID NO: 14), NGAN (SEQ ID NO:15), or NGNG (SEQ ID NO:16).

Clause 64. The DNA targeting system of any one of clauses 55-62, wherein the gene within the 15q11-13 locus is selected from SNRPN, SNORD115, SNORD116, SPA1, SPA2, and MAGEL2.

Clause 65. An isolated polynucleotide sequence comprising the gRNA of clause 54.

Clause 66. An isolated polynucleotide sequence encoding the DNA targeting system of any one of clauses 55-64.

Clause 67. A vector comprising the isolated polynucleotide sequence of clause 65 or 66.

Clause 68. A vector encoding the gRNA of clause 54 and a Clustered Regularly Interspaced Short Palindromic Repeats associated (Cas) protein.

Clause 69. The vector of clause 68, wherein the Cas protein comprises a Streptococcus pyogenes Cas9 protein, or variant thereof.

Clause 70. The vector of clause 69, wherein the Cas protein comprises a VQR variant of the S. pyogenes Cas9 protein.

Clause 71. A cell comprising the gRNA of clause 54, the DNA targeting system of any one of clauses 55-64, the isolated polynucleotide sequence of clause 65 or 66, or the vector of any one of clauses 67-70, or a combination thereof.

Clause 72. A pharmaceutical composition comprising the gRNA of clause 54, the DNA targeting system of any one of clauses 55-64, the isolated polynucleotide sequence of clause 65 or 66, or the vector of any one of clauses 67-70, or the cell of clause 71, or a combination thereof.

Clause 73. A method for treating Prader-Willi Syndrome (PWS) in a subject, the method comprising administering to the subject the DNA targeting system of any one of clauses 55-64, the isolated polynucleotide sequence of clause 65 or 66, the vector of any one of clauses 67-70, or the cell of clause 71, or a combination thereof.

Clause 74. The method of clause 73, wherein the expression of at least one gene within the 15q11-q13 locus is increased.

Clause 75. The method of clause 74, wherein the gene within the 15q11-13 locus is selected from SNRPN, SNORD115, SNORD116, SNORD109A, IPW, SPA1, SPA2, and MAGEL2.

Clause 76. The method of any one of clauses 73-75, wherein the expression of at least one RNA transcript selected from SNRPN, SNORD115, SNORD116, SPA1, SPA2, and MAGEL2, or a combination thereof, is increased.

Clause 77. The method of any one of clauses 73-76, wherein the initiation of transcription from the SNRPN promoter, SNORD115 promoter, SNORD116 promoter, or a combination thereof, is increased.

Clause 78. The method of clause 1, wherein the DNA Targeting System has demethylase activity.

Clause 79. The method of clause 78 wherein administration of the DNA Targeting System results in increased expression of one or more of the following gene or gene products: MKRN3, MAGEL2, NDN, C15ORF2, SNURF-SNRPN, SNORD107, SNORD64, SNORD109A, SNORD116, SNORD116@, SPA1, SPA2, 116HG, SNORD116-1 to 30, Sno-Inc RNA 1 to 5, IPW, SNORD115, SNORD115Q, 115HG, SNORD115-1 to 48, SNORD109B, and/or SNG14.

Clause 80. The method of any of clauses 78-79 wherein the DNA Targeting System binds to a target region selected from the group consisting of: nucleotide positions −297 to +846 [mat3]; and nucleotide positions +13358 to +13526 [mat4]; wherein the nucleotide position is relative to position 1 being the start site of SNRPN exon 1 of the PWS imprinting center on chromosome 15.

Clause 81. The method of any of clauses 78-80, wherein the subject has a PWS Type 1 large deletion, PWS Type 2 large deletion, PWS imprinting center mutation or PWS uniparental disomy.

Clause 82. The method of any of clauses 78-80, wherein the subject has a PWS microdeletion encompassing SNORD116, but not MAGEL2.

Clause 83. The method of any of clauses 78-80, wherein the subject has a PWS or PWS-like atypical deletion encompassing MAGEL2, but not SNORD116.

Clause 84. The method of any of clauses 78-80, wherein the subject has heterozygous Schaaf-Yang syndrome or MAGEL2 disorder.

Clause 85. The method of any of clauses 78-84, wherein the DNA Targeting System is a CRISPR-Cas Type II system.

Clause 86. The method of clause 85, wherein the DNA Targeting System comprises: (a) a fusion protein comprising: (i) a Cas9 polypeptide with reduced nuclease activity, and (ii) a polypeptide with demethylase activity; and (b) one or more guide RNAs (gRNA) that bind to a target region in the 15q11-13 PWS-associated locus.

Clause 87. The method of clause 86, wherein the subject is administered: (a) said fusion protein and said one or more gRNAs; or (b) a nucleic acid sequence encoding said fusion protein, and said one or more gRNAs; or (c) a nucleic acid sequence encoding said fusion protein, and nucleic acid sequence(s) encoding said one or more gRNAs.

Clause 88. The method of clause 86, wherein the subject is administered a vector comprising a nucleic acid encoding said fusion protein and/or a vector comprising a nucleic acid encoding said gRNA.

Clause 89. The method of clause 88 wherein the vector is a viral vector, optionally a retroviral, lentiviral, adenovirus, adeno-associated virus vector, synthetic vector, or is a vector within a lipid nanoparticle.

Clause 90. The method of any of clauses 78-89, wherein the DNA Targeting System is a CRISPR-Cas Type II system comprising one or more gRNAs, wherein at least one gRNA comprises a nucleotide sequence at least 80% complementary to at least 10-20 consecutive nucleotides of said target region.

Clause 91. The method of any of clauses 78-90, wherein the at least one gRNA comprises a nucleotide sequence at least 80% complementary to at least 18 consecutive nucleotides of said target region.

Clause 92. The method of any of clauses 86-91, wherein the at least one gRNA specifically hybridizes to any of SEQ ID NOs: 47-86, or a corresponding allelic variant thereof, or the complement of any of SEQ ID Nos: 47-86 or a corresponding allelic variant thereof, or wherein the at least one gRNA comprises at least 10, 11, 12, 13, 14, 15, 16, 17, 18, 19, or 20 consecutive nucleotides of any of SEQ ID NOs: 591, 585, 685, 697, 750, 752, 763, 771, 196, 812, 861, or 1069, or a corresponding complement and/or allelic variant thereof.

Clause 93. The method of clause 92 wherein the at least one gRNA is fully complementary to at least 10, 11, 12, 13, 14, 15, 16, 17, 18, 19, or 20 consecutive nucleotides of any of SEQ ID NOs: 47-86, or a corresponding allelic variant thereof, or the complement of any of SEQ ID Nos: 47-86 or a corresponding allelic variant thereof, or wherein the at least one gRNA is fully complementary to at least 10, 11, 12, 13, 14, 15, 16, 17, 18, 19, or 20 consecutive nucleotides of any of SEQ ID NOs: 47, 49, 53, 55, 64, 65, 68, 70, 196, 291, 335, or 528, or a corresponding complement and/or allelic variant thereof.

Clause 94. The method of any of clauses 85-93 wherein the Cas9 is a S. pyogenes Cas9.

Clause 95. The method of any of clauses 78-84, wherein the DNA Targeting System is a ZF-based and/or TALE-based system.

Clause 96. The method of clause 95, wherein the DNA Targeting System comprises one or more fusion proteins comprising (a) a TALE that binds to the target region of the 15q11-13 PWS-associated locus and (b) a demethylase.

Clause 97. The method of clause 95, wherein the DNA Targeting System comprises one or more fusion proteins comprising (a) a ZF that binds to the target region of the 15q11-13 PWS-associated locus and (b) a demethylase.

Clause 98. The method of any of clauses 78-84 or 95-97 comprising administering a second DNA Targeting System targeting a second target region.

Clause 99. A DNA Targeting System that has demethylase activity and binds to a target region selected from the group consisting of: nucleotide positions −297 to +846 [mat3]; and nucleotide positions +13358 to +13526 [mat4]; wherein the nucleotide position is relative to position 1 being the start site of SNRPN exon 1 of the PWS imprinting center on chromosome 15.

Clause 100. The DNA Targeting System of clause 99 that targets a target region that results in increased expression of one of more of: SNORD116 or its products, or MAGEL2.

Clause 101. The DNA Targeting System of clause 99 that targets a target region that results in increased expression of one of more of the following gene or gene products: MKRN3, MAGEL2, NDN, C15ORF2, SNURF-SNRPN, SNORD107, SNORD64, SNORD109A, SNORD116, SNORD116@, SPA1, SPA2, 116HG, SNORD116-1 to 30, Sno-Inc RNA 1 to 5, IPW, SNORD115, SNORD115@, 115HG, SNORD115-1 to 48, SNORD109B, and/or SNG14.

Clause 102. The DNA Targeting System of any of clauses 99-101 that is a CRISPR-Cas Type II System.

Clause 103. The DNA Targeting System of clause 102 comprising: (a) a Cas9 fusion protein comprising: (i) a Cas9 polypeptide with reduced nuclease activity, and (ii) a polypeptide with demethylase activity; and (b) one or more guide RNAs (gRNA) that bind to a target region in the 15q11-13 PWS-associated locus.

Clause 104. The DNA Targeting System of clause 102 or clause 103 wherein the demethylase activity is Tet1CD (SEQ ID NO: 90).

Clause 105. The DNA Targeting System of clause 103 or 104 that comprises a S. pyogenes Cas9.

Clause 106. The DNA targeting system of clause 103 or 104 that comprises a VQR variant of the S. pyogenes Cas9 protein.

Clause 107. The DNA Targeting System of clause 103 or 104 that comprises a S. aureus Cas9.

Clause 108. The DNA Targeting System of clause 103 or 104 that comprises a Cas9 that recognizes a Protospacer Adjacent Motif (PAM) of NGG (SEQ ID NO: 13), NGA (SEQ ID NO: 14), NGAN (SEQ ID NO:15), or NGNG (SEQ ID NO:16).

Clause 109. The DNA Targeting System of any of clauses 101-103 wherein at least one gRNA comprises a nucleotide sequence at least 80% complementary to at least 10-20 consecutive nucleotides of said target region.

Clause 110. The DNA Targeting System of any of clauses 103-109 wherein the at least one gRNA comprises a nucleotide sequence at least 80% complementary to at least 18 consecutive nucleotides of said target region, or wherein the at least one gRNA comprises at least 10, 11, 12, 13, 14, 15, 16, 17, 18, 19, or 20 consecutive nucleotides of any of SEQ ID NOs: 591, 585, 685, 697, 750, 752, 763, 771, 196, 812, 861, or 1069, or a corresponding complement and/or allelic variant thereof.

Clause 111. A guide RNA (gRNA) comprising a polynucleotide sequence that specifically hybridizes to any of SEQ ID NOs: 47-86, or a corresponding allelic variant thereof, or the complement of any of SEQ ID Nos: 47-86 or a corresponding allelic variant thereof, or is fully complementary to at least 10, 11, 12, 13, 14, 15, 16, 17, 18, 19, or 20 consecutive nucleotides of any of SEQ ID NOs: 47, 49, 53, 55, 64, 65, 68, 70, 196, 291, 335, or 528, or a corresponding complement and/or allelic variant thereof, or comprises at least 10, 11, 12, 13, 14, 15, 16, 17, 18, 19, or 20 consecutive nucleotides of any of SEQ ID NOs: 591, 585, 685, 697, 750, 752, 763, 771, 196, 812, 861, or 1069, or a corresponding complement and/or allelic variant thereof.

Clause 112. The gRNA of clause 111 that is fully complementary to at least 10, 11, 12, 13, 14, 15, 16, 17, 18, 19, or 20 consecutive nucleotides of any of SEQ ID NOs: 47-86, or a corresponding allelic variant thereof, or the complement of any of SEQ ID Nos: 47-86 or a corresponding allelic variant thereof.

Clause 113. The DNA Targeting System of any of clauses 103−110 comprising a guide RNA of clause 111 of 112.

Clause 114. A nucleic acid encoding at least one component of the DNA Targeting System of any of clauses 103−110 or 113, or encoding a guide RNA of clause 111 or 112.

Clause 115. A first nucleic acid encoding at least one gRNA of the DNA Targeting System of any of clauses 103−110 or a gRNA of clause 111 of 112; and a second nucleic acid encoding the Cas9 fusion protein of the DNA Targeting System of any of clauses 103−110, wherein the first and second nucleic acids are on the same or different vectors.

Clause 116. A vector comprising the nucleic acid of clause 111 or 112.

Clause 117. The vector of clause 113 wherein the vector is a viral vector, optionally a retroviral, lentiviral, adenovirus, adeno-associated virus vector, synthetic vector, or is a vector within a lipid nanoparticle.

Clause 118. The DNA Targeting System of any of clauses 103-110 which is a ribonucleoprotein comprising a Cas9 fusion protein, and at least one gRNA.

Clause 119. A cell comprising said DNA Targeting System of any of clauses 103-110, 113 or 118, said gRNA of clause 111 or 112, said nucleic acids of clause 114 or 115, or said vector of clause 116 or 117.

Clause 120. A pharmaceutical composition comprising said DNA Targeting System of any of clauses 103-110, 113 or 118, said gRNA of clause 111 or 112, said nucleic acids of clause 114 or 115, or said vector of clause 116 or 117.

Clause 121. A method for treating Prader-Willi Syndrome (PWS) in a subject, the method comprising administering to the subject said DNA Targeting System of any of clauses 103-110, 113 or 118, said gRNA of clause 111 or 112, said nucleic acids of clause 114 or 115, or said vector of clause 116 or 117, or a combination thereof.

Clause 122. The method of clause 121, wherein the expression of at least one gene within the 15q11-q13 locus is increased.

Clause 123. The method of clause 121, wherein the gene within the 15q11-13 locus is selected from SNRPN, SNORD115, SNORD116, SNORD109A, IPW, SPA1, SPA2, and MAGEL2.

Clause 124. The method of any one of clauses 121-122, wherein the expression of at least one RNA transcript selected from SNRPN, SNORD115, SNORD116, SPA1, SPA2, and MAGEL2, or a combination thereof, is increased.

Clause 125. The method of any one of clauses 73-76, wherein the initiation of transcription from the SNRPN promoter, SNORD115 promoter, SNORD116 promoter, or a combination thereof, is increased.

SEQUENCES

| gRNA name | SEQ ID NO | gRNA sequence |
|---|---|---|
| PWS_G.3511 | 1 | ctaagcagtggactaaggat |
| PWS_G.3549 | 2 | ctactttagattctattgta |
| PWS_G.4662 | 3 | gcagctcagaaagtgatgca |
| PWS_G.4738 | 4 | gccaggttactgattcatag |
| PWS_G.4862 | 5 | gcgcactgcagcgcagacca |
| PWS_G.5117 | 6 | ggaaccagtcagaacaggtg |
| PWS_G.5706 | 7 | ggtcggcttcaggagggaag |
| PWS_G.6131 | 8 | gtggataggtgcgttcaagg |
| PWS_G.6318 | 9 | gtttcaggcctggactgggt |
| PWS_G.8738 | 10 | acagttcagggcatgagata |
| PWS_G.9414 | 11 | cagttcagggcatgagataa |
| PWS_G.10218 | 12 | gggcatgagataagggcagt |

SEQ ID NO: 13
NGG
SEQ ID NO: 14
NGA
SEQ ID NO: 15
NGAN
SEQ ID NO: 16
NGNG
SEQ ID NO: 17
NNAGAAW (W = A or T)
SEQ ID NO: 18
NAAR (R = A or G)
SEQ ID NO: 19
NNGRR (R = A or G; N can be any nucleotide residue, such as, any of A, G, C, or T)
SEQ ID NO: 20
NNGRRN (R = A or G; N can be any nucleotide residue, such as, any of A, G, C, or T)
SEQ ID NO: 21
NNGRRT (R = A or G; N can be any nucleotide residue, such as, any of A, G, C, or T)
SEQ ID NO: 22
NNGRRV (R = A or G; N can be any nucleotide residue, such as, any of A, G, C, or T)
SEQ ID NO: 23
codon optimized polynucleotide encoding S. pyogenes Cas9
atggataaaa agtacagcat cgggctggac atcggtacaa actcagtggg gtgggccgtg
attacggacg agtacaaggt accctccaaa aaatttaaag tgctgggtaa cacggacaga
cactctataa agaaaaatct tattggagcc ttgctgttcg actcaggcga gacagccgaa
gccacaaggt tgaagcggac cgccaggagg cggtatacca ggagaaagaa ccgcatatgc
tacctgcaag aaatcttcag taacgagatg gcaaaggttg acgatagctt tttccatcgc
ctggaagaat cctttcttgt tgaggaagac aagaagcacg aacggcaccc catctttggc
aatattgtcg acgaagtggc atatcacgaa aagtacccga ctatctacca cctcaggaag
aagctggtgg actctaccga taaggcggac ctcagactta tttatttggc actcgcccac
atgattaaat ttagaggaca tttcttgatc gagggcgacc tgaacccgga caacagtgac
gtcgataagc tgttcatcca acttgtgcag acctacaatc aactgttcga agaaaaccct
ataaatgctt caggagtcga cgctaaagca atcctgtccg cgcgcctctc aaaatctaga
agacttgaga atctgattgc tcagttgccc ggggaaaaga aaaatggatt gtttggcaac
ctgatcgccc tcagtctcgg actgacccca aatttcaaaa gtaacttcga cctggccgaa
gacgctaagc tccagctgtc caaggacaca tacgatgacg acctcgacaa tctgctggcc
cagattgggg atcagtacgc cgatctcttt ttggcagcaa agaacctgtc cgacgccatc
ctgttgagcg atatcttgag agtgaacacc gaaattacta aagcacccct tagcgcatct
atgatcaagc ggtacgacga gcatcatcag gatctgaccc tgctgaaggc tcttgtgagg
caacagctcc ccgaaaaata caaggaaatc ttctttgacc agagcaaaaa cggctacgct
ggctatatag atggtggggc cagtcaggag gaattctata aattcatcaa gcccattctc
gagaaaatgg acggcacaga ggagttgctg gtcaaactta acagggagga cctgctgcgg
aagcagcgga cctttgacaa cgggtctatc ccccaccaga ttcatctggg cgaactgcac
gcaatcctga ggaggcagga qgatttttat ccttttctta aagataaccg cgagaaaata
gaaaagattc ttacattcag gatcccgtac tacgtgggac ctctcgcccg gggcaattca
cggtttgcct ggatgacaag gaagtcagag gagactatta caccctggaa cctcgaagaa
gtggtggaca agggtgcatc tgcccagtct ttcatcgagc ggatgacaaa ttttgacaag
aacctcccta atgagaaggt gctgcccaaa cattctctgc tctacgagta ctttaccgtc
tacaatgaac tgactaaagt caagtacgtc accgagggaa tgaggaagcc ggcattcctt
agtggagaac agaagaaggc gattgtagac ctgttgttca agaccaacag gaaggtgact
gtgaagcaac ttaaagaaga ctactttaag aagatcgaat gttttgacag tgtggaaatt
tcaggggttg aagaccgctt caatgcgtca ttggggactt accatgatct tctcaagatc
ataaaggaca aagacttcct ggacaacgaa gaaaatgagg atattctcga agacatcgtc
ctcaccctga ccctgttcga agacagggaa atgatagaag agcgcttgaa aacctatgcc
cacctcttcg acgataaagt tatgaagcag ctgaagcgca ggagatacac aggatgggga
agattgtcaa ggaagctgat caatggaatt agggataaac agagtggcaa gaccatactg
gatttcctca aatctgatgg cttcgccaat aggaacttca tgcaactgat tcacgatgac
tctcttacct tcaaggagga cattcaaaag gctcaggtga gcgggcaggg agactccctt
catgaacaca tcgcgaattt ggcaggttcc cccgctatta aaaagggcat ccttcaaact
gtcaaggtgg tggatgaatt ggtcaaggta atgggcagac ataagccaga aaatattgtg
atcgagatgg cccgcgaaaa ccagaccaca cagaagggcc agaaaaatag tagagagcgg
atgaagagga tcgaggaggg catcaaagag ctgggatctc agattctcaa agaacacccc
gtagaaaaca cacagctgca gaacgaaaaa ttgtacttgt actatctgca gaacggcaga
gacatgtacg tcgaccaaga acttgatatt aatagactgt ccgactatga cgtagaccat
atcgtgcccc agtccttcct gaaggacgac tccattgata acaaagtctt gacaagaagc
gacaagaaca ggggtaaaag tgataatgtg cctagcgagg aggtggtgaa aaaaatgaag
aactactggc gacagctgct taatgcaaag ctcattacac aacggaagtt cgataatctg
acgaaagcag agagaggtgg cttgtctgag ttggacaagg cagggtttat taagcggcag
ctggtggaaa ctaggcaaat cacaaagcac gtggcgcaga ttttggacag ccggatgaac
acaaaatacg acgaaaatga taaactgata cgagaggtca aagttatcac gctgaaaagc
aagctggtgt ccgattttcg gaaagacttc cagttctaca aagttcgcga gattaataac
taccatcatg ctcacgatgc gtacctgaac gctgttgtcg ggaccgcctt gataaagaag
tacccaaagc tggaatccga gttcgtatac ggggattaca aagtgtacga tgtgaggaaa
atgatagcca agtccgagca ggagattgga aaggccacag ctaagtactt cttttattct
aacatcatga atttttttaa gacggaaatt accctggcca acggagagat cagaaagcgg
ccccttatag agacaaatgg tgaaacaggt gaaatcgtct gggataaggg cagggatttc
gctactgtga ggaaggtgct gagtatgcca caggtaaata tcgtgaaaaa aaccgaagta
cagaccggag gattttccaa ggaaagcatt ttgcctaaaa gaaactcaga caagctcatc
gcccgcaaga aagattggga ccctaagaaa tacgggggat ttgactcacc caccgtagcc
tattctgtgc tggtggtagc taaggtggaa aaaggaaagt ctaagaagct gaagtccgtg
aaggaactct tgggaatcac tatcatggaa agatcatcct ttgaaaagaa ccctatcgat
ttcctggagg ctaagggtta caaggaggtc aagaaagacc tcatcattaa actgccaaaa
tactctctct tcgagctgga aaatggcagg aagagaatgt tggccagcgc cggagagctg
caaaagggaa acgagcttgc tctgccctcc aaatatgtta attttctcta tctcgcttcc
cactatgaaa agctgaaagg gtctcccgaa gataacgagc agaagcagct gttcgtcgaa
cagcacaagc actatctgga tgaaataatc gaacaaataa gcgagttcag caaaagggtt
atcctggcgg atgctaattt qgacaaagta ctgtctgctt ataacaagca ccgggataag
cctattaggg aacaagccga gaatataatt cacctcttta cactcacgaa tctcggagcc
cccgccgcct tcaaatactt tgatacgact atcgaccgga aacggtatac cagtaccaaa
gaggtcctcg atgccaccct catccaccag tcaattactg gcctgtacga
aacacggatcgacctctctc aactgggcgg cgactag
SEQ ID NO: 24
Amino acid sequence of codon optimized polynucleotide encoding
S. pyogenes Cas9
MDKKYSIGLDIGTNSVGWAVITDEYKVPSKKFKVLGNTDRHSIKKNLIGALLFDSGETAEATRLKRTA
RRRYTRRKNRICYLQEIFSNEMAKVDDSFFHRLEESFLVEEDKKHERHPIFGNIVDEVAYHEKYPTIY
HLRKKLVBSTDKADLRLIYLALAHMIKFRGHFLIEGDLNPDNSDVDKLFIQLVQTYNQLFEENPINAS
GVDAKAILSARLSKSRRLENLIAQLPGEKKNGLFGNLIALSLGLTPNFKSNFDLAEDAKLQLSKDTYD
DDLDNLIAQIGDQYADLFLAAKNLSDAILLSDILRVNTEITKAPLSASMIKRYDEHHQDLTLLKALVR
QQLPEKYKEIFFDQSKNGYAGYIDGGASQEEFYKFIKPILEKMDGTEELLVKLNREDLLRKQRTFDNG
SIPHQIHLGELHAILRRQEDFYPFLKDNREKIEKILTFRIPYYVGPLARGNSRFAWMTRKSEETITPW
NFEEVVDKGASAQSFIERMTNFDKNLPNEKVLPKHSLLYEYFTVYNELTKVKYVTEGMRKPAFLSGEQ
KKAIVDLLFKTNRKVTVKQLKEDYFKKIECFDSVEISGVEDRFNASLGTYHDLLKIIKDKDFLDNEEN
EDILEDIVLTLTLFEDREMIEERLKTYAHLFDDKVMKQLKRRRYTGWGRLSRKLINGIRDKQSGKTIL
DFLKSDGFANRNFMQLIHDDSLTFKEDIQKAQVSGQGDSLHEHIANLAGSPAIKKGILQTVKVVDELV
KVMGRHKPENIVIEMARENQTTQKGQKNSRERMKRIEEGIKELGSQILKEHPVENTQLQNEKLYLYYL
QNGRDMYVDQELDINRLSDYDVDHIVPQSFLKDDSIDNKVLTRSDKNRGKSDNVPSEEVVKKMKNYWR
QLLNAKLITQRKFDNLTKAERGGLSELDKAGFIKRQLVETRQITKHVAQILDSRMNTKYDENDKLIRE
VKVITLKSKLVSDFRKDFQFYKVREINNYHHAHDAYLNAVVGTALIKKYPKLESEFVYGDYKVYDVRK
MIAKSEQEIGKATAKYFFYSNIMNFFKTEITLANGEIRKRPLIETNGETGEIVWDKGRDFATVRKVLS
MPQVNIVKKTEVQTGGFSKESILPKRNSDKLIARKKDWDPKKYGGFDSPTVAYSVLVVAKVEKGKSKK
LKSVKEILGITIMERSSFEKNPIDFLEAKGYKEVKKDLIIKLPKYSLFELENGRKRMLASAGELQKGN
ELALPSKYVNFLYLASHYEKLKGSPEDNEQKQLFVEQHKHYLDEIIEQISEFSKRVILADANLDKVLS
AYNKHRDKPIREQAENIIHLFTLTNLGAPAAFKYFDTTIDRKRYTSTKEVLDATLIHQSITGLYETRI
DLSQLGGD
SEQ ID NO: 25
codon optimized nucleic acid sequences encoding S. aureus Cas9
atgaaaagga actacattct ggggctggac atcgggatta caagcgtggg gtatgggatt
attgactatg aaacaaggga cgtgatcgac gcaggcgtca gactgttcaa ggaggccaac
gtggaaaaca atgagggacg gagaagcaag aggggagcca ggcgcctgaa acgacggaga
aggcacagaa tccagagggt gaagaaactg ctgttcgatt acaacctgct gaccgaccat
tctgagctga gtggaattaa tccttatgaa gccagggtga aaggcctgag tcagaagctg
tcagaggaag agttttccgc agctctgctg cacctggcta agcgccgagg agtgcataac
gtcaatgagg tggaagagga caccggcaac gagctgtcta caaaggaaca gatctcacgc
aatagcaaag ctctggaaga gaagtatgtc gcagagctgc agctggaacg gctgaagaaa
gatggcgagg tgagagggtc aattaatagg ttcaagacaa gcgactacgt caaagaagcc
aagcagctgc tgaaagtgca gaaggcttac caccagctgg atcagagctt catcgatact
tatatcgacc tgctggagac tcggagaacc tactatgagg gaccaggaga agggagcccc
ttcggatgga aagacatcaa ggaatggtac gagatgctga tgggacattg cacctatttt
ccagaagagc tgagaagcgt caagtacgct tataacgcag atctgtacaa cgccctgaat
gacctgaaca acctggtcat caccagggat gaaaacgaga aactggaata ctatgagaag
ttccagatca tcgaaaacgt gtttaagcag aagaaaaagc ctacactgaa acagattgct
aaggagatcc tggtcaacga agaggacatc aagggctacc gggtgacaag cactggaaaa
ccagagttca ccaatctgaa agtgtatcac gatattaagg acatcacagc acggaaagaa
atcattgaga acgccgaact gctggatcag attgctaaga tcctgactat ctaccagagc
tccgaggaca tccaggaaga gctgactaac ctgaacagcg agctgaccca ggaagagatc
gaacagatta gtaatctgaa ggggtacacc ggaacacaca acctgtccct gaaagctatc
aatctgattc tggatgagct gtggcataca aacgacaatc agattgcaat ctttaaccgg
ctgaagctgg tcccaaaaaa ggtggacctg agtcagcaga aagagatccc aaccacactg
gtggacgatt tcattctgtc acccgtggtc aagcggagct tcatccagag catcaaagtg
atcaacgcca tcatcaagaa gtacggcctg cccaatgata tcattatcaa gctggctagg
gagaagaaca gcaaggacgc acagaagatg atcaatgaga tgcagaaacg aaaccggcag
accaatgaac gcattgaaga gattatccga actaccggga aagagaacgc aaagtacctg
attgaaaaaa tcaagctgca cgatacgcag gagggaaagt gtctgtattc tctggaggcc
tccccctgg aggacctgct gaacaatcca ttcaactacg aggtcgatca tattatcccc
agaagcgtgt ccttcgacaa ttcctttaac aacaaggtgc tggtcaagca ggaagagaac
tctaaaaagg gcaataggac tcctttccag tacctgtcta gttcagattc caagatctct
tacgaaacct ttaaaaagca cattctgaat ctggccaaag gaaagggccg catcagcaag
accaaaaagg agtacctgct ggaagagcgg gacatcaaca gattctccgt ccagaaggat
tttattaacc ggaatctggt ggacacaaga tacgctactc gcggcctgat gaatctgctg
cgatcctatt tccgggtgaa caatctggat gtgaaagtca agtccatcaa cggcgggttc
acatcttttc tgaggcgcaa atggaagttt aaaaaggagc gcaacaaagg gtacaagcac
catgccgaag atgctctgat tatcgcaaat gccgacttca tctttaagga gtggaaaaag
ctggacaaag ccaagaaagt gatggagaac cagatgttcg aagagaagca ggccgaatct
atgcccgaaa tcgagacaga acaggagtac aaggagattt tcatcactcc tcaccagatc
aagcatatca aggatttcaa ggactacaag tactctcacc gggtggataa aaagcccaac
agagagctga tcaatgacac cctgtatagt acaagaaaag acgataaggg gaataccctg
attgtgaaca atctgaacgg actgtacgac aaagataatg acaagctgaa aaagctgatc
aacaaaagtc ccgagaagct gctgatgtac caccatgatc ctcagacata tcagaaactg
aagctgatta tggagcagta cggcgacgag aagaacccac tgtataagta ctatgaagag
actgggaact acctgaccaa gtatagcaaa aaggataatg gccccgtgat caagaagatc
aagtactatg ggaacaagct gaatgcccat ctggacatca cagacgatta ccctaacagt
cgcaacaagg tqgtcaagct gtcactgaag ccatacagat tcgatgtcta tctggacaac
ggcgtgtata aatttgtgac tgtcaagaat ctggatgtca tcaaaaagga gaactactat
gaagtgaata gcaagtgcta cgaagaggct aaaaagctga aaaagattag caaccaggca
gagttcatcg cctcctttta caacaacgac ctgattaaga tcaatggcga actgtatagg
gtcatcgggg tgaacaatga tctgctgaac cgcattgaag tgaatatgat tgacatcact
taccgagagt atctggaaaa catgaatgat aagcgccccc ctcgaattat caaaacaatt
gcctctaaga ctcagagtat caaaaagtac tcaaccgaca ttctgggaaa cctgtatgag
gtgaagagca aaaagcaccc tcagattatc aaaaagggc
SEQ ID NO: 26
codon optimized nucleic acid sequences encoding S. aureus Cas9
atgaagcgga actacatcct gggcctggac atcggcatca ccagcgtggg ctacggcatc
atcgactacg agacacggga cgtgatcgat gccggcgtgc ggctgttcaa agaggccaac
gtggaaaaca acgagggcag gcggagcaag agaggcgcca gaaggctgaa gcggcggagg
cggcatagaa tccagagagt gaagaagctg ctgttcgact acaacctgct gaccgaccac
agcgagctga gcggcatcaa cccctacgag gccagagtga agggcctgag ccagaagctg
agcgaggaag agttctctgc cgccctgctg cacctggcca agagaagagg cgtgcacaac
gtgaacgagg tggaagagga caccggcaac gagctgtcca ccaaagagca gatcagccgg
aacagcaagg ccctggaaga gaaatacgtg gccgaactgc agctggaacg gctgaagaaa
gacggcgaag tgcggggcag catcaacaga ttcaagacca gcgactacgt gaaagaagcc
aaacagctgc tgaaggtgca gaaggcctac caccagctgg accagagctt catcgacacc
tacatcgacc tgctggaaac ccggcggacc tactatgagg gacctggcga gggcagcccc
ttcggctgga aggacatcaa agaatggtac gagatgctga tgggccactg cacctacttc
cccgaggaac tgcggagcgt gaagtacgcc tacaacgccg acctgtacaa cgccctgaac
gacctgaaca atctcgtgat caccagggac gagaacgaga agctggaata ttacgagaag
ttccagatca tcgagaacgt gttcaagcag aagaagaagc ccaccctgaa gcagatcgcc
aaagaaatcc tcgtgaacga agaggatatt aagggctaca gagtgaccag caccggcaag
cccgagttca ccaacctgaa ggtgtaccac gacatcaagg acattaccgc ccggaaagag
attattgaga acgccgagct gctggatcag attgccaaga tcctgaccat ctaccagagc
agcgaggaca tccaggaaga actgaccaat ctgaactccg agctgaccca ggaagagatc
gagcagatct ctaatctgaa gggctatacc ggcacccaca acctgagcct gaaggccatc
aacctgatcc tggacgagct gtggcacacc aacgacaacc agatcgctat cttcaaccgg
ctgaagctgg tgcccaagaa ggtggacctg tcccagcaga aagagatccc caccaccctg
gtggacgact tcatcctgag ccccgtcgtg aagagaagct tcatccagag catcaaagtg
atcaacgcca tcatcaagaa gtacggcctg cccaacgaca tcattatcga gctggcccgc
gagaagaact ccaaggacgc ccagaaaatg atcaacgaga tgcagaagcg gaaccggcag
accaacgagc ggatcgagga aatcacccgg accaccggca aagagaacgc caagtacctg
atcgagaaga tcaagctgca cgacatgcag gaaggcaagt gcctgtacag cctggaagcc
atccctctgg aagatctgct gaacaacccc ttcaactatg aggtggacca catcatcccc
agaagcgtgt ccttcgacaa cagcttcaac aacaaggtgc tegtgaagca ggaagaaaac
agcaagaagg gcaaccggac cccattccag tacctgagca gcagcgacag caagatcagc
tacgaaacct tcaagaagca catcctgaat ctggccaagg gcaagggcag aatcagcaag
accaagaaag agtatctgct ggaagaacgg gacatcaaca ggttctccgt gcagaaagac
ttcatcaacc ggaacctggt ggataccaga tacgccacca gaggcctgat gaacctgctg
cggagctact tcagagtgaa caacctggac gtgaaagtga agtccatcaa tggcggcttc
accagctttc tgcggcggaa gtggaagttt aagaaagagc ggaacaaggg gtacaagcac
cacgccgagg acgccctgat cattgccaac gccgatttca tcttcaaaga gtggaagaaa
ctggacaagg ccaaaaaagt gacggaaaac cagatgttcg aggaaaagca ggccgagagc
atgcccgaga tcgaaaccga gcaggagtac aaagagatct tcatcacccc ccaccagatc
aagcacatta aggacttcaa ggactacaag tacagccacc gggtggacaa gaagcctaat
agagagctga ttaacgacac cctgtactcc acccggaagg acgacaaggg caacaccctg
atcgtgaaca atctgaacgg cctgtacgac aaggacaatg acaagctgaa aaagctgatc
aacaagagcc ccgaaaagct gctgatgtac caccacgacc cccagaccta ccagaaactg
aagctgatta tggaacagta cggcgacgag aagaatcccc tgtacaagta ctacgaggaa
accgggaact acctgaccaa gtactccaaa aaggacaacg gccccgtgat caagaagatt
aagtattacg gcaacaaact gaacgcccat ctggacatca ccgacgacta ccccaacagc
agaaacaagg tcgtgaagct gtccctgaag ccctacagat tcgacgtgta cctggacaat
ggcgtgtaca agttcgtgac cgtgaagaat ctggatgtga tcaaaaaaga aaactactac
gaagtgaata gcaagtgcta tgaggaagct aagaagctga agaagatcag caaccaggcc
gagtttatcg cctccttcta caacaacgat ctgatcaaga tcaacggcga gctgtataga
gtgatcggcg tgaacaacga cctgctgaac cggatcgaag tgaacatgat cgacatcacc
taccgcgagt acctggaaaa catgaacgac aagaggcccc ccaggatcat taagacaatc
gcctccaaga cccagagcat taagaagtac agcacagaca ttctgggcaa cctgtatgaa
gtgaaatcta agaagcaccc tcagatcatc aaaaagggc
SEQ ID NO: 27
codon optimized nucleic acid sequences encoding S. aureus Cas9
atgaagcgca actacatcct cggactggac atcggcatta cctccgtggg atacggcatc
atcgattacg aaactaggga tgtgatcgac gctggagtca ggctgttcaa agaggcgaac
gtggagaaca acgaggggcg gcgctcaaag aggggggccc gccggctgaa gcgccgccgc
agacatagaa tccagcgcgt gaagaagctg ctgttcgact acaaccttct gaccgaccac
tccgaacttt ccggcatcaa cccatatgag gctagagtga agggattgtc ccaaaagctg
tccgaggaag agttctccgc cgcgttgctc cacctcgcca agcgcagggg agtgcacaat
gtgaacgaag tggaagaaga taccggaaac gagctgtcca ccaaggagca gatcagccgg
aactccaagg ccctggaaga gaaatacgtg gcggaaccgc aactggagcg gctgaagaaa
gacggagaag tgcgcggctc gatcaaccgc ttcaagacct cggactacgt gaaggaggcc
aagcagctcc tgaaagtgca aaaggcotat caccaacttg accagtcctt tatcgatacc
tacatcgatc tgctcgagac tcggcggact tactacgagg gtccagggga gggctcccca
tttggttgga aggatattaa ggagtggtac gaaatgctga tgggacactg cacatacttc
cctgaggagc tgcggagcgt gaaatacgca tacaacgcaa acctgtacaa cgcgctgaac
gacctgaaca atctcgtgat cacccgggac gagaacgaaa agctcgagta ttacgaaaag
ttccagatta ttgagaacgt gttcaaacag aagaagaagc cgacactgaa gcagattgcc
aaggaaatcc tcgtgaacga agaggacacc aagggctatc gagtgacctc aacgggaaag
ccggagttca ccaatctgaa ggtctaccac gacatcaaag acattaccgc ccggaaggag
atcattgaga acgcggagct gttggaccag attgcgaaga ttctgaccat ctaccaatcc
tccgaggata ttcaggaaga actcaccaac ctcaacagcg aactgaccca ggaggagata
gagcaaatct ccaacctgaa gggctacacc ggaactcata acctgagcct gaaggccatc
aacttgatcc tggacgagct gtggcacacc aacgataacc agatcgctat tttcaatcgg
ctgaagctgg tccccaagaa agtggacctc tcacaacaaa aggagatccc tactaccctt
gtggacgatt tcattctgtc ccccgtggtc aagagaagct tcatacagtc aatcaaagtg
atcaatgcca ttatcaagaa atacggtctg cccaacgaca ttatcattga gctcgcccgc
gagaagaact cgaaggacgc ccagaagatg attaacgaaa tgcagaagag gaaccgacag
actaacgaac ggatcgaaga aatcacccgg accaccggga aggaaaacgc gaagtacctg
atcgaaaaga tcaagctcca tgacatgcag gaaggaaagt gtctgtactc gctggaggcc
attccgctgg aqgacttgct gaacaaccct tttaactacg aagtggatca tatcattccg
aggagcgtgt cattcgacaa ttccttcaac aacaaggtcc tcgtgaagca ggaggaaaac
tcgaagaagg gaaaccgcac gccgttccag tacctgagca gcagcgactc caagatttcc
tacgaaaccC tcaagaagca catcctcaac ctggcaaagg ggaagggtcg catctccaag
accaagaagg aatatctgct ggaagaaaga gacatcaaca gattctccgt gcaaaaggac
ttcatcaacc gcaacctcgt ggatactaga tacgctactc ggggtctgat gaacctcctg
agaagctatc ttagagtgaa caatctggac gtgaaggtca agtcgattaa cggaggtttc
acctccttcc tgcggcgcaa gtggaagttc aagaaggaac ggaacaaggg ctacaagcac
cacgccgagg acgccctgat cattgccaac gccgacttca tcttcaaaga atggaagaaa
cttgacaagg ctaaqaaggt catggaaaac cagatgttcg aagaaaagca ggccgagtct
atgcctgaaa tcgagactga acaggagtac aaggaaatct ttattacgcc acaccagatc
aaacacatca aggatttcaa ggattacaag tactcacatc gcgtggacaa aaagccgaac
agggaactga tcaacgacac cctctactcc acccggaagg atgacaaagg gaataccctc
atcgtcaaca accttaacgg cctgtacgac aaggacaacg ataagctgaa gaagctcatt
aacaagtcgc ccgaaaagtt gctgatgtac caccacgacc ctcagactta ccagaagctc
aagctgatca tggagcagta tggggacgag aaaaacccgt tgtacaagta ctacgaagaa
actgggaatt atctgactaa gtactccaag aaagataacg gccccgtgat taagaagatt
aagtactacg gcaacaagct gaacgcccat ctggacatca ccgatgacta ccctaattcc
cgcaacaagg tcgtcaagct gagcctcaag ccctaccggt ttgatgtgta cctcgacaat
ggagtgtaca agttcgtgac tgtgaagaac cttgacgtga ccaagaagga gaactactac
gaagtcaact ccaagtgcta cgaggaagca aagaagttga agaagatctc gaaccaggcc
gagttcattg cctccttcta taacaacgac ctgattaaga tcaacggcga actgtaccgc
gtcattggcg tgaacaacga tctcctgaac cgcatcgaag tgaacatgat cgacatcact
taccgggaat acctggagaa tatgaacgac aagcgcccgc cccggatcat taagactatc
gcctcaaaga cccagtcgat caaaaagtac agcaccaaca tcctgggcaa cctgtacgag
gtcaaatcga agaagcaccc ccagatcatc aagaaggga
SEQ ID NO: 28
codon optimized nucleic acid sequences encoding S. aureus Cas9
atggccccaaagaagaagcggaaggtcggtatccacggagtcccagcagccaagcggaactacatcct
gggcctggacatcggcatcaccagcgtgggctacggcatcatcgactacgagacacgggacgtgatcg
atgccggcgtgcggctgttcaaagaggccaacgtggaaaacaacgagggcaggcggagcaagagaggc
gccagaaggctgaagcggcggaggcggcatagaatccagagagtgaagaagctgctgttcgactacaa
cctgctgaccgaccacagcgagctgagcggcatcaacccctacgaggccagagtgaagggcctgagcc
agaagctgagcgaggaagagttctctgccgccctgctgcacctggccaagagaagaggcgtgcacaac
gtgaacgaggtggaagaggacaccggcaacgagctgtccaccagagagcagatcagccggaacagcaa
ggccctggaagagaaatacgtggccgaactgcagctggaacggctgaagaaagacggcgaagtgcggg
gcagcatcaacagattcaagaccagcgactacgtgaaagaagccaaacagctgcbgaaggtgcagaag
gcctaccaccagctggaccagagcttcatcgacacctacatcgacctgctggaaacccggcggaccta
ctatgagggacctggcgagggcagacccttcggctggaaggacatcaaagaatggtacgagatgctga
tgggccactgcacctacttccccgaggaactgcggagegtgaagtacgcctacaacaccgacctgtac
aacgccctgaacgacctgaacaatctcgtgatcaccagggacgagaacgagaagctggaatattacga
gaagttccagatcatcgagaacgtgttcaagcagaagaagaagcccaccctgaagcagatcgccaaag
aaatcctcgtgaacgaagaggatattaagggctacagagtgaccagcaccggcaagcccgagttcacc
aacctgaaggtgtaccacgacatcaaggacattaccgcccggaaagagattattgagaacgccgagct
gctggatcagattgocaagatcctgaccatctaccagagcagogaggacatocaggaagaactgacca
atctgaactccgagctgacccaggaagagatcgagcagatctctaatctgaagggctataccggcacc
cacaacctgagcctgaaggccatcaacctgatcctggacgagctgtggcacaccaacgacaaccagat
cgctatcttcaaccggctgaagctggtgcccaagaaggtggacctgtcccagcagaaagagatcccca
ccaccctggtggacgacttcatcctaagccccgtcgtgaagagaagcttcatccagagcatcaaagtg
atcaacgccatcatcaagaagtacggcctgcccaacgacaccattatcgagctggcccgcgagaagaa
ctccaaggacgcccagaaaatgatcaacgagatgcagaagcggaaccggcagaccaacgagcggatcg
aggaaatcatccggaccaccggcaaagagaacgccaagtacctgatcgagaagatcaagctgcacgac
atgcaggaaggcaagtgcctgtacagcctggaagccatccctctggaagatctgctgaacaacccctt
caactatgaggtggaccacatcatccccagaagcgtgtccttcgacaacagcttcaacaacaaggtgc
tcgtgaagcaggaagaaaacagcaagaagggcaaccggaccccattccagtacctgagcagcagcgac
agcaagatcagctacgaaaccttcaagaagcacatcctgaatctggccaagggcaagggcagaatcag
caagaccaagaaagagtatctgctggaagaacgggacatcaacaggttctccgtgcagaaagacttca
tcaaccggaacctggtggataccagatacgccaccagaggcctgatgaacctgctgcggagctacttc
agagtgaacaacctggacgtgaaagtgaagcccatcaatggcggcttcaccagctttctgcggcggaa
gtggaagtttaagaaagagcggaacaaggggtacaagcaccacgccgaggacgccctgatcattgcca
acgccgatttcatcttcaaagagtggaagaaactggacaaggccaaaaaagtgatggaaaaccagatg
ttcgaggaaaggcaggccgagagcatgcccgagatcgaaaccgagcaggagtacaaagagatcttcat
caccccccaccagatcaagcacattaaggacttcaaggactacaagtacagccaccgggtggacaaga
agcctaatagagagctgattaacgacaccctgtactccacccgqaaggacgacaaggqcaacaccctg
atcgtgaacaatctgaacggcctgtacgacaaggacaatgacaagctgaaaaagccgatcaacaagag
ccccgaaaagctgctgatgtaccaccacgacccccagacccaccagaaactgaagctgattatggaac
agtacggcgacgagaagaatcccctgtacaagtactacgaggaaaccgggaactacctgaccaagtac
tccaaaaaggacaacggccccgtgatcaagaagattaagtattacggcaacaaactgaacgcccatct
ggacatcaccgacgactaccccaacagcagaaacaaggtcgtgaagctgtccctgaagccctacagat
tcgacgtgtacctggacaatggcgtgtacaagttcgtgaccgtgaagaatctggatgtgatcaaaaaa
gaaaactactacgaagtgaatagcaagtgctatgaggaagctaagaagctgaagaagatcagcaacca
ggccgagtttatcgcctccttctacaacaacgatctgatcaagatcaacggcgagctgtatagagtga
tcggcgtgaacaacgacctgctgaaccggatcgaagtgaacatgatcgacatcacctaccgcgagtac
ctggaaaacatgaacgacaagaggccccccaggatcattaagacaatcgcctccaagacccagagcat
taagaagtacagcacagacattctgggcaacctgtatgaagtgaaatctaagaagcaccctcagatca
tcaaaaagggcaaaaggccggcggccacgaaaaaggccggccaggcaaaaaagaaaaag
SEQ ID NO: 29
codon optimized nucleic acid sequences encoding S. aureus Cas9
accggtgcca ccatgcaccc atacgatgct ccagattacg cttcgccgaa gaaaaagcgc
aaggtcgaag cgtccatgaa aaggaactac attctggggc tggacatcgg gattacaagc
gtggggtatg ggattattga ctatgaaaca agggacgtga tcgacgcagg cgtcagactg
ttcaaggagg ccaacgtgga aaacaatqag ggacggagaa gcaagagggg agccaggcgc
ctgaaacgac ggagaaggca cagaatccag agggtgaaga aactgctgtt cgattacaac
ctgctgaccg accattctga gctgagtgga attaatcctt atgaagccag ggtgaaaggc
ctgagtcaga agctgtcaga ggaagagttt tccgcagctc tgctgcacct ggctaagcgc
cgaggagtgc ataacgtcaa tgaggtggaa gaggacaccg gcaacgagct gtctacaaag
gaacagatct cacgcaatag caaagctctg gaagagaagt atgtcgcaga gctgcagctg
gaacggctga agaaagatgg cgaggtgaga gggtcaatta ataggttcaa gacaaacgac
tacgtcaaag aagccaagca gctgctgaaa gtgcagaagg cttaccacca gctggatcag
agcttcatcg atacttatat cgacctgctg gagactcgga gaacctacta tgagggacca
ggagaaggga gccccttcgg atggaaagac atcaaggaat ggtacgagat gctgatggga
cattgcacct attttccaga agagctgaga agcgtcaagt acgcttataa cgcagatct
tacaacgccc tgaatgacct gaacaacctg gtcatcacca gggatgaaaa cgagaaactg
gaatactatg agaagttcca gatcatcgaa aacgtgttta agcagaagaa aaagcctaca
ctgaaacaga ttgctaagga gatcctggtc aacgaagagg acatcaaggg ctaccgggtg
acaagcactg gaaaaccaga gttcaccaat ctgaaagtgt atcacgatat taaggacatc
acagcacgga aagaaatcat tgagaacgcc gaactgctgg atcagattgc taagatcctg
actatctacc agagctccga ggacatccag gaagagctga ctaacctgaa cagcgagctg
acccaggaag agatcgaaca gattagtaat ctgaaggggt acaccggaac acacaacctg
tccctgaaag ctatcaatct gattctggat gagctgtggc atacaaacga caatcagatt
gcaatcttta accggctgaa gctggtccca aaaaaggtgg acctgagtca gcagaaagag
atcccaacca cactggtgga cgatttcatt ctgtcacccg tggtcaagcg gagcttcatc
cagagcatca aagtgatcaa cgccatcatc aagaagtacg gcctgcccaa tgatatcatt
atcgagctgg ctagggagaa gaacagcaag gacgcacaga agatgatcaa tgagatgcag
aaacgaaacc ggcagaccaa tgaacgcatt gaagagatta tccgaactac cgggaaagag
aacgcaaagt acctgattga aaaaatcaag ctgcacgata tgcaggaggg aaagtgtctg
tattctctgg aggccatccc cctggaggac ctgctgaaca atccattcaa ctacgaggtc
gatcatatta tccccagaag cgtgtccttc gacaattcct ttaacaacaa ggtgctggtc
aagcaggaag agaactctaa aaagggcaat aggactcctt tccagtacct gtctagttca
gattccaaga tctcttacga aacctttaaa aagcacattc tgaatctggc caaaggaaag
ggccgcatca gcaagaccaa aaaggagtac ctgctggaag agcgggacat caacagattc
tccgtccaga aggattttat taaccggaat ctggcggaca caagatacgc tactcgcggc
ctgatgaatc tgctgcgatc ctatttccgg gtgaacaatc tggatgtgaa agtcaagtcc
atcaacggcg ggttcacatc ttttctgagg cgcaaatgga agtttaaaaa ggagcgcaac
aaagggtaca agcaccatgc cgaagatgct ctgattatcg caaatgccga cttcatcttt
aaggagtgga aaaagctgga caaagccaag aaagtgatgg agaaccagat gttcgaagag
aagcaggccg aatctatgcc cgaaatcgag acagaacagg agtacaagga gattttcatc
actcctcacc agatcaagca tatcaaggat ttcaaggact acaagtactc tcaccgggtg
gataaaaagc ccaacagaga gctgatcaat gacaccctgt atagtacaaa aaaagacgat
aaggggaata ccctgattgt gaacaatctg aacggactgt acgacaaaga taatgacaag
ctgaaaaagc tgatcaacaa aagtcccgag aagctgctga tgtaccacca tgatcctcag
acatatcaga aactgaagct gattatggag cagtacggcg acgagaagaa cccactgtat
aagtactatg aagagactgg gaactacctg accaagtata gcaaaaagga taatggcccc
gtgatcaaga agatcaagta ctatgggaac aagctgaatg cccatctgga catcacagac
gattacccta acagtcgcaa caaggtggtc aagctgtcac tgaagccata cagattcgat
gtctatctgg acaacggcgt gtataaattt gtgactgtca agaatctgga tgtcatcaaa
aaggagaact actatgaagt gaatagcaag tgctacgaag aggctaaaaa gctgaaaaag
attagcaacc aggcagagtt catcgcctcc ttttacaaca acgacctgat taagatcaat
ggcgaactgt atagggtcat cggggtgaac aatgatctgc tgaaccgcat tgaagtgaat
atgattgaca tcacttaccg agagtatctg gaaaacatga atgataagcg cccccctcga
atcatcaaaa caattgcctc taagactcag agtaccaaaa agtactcaac cgacattctg
ggaaacctgt atgaggtgaa gagcaaaaag caccctcaga ttatcaaaaa gggctaagaa
ttc
SEQ ID NO: 30
Amino acid sequence of codon optimized nucleic acid sequences encoding
S. aureus Cas9
MKRNYILGLDIGITSVGYGIIDYETRDVIDAGVRLFKEANVENNEGRRSKRGARRLKRRRRHRIQRVK
KLLFDYNLLTDHSELSGINPYEARVKGLSQKLSEEEFSAALLHLAKRRGVHNVNEVEEDTGNELSTKE
QISRNSKALEEKYVAELQLERLKKDGEVRGSINRFKTSDYVKEAKQLLKVQKAYHQLDQSFIDTYIDL
LETRRTYYEGPGEGSPFGWKDIKEWYEMLMGHCTYFPEELRSVKYAYNADLYNALNDLNNLVITRDEN
EKLEYYEKFQIIENVFKQKKKPTLKQIAKEILVNEEDIKGYRVTSTGKPEFTNLKVYHDIKDITARKI
IIENAELLDQIAKILTIYQSSEDIQEELTNLNSELTQEEIEQISNLKGYTGTHNLSLKAINLILDELW
HTNDNQIAIFNRLKLVPKKVDLSQQKEIPTTLVDDFILSPVVKRSFIQSIKVINAIIKKYGLPNDIII
ELAREKNSKDAQKMINEMQKRNRQTNERIEEIIRTTGKENAKYLIEKIKLHDMQEGKCLYSLEAIPLE
DLLNNPFNYEVDHIIPRSVSFDNSFNNKVLVKQEENSKKGNRTPFQYLSSSDSKISYETFKKHILNLA
KGKGRISKTKKEYLLEERDINRFSVQKDFINRNLVDTRYATRGLMNLLRSYFRVNNLDVKVKSINGGF
TSFLRRKWKFKKERNKGYKHHAEDALIIANADFIFKEWKKLDKAKKVMENQMFEEKQAESMPEIETEQ
EYKEIFITPHQIKHIKDFKDYKYSHRVDKKPNRELINDTLYSTRKDDKGNTLIVNNLNGLYDKDNDKL
KKLINKSPEKLLMYKHDPQTYQKLKLIMEQYGDEKNPLYKYYEETGNYLTKYSKKDNGPVIKKIKYYG
NKLNAHLDITDDYPNSRNKVVKLSLKPYRFDVYLDNGVYKFVTVKNLDVIKKENYYEVNSKCYEEAKK
LKKISNQAEFIASFYNNDLIKINGELYRVIGVNNDLLNRIEVNMIDITYREYLENMNDKRPPRIIKTI
ASKTQSIKKYSTDILGNLYEVKSKKHPQIIKKG
SEQ ID NO: 31
D10A mutant of S. aureus Cas9
atgaaaagga actacattct ggggctggcc atcgggatta caagcgtggg gtatgggatt
attgactatg aaacaaggga cgtgatcgac gcaggcgtca gactgttcaa ggaggccaac
gtggaaaaca atgagggacg gagaagcaag aggggagcca ggcgcctgaa acgacggaga
aggcacagaa tccagagggt gaagaaactg ctgttcgatt acaacctgct gaccgaccat
tctgagctga gtggaattaa tccttatgaa gccagggtga aaggcctgag tcagaagctg
tcagaggaag agttttccgc agctctgctg cacctggcta agcgccgagg agtgcataac
gtcaatgagg tggaagagga caccggcaac gagctgtcta caaaggaaca gatctcacgc
aatagcaaag ctctggaaga gaagtatgtc gcagagctgc agctggaacg gctgaagaaa
gatggcgagg tgagagggtc aattaatagg ttcaagacaa gcgactacgt caaagaagcc
aagcagctgc tgaaagtgca gaaggcttac caccagctgg atcagagctt catcgatact
tatatcgacc tgctggagac tcggagaacc tactatgagg gaccaggaga agggagcccc
ttcggatgga aagacatcaa ggaatggtac gagatgctga tgggacattg cacctatttt
ccagaagagc tgagaagcgt caagtacgct tataacgcag atctgtacaa caccctgaat
gacctgaaca acctggtcat caccagggat gaaaacgaga aactggaata ctatgagaag
ttccagatca tcgaaaacgt gtttaagcag aagaaaaagc ctacactgaa acagattgct
aaggagatcc tggtcaacga agaggacatc aagggctacc gggtgacaag cactggaaaa
ccagagttca ccaatctgaa agtgtatcac gatattaagg acatcacagc acggaaagaa
atcattgaga acgccgaact gctggatcag attgctaaga tcctgactat ctaccagagc
tccgaggaca tccaggaaga gctgactaac ctgaacagcg agctgaccca ggaagagatc
gaacagatta gtaatctgaa ggggtacacc ggaacacaca acctgtccct gaaagctatc
aatctgattc tggatgagct gtggcataca aacgacaatc agattgcaat ctttaaccgg
ctgaagctgg tcccaaaaaa ggtggacctg agtcagcaga aagagatccc aaccacactg
gtggacgatt tcattctgtc acccgtggtc aagcggagct tcatccagag catcaaagtq
atcaacgcca tcatcaagaa gtacggcctg cccaatgata tcattatcga gctggctagg
gagaagaaca gcaaggacgc acagaagatg atcaatgaga tgcagaaacg aaaccggcag
accaatgaac gcattgaaga gattatccga actaccggga aagagaacgc aaagtacctg
attgaaaaaa tcaagctgca cgatatgcag gagggaaagt gtctgtattc tctggaggcc
atccccctgg aggacctgct gaacaatcca ttcaactacg aggtcgatca tattatcccc
agaagcgtgt ccttcgacaa ttcctttaac aacaaggtgc tggtcaagca ggaagagaac
tctaaaaagg gcaataggac tcctttccag tacctgtcta gttcagattc caagatctct
tacgaaacct ttaaaaagca cattctgaat ctggccaaag gaaagggccg catcagcaag
accaaaaagg agtacctgct ggaagagcgg gacatcaaca gattctccgt ccagaaggat
tttattaacc ggaatctggt ggacacaaga tacgctactc gcggcctgat gaatctgctg
cgatcctatt tccgggtgaa caatctggat gtgaaagtca agtccatcaa cggcgggttc
acatcttttc tgaggcgcaa atggaagttt aaaaaggagc gcaacaaagg gtacaagcac
catgccgaag atgctctgat tatcgcaaat gccgacttca tctttaagga gtggaaaaag
ctggacaaag ccaaqaaagt gatggagaac cagatgttcg aagagaagca ggccgaatct
atgcccgaaa tcgagacaga acaggagtac aaggagattt tcatcactcc tcaccagatc
aagcatatca aggatttcaa ggactacaag tactctcacc gggtggataa aaagcccaac
agagagctga tcaatgacac cctgtatagt acaagaaaag acgataaggg gaataccctg
attgtgaaca atctgaacgg actgtacgac aaagataatg acaagctgaa aaagctgatc
aacaaaagtc ccgagaagct gctgatgtac caccatgatc ctcagacata tcagaaactg
aagctgatta tggagcagta cggcgacgag aagaacccac tgtataagta ctatgaagag
actgggaact acctgaccaa gtatagcaaa aaggataatg gccccgtgat caagaagatc
aagtactatg ggaacaagct gaatgcccat ctggacatca cagacgatta ccctaacagt
cgcaacaagg tggtcaagct gtcactgaag ccatacagat tcgatgtcta tctggacaac
ggcgtgtata aacttgtgac tgtcaagaat ctggatgtca tcaaaaagga gaactactat
gaagtgaata gcaagtgcta cgaagaggct aaaaagctga aaaagattag caaccaggca
gagttcatcg cctcctttta caacaacgac ctgattaaga tcaatggcga actgtatagg
gtcatcgggg tgaacaatga tctgctgaac cgcattgaag tgaatatgat tgacatcact
taccgagagt atctggaaaa catgaatgat aagcgccccc ctcgaattat caaaacaatt
gcctctaaga ctcagagtat caaaaagtac tcaaccgaca ttctgggaaa cctgtatgag
gtgaagagca aaaagcaccc tcagattatc aaaaagggc
SEQ ID NO: 32
N580A mutant of S. aureus Cas9
atgaaaagga actacattct ggggctggac atcgggatta caagcgtggg gtatgggatt
attgactatg aaacaaggga cgtgatcgac gcaggcgtca gactgttcaa ggaggccaac
gtggaaaaca atgagggacg gagaagcaag aggggagcca ggcgcctgaa acgacggaga
aggcacagaa tccagagggt gaagaaactg ctgttcgatt acaacctgct gaccgaccat
tctgagctga gtggaattaa tccttatgaa gccagggtga aaggcctgag tcagaagctg
tcagaggaag agttttccgc agctctgctg cacctggcta agcgccgagg agtgcataac
gtcaatgagg tggaagagga caccggcaac gagctgtcta caaaggaaca gatctcacgc
aatagcaaag ctctggaaga gaagtatgtc gcagagctgc agctggaacg gctgaagaaa
gatggcgagg tgagagggtc aattaatagg ttcaagacaa gcgactacgt caaagaagcc
aagcagctgc tgaaagtgca gaaggcttac caccagctgg atcagagctt catcgatact
tatatcgacc tgctggagac tcggagaacc tactatgagg gaccaggaga agggagcccc
ttcggatgga aagacatcaa ggaatggtac gagatgctga tgggacattg cacctatttt
ccagaagagc tgagaagcgt caagtacgct tataacgcag atctgtacaa cgccctgaat
gacctgaaca acctggtcat caccagggat gaaaacgaga aactggaata ctatgagaag
ttccagatca tcgaaaacgt gtttaagcag aagaaaaagc ctacactgaa acagattgct
aaggagatcc tggtcaacga agaggacatc aagggctacc gggtgacaag cactggaaaa
ccagagttca ccaatctgaa agtgtatcac gatattaagg acatcacagc acggaaagaa
atcattgaga acgccgaact gctggatcag attgctaaga tcctgactat ctaccagagc
tccgaggaca tccaggaaga gctgactaac ctgaacagcg agctgaccca ggaagagatc
gaacagatta gtaatctgaa ggggtacacc ggaacacaca acctgtccct gaaagctatc
aatctgattc tggatgagct gtggcataca aacgacaatc agattgcaat ctttaaccgg
ctgaagctgg tcccaaaaaa ggtggacctg agtcagcaga aagagatccc aaccacactg
gtggacgatt tcattctgtc acccgtggtc aagcggagct tcatccagag catcaaagtg
atcaacgcca tcatcaagaa gtacggcctg cccaatgata tcattatcga gctggctagg
gagaagaaca gcaaggacgc acagaagatg atcaatgaga tgcagaaacg aaaccggcag
accaatgaac gcattgaaga gattatccga actaccggga aagagaacgc aaagtacctg
attgaaaaaa tcaagctgca cgatatgcag gagggaaagt gtctgtattc tctggaggcc
atccccctgg aggacctgct gaacaatcca ttcaactacg aggtcgatca tattatcccc
agaagcgtgt ccttcgacaa ttcctttaac aacaaggtgc tggtcaagca ggaagaggcc
tctaaaaagg gcaataggac tcctttccag tacctgtcta gttcagattc caagatctct
tacgaaacct ttaaaaagca cattctgaat ctggccaaag gaaagggccg catcagcaag
accaaaaagg agtacctgct ggaagagcgg gacatcaaca gattctccgt ccagaaggat
tttattaacc ggaatctggt ggacacaaga tacgctactc gcggcctgat gaatctgctg
cgatcctatt tccgggtgaa caatctggat gtgaaagtca agtccatcaa cggcgggttc
acatcttttc tgaggcgcaa atggaagttt aaaaaggagc gcaacaaagg gtacaagcac
catgccgaag atgctctgat tatcgcaaat gccgacttca tctttaagga gtggaaaaag
ctggacaaag ccaagaaagt qatggagaac cagatgttcg aagagaagca ggccgaatct
atgcccgaaa tcgagacaga acaggagtac aaggagattt tcatcactcc tcaccagatc
aagcatatca aggatttcaa ggactacaag tactctcacc gggtggataa aaagcccaac
agagagctga tcaatgacac cctgtatagt acaagaaaag acgataaggg gaataccctg
attgtgaaca atctgaacgg actgtacgac aaagataatg acaagctgaa aaagctgatc
aacaaaagtc ccgagaaact gctgatgtac caccatgatc ctcagacata tcagaaactg
aagctgatta tggagcaata cggcgacgag aagaacccac tgtataagta ctatgaagag
actgggaact acctgaccaa gtatagcaaa aaggataatg gccccgtgat caagaagatc
aagtactatg ggaacaagct gaatgcccat ctggacatca cagacgatta ccctaacagt
cgcaacaagg tggtcaagct gtcactgaag ccatacagat tcgatgtcta tctggacaac
ggcgtgtata aatttgtgac tgtcaagaat ctgattaaga tcaaaaagga gaactactat
gaagtgaata gcaagtgcta cgaagaggct aaaaagctga aaaagattag caaccaggca
gagttcatcg cctcctttta caacaacgac ctgattaaga tcaatggcga actgtatagg
gtcatcgggg tgaacaatga tctgctgaac cgcattgaag tgaatatgat tgacatcact
taccgagagt atctggaaaa catgaatgat aagcgccccc ctcgaattat caaaacaatt
gcctctaaga ctcagagtat caaaaagtac tcaaccgaca ttctgggaaa cctgtatgag
gtgaagagca aaaagcaccc tcagattatc aaaaagggc
SEQ ID NO: 33
NGGNG
SEQ ID NO: 34
codon optimized nucleic acid sequences encoding S. aureus Cas9
atggccccaaagaagaagcggaaggtcggtatccacggagtcccagcagccaagcggaactacatcct
gggcctggacatcggcatcaccagcgtgggctacggcatcatcgactacgagacacgggacgtgatcg
atgccggcgtgcggctgttcaaagaggccaacgtggaaaacaacgagggcaggcggagcaagagaggc
gccagaaggctgaagcggcggaggcggcatagaatccagagagtgaagaagctgctgttcgactacaa
cctgctgaccgaccacagcgagctgagcggcatcaacccctacgaggccagagtgaagggcctgagcc
agaagctgagcgaggaagagttctctgccgccctgctgcacctggccaagagaagaggcgtgcacaac
gtgaacgaggtggaagaggacaccggcaacgagctgtccaccaaagagcagatcagccggaacagcaa
ggccctggaagagaaatacgtggccgaactgcagctggaacggctgaagaaagacggcgaagtgcggg
gcagcatcaacagattcaagaccagcgactacgtgaaagaagccaaacagctgctgaaggtgcagaag
gcctaccaccagctggaccagagcttcatcgacacctacatcgacctgctggaaacccggcggaccta
ctatgagggacctggcgagggcagccccttcggctggaaggacatcaaagaatggtacgagatgctga
tgggccactgcacctacttccccgaggaactgcggagcgtgaagtacgcctacaacgccgacctgtac
aacgccctgaacgacctgaacaatctcgtgatcaccagggacgagaacgagaagctggaatattacga
gaagttccagatcatcgagaacgtgttcaagcagaagaagaagcccaccctgaagcagatcgccaaag
aaatcctcgtgaacgaagaggatattaagggctacagagtgaccagcaccggcaagcccgagttcacc
aacctgaaggtgtaccacgacatcaaggacattaccgcccggaaagagattattgagaacgccgagct
gctggatcagattgccaagatcctgaccatctaccagagcagcgaggacatccaggaagaactgacca
atctgaactccgagctgacccaggaagagatcgagcagatctctaatctgaagggctataccggcacc
cacaacctgagcctgaaggccatcaacctgatcctggacgagctgtggcacaccaacgacaaccagat
cgctatcttcaaccggctgaagctggtgcccaagaaggtggacctgtcccagcagaaagagatcccca
ccaccctggtggacgacttcatcctgagccccgtcgtgaagagaagcttcatccagagcatcaaagtg
atcaacgccatcatcaagaagtacggcctgcccaacgacatcattatcgagctggcccgcgagaagaa
ctccaaggacgcccagaaaatgatcaacgagatgcagaagcggaaccggcagaccaacgagcggatcg
aggaaatcatccggaccaccggcaaagagaacgccaagtacctgatcgagaagatcaagctgcacgac
atgcaggaaggcaagtgcctgtacagcctggaagccatccctctggaagatctgctgaacaacccctt
caactatgaggtggaccacatcatccccagaagcgtgtccttcgacaacagcttcaacaacaaggtgc
tcgtgaagcaggaagaaaacagcaagaagggcaaccggaccccattccagtacctgagcagcagcgac
agcaagatcagctacgaaaccttcaagaagcacatcctgaatctggccaagggcaagggcagaatcag
caagaccaagaaagagtatctgctggaagaacgggacatcaacaggttctccgtgcagaaagacttca
tcaaccggaacctggtggataccagatacgccaccagaggcctgatgaacctgctgcggagctacttc
agagtgaacaacctggacgtgaaagtgaagtccatcaatggcggcttcaccagctttctgcggcggaa
gtggaagttcaagaaagagcggaacaaggggtacaagcaccacgccgaggacgccctgatcattgccac
acgccgatttcatcttcaaagagtggaagaaactggacaaggccaaaaaagtgatggaaaaccagatg
ttcgaggaaaagcaggccgagagcatgcccgagatogaaaccgagcaggagtacaaagagatcttcat
caccccccaccagatcaagcacattaaggacttcaaggactacaagtacagccaccgggtggacaaga
agcctaatagagagctgattaacgacaccctgtactccacccggaaggacgacaagggcaacaccctg
atcgtgaacaatctgaacggcctgtacgacaaggacaatgacaagctgaaaaagctgatcaacaagag
ccccgaaaagctgctgatgtaccaccacgacccccagacctaccagaaactgaagctgattatggaac
agtacggcgacgagaagaatcccctgtacaagtactacgaggaaaccgggaactacctgaccaagtac
tccaaaaaggacaacggccccgtgatcaagaagattaagtattacggcaacaaactgaacgcccatct
ggacatcaccgacgactaccccaacagcagaaacaaggtcgtgaagctgtccctgaagccctacagat
tcgacgtgtacctggacaatggcgtgtacaagttcgtgaccgtgaagaatctggatgtgatcaaaaaa
gaaaactactacgaagtgaatagcaagtgctatgaggaagctaagaagctgaagaagatcagcaacca
ggccgagtttatcgcctccttctacaacaacgatctgatcaagatcaacggcgagctgtatagagtga
tcggcgtgaacaacgacctgctgaaccggatcgaagtgaacatgatcgacatcacctaccgcgagtac
ctggaaaacatgaacgacaagaggccccccaggatcattaagacaatcgcctccaagacccagagcat
taagaagtacagcacagacattctgggcaacctgtatgaagtgaaatctaagaagcaccctcagatca
tcaaaaagggcaaaaggccggcggccacgaaaaaggccggccaggcaaaaaagaaaaag
SEQ ID NO: 35
codon optimized nucleic acid sequences encoding S. aureus Cas9
aagcggaactacatcctgggcctggacatcggcatcaccagcgtgggctacggcatcatcgactacga
gacacgggacgtgatcgatgccggcgtgcggctgttcaaagaggccaacgtggaaaacaacgagggca
ggcggagcaagagaggcgccagaaggctgaagcggcggaggcggcatagaatccagagagtgaagaag
ctgctgttcgactacaacctgctgaccgaccacagcgagctgagcggcatcaacccctacgaggccag
agtgaagggcctgagccagaagctgagcgaggaagagttctctgccgccctgctgcacctggccaaga
gaagaggcgtgcacaacgtgaacgaggtggaagaggacaccggcaacgagctgtccaccaaagaqcag
atcagccggaacagcaaggccctggaagagaaatacgtggccgaactgcagctggaacggctgaagaa
agacggcgaagtgcggggcagcatcaacagattcaagaccagcgactacgtgaaagaagccaaacagc
tgctgaaggtgcagaaggcctaccaccagctggaccagagcttcatcgacacctacatcgacctgctg
gaaacccggcggacctactatgagggacctggcgagggcagccccttcggctggaaggacatcaaaga
atggtacgagatgctgatgggccactgcacctacttccccgaggaactgcggagcgtgaagtacgcct
acaacgccgacctgtacaacgccctgaacgacctgaacaatctcgtgatcaccagggacgagaacgag
aagctggaatattacgagaagttccagatcatcgagaacgtgttcaagcagaagaagaagcccaccct
gaagcagatcgccaaagaaatcctcgtqaacgaagaggatattaagggctacagagtgaccagcaccg
gcaagcccgagttcaccaacctgaaggtgtaccacgacatcaaggacattaccgcccggaaagagatt
attgagaacgccgagctgctagatcagattgccaagatcctgaccatctaccagagcagcgaggacat
ccaggaagaactgaccaatctgaactccgagctgacccaggaagagatcgagcagatctctaatctga
agggctataccggcacccacaacctgagcctgaaggccatcaacctgatcctggacgagctgtggcac
accaacgacaaccagatcgctatcttcaaccggctgaagctggtgcccaagaaggtggacctgtccca
gcagaaagagatccccaccaccctggtggacgacttcatcctgagccccgtcgtgaagagaagcttca
tccagagcatcaaagtgatcaacgccatcatcaagaagtacggcctgcccaacgacatcattatcgag
ctggcccgcgagaagaactccaaggacgcccagaaaatgatcaacgagatgcagaagcggaaccggca
gaccaacgagcggatcgaggaaatcatccggaccaccggcaaagagaacgccaagtacctgatcgaga
agatcaagctgcacgacatgcaggaaggcaagtgcctgtacagcctggaagccatccctctggaagat
ctgctgaacaaccccttcaactatgaggtggaccacatcatccccagaagcgtgtccttcgacaacag
cttcaacaacaaggtgctcgtgaagcaggaagaaaacagcaagaagggcaaccggaccccattccagt
acctgagcagcagcgacagcaagatcagctacgaaaccttcaagaagcacatcctgaatctggccaag
ggcaagggcagaatcagcaagaccaagaaagagtatctgctggaagaacgggacatcaacaggttctc
cgtgcagaaagacttcatcaaccggaacctggtggataccagatacgccaccagaggcctgatgaacc
tgctgcggagctacttcagagtgaacaacctggacgtgaaagtgaagtccatcaatggcggcttcacc
agctttctgcggcggaagtggaagtttaagaaagagcggaacaaggggtacaagcaccacgccgagga
cgccctgatcattgccaacgccgatttcatcttcaaagagtggaagaaactggacaaggccaaaaaag
tgatggaaaaccagatgttcgaggaaaagcaggccgagagcatgcccgagatcgaaaccgagcaggag
tacaaagagatcttcatcaccccccaccagatcaagcacattaaggacttcaaggactacaagtacag
ccaccgggtggacaagaagcctaatagagagctgattaacgacaccctgtactccacccggaaggacg
acaagggcaacaccctgatcgtgaacaatctgaacggcctgtacgacaagqacaatgacaagctgaaa
aagctgatcaacaagagccccgaaaagctgctgatgtaccaccacgacccccagacctaccagaaact
gaagctgattatggaacagtacqgcgacgagaagaatcccctgtacaagtactacgaggaaaccggga
actacctgaccaagtactccaaaaaggacaacggccccgtgatcaagaagattaagtattacggcaac
aaactgaacgcccatctggacatcaccgacgactaccccaacagcagaaacaaggtcgtgaagctgtc
cctgaagccctacagattcgacgtgtacctggacaatggcgtgtacaagttcgtgaccgtgaagaatc
tggatgtgatcaaaaaagaaaactactacgaagtgaatagcaagtgctatgaggaagctaagaagctg
aagaagatcagcaaccaggccgagtttatcgcctccttctacaacaacgatctgatcaagatcaacgg
cgagctgtatagagtgatcggcgtgaacaacgacctgctgaaccggatcgaagtgaacatgatcgaca
tcacctaccgcgagtacctggaaaacatgaacgacaagaggccccccaggatcattaagacaatcgcc
tccaagacccagagcattaagaagtacagcacagacattctgggcaacctgtatgaagtgaaatctaa
gaagcaccctcagatcatcaaaaagggc
SEQ ID NO: 36
Amino acid sequence of codon optimized nucleic acid sequences
encoding S. aureus Cas9
KRNYILGLDIGITSVGYGIIDYETRDVIDAGVRLFKEANVENNEGRRSKRGARRLKRRRRHRIQRVKK
LLFDYNLLTDHSELSGINPYEARVKGLSQKLSEEEFSAALLHLAKRRGVHMVNEVEEDTGNELSTKEQ
ISRNSKALEEKYVAELQLERLKKDGEVRGSINRFKTSDYVKEAKQLLKVQKAYHQLDQSFIDTYIDLL
ETRRTYYEGPGEGSPFGWKDIKEWYEMLMGHCTYFPEELRSVKYAYNADLYNALNDLNNLVITRDENE
KLEYYEKFQIIENVFKQKKKPTLKQIAKEILVNEEDIKGYRVTSTGKPEFTNLKVYHDIKDITARKEI
IENAELLDQIAKILTIYQSSEDIQEELTNLNSELTQEEIEQISNLKGYTGTHNLSLKAINLILDELWH
TNDNQIAIFNRLKLVPKKVDLSQQKEIPTTLVDDFILSPVVKRSFIQSIKVINAIIKKYGLPNDIIIE
LAREKNSKDAQKMINEMQKRNRQTNERIEEIIRTTGKENAKYLIEKIKLHDMQEGKCLYSLEAIPLED
LLNNPFNYEVDHIIPRSVSFDNSFNNKVLVKQEENSKKGNRTPFQYLSSSDSKISYETFKKHILNLAK
GKGRISKTKKEYLLEERDINRFSVQKDFINRNLVDTRYATRGLMNLLRSYFRVNNLDVKVKSINGGFT
SFLRRKWKFKKERNKGYKHHAEDALIIANADFIFKEWKKLDKAKKVMENQMFEEKQAESMPEIETEQE
YKEIFITPHQIKHIKDFKDYKYSHRVDKKPNRELINDTLYSTRKDDKGNTLIVNNLNGLYDKDNDKLK
KLINKSPEKLLMYHHDPQTYQKLKLIMEQYGDEKNPLYKYYEETGNYLTKYSKKDNGPVIKKIKYYGN
KLNAHLDITDDYPNSRNKVVKLSLKPYRFDVYLDNGVYKFVTVKNLDVIKKENYYEVNSKCYEEAKKL
KKISNQAEFIASFYNNDLIKINGELYRVIGVNNDLLNRIEVNMIDITYREYLENMNDKRPPRIIKTIA
SKTQSIKKYSTDILGNLYEVKSKKHPQIIKKG
SEQ ID NO: 37
Vector (pDO242) encoding codon optimized nucleic acid sequences
encoding S. aureus Cas9
ctaaattgtaagcgttaatattttgttaaaattcgcgttaaatttttgttaaatcagctcatttttta
accaataggccgaaatcggcaaaatcccttataaatcaaaagaatagaccgagatagggttgagtgtt
gttccagtttggaacaagagtccactattaaagaacgtggactccaacgtcaaagggcgaaaaaccgt
ctatcagggcgatggcccactacgtgaaccatcaccctaatcaagttttttggggtcgaggtgccgta
aagcactaaatcggaaccctaaagggagcccccgatttagagcttgacggggaaagccggcgaacgtg
gcgagaaaggaagggaagaaagcgaaaggagcgggcgctagggcgctggcaagtgtagcggtcacgct
gcgcgtaaccaccacacccgccgcgcttaatgcgccgctacagggcgcgtcccattcgccattcaggc
tgcgcaactgttgggaagggcgatcggtgcgggcctcttcgctattacgccagctggcgaaaggggga
tgtgctgcaaggcgattaagttgggtaacgccagggttttcccagtcacgacgttgtaaaacgacggc
cagtgagcgcgcgtaatacgactcactatagggcgaattgggtacctttaattctagtactatgcatg
cgttgacattgattattgactagttattaatagtaatcaattacggggtcattagttcatagcccata
tatggagttccgcgttacataacttacggtaaatggcccgcctggctgaccgcccaacgacccccgcc
cattgacgtcaataatgacgtatgttcccatagtaacgccaatagggactttccattgacgtcaatgg
gtggagtatttacggtaaactgcccacttggcagtacatcaagtgtatcatatgccaagtacgccccc
tattgacgtcaatgacggtaaatggcccgcctggcattatgcccagtacatgaccttatgggactttc
ctacttggcagtacatctacgtattagtcatcgctattaccatggtgatgcggttttggcagtacatc
aatgggcgtggatagcggtttgactcacggggatttccaagtctccaccccattgacgtcaatgggag
tttgttttggcaccaaaatcaacgggactttccaaaatgtcgtaacaactccgccccattgacgcaaa
tgggcggtaggcgtgtacggtgggaggtctatataagcagagctctctggctaactaccggtgccacc
ATGAAAAGGAACTACATTCTGGGGCTGGACATCGGGATTACAAGCGTGGGGTATGGGATTATTGACTA
TGAAACAAGGGACGTGATCGACGCAGGCGTCAGACTGTTCAAGGAGGCCAACGTGGAAAACAATGAGG
GACGGAGAAGCAAGAGGGGAGCCAGGCGCCTGAAACGACGGAGAAGGCACAGAATCCAGAGGGTGAAG
AAACTGCTGTTCGATTACAACCTGCTGACCGACCATTCTGAGCTGAGTGGAATTAATCCTTATGAAGC
CAGGGTGAAAGGCCTGAGTCAGAAGCTGTCAGAGGAAGAGTTTTCCGCAGCTCTGCTGCACCTGGCTA
AGCGCCGAGGAGTGCATAACGTCAATGAGGTGGAAGAGGACACCGGCAACGAGCTGTCTACAAAGGAA
CAGATCTCACGCAATAGCAAAGCTCTGGAAGAGAAGTATGTCGCAGAGCTGCAGCTGGAACGGCTGAA
GAAAGATGGCGAGGTGAGAGGGTCAATTAATAGGTTCAAGACAAGCGACTAGGTCAAAGAAGCCAAGC
AGCTGCTGAAAGTGCAGAAGGCTTACCACCAGCTGGATCAGAGCTTCATCGATACTTATATCGACCTG
CTGGAGACTCGGAGAACCTACTATGAGGGACCAGGAGAAGGGAGCCCCTTCGGATGGAAAGACATCAA
GGAATGGTACGAGATGCTGATGGGACATTGCACCTATTTTCCAGAAGAGCTGAGAAGCGTCAAGTACG
CTTATAACGCAGATCTGTACAACGCCCTGAATGACCTGAACAACCTGGTCATCACCAGGGATGAAAAC
GAGAAACTGGAATACTATGAGAAGTTCCAGATCATCGAAAACGTGTTTAAGCAGAAGAAAAAGCCTAC
ACTGAAACAGATTGCTAAGGAGATCCTGGTCAACGAAGAGGACATCAAGGGCTACCGGGTGACAAGCA
CTGGAAAACCAGAGTTCACCAATCTGAAAGTGTATCACGATATTAAGGACATCACAGCACGGAAAGAA
ATCATTGAGAACGCCGAACTGCTGGATCAGATTGCTAAGATCCTGACTATCTACCAGAGCTCCGAGGA
CATCCAGGAAGAGCTGACTAACCTGAACAGCGAGCTGACCCAGGAAGAGATCGAACAGATTAGTAATC
TGAAGGGGTACACCGGAACACACAACCTGTCCCTGAAAGCTATCAATCTGATTCTGGATGAGCTGTGG
CATACAAACGACAATCAGATTGCAATCTTTAACCGGCTGAAGCTGGTCCCAAAAAAGGTGGACCTGAG
TCAGCAGAAAGAGATCCCAACCACACTGGTGGACGATTTCATTCTGTCACCCGTGGTCAAGCGGAGCT
TCATCCAGAGCATCAAAGTGATCAACGCCATCATCAAGAAGTACGGCCTGCCCAATGATATCATTATC
GAGCTGGCTAGGGAGAAGAACAGCAAGGACGCACAGAAGATGATCAATGAGATGCAGAAACGAAACCG
GCAGACCAATGAACGCATTGAAGAGATTATCCGAACTACCGGGAAAGAGAACGCAAAGTACCTGATTG
AAAAAATCAAGCTGCACGATATGCAGGAGGGAAAGTGTCTGTATTCTCTGGAGGCCATCCCCCTGGAG
GACCTGCTGAACAATCCATTCAACTACGAGGTCGATCATATTATCCCCAGAAGCGTGTCCTTCGACAA
TTCCTTTAACAACAAGGTGCTGGTCAAGCAGGAAGAGAACTCTAAAAAGGGCAATAGGACTCCTTTCC
AGTACCTGTCTAGTTCAGATTCCAAGATCTCTTACGAAACCTTTAAAAAGCACATTCTGAATCTGGCC
AAAGGAAAGGGCCGCATCAGCAAGACCAAAAAGGAGTACCTGCTGGAAGAGCGGGACATCAACAGATT
CTCCGTCCAGAAGGATTTTATTAACCGGAATCTGGTGGACACAAGATACGCTACTCGCGGCCTGATGA
ATCTGCTGCGATCCTATTTCCGGGTGAACAATCTGGATGTGAAAGTCAAGTCCATCAACGGCGGGTTC
ACATCTTTTCTGAGGCGCAAATGGAAGTTTAAAAAGGAGCGCAACAAAGGGTACAAGCACCATGCCGA
AGATGCTCTGATTATCGCAAATGCCGACTTCATCTTTAAGGAGTGGAAAAAGCTGGACAAAGCCAAGA
AAGTGATGGAGAACCAGATGTTCGAAGAGAAGCAGGCCGAATCTATGCCCGAAATCGAGACAGAACAG
GAGTACAAGGAGATTTTCATCACTCCTCACCAGATCAAGCATATCAAGGATTTCAAGGACTACAAGTA
CTCTCACCGGGTGGATAAAAAGCCCAACAGAGAGCTGATCAATGACACCCTGTATAGTACAAGAAAAG
ACGATAAGGGGAATACCCTGATTGTGAACAATCTGAACGGACTGTACGAGAAAGATAATGACAAGCTG
AAAAAGCTGATCAACAAAAGTCCCGAGAAGCTGCTGATGTACCACCATGATCCTCAGACATATCAGAA
ACTGAAGCTGATTATGGAGCAGTACGGCGACGAGAAGAACCCACTGTATAAGTACTATGAAGAGACTG
GGAACTACCTGACCAAGTATAGCAAAAAGGATAATGGCCCCGTGATCAAGAAGATCAAGTACTATGGG
AACAAGCTGAATGCCCATCTGGACATCACAGACGATTACCCTAACAGTCGCAACAAGGTGGTCAAGCT
GTCACTGAAGCCATACAGATTCGATGTCTATCTGGACAACGGCGTGTATAAATTTGTGACTGTCAAGA
ATCTGGATGTCATCAAAAAGGAGAACTACTATGAAGTGAATAGCAAGTGCTACGAAGAGGCTAAAAAG
CTGAAAAAGATTAGCAACCAGGCAGAGTTCATCGCCTCCTTTTACAACAACGACCTGATTAAGATCAA
TGGCGAACTGTATAGGGTCATCGGGGTGAACAATGATCTGCTGAACCGCATTGAAGTGAATATGATTG
ACATCACTTACCGAGAGTATCTGGAAAACATGAATGATAAGCGCCCCCCTCGAATTATCAAAACAATT
GCCTCTAAGACTCAGAGTATCAAAAAGTACTCAACCGACATTCTGGGAAACCTGTATGAGGTGAAGAG
CAAAAAGCACCCTCAGATTATCAAAAAGGGCagcggaggcaagcgtcctgctgctactaagaaagctg
gtcaagctaagaaaaagaaaggatcctacccatacgatgttccagattacgcttaagaattcctagag
ctcgctgatcagcctcgactgtgccttctagttgccagccatctgttgtttgcccctcccccgtgcct
tccttgaccctggaaggtgccactcccactgtcctttcctaataaaatgaggaaattgcatcgcattg
tctgagtaggtgtcattctattctggggggtggggtggggcaggacagcaagggggaggattgggaag
agaatagcaggcatgctggggaggtagcggccgcCCgcggtggagctccagcttttgttccctttagt
gagggttaattgcgcgcttggcgtaatcatggtcatagctgtttcctgtgtgaaattgttatccgctc
acaattccacacaacatacgagccggaagcataaagtgtaaagcctggggtgcctaatgagtgaqcta
actcacattaattgcgttgcgctcactgcccgctttccagtcgggaaacctgtcgtgccagctgcatt
aatgaatcggccaacgcgcggggagaggcggtttgcgtattgggcgctcttccgcttcctcgctcact
gactcgctgcgctcggtcgttcggctgcggcgagcggtatcagctcactcaaaggcggtaatacggtt
atccacagaatcaggggataacgcaggaaagaacatgtgagcaaaaggccagcaaaaggccaggaacc
gtaaaaaggccgcgttgctggcgtttttccataggctccgcccccctgacgagcatcacaaaaatcga
cgctcaagtcagaggtggcgaaacccgacaggactataaagataccaggcgtttccccctggaagctc
ccccgtgcgctctcctgttccgaccctgccgcttaccggatacctgtccgcctttctcccttcgggaa
gcgtggcgctttctcatagctcacgctgtaggtatctcagttcggtgtaggtcgttcgctccaagctg
ggctgtgtgcacgaaccccccgttcagcccgaccgctgcgccttatccggtaactatcgtcttgagtc
caacccggtaagacacgacttatcgccactggcagcagccactggtaacaggattagcagagcgaggt
atgtaggcggtgctacagagttcttgaagtggtggcctaactacggctacactagaaggacagtattt
ggtatctgcgctctgctgaagccagttaccttcggaaaaagagttggtagctcttgatccggcaaaca
aaccaccgctggtagcggtggtttttttgtttgcaagcagcagattacgcgcagaaaaaaaggatctc
aagaagatcctttgatcttttctacggggtctgacgctcagtggaacgaaaactcacgttaagggatt
ttggtcatgagattatcaaaaaggatcttcacctagatccttttaaattaaaaatgaagttttaaatc
aatctaaagtatatatgagtaaacttggtctgacagttaccaatgcttaatcagtgaggcacctatct
cagcgatctgtetatttcgtteatccatagttgcctgactccccgtcgtgtagataactacgatacgg
gagggcttaccatctggccccagtgctgcaatgataccgcgagacccacgctcaccggctccagattt
atcagcaataaaccagccagccggaagggccgagcgcagaagtggtcctgcaactttatccgcctcca
tccagtctattaattgttgccgggaagctagagtaagtagttcgccagttaatagtttgcgcaacgtt
gttgccattgctacaggcatcgtggtgtcacgctcgtcgtttggtatggcttcattcagctccggttc
ccaacgatcaaggcgagttacatgatcccccatgttgtgcaaaaaagcggttagctccttcggtcctc
cgatcgttgtcagaagtaagttggccgcagtgttatcactcatggttatggcagcactgcataattct
cttactgtcatgccatccgtaagatgcttttctgtgactggtgagtactcaaccaagtcattctgaga
atagtgtatgcggcgaccgagttgctcttgcccggcgtcaatacgggataataccgcgccacatagca
gaactttaaaagtgctcatcattggaaaacgttcttcggggcgaaaactctcaaggatcttaccgctg
ttgagatccagttcgatgtaacccactcgtgcacccaactgatcttcagcatcttttactttcaccag
cgtttctgggtgagcaaaaacaggaaggcaaaatgccgcaaaaaagggaataagggcgacacggaaat
gttgaatactcatactcttcctttttcaatattattgaagcatttatcagggttattgtctcatgagc
ggatacatatttgaatgtatttagaaaaataaacaaataggggttccgcgcacacttccccgaaaagt
gccac
SEQ ID NO: 38
Fwd primer
5′-aatgatacggcgaccaccgagatctacacaatttcttgggtagtttgcagtt
SEQ ID NO: 39
Revrse primer
5′-caagcagaagacggcatacgagat-(6-bp index sequence)-
actcggtgccactttttcaa
SEQ ID NO: 40
Read1
5′-gatttcttggctttatatatcttgtggaaaggacgaaacaccg
SEQ ID NO: 41
Index
5′-gctagtccgttatcaacttgaaaaagtggcaccgagtc
SEQ ID NO: 42
Read2
5′-gttgataacggactagccttattttaacttgctatttctagctctaaaac
SEQ ID NO: 43
VP64-dCas9-VP64 protein
RADALDDFDLDMLGSDALDDFDLDMLGSDALDDFDLDMLGSDALDDFDLDMVNPKKKRKVGRGMDKKY
SIGLAIGTNSVGWAVITDEYKVPSKKFKVLGNTDRHSIKKNLIGALLFDSGETAEATRLKRTARRRYT
RRKNRICYLQEIFSNEMAKVDDSFFHRLEESFLVEEDKKHERHPIFGNIVDEVAYHEKYPTIYHLRKK
LVDSTDKADLRLIYLALAHMIKFRGHFLIEGDLNPDNSDVDKLFIQLVQTYNQLFEENPINASGVDAK
AILSARLSKSRRLENLIAQLPGEKKNGLFGNLIALSLGLTPNFKSNFDLAEDAKLQLSKDTYDDDLDN
LIAQIGDQYADLFLAAKNLSDAILLSDILRVNTEITKAPLSASMIKRYDEHHQDLTLLKALVRQQLPE
KYKEIFFDQSKNGYAGYIDGGASQEEFYKFIKPILEKMDGTEELLVKLNREDLLRKQRTFDNGSIPHQ
IHLGELHAILRRQEDFYPFLKDNREKIEKILTFRIPYYVGPLARGNSRFANMTRKSEETITPWNFEEV
VDKGASAQSFIERMTNFDKNLPNEKVLPKHSLLYEYFTVYNELTKVKYVTEGMRKPAFLSGEQKKAIV
DLLFKTNRKVTVKQLKEDYFKKIECFDSVEISGVEDRFNASLGTYHDLLKIIKDKDFLDNEENEDILE
DIVLTLTLFEDREMIEERLKTYAHLFDDKVMKQLKRRRYTGWGRLSRKLINGIRDKQSGKTILDFLKS
DGFANRNFMQLIHDDSLTFKEDIQKAQVSGQGDSLHEHIANIAGSPAIKKGILQTVKVVDELVKVMGR
HKPENIVIEMARENQTTQKGQKNSRERMKRIEEGIKELGSQILKEHPVENTQLQNEKLYLYYLQNGRD
MYVDQELDINRLSDYDVDAIVPQSFLKDDSIDNKVLTRSDKNRGKSDNVPSEEVVKKMKKYWRQLLNA
KLITQRKFDNLTKAERGGLSELDKAGFIKRQLVETRQITKHVAQILDSRMNTKYDENDKLIREVKVIT
LKSKLVSDFRKDFQFYKVREINNYHHAHDAYLNAVVGTALIKKYPKLESEFVYGDYKVYDVRKMIAKS
EQEIGKATAKYFFYSNIMNFFKTEITLANGEIRKRPLIETNGETGEIVWDKGRDFATVRKVLSMPQVN
IVKKTEVQTGGFSKESILPKRNSDKLIARKKDWDPKKYGGFDSPTVAYSVLVVAKVEKGKSKKLKSVK
ELLGITIMERSSFEKNPIDFLEAKGYKEVKKDLIIKLPKYSLFELENGRKRMLASAGELQKGNELALP
SKYVNFLYLASHYEKLKGSPEDNEQKQLFVEQHKHYLDEIIEQISEFSKRVILADANLDKVLSAYNKH
RDKPIREQAENIIHLFTLTNLGAPAAFKYFDTTIDRKRYTSTKEVLDATLIHQSITGLYETRIDLSQL
GGDSRADPKKKRKVASRADALDDFDLDMLGSDALDDFDLDMLGSDALDDFDLDMLGSDALDDFDLDML
I
SEQ ID NO: 44
VP64-dCas9-VP64 DNA
cgggctgacgcattggacgattttgatctggatatgctgggaagtgacgccctcgatgattttgacct
tgacatgcttggttcggatgcccttgatgactttgacctcgacatgctcggcagtgacgcccttgatg
atttcgacctggacatggttaaccccaagaagaagaggaaggtgggccgcggaatggacaagaagtac
tccattgggctcgccatcgqcacaaacagcgtcggctgggccgtcattacggacgagtacaaggtgcc
gagcaaaaaattcaaagttctgggcaataccgatcgccacagcataaagaagaacctcattggcgccc
tcctgttcgactccggggaaaccgccgaagccacgcggctcaaaagaacagcacggcgcagatatacc
cgcagaaagaatcggatctgctacctgcaggagatctttagtaatgagatggctaaggtggatgactc
tttcttccataggctggaggagtcctttttggtggaggaggataaaaagcacgagcgccacccaatct
ttggcaatatcgtggacgaggtggcgtaccatgaaaagtacccaaccatatatcatctgaggaagaag
cttgtagacagtactgataaggctgacttgcggttgatctatctcgcgctggcgcatatgatcaaatt
tcggggacacttcctcatcgagqgggacctgaacccagacaacagcgatgtcgacaaactctttatcc
aactggttcagacttacaatcagcttttcgaagagaacccgatcaacgcatccggagttgacgccaaa
gcaatcctgagcgctaggctgtccaaatcccggcggctcgaaaacctcatcgcacagctccctgggga
gaagaagaacggcctgtttggtaatcttatcgccctgtcactcgggctgacccccaactttaaatcta
acttcgacctggccgaagatgccaagcttcaactgagcaaagacacctacgatgatgatctcgacaat
ctgctggcccagatcggcgaccagtacgcagacctttttttggcggcaaagaacctgtcagacgccat
tctgctgagtgatattctgcgagtgaacacggagatcaccaaagctccgctgagcgctagtatgatca
agcgctatgatgagcaccaccaagacttgactttgctgaaggcccttgtcagacagcaactgcctgag
aagtacaaggaaattttcttcgatcagtctaaaaatggctacgccggatacattgacggcggagcaag
ccaggaggaattttacaaatttattaagcccatcttggaaaaaatggacggcaccgaggagctgctgg
taaagcttaacagagaagatctgttgcgcaaacagcgcactttcgacaatggaagcatcccccaccag
attcacctgggcgaactgcacgctatcctcaggcggcaagaggatttctacccctttttgaaagataa
cagggaaaagattgagaaaatcctcacatttcggataccctactatgtaggccccctcgcccggggaa
attccagattcgcgtggatgactcgcaaatcagaagagaccatcactccctggaacttcgaggaagtc
gtggataagggggcctctgcccagtccttcatcgaaaggatgactaactttgataaaaatctgcctaa
cgaaaaggtgcttcctaaacactctctgctgtacgaqtacttcacagtttataacgagctcaccaagg
tcaaatacgtcacagaagggatqagaaagccagcattcctgtctggagagcagaagaaagctatcgtg
gacctcctcttcaagacgaaccggaaagttaccgtgaaacagctcaaagaagactatttcaaaaagat
tgaatgtttcgactctgttgaaatcagcggagtggaggatcgcttcaacgcatccctgggaacgtatc
acgatctcctgaaaatcattaaagacaaggacttcctggacaatgaggagaacgaggacattcttgag
gacattgtcctcacccttacgttgtttgaagatagggagatgattgaagaacgcttgaaaacttacgc
tcatctcttcgacgacaaagtcatgaaacagctcaagaggcgccgatatacaggatgggggcggctgt
caagaaaactgatcaatgggatccgagacaagcagagtggaaagacaatcctggattttcttaagtcc
gatggatttgccaaccggaacttcatgcagttgatccatgatgactctctcacctttaaggaggacat
ccagaaagcacaagtttctggccagggggacagtcttcacgagcacatcgctaatcttgcaggtagcc
cagctatcaaaaagggaatactgcagaccgttaaggtcgtggatgaactcgtcaaagtaatgggaagg
cataagcccgagaatatcgttatcgagatgqcccgaqagaaccaaactacccagaagggacagaagaa
cagtagggaaaggatgaagaggattgaagagggtataaaagaactggggtcccaaatccttaaggaac
acccagctgaaaacacccagcttcagaatgagaagctctacctgtactacctgcagaacggcagggac
atgtacgtggatcaggaactggacatcaatcggctctccgactacgacgtggatgccatcgtgcccca
gtcttttctcaaagatgattctattgataataaagtgttgacaagatccgataaaaatagagggaaga
gtgataacgtcccctcagaagaagttgtcaagaaaatgaaaaattattggcggcagctgctgaacgcc
aaactgatcacacaacggaagttcgataatctgactaaggctgaacgaggtggcctgtctgagttgga
taaagccggcttcatcaaaaggcagcttgttgagacacgccagatcaccaagcacgtggcccaaattc
tcgattcacgcatgaacaccaagtacgatgaaaatgacaaactgattcgagaggtgaaagttattact
ctgaaqtctaagctggtctcagatttcagaaaggactttcagttttataaggtgagagagatcaacaa
ttaccaccatgcgcatgatgcctacctgaatgcagtggtaggcactgcacttatcaaaaaatatccca
agcttgaatctgaatttgtttacggagactataaagtgtacgatgttaggaaaatgatcgcaaagtct
gagcaggaaataggcaaggccaccgctaagtacttcttttacagcaatattatgaattttttcaagac
cgagattacactggccaatggagagattcggaagcgaccacttatcgaaacaaacggagaaacaggag
aaatcgtgtgggacaagggtagggatttcgcgacagtccggaaggtcctgtccatgccgcaggtgaac
atcgttaaaaagaccgaagtacagaccggaggcttctccaaggaaagtatcctcccgaaaaggaacag
cqacaagctgatcgcacgcaaaaaagattqggaccccaagaaatacggcggattcgattctcctacag
tcgcttacagtgtactggttgtqgccaaaqtggagaaagggaagtctaaaaaactcaaaagcgtcaag
gaactgctgggcatcacaatcatggagcgatcaagcttcgaaaaaaaccccatcgactttctcgaggc
gaaaggatataaagaggtcaaaaaagacctcatcattaagcttcccaagtactctctctttgagcttg
aaaacggccggaaacgaatgctcgctagtgcgggcgagctgcagaaaggtaacgagctggcactgccc
tctaaatacgttaatttcttgtatctggccagccactatgaaaagctcaaagggtctcccgaagataa
tgagcagaagcagctgttcgtggaacaacacaaacactaccttgatgagatcatcgagcaaataagcg
aattctccaaaagagtgatcctcgccgacgctaacctcgataaggtgctttctgcttacaataagcac
agggataagcccatcagggagcaggcagaaaacattatccacttgtttactctgaccaacttgggcgc
gcctgcagccttcaagtacttcgacaccaccatagacagaaagcggtacacctctacaaaggaggtcc
tggacgccacactgattcatcagtcaattacggggctctatgaaacaagaatcgacctctctcagctc
ggtggagacagcagggctgaccccaagaagaagaggaaggtggctagccgcgccgacgcgctggacga
tttcgatctcgacatgctgggttctgatgccctcgatgactttgacctggatatgttgggaagcgacg
cattggatgactttgatctggacatgctcggctccgatgctctggacgatttcgatctcgatatgtta
atc
SEQ ID NO: 45
Human p300 (with L553M mutation) protein
MAENVVEPGPPSAKRPKLSSPALSASASDGTDFGSLFDLEHDLPDELINSTELGLTNGGDINQLQTSL
GMVQDAASKHKQLSELLRSGSSPNLNKGVGGPGQVMASQAQQSSPGLGLINSMVKSPMTQAGLTSPNM
GMGTSGPNQGPTQSTGMMNSPVNQPAMGMNTGMNAGMNPGMLAAGNGQGIMPNQVMNGSIGAGRGRQN
MQYPNPGMGSAGNLLTEPLQQGSPQMGGQTGLRGPQPLKMGMMNNPKPYGSPYTQNPGQQIGASGLGL
QIQTKTVLSNNLSPFAMDKKAVPGGGMPNMGQQPAPQVQQPGLVTPVAQGMGSGAHTADPEKRKLIQQ
QLVLLLHAHKCQRREQANGEVRQCNLPHCRTMKNVLNHMTHCQSGKSCQVAHCASSRQIISHWKNCTR
HDCPVCLPLKNAGDKRNQQPILTGAPVGLGNPSSLGVGQQSAPNLSTVSQIDPSSIERAYAALGLPYQ
VNQMPTQPQVQAKNQQNQQPGQSPQGMRPMSNMSASPMGVNGGVGVQTPSLLSDSMLHSAINSQNPMM
SENASVPSMGPMPTAAQPSTTGIRKQWHEDITQDLRNHLVHKLVQAIFPTPDPAALKDRRMEKLVAYA
RKVEGDMYESANNRAEYYHLLAEKIYKIQKELEEKRRTRLQKQNMLPNAAGMVPVSMNPGPNMGQPQP
GMTSNGPLPDPSMIRGSVPNQMMPRITPQSGLNQFGQMSMAQPPIVPRQTPPLQHKGQLAQPGALNPP
MGYGPRMQQPSKQGQFLPQTQFPSQGMNVTNIPLAPSSGQAPVSQAQKSSSSCPVKSPIMPPGSQGSH
IHCPQLPQPALHQNSPSPVPSRTPTPHHTPPSIGAQQPPATTIPAPVPTPPAMPPGPQSQALKPPPRQ
TPTPPTTQLPQQVQPSLPAAPSADQPQQQPRSQQSTAASVPTPTAPLLPPQFATPLSQPAVSIEGQVS
NPPSTSSTEVNSQAIAEKQPSQEVKMEAKMEVDQPEPADTQPEDISESKVEDCKMESTETEERSTELK
TEIKEEEDQPSTSATQSSPAPGQSKKKIFKPEELRQALMPTLEALYRQDPESLPFRQPVDPQLLGIPD
YFDIVKSPMDLSTIKRKLDTGQYQEPWQYVDDIWLMFNNAWLYNRKTSRVYKYCSKLSEVFEQEIDPV
MQSLGYCCGRKLEFSPQTLCCYGKQLCTIPRDATYYSYQNRYHFCEKCFNEIQGESVSLGDDPSQPQT
TINKEQFSKRKKDTLDPELFVECTECGRKMHQICVLHHEIIWPAGFVCDGCLKKSARTRKENKFSAKR
LPSTRLGTFLENRVNDFLRRQNHPESGEVTVRVVHASDKTVEVKPGMKARFVDSGEMAESFPYRTKAL
FAEEEIDGVDLCFFGMHVQEYGSDCPPPNQRRVYISYLDSVHFFRPKCLRTAVYHEIEIGYLEYVKKL
GYTTGH1WACPPSEGDDYIFKCHPPDQKIPKPKRLQEWYKKMLDKAVSERIVKDYKDIFKQATEDRLT
SAKELPYFEGDFWPNVLEESIKELEQEEEERKREENTSNESTDVTKGDSKNAKKKNNKKTSKKKSSLS
RGNKKKPGMPNVSNDLSQKLYATMEKHKEVFFVIRLIAGPAANSLPPIVDPDPLIPCDLMDGRDAFLT
LARDKHLEFSSLRRAQWSTMCMLVELHTQSQDRFVYTCKECKHHVETRWHCTVCEDYDLCITCYNTKN
HDHKMEKLGLGLDDESNNQQAAATQSPGDSRRLSIQRCIQSLVHACQCRNANCSLPSCQKMKRVVQHT
KGCKRKTNGGCPICKQLIALCCYHAKHCQENKCPVPFCLNIKQKLRQQQLQHRLQQAQMLRRRMASMQ
RTGVVGQQQGLPSPTPATPTTPTGQQPTTPQTPQPTSQPQPTPPNSMPPYLPRTQAAGPVSQGKAAGQ
VTPPTPPQTAQPPEPGPPPAAVEMAMQIQRAAETQRQMAHVQIFQRPIQHQMPPMTPMAPMGMNPPPM
TRGPSGHLEPGMGPTGMQQQPPWSQGGLPQPQQLQSGMPRPAMMSVAQHGQPLNMAPQPGLGQVGISP
LKPGTVSQQALQNLLRTLRSPSSPLQQQQVLSILHANPQLLAAFIKQRAAKYANSNPQPIPGQPGMPQ
GQPGLQPPTMPGQQGVHSNPAMQNMNPMQAGVQRAGLPQQQPQQQLQPPMGGMSPQAQQMKMNHNTMP
SQFRDILRRQQMMQQQQQQGAGPGIGPGMANHNQFQQPQGVGYPPQQQQRMQHHMQQMQQGNMGQIGQ
LPQALGAEAGASLQAYQQRLLQQQMGSPVQPKPMSPQQKMLPNQAQSPHLQGQQIPNSLSNQVRSPQP
VPSPRPQSQPPHSSPSPRMQPQPSPHHVSPQTSSPHPGLVAAQANPMEQGHFASPDQNSMLSQLASNP
GMAMLHGASATDLGLSTDNSDLNSNLSQSTLDIH
SEQ ID NO: 46
Human p300 Core Effector protein (aa 1048-1664 of SEQ ID NO: 134)
IFKPEELRQALMPTLEALYPQDPESLPFRQPVDPQELGIPDYFDIVKSPMDLSTIKRKLDTGQYQEPW
QYVDDIWLMFNNAWLYNRKTSRVYKYCSKLSEVFEQEIDPVMQSLGYCCGRKLEFSPQTLCCYGKQLC
TIPRDATYYSYQNRYHFCEKCFNEIQGESVSLGDDPSQPQTTINKEQFSKRKNDTLDPELFVECTECG
RKMHQICVLHHEIIWPAGEVCDGCLKKSARTRKENKFSAKRLPSTRLGTFLENRVKDFLRRQNHPESG
EVTVRVVKASDKTVEVKPGMKARFVD3GEMAE3FPYRTKALFAFEEIDGVDLCFFGMHVQEYGSDCPP
PNQRRVYISYLDSVHFFRPKCLRTAVYHEILIGYLEYVKKLGYTTGHIWACPPSEGDDYIFHCHPPDQ
KIPKPKRLQEWYKKMLDKAVSERIVHDYKDIFKQATEDRLTSAKELPYFEGDFWPNVLEESIKELEQE
EEERKREENTSNESTDVTKGDSKNAKKKNNKKTSKNKSSLSRGNKKKPGMPNVSNDLSQKLYATMEKH
KEVFFVIRLIAGPAANSLPPIVDPDPLIPCDLMDGRDAFLTLARDKHLEFSSLRRAQWSTMCMLVELH
TQSQD
SEQ ID NO: 87
Polynucleotide sequence encoding Streptococcus pyogenes dCas9-KRAB
atggactacaaagaccatgacggtgattataaagatcatgacatcgattacaaggatgacga
tgacaagatggcccccaagaagaagaggaaggtgggccgcggaatggacaagaagtactcca
ttgggctcgccatcggcacaaacagcgtcggctgggccgtcattacggacgagtacaaggtg
ccgagcaaaaaattcaaagttctgggcaataccgatcgccacagcataaagaagaacctcat
tggcgccctcctgttcgactccggggaaaccgccgaagccacgcggctcaaaagaacagcac
ggcgcagatatacccgcagaaagaatcggatctgctacctgcaggagatctttagtaatgag
atggctaaggtggatgactctttcttccataggctggaggagtcctttttggtggaggagga
taaaaagcacgagcgccacccaatctttggcaatatcgtggacgaggtggcgtaccatgaaa
agtacccaaccatatatcatctgaggaagaagcttgtagacagtactgataaggctgacttg
cggttgatctatctcgcgctggcgcatatgatcaaatttcggggacacttcctcatcgaggg
ggacctgaacccagacaacagcgatgtcgacaaactctttatccaactggttcagacttaca
atcagcttttcgaagagaacccgatcaacgcatccggagttgacgccaaagcaatcctgagc
gctaggctgtccaaatcccggcggctcgaaaacctcatcgcacagctccctggggagaagaa
gaacggcctgtttggtaatcttatcgccctgtcactcgggctgacccccaactttaaatcta
acttcgacctggccgaagatgccaagcttcaactgagcaaagacacctacgatgatgatctc
gacaatctgctggcccagatcggcgaccagtacgcagacctttttttggcggcaaagaacct
gtcagacgccattctgctgagtgatattctgcgagtgaacacggagatcaccaaagctccgc
tgagcgctagtatgatcaagcgctatgatgagcaccaccaagacttgactttgctgaaggcc
cttgtcagacagcaactgcctgagaagtacaaggaaattttcttcgatcagtctaaaaatgg
ctacgccggatacattgacggcggagcaagccaggaggaattttacaaatttattaagccca
tcttggaaaaaatggacggcaccgaggagctgctggtaaagcttaacagagaagatctgttg
cgcaaacagcgcactttcgacaatggaagcatcccccaccagattcacctgggcgaactgca
cgctatcctcaggcggcaagaggatttctacccctttttgaaagataacagggaaaagattg
agaaaatcctcacatttcggataccctactatgtaggccccctcgcccggggaaattccaga
ttcgcgtggatgactcgcaaatcagaagagaccatcactccctggaacttcgaggaagtcgt
ggataagggggcctctgcccagtccttcatcgaaaggatgactaactttgataaaaatctgc
ctaacgaaaaggtgcttcctaaacactctctgctgtacgagtacttcacagtttataacgag
ctcaccaaggtcaaatacgtcacagaagggatgagaaagccagcattcctgtctggagagca
gaagaaagctatcgtggacctcctcttcaagacgaaccggaaagttaccgtgaaacagctca
aagaagactatttcaaaaagattgaatgtttcgactctgttgaaatcagcggagtggaggat
cgcttcaacgcatccctgggaacgtatcacgatctcctgaaaatcattaaagacaaggactt
cctggacaatgaggagaacgaggacattcttgaggacattgtcctcacccttacgttgtttg
aagatagggagatgattgaagaacgcttgaaaacttacgctcatctcttcgacgacaaagtc
atgaaacagctcaagaggcgccgatatacaggatgggggcggctgtcaagaaaactgatcaa
tgggatccgagacaagcagagtggaaagacaatcctggattttcttaagtccgatggatttg
ccaaccggaacttcatgcagttgatccatgatgactctctcacctttaaggaggacatccag
aaagcacaagtttctggccagggggacagtcttcacgagcacatcgctaatcttgcaggtag
cccagctatcaaaaagggaatactgcagaccgttaaggtcgtggatgaactcgtcaaagtaa
tgggaaggcataagcccgagaatatcgttatcgagatggcccgagagaaccaaactacccag
aagggacagaagaacagtagggaaaggatgaagaggattgaagagggtataaaagaactggg
gtcccaaatccttaaggaacacccagttgaaaacacccagcttcagaatgagaagctctacc
tgtactacctgcagaacagcagggacatgtacgtggatcaggaactggacatcaatcggctc
tccgactacgacgtggatgccatcgtgccccagtcttttctcaaagatgattctattgataa
taaagtgttgacaagatccgataaaaatagagggaagagtgataacgtcccctcagaagaag
ttgtcaagaaaatgaaaaattattggcggcagctgctgaacgccaaactgatcacacaacgg
aagttcgataatctgactaaggctgaacgaggtggcctgtctgagttggataaagccggctt
catcaaaaggcagcttgttgagacacgccagatcaccaagcacgtggcccaaattctcgatt
cacgcatgaacaccaagtacgatgaaaatgacaaactgattcgagaggtgaaagttattact
ctgaagtctaagctggtctcagatttcagaaaggactttcagttttataaggtgagagagat
caacaattaccaccatgcgcatgatgcctacctgaatgcagtggtaggcactgcacttatca
aaaaatatcccaagcttgaatctgaatttgtttacggagactataaagtgtacgatgttagg
aaaatgatcgcaaagtctgagcaggaaataggcaaggccaccgctaagtacttcttttacag
caatattatgaattttttcaagaccgagattacactggccaatggagagattcggaagcgac
cacttatcgaaacaaacggagaaacaggagaaatcgtgtgggacaagggtagggatttcgcg
acagtccggaaggtcctgtccatgccgcaggtgaacatcgttaaaaagaccgaagtacagac
cggaggcttctccaaggaaagtatcctcccgaaaaggaacagcgacaagctgatcgcacgca
aaaaagattgggaccccaagaaatacggcggattcgattctcctacagtcgcttacagtgta
ctggttgtggccaaagtggagaaagggaagtctaaaaaactcaaaagcgtcaaggaactgct
gggcatcacaatcatggagcgatcaagcttcgaaaaaaaccccatcgactttctcgaggcga
aaggatataaagaggtcaaaaaagacctcatcattaagcttcccaagtactctctctttgag
cttgaaaacggccggaaacgaatgctcgctagtgcgggcgagctgcagaaaggtaacgagct
ggcactgccctctaaatacgttaatttcttgtatctgaccagccactatgaaaagctcaaag
ggtctcccgaagataatgagcagaagcagctgttcgtggaacaacacaaacactaccttgat
gagatcatcgagcaaataagcgaattctccaaaagagtgatcctcgccgacgctaacctcga
taaggtgctttctgcttacaataagcacagggataagcccatcagggagcaggcagaaaaca
ttatccacttgtttactctgaccaacttgggcgcgcctgcagccttcaagtacttcgacacc
accatagacagaaagcggtacacctctacaaaggaggtcctggacgccacactgattcatca
gtcaattacggggctctatgaaacaagaatcgacctctctcagctcggtggagacagcaggg
ctgaccccaagaagaagaggaaggtggctagcgatgctaagtcactgactgcctggtcccgg
acactggtgaccttcaaggatgtgtttgtggacttcaccagggaggagtggaagctgctgga
cactgctcagcagatcctgtacagaaatgtgatgctggagaactataagaacctggtttcct
tgggttatcagcttactaagccagatgtgatcctccggttggagaagggagaagagccctgg
ctggtggagagagaaattcaccaagagacccatcctgattcagagactgcatttgaaatcaa
atcatcagttccgaaaaagaaacgcaaagtttga
SEQ ID NO: 88
Polypeptide sequence of Streptococcus pyogenes dCas9-KRA8 protein
MDYKDHDGDYKDHDIDYKDDDDKMAPKKKRKVGRGMDKKYSIGLAIGTNSVGWAVITDEYKV
PSKKFKVLGNTDRHSIKKNLIGALLFDSGETAEATRLKRTARRRYTRRKNRICYLQEIFSNE
MAKVDDSFFHRLEESFLVEEDKKHERHPIFGNIVDEVAYHEKYPTIYHLRKKLVDSTDKADL
RLIYLALAHMIKFRGHFLIEGDLNPDNSDVDKLFIQLVQTYNQLFEENPINASGVDAKAILS
ARLSKSRRLENLIAQLPGEKKNGLFGNLIALSLGLTPNFKSNFDLAEDAKLQLSKDTYDDDL
DNLLAQIGDQYADLFLAAKNLSDAILLSDILRVNTEITKAPLSASMIKRYDEHHQDLTLLKA
LVRQQLPEKYKEIFFDQSKNGYAGYIDGGASQEEFYKFIKPILEKMDGTEELLVKLNREDLL
RKQRTFDNGSIPHQIHLGELHAILRRQEDFYPFLKDNREKIEKILTFRIPYYVGPLARGNSR
FAWMTRKSEETITPWNFEEVVDKGASAQSFIERMTNFDKNLPNEKVLPKHSLLYEYFTVYNE
LTKVKYVTEGMRKPAFLSGEQKKAIVDLLFKTNRKVTVKQLKEDYFKKIECFDSVEISGVED
RFNASLGTYHDLLKIIKDKDFLDNEENEDILEDIVLTLTLFEDREMIEERLKTYAHLFDDKV
MKQLKRRRYTGWGRLSRKLINGIRDKQSGKTILDFLKSDGFANRNFMQLIHDDSLTFKEDIQ
KAQVSGQGDSLHEHIANLAGSPAIKKGILQTVKVVDSLVKVMGRHKPENIVIEMARENQTTQ
KGQKNSRERMKRIEEGIKELGSQILKEHPVENTQLQNEKLYLYYLQNGRDMYVDQELDINRL
SDYDVDAIVPQSFLKDDSIDNKVLTRSDKNRGKSDNVPSEEVVKKMKNYWRQLLNAKLITQR
KFDNLTKAERGGLSELDKAGFIKRQLVETRQITKHVAQILDSRMNTKYDENDKLIREVKVIT
LKSKLVSDFRKDFQFYKVREINNYHHAHDAYLNAVVGTALIKKYPKLESEFVYGDYKVYDVR
KMIAKSEQEIGKATAKYFFYSNIMNFFKTEITLANGEIRKRPLIETNGETGEIVWDKGRDFA
TVRKVLSMPQVNIVKKTEVQTGGFSKESILPKRNSDKLIARKKDWDPKKYGGFDSPTVAYSV
LVVAKVEKGKSKKLKSVKELLGITIMERSSFEKNPIDFLEAKGYKEVKKDLIIKLPKYSLFE
LENGRKRMLASAGELQKGNELALPSKYVNFLYLASHYEKLKGSPEDNEQKQLFVEQHKHYLD
EIIEQISEFSKRVILADANLDKVLSAYNKHRDKPIREQAENIIHLFTLTNLGAPAAFKYFDT
TIDRKRYTSTKEVLDATLIHQSITGLYETRIDLSQLGGDSRADPKKKRKVASDAKSLTAWSR
TLVTFKDVFVDFTREEWKLLDTAQQILYRNVMLENYKNLVSLGYQLTKPDVILRLEKGEEPW
LVEREIHQETHPDSETAFEIKSSVPKKKRKV
SEQ ID NO: 1138
Polynucleotide sequence of Tet1CD (or of Tet1CD-dCas9)
CTGCCCACCTGCAGCTGTCTTGATCGAGTTATACAAAAAGACAAAGGCCCATATTATACACA
CCTTGGGGCAGGACCAAGTGTTGCTGCTGTCAGGGAAATCATGGAGAATAGGTATGGTCAAA
AAGGAAACGCAATAAGGATAGAAATAGTAGTGTACACCGGTAAAGAAGGGAAAAGCTCTCAT
GGGTGTCCAATTGCTAAGTGGGTTTTAAGAAGAAGCAGTGATGAAGAAAAAGTTCTTTGTTT
GGTCCGGCAGCGTACAGGCCACCACTGTCCAACTGCTGTGATGGTGGTGCTCATCATGGTGT
GGGATGGCATCCCTCTTCCAATGGCCGACCGGCTATACACAGAGCTCACAGAGAATCTAAAG
TCATACAATGGGCACCCTACCGACAGAAGATGCACCCTCAATGAAAATCGTACCTGTACATG
TCAAGGAATTGATCCAGAGACTTGTGGAGCTTCATTCTCTTTTGGCTGTTCATGGAGTATGT
ACTTTAATGGCTGTAAGTTTGGTAGAAGCCCAAGCCCCAGAAGATTTAGAATTGATCCAAGC
TCTCCCTTACATGAAAAAAACCTTGAAGATAACTTACAGAGTTTGGCTACACGATTAGCTCC
AATTTATAAGCAGTATGCTCCAGTAGCTTACCAAAATCAGGTGGAATATGAAAATGTTGCCC
GAGAATGTCGGCTTGGCAGCAAGGAAGGTCGACCCTTCTCTGGGGTCACTGCTTGCCTGGAC
TTCTGTGCTCATCCCCACAGGGACATTCACAACATGAATAATGGAAGCACTGTGGTTTGTAC
CTTAACTCGAGAAGATAACCGCTCTTTGGGTGTTATTCCTCAAGATGAGCAGCTCCATGTGC
TACCTCTTTATAAGCTTTCAGACACAGATGAGTTTGGCTCCAAGGAAGGAATGGAAGCCAAG
ATCAAATCTGGGGCCATCGAGGTCCTGGCACCCCGCCGCAAAAAAAGAACGTGTTTCACTCA
GCCTGTTCCCCGTTCTGGAAAGAAGAGGGCTGCGATGATGACAGAGGTTCTTGCACATAAGA
TAAGGGCAGTGGAAAAGAAACCTATTCCCCGAATCAAGCGGAAGAATAACTCAACAACAACA
AACAACAGTAAGCCTTCGTCACTGCCAACCTTAGGGAGTAACACTGAGACCGTGCAACCTGA
AGTAAAAAGTGAAACCGAACCCCATTTTATCTTAAAAAGTTCAGACAACACTAAAACTTATT
CGCTGATGCCATCCGCTCCTCACCCAGTGAAAGAGGCATCTCCAGGCTTCTCCTGGTCCCCG
AAGACTGCTTCAGCCACACCAGCTCCACTGAAGAATGACGCAACAGCCTCATGCGGGTTTTC
AGAAAGAAGCAGCACTCCCCACTGTACGATGCCTTCGGGAAGACTCAGTGGTGCCAATGCTG
CAGCTGCTGATGGCCCTGGCATTTCACAGCTTGGCGAAGTGGCTCCTCTCCCCACCCTGTCT
GCTCCTGTGATGGAGCCCCTCATTAATTCTGAGCCTTCCACTGGTGTGACTGAGCCGCTAAC
GCCTCATCAGCCAAACCACCAGCCCTCCTTCCTCACCTCTCCTCAAGACCTTGCCTCTTCTC
CAATGGAAGAAGATGAGCAGCATTCTGAAGCAGATGAGCCTCCATCAGACGAACCCCTATCT
GATGACCCCCTGTCACCTGCTGAGGAGAAATTGCCCCACATTGATGAGTATTGGTCAGACAG
TGAGCACATCTTTTTGGATGCAAATATTGGTGGGGTGGCCATCGCACCTGCTCACGGCTCGG
TTTTGATTGAGTGTGCCCGGCGAGAGCTGCACGCTACCACTCCTGTTGAGCACCCCAACCGT
AATCATCCAACCCGCCTCTCCCTTGTCTTTTACCAGCACAAAAACCTAAATAAGCCCCAACA
TGGTTTTGAACTAAACAAGATTAAGTTTGAGGCTAAAGAAGCTAAGAATAAGAAAATGAAGG
CCTCAGAGCAAAAAGACCAGGCAGCTAATGAAGGTCCAGAACAGTCCTCTGAAGTAAATGAA
TTGAACCAAATTCCTTCTCATAAAGCATTAACATTAACCCATGACAATGTTGTCACCGTGTC
CCCTTATGCTCTCACACACGTTGCGGGGCCCTATAACCATTGGGTC
SEQ ID NO: 1139
Polypeptide sequence of Tet1CD (or of Tet1CD-dCas9)
LPTCSCLDRVIQKDKGPYYTHLGAGPSVAAVREIMENRYGQKGNAIRIEIVVYTGKEGKSSH
GCPIAKWVLRRSSDEEKVLCLVRQRTGHHCPTAVMVVLIMVWDGIPLPMADRLYTELTENLK
SYNGHPTDRRCTLNENRTCTCQGIDPETCGASFSFGCSWSMYFNGCKFGRSPSPRRFRIDPS
SPLHEKNLEDNLQSLATRLAPIYKQYAPVAYQNQVEYENVARECRLGSKEGRPFSGVTACLD
FCAHPHRDIHNMNNGSTVVCTLTREDNRSLGVIPQDEQLHVLPLYKLSDTDEFGSKEGMEAK
IKSGAIEVLAPRRKKRTCFTQPVPRSGKKRAAMMTEVLAHKIRAVEKKPIPRIKRKNNSTTT
NNSKPSSLPTLGSNTETVQPEVKSETEPHFILKSSDNTKTYSLMPSAPHPVKEASPGFSWSP
KTASATPAPLKNDATASCGFSERSSTPHCTMPSGRLSCANAAAADGPGISQLGEVAPLPTLS
APVMEPLIMSEPSTGVTEPLTPHQPNHQPSFLTSPQDLASSPMEEDEQHSEADEPPSDEPLS
DDPLSPAEEKLPHIDEYWSDSEHIFLDANIGGVAIAPAHGSVLIECARRELHATTPVEHPNR
NHPTRLSLVFYQHKNLNKPQHGFELNKIKFEAKEAKNKKMKASEQKDQAANEGPEQSSEVNE
LNQIPSHKALTLTHDNVVTVSPYALTHVAGPYNHWV
SEQ ID NO: 1140
DNA sequence of the gRNA constant region
gtttaagagctatgctggaaacagcatagcaagtttaaataaggctagtccgttatcaactt
gaaaaagtggcaccgagtcggtgc
SEQ ID NO: 1141
RNA sequence of the gRNA constant region
guuuaagagcuaugcuggaaacagcauagcaaguuuaaauaaggcuaguccguuaucaacuu
gaaaaaguggcaccgagucggugc
SEQ ID NO: 1142
RNA sequence of the full gRNA, including SEQ ID NO: 588
(underlined)
ggaaccagucagaacaggugguauaagaqcuaugcuggaaacagcauagcaaguuuaaauaa
ggcuaguccguuaucaacuugaaaaaguggcaccgagucggugc

1. A method of treating a subject having Prader Willi Syndrome (PWS) or Prader-Willi-like disorder comprising administering to the subject a DNA Targeting System that targets the 15q11-13 PWS-associated locus.

2. The method of claim 1, wherein the DNA Targeting System is a Targeted Activator System that binds to a target region selected from the group consisting of:
nucleotide positions −127023 to −125023;
nucleotide positions −93065 to −91065;
nucleotide positions −1104 to +896;
nucleotide positions −126547 to −124695 [mat1];
nucleotide positions −131937 to −130580 [mat1A];
nucleotide positions −129415 to −127715 [mat1B];
nucleotide positions −123798 to −122440 [mat1C];
nucleotide positions −92568 to −91460 [mat2];
nucleotide positions −797 to +1346 [mat3]; and/or
nucleotide positions +12858 to +14026 [mat4]
wherein the nucleotide position is relative to position 1 being the start site of SNRPN exon 1 of the PWS imprinting center on chromosome 15.

3. The method of claim 1, wherein the subject is administered a Targeted Activator System that binds to a target region selected from the group consisting of:
nucleotide positions −126523 to −125523;
nucleotide positions −92565 to −91565;
nucleotide positions −804 to +395;
nucleotide positions −126047 to −125195 [mat1];
nucleotide positions −131437 to −131080 [mat1A];
nucleotide positions −128915 to −128215 [mat1B];
nucleotide positions −123298 to −122940 [mat1C];
nucleotide positions −92068 to −91960 [mat2];
nucleotide positions −297 to +846 [mat3]; and/or
nucleotide positions +13358 to +13526 [mat4],
wherein the nucleotide position is relative to position 1 being the start site of SNRPN exon 1 of the PWS imprinting center on chromosome 15.

4. The method of any of claims 1-3, wherein the Targeted Activator System targets a target region that results in increased expression of SNORD116 or its products.

5. The method of any of claims 1-3, wherein the Targeted Activator System targets a target region that results in increased expression of MAGEL2.

6. The method of any of claims 1-3, wherein at least a first Targeted Activator System targets a target region that results in increased expression of SNORD116 and at least a second Targeted Activator System targets a region that results in increased expression of MAGEL2.

7. The method of claim 1, wherein DNA Targeting System is a Targeted Repressor System that binds to a target region selected from the group consisting of:
nucleotide positions +23022 to +25022;
nucleotide positions +34734 to +36734;
nucleotide positions −101358 to −94223 [pat1];
nucleotide positions −58232 to −51914 [pat2];
nucleotide positions −4847 to −3047 [pat3];
nucleotide positions −1774 to +2421 [pat4];
nucleotide positions +2446 to +24016 [pat5];
nucleotide positions +23346 to +25082 [pat6];
nucleotide positions +24340 to +35718 [pat7]; and/or
nucleotide positions +35206 to +36668 [pat8],
wherein the nucleotide position is relative to position 1 being the start site of SNRPN exon 1 of the PWS imprinting center on chromosome 15.

8. The method of claim 1, wherein the subject is administered a Targeted Repressor System that binds to a target region selected from the group consisting of:
nucleotide positions +23522 to +24522;
nucleotide positions +35234 to +36234;
nucleotide positions −100858 to −94723[pat1];
nucleotide positions −57732 to −52414 [pat2];
nucleotide positions −4347 to −3547 [pat3];
nucleotide positions −1275 to +1921 [pat4];
nucleotide positions +2946 to +23516 [pat5];
nucleotide positions +23846 to +24582 [pat6];
nucleotide positions +24840 to +35218 [pat7]; and/or
nucleotide positions +35706 to +36168 [pat8];
wherein the nucleotide position is relative to position 1 being the start site of SNRPN exon 1 of the PWS imprinting center on chromosome 15.

9. The method of any of claims 1-8, wherein the administering to said subject results in increased expression of one or more of the following gene or gene products: MKRN3, MAGEL2, NDN, C15ORF2, SNURF-SNRPN, SNORD107, SNORD64, SNORD109A, SNORD116, SNORD116@, SPA1, SPA2, 116HG, SNORD116-1 to 30, Sno-Inc RNA 1 to 5, IPW, SNORD115, SNORD115@, 115HG, SNORD115-1 to 48, SNORD109B, and/or SNG14.

10. The method of any of claims 1-9, wherein the subject has a PWS Type 1 large deletion, PWS Type 2 large deletion, PWS imprinting center mutation or PWS uniparental disomy.

11. The method of any of claims 1-9, wherein the subject has a PWS microdeletion encompassing SNORD116, but not MAGEL2.

12. The method of any of claims 1-9, wherein the subject has a PWS or PWS-like atypical deletion encompassing MAGEL2, but not SNORD116.

13. The method of any of claims 1-9, wherein the subject has heterozygous Schaaf-Yang syndrome or MAGEL2 disorder.

14. The method of any of claims 1-13, wherein the Targeted Activator System is a CRISPR-Cas Type II system.

15. The method of claim 14, wherein the Targeted Activator System comprises:
(a) a fusion protein comprising:
(i) a Cas9 polypeptide with reduced nuclease activity, and
(ii) an activator; and
(b) one or more guide RNAs (gRNA) that bind to a target region in the 15q11-13 PWS-associated locus.

16. The method of claim 15, wherein at least one gRNA comprises at least 10, 11, 12, 13, 14, 15, 16, 17, 18, 19 or 20 consecutive nucleotides of SEQ ID NOs: 1-12, or of any one of SEQ ID NOs: 47-86 and 91-1122 or a corresponding allelic variant thereof, or the complement of any of SEQ ID NOs: 1-12 or a corresponding allelic variant thereof.

17. The method of claim 15 wherein the Cas9 is a S. pyogenes Cas9 (SEQ ID NO: 24).

18. The method of claim 15, wherein the subject is administered:
(a) said fusion protein and said one or more gRNAs; or
(b) a nucleic acid sequence encoding said fusion protein, and said one or more gRNAs; or
(c) a nucleic acid sequence encoding said fusion protein, and nucleic acid sequence(s) encoding said one or more gRNAs.

19. The method of claim 18, wherein the subject is administered a vector comprising a nucleic acid encoding said fusion protein and/or a vector comprising a nucleic acid encoding said gRNA.

20. The method of claim 19 wherein the vector is a viral vector, optionally a retroviral, lentiviral, adenovirus, adeno-associated virus vector, synthetic vector, or is a vector within a lipid nanoparticle.

21. The method of claim 14, wherein the Targeted Activator System comprises
(a) a Cas9 polypeptide, and
(b) one or more dead gRNAs that bind to a target region in the 15q11-13 PWS-associated locus.

22. The method of any of claims 15-20, wherein the Targeted Activator System is a CRISPR-Cas Type II system comprising one or more gRNAs, wherein at least one gRNA comprises a nucleotide sequence at least 80% complementary to at least 10-20 consecutive nucleotides of said target region.

23. The method of any of claims 15-20 or 22, wherein the at least one gRNA comprises a nucleotide sequence at least 80% complementary to at least 18 consecutive nucleotides of said target region.

24. The method any of claims 1-13, wherein the Targeted Activator System is a ZF-based and/or TALE-based system.

25. The method of claim 24, wherein the Targeted Activator System comprises one or more fusion proteins comprising (a) a TALE that binds to the target region of the 15q11-13 PWS-associated locus and (b) an activator.

26. The method of claim 24, wherein the Targeted Activator System comprises one or more fusion proteins comprising (a) a ZF that binds to the target region of the 15q11-13 PWS-associated locus and (b) an activator.

27. The method of any of claims 15-26 wherein the activator is VP64, VP16; GAL4; p65 subdomain (NFkB); KMT2 family transcriptional activators: hSET1A, hSET1B, MLL1 to 5, ASH1, and homologs (Trx, Trr, Ash1); KMT3 family: SYMD2, NSD1; KMT4 family: DOT1L and homologs; KDM1: LSD1/BHC110 and homologs (SpLsd1/Swm1/Saf110, Su(var)3-3); KDM3 family: JHDM2a/b; KDM4 family: JMJD2A/JHDM3A, JMJD2B, JMJD2C/GASC1, JMJD2D, and homologs (Rph1); KDM6 family: UTX, JMJD3, VP64-p65-Rta (VPR); synergistic action mediator (SAM); p300; VP160; VP64-dCas9-BFP-VP64; KAT2 family: hGCN5, PCAF, and homologs (dGCN5/PCAF, Gcn5; KAT3 family: CBP, p300 and homologs (dCBP/NEJ); KAT4: TAF1 and homologs (dTAF1); KAT5: TIP60/PLIP, and homologs; KAT6: MOZ/MYST3, MORF/MYST4, and homologs (Mst2, Sas3, CG1894); KAT7: HBO1/MYST2, and homologs (CHM, Mst2); KAT8: HMOF/MYST1, and homologs (dMOF, CG1894, Sas2, Mst2); KAT13 family: SRC1, ACTR, P160, CLOCK, and homologs; AID/Apobed deaminase family: AID; TET dioxygenase family: TET1; DEMETER glycosylase family: DME, DML1, DML2, or ROS1.

28. The method of any of claims 1−13, wherein the Targeted Repressor System is a CRISPR-Cas Type II system.

29. The method of claim 28, wherein the Targeted Repressor System comprises
(a) a fusion protein comprising:
(i) a Cas9 polypeptide with reduced nuclease activity, and
(ii) a repressor, and
(b) one or more guide RNAs (gRNA) that bind to the target region of the 15q11-13 PWS-associated locus.

30. The method of claim 29 wherein the Cas9 is a S. pyogenes Cas9 (SEQ ID NO: 24).

31. The method of claim 29, wherein the subject is administered:
(a) said fusion protein and said one or more gRNAs; or
(b) a nucleic acid sequence encoding said fusion protein, and said one or more gRNAs: or
(c) a nucleic acid sequence encoding said fusion protein, and nucleic acid sequence(s) encoding said one or more gRNAs.

32. The method of claim 31, wherein the subject is administered a vector comprising a nucleic acid encoding said fusion protein and/or a vector comprising a nucleic acid encoding said gRNA.

33. The method of claim 32 wherein the vector is a viral vector, optionally a retroviral, lentiviral, adenovirus, adeno-associated virus vector, synthetic vector, or is a vector within a lipid nanoparticle.

34. The method of claim 28, wherein the Targeted Repressor System comprises
(a) a Cas9 polypeptide with reduced nuclease activity, and
(b) one or more gRNAs that bind to the target region of the 15q11-13 PWS-associated locus.

35. The method of any of claims 28-34, wherein the Targeted Repressor System is a CRISPR-Cas Type II system comprising one or more gRNAs, wherein at least one gRNA comprises a nucleotide sequence at least 80% complementary to at least 10-20 consecutive nucleotides of said target region.

36. The method of claim 35, wherein the at least one gRNA comprises a nucleotide sequence at least 80% complementary to at least 18 consecutive nucleotides of said target region.

37. The method any of claims 1-13, wherein the Targeted Repressor System is a ZF-based and/or TALE-based system.

38. The method of claim 37, wherein the Targeted Repressor System comprises one or more fusion proteins comprising (a) a TALE that binds to the target region of the 15q11-13 PWS-associated locus and (b) a repressor.

39. The method of claim 37, wherein the Targeted Repressor System comprises one or more fusion proteins comprising (a) a ZF that binds to the target region of the 15q11-13 PWS-associated locus and (b) a repressor.

40. The method of any of claims 29-39, wherein the repressor is KRAB, Mad mSIN3 interaction domain (SID); the ERF repressor domain (ERD); KMT1 family: SUV39H1, SUV39H2, G9A, ESET/SETBD1, and homologs (Cir4, Su(var)3-9); KMT5 family: Pr-SET7/8, SUV4-20H1, and homologs (PR-set7, Suv4-20, and Set9); KMT6: EZH2, KMT8: RIZ1, KDM4 family: JMJD2A/JHDM3A, JMJD2B, JMJ2D2C/GASC1, JMJD2D, and homologs (Rph1); KDM5 family JARID1A/RBP2, JARID1B/PLU-1, JARID1C/SMCX, JARID1D/SMCY, and homologs (Lid, Jhn2, Jmj2); HDAC1, HDAC2, HDAC3, HDAC8, and its homologs (Rpd3, Hos1, Cir6); HDAC4, HDAC5, HDAC7, HDAC9, and its homologs (Hda1, Cir3); SIRT1, SIRT2, and its homologs (Sir2, Hst1, Hst2, Hst3, and Hst4); HDAC11, DNMT1, DNMT3a/3b, MET1, DRM3, and homologs, ZMET2, CMT1, CMT2, Laminin A, Laminin B, or CTCF.

41. A DNA Targeting System that binds to a target region selected from the group consisting of:
nucleotide positions −127023 to −125023;
nucleotide positions −93065 to −91065;
nucleotide positions −1104 to +896;
nucleotide positions −126547 to −124695 [mat1];
nucleotide positions −131937 to −130580 [mat1A];
nucleotide positions −129415 to −127715 [mat1B];
nucleotide positions −123798 to −122440 [mat1C];
nucleotide positions −92568 to −91460 [mat2];
nucleotide positions −797 to +1346 [mat3]; and/or
nucleotide positions +12858 to +14026 [mat4],
wherein the nucleotide position is relative to position 1 being the start site of SNRPN exon 1 of the PWS imprinting center on chromosome 15.

42. A DNA Targeting System that binds to a target region selected from the group consisting of:
nucleotide positions −126523 to −125523;
nucleotide positions −92565 to −91565;
nucleotide positions −604 to +395;
nucleotide positions −126047 to −125195 [mat1];
nucleotide positions −131437 to −131080 [mat1A];
nucleotide positions −128915 to −128215 [mat1B];
nucleotide positions −123298 to −122940 [mat1C];
nucleotide positions −92068 to −91960 [mat2];
nucleotide positions −297 to +846 [mat3]; and/or
nucleotide positions +13358 to +13526 [mat4],
wherein the nucleotide position is relative to position 1 being the start site of SNRPN exon 1 of the PWS imprinting center on chromosome 15.

43. The DNA Targeting System of claim 41 or 42 that is a Targeted Activator System.

44. A DNA Targeting System that binds to a target region selected from the group consisting of:
nucleotide positions +23022 to +25022;
nucleotide positions +34734 to +36734;
nucleotide positions −101358 to −94223 [pat1];
nucleotide positions −58232 to −51914 [pat2];
nucleotide positions −4847 to −3047 [pat3];
nucleotide positions −1774 to +2421 [pat4];
nucleotide positions +2446 to +24016 [pat5];
nucleotide positions +23346 to +25082 [pat6];
nucleotide positions +24340 to +35718 [pat7]; and/or
nucleotide positions +35206 to +36668 [pat8];
wherein the nucleotide position is relative to position 1 being the start site of SNRPN exon 1 of the PWS imprinting center on chromosome 15.

45. A DNA Targeting System that binds to a target region selected from the group consisting of:
nucleotide positions +23522 to +24522:
nucleotide positions +35234 to +36234;
nucleotide positions −100858 to −94723[pat1];
nucleotide positions −57732 to −52414 [pat2];
nucleotide positions −4347 to −3547 [pat3];
nucleotide positions −1275 to +1921 [pat4];
nucleotide positions +2946 to +23516 [pat5];
nucleotide positions +23846 to +24582 [pat6];
nucleotide positions +24840 to +35218 [pat7]; and/or
nucleotide positions +35706 to +36168 [pat8];
wherein the nucleotide position is relative to position 1 being the start site of SNRPN exon 1 of the PWS imprinting center on chromosome 15.

46. The DNA Targeting System of claims 44 or 45 that is a Targeted Repressor System.

47. The DNA Targeting System of any of claims 41-46 that is a CRISPR-Cas Type II system comprising one or more gRNAs, wherein at least one gRNA comprises a nucleotide sequence at least 80% complementary to at least 10-20 consecutive nucleotides of said target region.

48. A nucleic acid encoding at least one component of said DNA Targeting System.

49. A vector comprising the nucleic acid of claim 48.

50. A first nucleic acid encoding at least one gRNA comprises a nucleotide sequence at least 80% complementary to at least 10-20 consecutive nucleotides of said target region, and a second nucleic acid encoding a Cas9 protein, or a Cas9 fusion protein, wherein the first and second nucleic acids are on the same or different vectors.

51. The vector of claim 49 wherein the vector is a viral vector, optionally a retroviral, lentiviral, adenovirus, adeno-associated virus vector, synthetic vector, or is a vector within a lipid nanoparticle.

52. A ribonucleoprotein comprising a Cas9 protein, or a Cas9 fusion protein, and at least one gRNA comprises a nucleotide sequence at least 80% complementary to at least 10-20 consecutive nucleotides of said target region.

53. A pharmaceutical composition comprising said DNA Targeting System of any of claims 41-47, said nucleic acids of claim 48 or 50, said vector of claim 49 or 51, or said ribonucleoprotein of claim 52.

54. A guide RNA (gRNA) comprising a polynucleotide sequence corresponding to at least one of SEQ ID NOs: 1-12, or at last one of SEQ ID NOs: 47-86 and 91-1122, or an allelic variant thereof.

55. A DNA Targeting System that binds to a regulatory element of a gene within the 15q11-13 locus, the DNA Targeting System comprising at least one gRNA that binds and targets a polynucleotide sequence comprising a nucleotide sequence corresponding to a complement of at least one of SEQ ID NOs: 1-12, or at least one of SEQ ID NOs: 47-86 and 91-1122, or an allelic variant thereof.

56. The DNA Targeting System of claim 55, wherein the at least one gRNA comprises a polynucleotide sequence corresponding to at least one of SEQ ID NOs: 1-12, or at least one of SEQ ID NOs: 47-86 and 91-1122, or a variant thereof.

57. The DNA Targeting System of any one of claims 55-56, further comprising a Clustered Regularly Interspaced Short Palindromic Repeats associated (Cas) protein or a fusion protein,
wherein the fusion protein comprises two heterologous polypeptide domains, wherein the first polypeptide domain comprises a Cas protein and the second polypeptide domain has an activity selected from the group consisting of transcription activation activity, transcription repression activity, transcription release factor activity, histone modification activity, nuclease activity, nucleic acid association activity, methylase activity, demethylase activity, acetylation activity, and deacetylation activity.

58. The DNA targeting system of claim 57, wherein the Cas protein comprises a Streptococcus pyogenes Cas9 protein, or a variant thereof.

59. The DNA targeting system of claim 58, wherein the Cas protein comprises a VQR variant of the S. pyogenes Cas9 protein.

60. The DNA targeting system of claim 59, wherein the DNA targeting system comprises a fusion protein.

61. The DNA targeting system of claim 60, wherein the second polypeptide domain has transcription activation activity, transcription repression activity, histone modification activity, or a combination thereof.

62. The DNA targeting system of claim 60, wherein the fusion protein comprises dCas9-VP64, VP64-dCas9-VP64, dCas9-p300, or dCas9-KRAB.

63. The DNA targeting system of any one of claims 57-62, wherein the Cas protein comprises a Cas9 that recognizes a Protospacer Adjacent Motif (PAM) of NGG (SEQ ID NO:
13), NGA (SEQ ID NO: 14), NGAN (SEQ ID NO:15), or NGNG (SEQ ID NO:16).

64. The DNA targeting system of any one of claims 55-62, wherein the gene within the 15q11-13 locus is selected from SNRPN, SNORD115, SNORD116, SPA1, SPA2, and MAGEL2.

65. An isolated polynucleotide sequence comprising the gRNA of claim 54.

66. An isolated polynucleotide sequence encoding the DNA targeting system of any one of claims 55-64.

67. A vector comprising the isolated polynucleotide sequence of claim 65 or 66.

68. A vector encoding the gRNA of claim 54 and a Clustered Regularly Interspaced Short Palindromic Repeats associated (Cas) protein.

69. The vector of claim 68, wherein the Cas protein comprises a Streptococcus pyogenes Cas9 protein, or variant thereof.

70. The vector of claim 69, wherein the Cas protein comprises a VQR variant of the S. pyogenes Cas9 protein.

71. A cell comprising the gRNA of claim 54, the DNA targeting system of any one of claims 55-64, the isolated polynucleotide sequence of claim 65 or 66, or the vector of any one of claims 67-70, or a combination thereof.

72. A pharmaceutical composition comprising the gRNA of claim 54, the DNA targeting system of any one of claims 55-64, the isolated polynucleotide sequence of claim 65 or 66, or the vector of any one of claims 67-70, or the cell of claim 71, or a combination thereof.

73. A method for treating Prader-Willi Syndrome (PWS) in a subject, the method comprising administering to the subject the DNA targeting system of any one of claims 55-64, the isolated polynucleotide sequence of claim 65 or 66, the vector of any one of claims 67-70, or the cell of claim 71, or a combination thereof.

74. The method of claim 73, wherein the expression of at least one gene within the 15q11-q13 locus is increased.

75. The method of claim 74, wherein the gene within the 15q11-13 locus is selected from SNRPN, SNORD115, SNORD116, SNORD109A, IPW, SPA1, SPA2, and MAGEL2.

76. The method of any one of claims 73-75, wherein the expression of at least one RNA transcript selected from SNRPN, SNORD115, SNORD116, SPA1, SPA2, and MAGEL2, or a combination thereof, is increased.

77. The method of any one of claims 73-76, wherein the initiation of transcription from the SNRPN promoter, SNORD115 promoter, SNORD116 promoter, or a combination thereof, is increased.

78. The method of claim 1, wherein the DNA Targeting System has demethylase activity.

79. The method of claim 78 wherein administration of the DNA Targeting System results in increased expression of one or more of the following gene or gene products: MKRN3, MAGEL2, NDN, C15ORF2, SNURF-SNRPN, SNORD107, SNORD64, SNORD109A, SNORD116, SNORD116@, SPA1, SPA2, 116HG, SNORD116-1 to 30, Sno-Inc RNA 1 to 5, IPW, SNORD115, SNORD115@, 115HG, SNORD115-1 to 48, SNORD109B, and/or SNG14.

80. The method of any of claims 78-79 wherein the DNA Targeting System binds to a target region selected from the group consisting of:
nucleotide positions −297 to +846 [mat3]; and
nucleotide positions +13358 to +13526 [mat4];
wherein the nucleotide position is relative to position 1 being the start site of SNRPN exon 1 of the PWS imprinting center on chromosome 15.

81. The method of any of claims 78-80, wherein the subject has a PWS Type 1 large deletion, PWS Type 2 large deletion, PWS imprinting center mutation or PWS uniparental disomy.

82. The method of any of claims 78-80, wherein the subject has a PWS microdeletion encompassing SNORD116, but not MAGEL2.

83. The method of any of claims 78-80, wherein the subject has a PWS or PWS-like atypical deletion encompassing MAGEL2, but not SNORD116.

84. The method of any of claims 78-80, wherein the subject has heterozygous Schaaf-Yang syndrome or MAGEL2 disorder.

85. The method of any of claims 78-84, wherein the DNA Targeting System is a CRISPR-Cas Type II system.

86. The method of claim 85, wherein the DNA Targeting System comprises:
(a) a fusion protein comprising:
(i) a Cas9 polypeptide with reduced nuclease activity, and
(ii) a polypeptide with demethylase activity; and
(b) one or more guide RNAs (gRNA) that bind to a target region in the 15q11-13 PWS-associated locus.

87. The method of claim 86, wherein the subject is administered:
(a) said fusion protein and said one or more gRNAs; or
(b) a nucleic acid sequence encoding said fusion protein, and said one or more gRNAs; or
(c) a nucleic acid sequence encoding said fusion protein, and nucleic acid sequence(s) encoding said one or more gRNAs.

88. The method of claim 86, wherein the subject is administered a vector comprising a nucleic acid encoding said fusion protein and/or a vector comprising a nucleic acid encoding said gRNA.

89. The method of claim 88 wherein the vector is a viral vector, optionally a retroviral, lentiviral, adenovirus, adeno-associated virus vector, synthetic vector, or is a vector within a lipid nanoparticle.

90. The method of any of claims 78-89, wherein the DNA Targeting System is a CRISPR-Cas Type II system comprising one or more gRNAs, wherein at least one gRNA comprises a nucleotide sequence at least 80% complementary to at least 10-20 consecutive nucleotides of said target region.

91. The method of any of claims 78-90, wherein the at least one gRNA comprises a nucleotide sequence at least 80% complementary to at least 18 consecutive nucleotides of said target region.

92. The method of any of claims 86-91, wherein the at least one gRNA specifically hybridizes to any of SEQ ID NOs: 47-86, or a corresponding allelic variant thereof, or the complement of any of SEQ ID Nos: 47-86 or a corresponding allelic variant thereof, or wherein the at least one gRNA comprises at least 10, 11, 12, 13, 14, 15, 16, 17, 18, 19, or 20 consecutive nucleotides of any of SEQ ID NOs: 591, 585, 685, 697, 750, 752, 763, 771, 196, 812, 861, or 1089, or a corresponding complement and/or allelic variant thereof.

93. The method of claim 92 wherein the at least one gRNA is fully complementary to at least 10, 11, 12, 13, 14, 15, 16, 17, 18, 19, or 20 consecutive nucleotides of any of SEQ ID NOs: 47-86, or a corresponding allelic variant thereof, or the complement of any of SEQ ID Nos: 47-86 or a corresponding allelic variant thereof, or wherein the at least one gRNA is fully complementary to at least 10, 11, 12, 13, 14, 15, 16, 17, 18, 19, or 20 consecutive nucleotides of any of SEQ ID NOs: 47, 49, 53, 55, 64, 65, 68, 70, 196, 291, 335, or 528, or a corresponding complement and/or allelic variant thereof.

94. The method of any of claims 85-93 wherein the Cas9 is a S. pyogenes Cas9.

95. The method of any of claims 78-84, wherein the DNA Targeting System is a ZF-based and/or TALE-based system.

96. The method of claim 95, wherein the DNA Targeting System comprises one or more fusion proteins comprising (a) a TALE that binds to the target region of the 15q11-13 PWS-associated locus and (b) a demethylase.

97. The method of claim 95, wherein the DNA Targeting System comprises one or more fusion proteins comprising (a) a ZF that binds to the target region of the 15q11-13 PWS-associated locus and (b) a demethylase.

98. The method of any of claims 78-84 or 95-97 comprising administering a second DNA Targeting System targeting a second target region.

99. A DNA Targeting System that has demethylase activity and binds to a target region selected from the group consisting of:
nucleotide positions −297 to +846 [mat3]; and
nucleotide positions +13358 to +13526 [mat4];
wherein the nucleotide position is relative to position 1 being the start site of SNRPN exon 1 of the PWS imprinting center on chromosome 15.

100. The DNA Targeting System of claim 99 that targets a target region that results in increased expression of one of more of: SNORD116 or its products, or MAGEL2.

101. The DNA Targeting System of claim 99 that targets a target region that results in increased expression of one of more of the following gene or gene products: MKRN3, MAGEL2, NDN, C15ORF2, SNURF-SNRPN, SNORD107, SNORD64, SNORD109A, SNORD116, SNORD116@, SPA1, SPA2, 116HG, SNORD116-1 to 30, Sno-Inc RNA 1 to 5, IPW, SNORD115, SNORD115@, 115HG, SNORD115-1 to 48, SNORD109B, and/or SNG14.

102. The DNA Targeting System of any of claims 99-101 that is a CRISPR-Cas Type II System.

103. The DNA Targeting System of claim 102 comprising:
(a) a Cas9 fusion protein comprising:
(i) a Cas9 polypeptide with reduced nuclease activity, and
(ii) a polypeptide with demethylase activity; and
(b) one or more guide RNAs (gRNA) that bind to a target region in the 15q11-13 PWS-associated locus.

104. The DNA Targeting System of claim 102 or claim 103 wherein the demethylase activity is Tet1CD (SEQ ID NO: 90).

105. The DNA Targeting System of claim 103 or 104 that comprises a S. pyogenes Cas9.

106. The DNA targeting system of claim 103 or 104 that comprises a VQR variant of the S. pyogenes Cas9 protein.

107. The DNA Targeting System of claim 103 or 104 that comprises a S. aureus Cas9.

108. The DNA Targeting System of claim 103 or 104 that comprises a Cas9 that recognizes a Protospacer Adjacent Motif (PAM) of NGG (SEQ ID NO: 13), NGA (SEQ ID NO: 14), NGAN (SEQ ID NO:15), or NGNG (SEQ ID NO:16).

109. The DNA Targeting System of any of claims 101-103 wherein at least one gRNA comprises a nucleotide sequence at least 80% complementary to at least 10-20 consecutive nucleotides of said target region.

110. The DNA Targeting System of any of claims 103-109 wherein the at least one gRNA comprises a nucleotide sequence at least 80% complementary to at least 18 consecutive nucleotides of said target region, or wherein the at least one gRNA comprises at least 10, 11, 12, 13, 14, 15, 16, 17, 18, 19, or 20 consecutive nucleotides of any of SEQ ID NOs: 591, 585, 685, 697, 750, 752, 763, 771, 196, 812, 861, or 1069, or a corresponding complement and/or allelic variant thereof.

111. A guide RNA (gRNA) comprising a polynucleotide sequence that specifically hybridizes to any of SEQ ID NOs: 47-86, or a corresponding allelic variant thereof, or the complement of any of SEQ ID Nos: 47-86 or a corresponding allelic variant thereof, or is fully complementary to at least 10, 11, 12, 13, 14, 15, 16, 17, 18, 19, or 20 consecutive nucleotides of any of SEQ ID NOs: 47, 49, 53, 55, 64, 65, 68, 70, 196, 291, 335, or 528, or a corresponding complement and/or allelic variant thereof, or comprises at least 10, 11, 12, 13, 14, 15, 16, 17, 18, 19, or 20 consecutive nucleotides of any of SEQ ID NOs: 591, 585, 685, 697, 750, 752, 763, 771, 196, 812, 861, or 1069, or a corresponding complement and/or allelic variant thereof.

112. The gRNA of claim 111 that is fully complementary to at least 10, 11, 12, 13, 14, 15, 16, 17, 18, 19, or 20 consecutive nucleotides of any of SEQ ID NOs: 47-86, or a corresponding allelic variant thereof, or the complement of any of SEQ ID Nos: 47-86 or a corresponding allelic variant thereof.

113. The DNA Targeting System of any of claims 103-110 comprising a guide RNA of claim 111 of 112.

114. A nucleic acid encoding at least one component of the DNA Targeting System of any of claims 103-110 or 113, or encoding a guide RNA of claim 111 or 112.

115. A first nucleic acid encoding at least one gRNA of the DNA Targeting System of any of claims 103-110 or a gRNA of claim 111 of 112; and a second nucleic acid encoding the Cas9 fusion protein of the DNA Targeting System of any of claims 103-110, wherein the first and second nucleic acids are on the same or different vectors.

116. A vector comprising the nucleic acid of claim 111 or 112.

117. The vector of claim 113 wherein the vector is a viral vector, optionally a retroviral, lentiviral, adenovirus, adeno-associated virus vector, synthetic vector, or is a vector within a lipid nanoparticle.

118. The DNA Targeting System of any of claims 103-110 which is a ribonucleoprotein comprising a Cas9 fusion protein, and at least one gRNA.

119. A cell comprising said DNA Targeting System of any of claims 103-110, 113 or 118, said gRNA of claim 111 or 112, said nucleic acids of claim 114 or 115, or said vector of claim 116 or 117.

120. A pharmaceutical composition comprising said DNA Targeting System of any of claims 103-110, 113 or 118, said gRNA of claim 111 or 112, said nucleic acids of claim 114 or 115, or said vector of claim 116 or 117.

121. A method for treating Prader-Willi Syndrome (PWS) in a subject, the method comprising administering to the subject said DNA Targeting System of any of claims 103-110, 113 or 118, said gRNA of claim 111 or 112, said nucleic acids of claim 114 or 115, or said vector of claim 116 or 117, or a combination thereof.

122. The method of claim 121, wherein the expression of at least one gene within the 15q11-q13 locus is increased.

123. The method of claim 121, wherein the gene within the 51q11-13 locus is selected from SNRPN, SNORD115, SNORD116, SNORD109A, IPW, SPA1, SPA2, and MAGEL2.

124. The method of any one of claims 121-122, wherein the expression of at least one RNA transcript selected from SNRPN, SNORD115, SNORD116, SPA1, SPA2, and MAGEL2, or a combination thereof, is increased.

125. The method of any one of claims 73-76, wherein the initiation of transcription from the SNRPN promoter, SNORD115 promoter, SNORD116 promoter, or a combination thereof, is increased.


SEQUENCE LISTING (1142 sequences)

SEQ ID NO: 1 - DNA, 20 bp, Artificial Sequence
  Synthetic polynucleotide
ctaagcagtggactaaggat

SEQ ID NO: 2 - DNA, 20 bp, Artificial Sequence
  Synthetic polynucleotide
ctactttagattctattgta

SEQ ID NO: 3 - DNA, 20 bp, Artificial Sequence
  Synthetic polynucleotide
gcagctcagaaagtgatgca

SEQ ID NO: 4 - DNA, 20 bp, Artificial Sequence
  Synthetic polynucleotide
gccaggttactgattcatag

SEQ ID NO: 5 - DNA, 20 bp, Artificial Sequence
  Synthetic polynucleotide
gcgcactgcagcgcagacca

SEQ ID NO: 6 - DNA, 20 bp, Artificial Sequence
  Synthetic polynucleotide
ggaaccagtcagaacaggtg

SEQ ID NO: 7 - DNA, 20 bp, Artificial Sequence
  Synthetic polynucleotide
ggtcggcttcaggagggaag

SEQ ID NO: 8 - DNA, 20 bp, Artificial Sequence
  Synthetic polynucleotide
gtggataggtgcgttcaagg

SEQ ID NO: 9 - DNA, 20 bp, Artificial Sequence
  Synthetic polynucleotide
gtttcaggcctggactgggt

SEQ ID NO: 10 - DNA, 20 bp, Artificial Sequence
  Synthetic polynucleotide
acagttcagggcatgagata

SEQ ID NO: 11 - DNA, 20 bp, Artificial Sequence
  Synthetic polynucleotide
cagttcagggcatgagataa

SEQ ID NO: 12 - DNA, 20 bp, Artificial Sequence
  Synthetic polynucleotide
gggcatgagataagggcagt

SEQ ID NO: 13 - DNA, 3 bp, Artificial Sequence
  Synthetic polynucleotide
  misc_feature (1)..(1): n is a, t, c or g
ngg

SEQ ID NO: 14 - DNA, 3 bp, Artificial Sequence
  Synthetic polynucleotide
  misc_feature (1)..(1): n is a, t, c or g
nga

SEQ ID NO: 15 - DNA, 4 bp, Artificial Sequence
  Synthetic polynucleotide
  misc_feature (1)..(1): n is a, t, c or g
  misc_feature (4)..(4): n is a, t, c or g
ngan

SEQ ID NO: 16 - DNA, 4 bp, Artificial Sequence
  Synthetic polynucleotide
  misc_feature (1)..(1): n is a, t, c or g
  misc_feature (3)..(3): n is a, t, c or g
ngng

SEQ ID NO: 17 - DNA, 7 bp, Streptococcus thermophilus
  misc_feature (1)..(2): n is a, t, c or g
nnagaaw

SEQ ID NO: 18 - DNA, 4 bp, Streptococcus mutans
  misc_feature (1)..(1): n is a, t, c or g
naar

SEQ ID NO: 19 - DNA, 5 bp, Staphylococcus aureus
  misc_feature (1)..(2): n is a, t, c or g
nngrr

SEQ ID NO: 20 - DNA, 6 bp, Staphylococcus aureus
  misc_feature (1)..(2): n is a, t, c or g
  misc_feature (6)..(6): n is a, t, c or g
nngrrn

SEQ ID NO: 21 - DNA, 6 bp, Staphylococcus aureus
  misc_feature (1)..(2): n is a, t, c or g
nngrrt

SEQ ID NO: 22 - DNA, 6 bp, Staphylococcus aureus
  misc_feature (1)..(2): n is a, t, c or g
nngrrv

SEQ ID NO: 23 - DNA, 4107 bp, Streptococcus pyogenes
atggataaaaagtacagcatcgggctggacatcggtacaaactcagtggggtgggccgtgattacggacgagtacaaggtaccctccaaaaaatttaaagtgctgggtaacacggacagacactctataaagaaaaatcttattggagccttgctgttcgactcaggcgagacagccgaagccacaaggttgaagcggaccgccaggaggcggtataccaggagaaagaaccgcatatgctacctgcaagaaatcttcagtaacgagatggcaaaggttgacgatagctttttccatcgcctggaagaatcctttcttgttgaggaagacaagaagcacgaacggcaccccatctttggcaatattgtcgacgaagtggcatatcacgaaaagtacccgactatctaccacctcaggaagaagctggtggactctaccgataaggcggacctcagacttatttatttggcactcgcccacatgattaaatttagaggacatttcttgatcgagggcgacctgaacccggacaacagtgacgtcgataagctgttcatccaacttgtgcagacctacaatcaactgttcgaagaaaaccctataaatgcttcaggagtcgacgctaaagcaatcctgtccgcgcgcctctcaaaatctagaagacttgagaatctgattgctcagttgcccggggaaaagaaaaatggattgtttggcaacctgatcgccctcagtctcggactgaccccaaatttcaaaagtaacttcgacctggccgaagacgctaagctccagctgtccaaggacacatacgatgacgacctcgacaatctgctggcccagattggggatcagtacgccgatctctttttggcagcaaagaacctgtccgacgccatcctgttgagcgatatcttgagagtgaacaccgaaattactaaagcaccccttagcgcatctatgatcaagcggtacgacgagcatcatcaggatctgaccctgctgaaggctcttgtgaggcaacagctccccgaaaaatacaaggaaatcttctttgaccagagcaaaaacggctacgctggctatatagatggtggggccagtcaggaggaattctataaattcatcaagcccattctcgagaaaatggacggcacagaggagttgctggtcaaacttaacagggaggacctgctgcggaagcagcggacctttgacaacgggtctatcccccaccagattcatctgggcgaactgcacgcaatcctgaggaggcaggaggatttttatccttttcttaaagataaccgcgagaaaatagaaaagattcttacattcaggatcccgtactacgtgggacctctcgcccggggcaattcacggtttgcctggatgacaaggaagtcagaggagactattacaccttggaacttcgaagaagtggtggacaagggtgcatctgcccagtctttcatcgagcggatgacaaattttgacaagaacctccctaatgagaaggtgctgcccaaacattctctgctctacgagtactttaccgtctacaatgaactgactaaagtcaagtacgtcaccgagggaatgaggaagccggcattccttagtggagaacagaagaaggcgattgtagacctgttgttcaagaccaacaggaaggtgactgtgaagcaacttaaagaagactactttaagaagatcgaatgttttgacagtgtggaaatttcaggggttgaagaccgcttcaatgcgtcattggggacttaccatgatcttctcaagatcataaaggacaaagacttcctggacaacgaagaaaatgaggatattctcgaagacatcgtcctcaccctgaccctgttcgaagacagggaaatgatagaagagcgcttgaaaacctatgcccacctcttcgacgataaagttatgaagcagctgaagcgcaggagatacacaggatggggaagattgtcaaggaagctgatcaatggaattagggataaacagagtggcaagaccatactggatttcctcaaatctgatggcttcgccaataggaacttcatgcaactgattcacgatgactctcttaccttcaaggaggacattcaaaaggctcaggtgagcgggcagggagactcccttcatgaacacatcgcgaatttggcaggttcccccgctattaaaaagggcatccttcaaactgtcaaggtggtggatgaattggtcaaggtaatgggcagacataagccagaaaatattgtgatcgagatggcccgcgaaaaccagaccacacagaagggccagaaaaatagtagagagcggatgaagaggatcgaggagggcatcaaagagctgggatctcagattctcaaagaacaccccgtagaaaacacacagctgcagaacgaaaaattgtacttgtactatctgcagaacggcagagacatgtacgtcgaccaagaacttgatattaatagactgtccgactatgacgtagaccatatcgtgccccagtccttcctgaaggacgactccattgataacaaagtcttgacaagaagcgacaagaacaggggtaaaagtgataatgtgcctagcgaggaggtggtgaaaaaaatgaagaactactggcgacagctgcttaatgcaaagctcattacacaacggaagttcgataatctgacgaaagcagagagaggtggcttgtctgagttggacaaggcagggtttattaagcggcagctggtggaaactaggcagatcacaaagcacgtggcgcagattttggacagccggatgaacacaaaatacgacgaaaatgataaactgatacgagaggtcaaagttatcacgctgaaaagcaagctggtgtccgattttcggaaagacttccagttctacaaagttcgcgagattaataactaccatcatgctcacgatgcgtacctgaacgctgttgtcgggaccgccttgataaagaagtacccaaagctggaatccgagttcgtatacggggattacaaagtgtacgatgtgaggaaaatgatagccaagtccgagcaggagattggaaaggccacagctaagtacttcttttattctaacatcatgaatttttttaagacggaaattaccctggccaacggagagatcagaaagcggccccttatagagacaaatggtgaaacaggtgaaatcgtctgggataagggcagggatttcgctactgtgaggaaggtgctgagtatgccacaggtaaatatcgtgaaaaaaaccgaagtacagaccggaggattttccaaggaaagcattttgcctaaaagaaactcagacaagctcatcgcccgcaagaaagattgggaccctaagaaatacgggggatttgactcacccaccgtagcctattctgtgctggtggtagctaaggtggaaaaaggaaagtctaagaagctgaagtccgtgaaggaactcttgggaatcactatcatggaaagatcatcctttgaaaagaaccctatcgatttcctggaggctaagggttacaaggaggtcaagaaagacctcatcattaaactgccaaaatactctctcttcgagctggaaaatggcaggaagagaatgttggccagcgccggagagctgcaaaagggaaacgagcttgctctgccctccaaatatgttaattttctctatctcgcttcccactatgaaaagctgaaagggtctcccgaagataacgagcagaagcagctgttcgtcgaacagcacaagcactatctggatgaaataatcgaacaaataagcgagttcagcaaaagggttatcctggcggatgctaatttggacaaagtactgtctgcttataacaagcaccgggataagcctattagggaacaagccgagaatataattcacctctttacactcacgaatctcggagcccccgccgccttcaaatactttgatacgactatcgaccggaaacggtataccagtaccaaagaggtcctcgatgccaccctcatccaccagtcaattactggcctgtacgaaacacggatcgacctctctcaactgggcggcgactag

SEQ ID NO: 24 - protein, 1368 aa, Streptococcus pyogenes [sequence omitted]

SEQ ID NO: 25 - DNA, 3158 bp, Staphylococcus aureus
atgaaaaggaactacattctggggctggacatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctgcagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatctgtacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagctccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtcccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggcctccccctggaggacctgctgaacaatccattcaactacgaggtcgatcatattatccccagaagcgtgtccttcgacaattcctttaacaacaaggtgctggtcaagcaggaagagaactctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaattgcctctaagactcagagtatcaaaaagtactcaaccgacattctgggaaacctgtatgaggtgaagagcaaaaagcaccctcagattatcaaaaagggc

SEQ ID NO: 26 - DNA, 3159 bp, Staphylococcus aureus
atgaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggc

SEQ ID NO: 27 - DNA, 3159 bp, Staphylococcus aureus
atgaagcgcaactacatcctcggactggacatcggcattacctccgtgggatacggcatcatcgattacgaaactagggatgtgatcgacgctggagtcaggctgttcaaagaggcgaacgtggagaacaacgaggggcggcgctcaaagaggggggcccgccggctgaagcgccgccgcagacatagaatccagcgcgtgaagaagctgctgttcgactacaaccttctgaccgaccactccgaactttccggcatcaacccatatgaggctagagtgaagggattgtcccaaaagctgtccgaggaagagttctccgccgcgttgctccacctcgccaagcgcaggggagtgcacaatgtgaacgaagtggaagaagataccggaaacgagctgtccaccaaggagcagatcagccggaactccaaggccctggaagagaaatacgtggcggaactgcaactggagcggctgaagaaagacggagaagtgcgcggctcgatcaaccgcttcaagacctcggactacgtgaaggaggccaagcagctcctgaaagtgcaaaaggcctatcaccaacttgaccagtcctttatcgatacctacatcgatctgctcgagactcggcggacttactacgagggtccaggggagggctccccatttggttggaaggatattaaggagtggtacgaaatgctgatgggacactgcacatacttccctgaggagctgcggagcgtgaaatacgcatacaacgcagacctgtacaacgcgctgaacgacctgaacaatctcgtgatcacccgggacgagaacgaaaagctcgagtattacgaaaagttccagattattgagaacgtgttcaaacagaagaagaagccgacactgaagcagattgccaaggaaatcctcgtgaacgaagaggacatcaagggctatcgagtgacctcaacgggaaagccggagttcaccaatctgaaggtctaccacgacatcaaagacattaccgcccggaaggagatcattgagaacgcggagctgttggaccagattgcgaagattctgaccatctaccaatcctccgaggatattcaggaagaactcaccaacctcaacagcgaactgacccaggaggagatagagcaaatctccaacctgaagggctacaccggaactcataacctgagcctgaaggccatcaacttgatcctggacgagctgtggcacaccaacgataaccagatcgctattttcaatcggctgaagctggtccccaagaaagtggacctctcacaacaaaaggagatccctactacccttgtggacgatttcattctgtcccccgtggtcaagagaagcttcatacagtcaatcaaagtgatcaatgccattatcaagaaatacggtctgcccaacgacattatcattgagctcgcccgcgagaagaactcgaaggacgcccagaagatgattaacgaaatgcagaagaggaaccgacagactaacgaacggatcgaagaaatcatccggaccaccgggaaggaaaacgcgaagtacctgatcgaaaagatcaagctccatgacatgcaggaaggaaagtgtctgtactcgctggaggccattccgctggaggacttgctgaacaacccttttaactacgaagtggatcatatcattccgaggagcgtgtcattcgacaattccttcaacaacaaggtcctcgtgaagcaggaggaaaactcgaagaagggaaaccgcacgccgttccagtacctgagcagcagcgactccaagatttcctacgaaaccttcaagaagcacatcctcaacctggcaaaggggaagggtcgcatctccaagaccaagaaggaatatctgctggaagaaagagacatcaacagattctccgtgcaaaaggacttcatcaaccgcaacctcgtggatactagatacgctactcggggtctgatgaacctcctgagaagctactttagagtgaacaatctggacgtgaaggtcaagtcgattaacggaggtttcacctccttcctgcggcgcaagtggaagttcaagaaggaacggaacaagggctacaagcaccacgccgaggacgccctgatcattgccaacgccgacttcatcttcaaagaatggaagaaacttgacaaggctaagaaggtcatggaaaaccagatgttcgaagaaaagcaggccgagtctatgcctgaaatcgagactgaacaggagtacaaggaaatctttattacgccacaccagatcaaacacatcaaggatttcaaggattacaagtactcacatcgcgtggacaaaaagccgaacagggaactgatcaacgacaccctctactccacccggaaggatgacaaagggaataccctcatcgtcaacaaccttaacggcctgtacgacaaggacaacgataagctgaagaagctcattaacaagtcgcccgaaaagttgctgatgtaccaccacgaccctcagacttaccagaagctcaagctgatcatggagcagtatggggacgagaaaaacccgttgtacaagtactacgaagaaactgggaattatctgactaagtactccaagaaagataacggccccgtgattaagaagattaagtactacggcaacaagctgaacgcccatctggacatcaccgatgactaccctaattcccgcaacaaggtcgtcaagctgagcctcaagccctaccggtttgatgtgtaccttgacaatggagtgtacaagttcgtgactgtgaagaaccttgacgtgatcaagaaggagaactactacgaagtcaactccaagtgctacgaggaagcaaagaagttgaagaagatctcgaaccaggccgagttcattgcctccttctataacaacgacctgattaagatcaacggcgaactgtaccgcgtcattggcgtgaacaacgatctcctgaaccgcatcgaagtgaacatgatcgacatcacttaccgggaatacctggagaatatgaacgacaagcgcccgccccggatcattaagactatcgcctcaaagacccagtcgatcaagaagtacagcaccgacatcctgggcaacctgtacgaggtcaaatcgaagaagcacccccagatcatcaagaaggga

SEQ ID NO: 28 - DNA, 3255 bp, Staphylococcus aureus
atggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccagagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaggcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaag

SEQ ID NO: 29 - DNA, 3239 bp, Staphylococcus aureus
accggtgccaccatgtacccatacgatgttccagattacgcttcgccgaagaaaaagcgcaaggtcgaagcgtccatgaaaaggaactacattctggggctggacatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctgcagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatcttacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagctccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtcccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggccatccccctggaggacctgctgaacaatccattcaactacgaggtcgatcatattatccccagaagcgtgtccttcgacaattcctttaacaacaaggtgctggtcaagcaggaagagaactctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaattgcctctaagactcagagtatcaaaaagtactcaaccgacattctgggaaacctgtatgaggtgaagagcaaaaagcaccctcagattatcaaaaagggctaagaa

SEQ ID NO: 30 - protein, 1053 aa, Staphylococcus aureus
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 31 - DNA, 3159 bp, Staphylococcus aureus
atgaaaaggaactacattctggggctggccatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctgcagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatctgtacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagctccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtcccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggccatccccctggaggacctgctgaacaatccattcaactacgaggtcgatcatattatccccagaagcgtgtccttcgacaattcctttaacaacaaggtgctggtcaagcaggaagagaactctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaattgcctctaagactcagagtatcaaaaagtactcaaccgacattctgggaaacctgtatgaggtgaagagcaaaaagcaccctcagattatcaaaaagggc

SEQ ID NO: 32 - DNA, 3159 bp, Staphylococcus aureus
atgaaaaggaactacattctggggctggacatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctgcagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatctgtacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagctccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtcccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggccatccccctggaggacctgctgaacaatccattcaactacgaggtcgatcatattatccccagaagcgtgtccttcgacaattcctttaacaacaaggtgctggtcaagcaggaagaggcctctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaattgcctctaagactcagagtatcaaaaagtactcaaccgacattctgggaaacctgtatgaggtgaagagcaaaaagcaccctcagattatcaaaaagggc

SEQ ID NO: 33 - DNA, 5 bp, Streptococcus thermophilus
  misc_feature (1)..(1): n is a, t, c or g
  misc_feature (4)..(4): n is a, t, c or g
nggng

SEQ ID NO: 34 - DNA, 3255 bp, Staphylococcus aureus
atggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaag

SEQ ID NO: 35 - DNA, 3156 bp, Staphylococcus aureus
aagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggc

SEQ ID NO: 36 - protein, 1052 aa, Staphylococcus aureus
KRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 37 - DNA, 7009 bp, Artificial Sequence
  Synthetic polynucleotide
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtacctttaattctagtactatgcatgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactaccggtgccaccatgaaaaggaactacattctggggctggacatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctgcagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatctgtacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagctccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtcccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggccatccccctggaggacctgctgaacaatccattcaactacgaggtcgatcatattatccccagaagcgtgtccttcgacaattcctttaacaacaaggtgctggtcaagcaggaagagaactctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaattgcctctaagactcagagtatcaaaaagtactcaaccgacattctgggaaacctgtatgaggtgaagagcaaaaagcaccctcagattatcaaaaagggcagcggaggcaagcgtcctgctgctactaagaaagctggtcaagctaagaaaaagaaaggatcctacccatacgatgttccagattacgcttaagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggaggtagcggccgcccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 38 - DNA, 52 bp, Artificial Sequence
  Synthetic polynucleotide
aatgatacggcgaccaccgagatctacacaatttcttgggtagtttgcagtt

SEQ ID NO: 39 - DNA, 51 bp, Artificial Sequence
  Synthetic polynucleotide
  misc_feature (25)..(30): n is a, t, c or g
caagcagaagacggcatacgagatnnnnnngactcggtgccactttttcaa

SEQ ID NO: 40 - DNA, 43 bp, Artificial Sequence
  Synthetic polynucleotide
gatttcttggctttatatatcttgtggaaaggacgaaacaccg

SEQ ID NO: 41 - DNA, 38 bp, Artificial Sequence
  Synthetic polynucleotide
gctagtccgttatcaacttgaaaaagtggcaccgagtc

SEQ ID NO: 42 - DNA, 50 bp, Artificial Sequence
  Synthetic polynucleotide
gttgataacggactagccttattttaacttgctatttctagctctaaaac

SEQ ID NO: 43 - protein, 1497 aa, Artificial Sequence
  Synthetic polypeptide
RADALDDFDLDMLGSDALDDFDLDMLGSDALDDFDLDMLGSDALDDFDLDMVNPKKKRKVGRGMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKVASRADALDDFDLDMLGSDALDDFDLDMLGSDALDDFDLDMLGSDALDDFDLDMLI

SEQ ID NO: 44 - DNA, 4491 bp, Artificial Sequence
  Synthetic polynucleotide
cgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatggttaaccccaagaagaagaggaaggtgggccgcggaatggacaagaagtactccattgggctcgccatcggcacaaacagcgtcggctgggccgtcattacggacgagtacaaggtgccgagcaaaaaattcaaagttctgggcaataccgatcgccacagcataaagaagaacctcattggcgccctcctgttcgactccggggaaaccgccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatgccatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtggctagccgcgccgacgcgctggacgatttcgatctcgacatgctgggttctgatgccctcgatgactttgacctggatatgttgggaagcgacgcattggatgactttgatctggacatgctcggctccgatgctctggacgatttcgatctcgatatgttaatc

SEQ ID NO: 45 - protein, 2414 aa, Artificial Sequence
  Synthetic polypeptide
MAENVVEPGPPSAKRPKLSSPALSASASDGTDFGSLFDLEHDLPDELINSTELGLTNGGDINQLQTSLGMVQDAASKHKQLSELLRSGSSPNLNMGVGGPGQVMASQAQQSSPGLGLINSMVKSPMTQAGLTSPNMGMGTSGPNQGPTQSTGMMNSPVNQPAMGMNTGMNAGMNPGMLAAGNGQGIMPNQVMNGSIGAGRGRQNMQYPNPGMGSAGNLLTEPLQQGSPQMGGQTGLRGPQPLKMGMMNNPNPYGSPYTQNPGQQIGASGLGLQIQTKTVLSNNLSPFAMDKKAVPGGGMPNMGQQPAPQVQQPGLVTPVAQGMGSGAHTADPEKRKLIQQQLVLLLHAHKCQRREQANGEVRQCNLPHCRTMKNVLNHMTHCQSGKSCQVAHCASSRQIISHWKNCTRHDCPVCLPLKNAGDKRNQQPILTGAPVGLGNPSSLGVGQQSAPNLSTVSQIDPSSIERAYAALGLPYQVNQMPTQPQVQAKNQQNQQPGQSPQGMRPMSNMSASPMGVNGGVGVQTPSLLSDSMLHSAINSQNPMMSENASVPSMGPMPTAAQPSTTGIRKQWHEDITQDLRNHLVHKLVQAIFPTPDPAALKDRRMENLVAYARKVEGDMYESANNRAEYYHLLAEKIYKIQKELEEKRRTRLQKQNMLPNAAGMVPVSMNPGPNMGQPQPGMTSNGPLPDPSMIRGSVPNQMMPRITPQSGLNQFGQMSMAQPPIVPRQTPPLQHHGQLAQPGALNPPMGYGPRMQQPSNQGQFLPQTQFPSQGMNVTNIPLAPSSGQAPVSQAQMSSSSCPVNSPIMPPGSQGSHIHCPQLPQPALHQNSPSPVPSRTPTPHHTPPSIGAQQPPATTIPAPVPTPPAMPPGPQSQALHPPPRQTPTPPTTQLPQQVQPSLPAAPSADQPQQQPRSQQSTAASVPTPTAPLLPPQPATPLSQPAVSIEGQVSNPPSTSSTEVNSQAIAEKQPSQEVKMEAKMEVDQPEPADTQPEDISESKVEDCKMESTETEERSTELKTEIKEEEDQPSTSATQSSPAPGQSKKKIFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLDSVHFFRPKCLRTAVYHEILIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQDRFVYTCNECKHHVETRWHCTVCEDYDLCITCYNTKNHDHKMEKLGLGLDDESNNQQAAATQSPGDSRRLSIQRCIQSLVHACQCRNANCSLPSCQKMKRVVQHTKGCKRKTNGGCPICKQLIALCCYHAKHCQENKCPVPFCLNIKQKLRQQQLQHRLQQAQMLRRRMASMQRTGVVGQQQGLPSPTPATPTTPTGQQPTTPQTPQPTSQPQPTPPNSMPPYLPRTQAAGPVSQGKAAGQVTPPTPPQTAQPPLPGPPPAAVEMAMQIQRAAETQRQMAHVQIFQRPIQHQMPPMTPMAPMGMNPPPMTRGPSGHLEPGMGPTGMQQQPPWSQGGLPQPQQLQSGMPRPAMMSVAQHGQPLNMAPQPGLGQVGISPLKPGTVSQQALQNLLRTLRSPSSPLQQQQVLSILHANPQLLAAFIKQRAAKYANSNPQPIPGQPGMPQGQPGLQPPTMPGQQGVHSNPAMQNMNPMQAGVQRAGLPQQQPQQQLQPPMGGMSPQAQQMNMNHNTMPSQFRDILRRQQMMQQQQQQGAGPGIGPGMANHNQFQQPQGVGYPPQQQQRMQHHMQQMQQGNMGQIGQLPQALGAEAGASLQAYQQRLLQQQMGSPVQPNPMSPQQHMLPNQAQSPHLQGQQIPNSLSNQVRSPQPVPSPRPQSQPPHSSPSPRMQPQPSPHHVSPQTSSPHPGLVAAQANPMEQGHFASPDQNSMLSQLASNPGMANLHGASATDLGLSTDNSDLNSNLSQSTLDIH

SEQ ID NO: 46 - protein, 617 aa, Artificial Sequence
  Synthetic polypeptide
IFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLDSVHFFRPKCLRTAVYHEILIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQD

SEQ ID NO: 47 - DNA, 20 bp, Artificial Sequence
  Synthetic polynucleotide
aaagcatgcgctacaataac

SEQ ID NO: 48 - DNA, 20 bp, Artificial Sequence
  Synthetic polynucleotide
acagatgcgtcaggcatctc

SEQ ID NO: 49 - DNA, 20 bp, Artificial Sequence
  Synthetic polynucleotide
acatcctctattctgatcat

SEQ ID NO: 50 - DNA, 20 bp, Artificial Sequence
  Synthetic polynucleotide
aggagcggtcagtgacgcga

SEQ ID NO: 51 - DNA, 20 bp, Artificial Sequence
  Synthetic polynucleotide
atgcctgacgcatctgtctg

SEQ ID NO: 52 - DNA, 20 bp, Artificial Sequence
  Synthetic polynucleotide
cataagcaacctgggatcaa

SEQ ID NO: 53 - DNA, 20 bp, Artificial Sequence
  Synthetic polynucleotide
ccaggtcattccggtgaggg

SEQ ID NO: 54 - DNA, 20 bp, Artificial Sequence
  Synthetic polynucleotide
ccctcaccggaatgacctgg

SEQ ID NO: 55 - DNA, 20 bp, Artificial Sequence
  Synthetic polynucleotide
ccctcaggtcttcctatgtg

SEQ ID NO: 56 - DNA, 20 bp, Artificial Sequence
  Synthetic polynucleotide
ccgcacataggaagacctga

SEQ ID NO: 57 - DNA, 20 bp, Artificial Sequence
  Synthetic polynucleotide
cctattgcgggtgtctgcgg

SEQ ID NO: 58 - DNA, 20 bp, Artificial Sequence
  Synthetic polynucleotide
cctgtcgcgacaccacagtt

SEQ ID NO: 59 - DNA, 20 bp, Artificial Sequence
  Synthetic polynucleotide
cgagcggacaggataccatc

SEQ ID NO: 60 - DNA, 20 bp, Artificial Sequence
  Synthetic polynucleotide
cggtcagtgacgcgatggag

SEQ ID NO: 61 - DNA, 20 bp, Artificial Sequence
  Synthetic polynucleotide
cgtggggggaccagtgcata

SEQ ID NO: 62 - DNA, 20 bp, Artificial Sequence
  Synthetic polynucleotide
cgtgtggcgagggtacgtgg

SEQ ID NO: 63 - DNA, 20 bp, Artificial Sequence
  Synthetic polynucleotide
ctgtcgcgacaccacagttg

SEQ ID NO: 64 - DNA, 20 bp, Artificial Sequence
  Synthetic polynucleotide
gagcggacaggataccatcg

SEQ ID NO: 65 - DNA, 20 bp, Artificial Sequence
  Synthetic polynucleotide
gatgcgtcaggcatctccgg

SEQ ID NO: 66 - DNA, 20 bp, Artificial Sequence
  Synthetic polynucleotide
gcacgcctgcgcggccgcag

SEQ ID NO: 67 - DNA, 20 bp, Artificial Sequence
  Synthetic polynucleotide
gcagcgagtctggcgcagag

SEQ ID NO: 68 - DNA, 20 bp, Artificial Sequence
  Synthetic polynucleotide
gcaggctggcgcgcatgctc

SEQ ID NO: 69 - DNA, 20 bp, Artificial Sequence
  Synthetic polynucleotide
gcgccccaatgcgagcggac

SEQ ID NO: 70 - DNA, 20 bp, Artificial Sequence
  Synthetic polynucleotide
gcggcgacagtgggtattgg

SEQ ID NO: 71 - DNA, 20 bp, Artificial Sequence
  Synthetic polynucleotide
ggcaaacaagcacgcctgcg

SEQ ID NO: 72 - DNA, 20 bp, Artificial Sequence
  Synthetic polynucleotide
gggacgcgccccaatgcgag

SEQ ID NO: 73 - DNA, 20 bp, Artificial Sequence
  Synthetic polynucleotide
ggtcagtgacgcgatggagc

SEQ ID NO: 74 - DNA, 20 bp, Artificial Sequence
  Synthetic polynucleotide
gtcgcgacaggtcctattgc

SEQ ID NO: 75 - DNA, 20 bp, Artificial Sequence
  Synthetic polynucleotide
gtgacgcgatggagcgggca

SEQ ID NO: 76 - DNA, 20 bp, Artificial Sequence
  Synthetic polynucleotide
gttgtgccgttctgccccga

SEQ ID NO: 77 - DNA, 20 bp, Artificial Sequence
  Synthetic polynucleotide
tccgtgtggcgagggtacgt

SEQ ID NO: 78 - DNA, 20 bp, Artificial Sequence
  Synthetic polynucleotide
tgaccgaggcgaggaggcta

SEQ ID NO: 79 - DNA, 20 bp, Artificial Sequence
  Synthetic polynucleotide
tgacgcatctgtctgaggag

SEQ ID NO: 80 - DNA, 20 bp, Artificial Sequence
  Synthetic polynucleotide
tgccgctgctgcagcgagtc

SEQ ID NO: 81 - DNA, 20 bp, Artificial Sequence
  Synthetic polynucleotide
tgcttgtttgccgcagtgca

SEQ ID NO: 82 - DNA, 20 bp, Artificial Sequence
  Synthetic polynucleotide
tgtcgcgacaccacagttgg

SEQ ID NO: 83 - DNA, 20 bp, Artificial Sequence
  Synthetic polynucleotide
ttaggcggaggtaggtatat

SEQ ID NO: 84 - DNA, 20 bp, Artificial Sequence
  Synthetic polynucleotide
ctcaaatcaatctccctgta

SEQ ID NO: 85 - DNA, 20 bp, Artificial Sequence
  Synthetic polynucleotide
ctgcaattcgttccttccag

SEQ ID NO: 86 - DNA, 20 bp, Artificial Sequence
  Synthetic polynucleotide
gtgagtggaggttgactaaa

SEQ ID NO: 87 - DNA, 4560 bp, Artificial Sequence
  Synthetic polynucleotide
atggactacaaagaccatgacggtgattataaagatcatgacatcgattacaaggatgacgatgacaagatggcccccaagaagaagaggaaggtgggccgcggaatggacaagaagtactccattgggctcgccatcggcacaaacagcgtcggctgggccgtcattacggacgagtacaaggtgccgagcaaaaaattcaaagttctgggcaataccgatcgccacagcataaagaagaacctcattggcgccctcctgttcgactccggggaaaccgccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatgccatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtggctagcgatgctaagtcactgactgcctggtcccggacactggtgaccttcaaggatgtgtttgtggacttcaccagggaggagtggaagctgctggacactgctcagcagatcctgtacagaaatgtgatgctggagaactataagaacctggtttccttgggttatcagcttactaagccagatgtgatcctccggttggagaagggagaagagccctggctggtggagagagaaattcaccaagagacccatcctgattcagagactgcatttgaaatcaaatcatcagttccgaaaaagaaacgcaaagtttga

SEQ ID NO: 88 - protein, 1519 aa, Artificial Sequence
  Synthetic polypeptide
MDYKDHDGDYKDHDIDYKDDDDKMAPKKKRKVGRGMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKVASDAKSLTAWSRTLVTFKDVFVDFTREEWKLLDTAQQILYRNVMLENYKNLVSLGYQLTKPDVILRLEKGEEPWLVEREIHQETHPDSETAFEIKSSVPKKKRKV

SEQ ID NO: 89 - DNA, 6459 bp, Artificial Sequence
  Synthetic polynucleotide
atggactacaaagaccatgacggtgattataaagatcatgacatcgattacaaggatgacgatgacaagcacgttaagcgacctgccgccacaaagaaggctggacaggctaagaagaagaaactggacgttctgcccacctgcagctgtcttgatcgagttatacaaaaagacaaaggcccatattatacacaccttggggcaggaccaagtgttgctgctgtcagggaaatcatggagaataggtatggtcaaaaaggaaacgcaataaggatagaaatagtagtgtacaccggtaaagaagggaaaagctctcatgggtgtccaattgctaagtgggttttaagaagaagcagtgatgaagaaaaagttctttgtttggtccggcagcgtacaggccaccactgtccaactgctgtgatggtggtgctcatcatggtgtgggatggcatccctcttccaatggccgaccggctatacacagagctcacagagaatctaaagtcatacaatgggcaccctaccgacagaagatgcaccctcaatgaaaatcgtacctgtacatgtcaaggaattgatccagagacttgtggagcttcattctcttttggctgttcatggagtatgtactttaatggctgtaagtttggtagaagcccaagccccagaagatttagaattgatccaagctctcccttacatgaaaaaaaccttgaagataacttacagagtttggctacacgattagctccaatttataagcagtatgctccagtagcttaccaaaatcaggtggaatatgaaaatgttgcccgagaatgtcggcttggcagcaaggaaggtcgacccttctctggggtcactgcttgcctggacttctgtgctcatccccacagggacattcacaacatgaataatggaagcactgtggtttgtaccttaactcgagaagataaccgctctttgggtgttattcctcaagatgagcagctccatgtgctacctctttataagctttcagacacagatgagtttggctccaaggaaggaatggaagccaagatcaaatctggggccatcgaggtcctggcaccccgccgcaaaaaaagaacgtgtttcactcagcctgttccccgttctggaaagaagagggctgcgatgatgacagaggttcttgcacataagataagggcagtggaaaagaaacctattccccgaatcaagcggaagaataactcaacaacaacaaacaacagtaagccttcgtcactgccaaccttagggagtaacactgagaccgtgcaacctgaagtaaaaagtgaaaccgaaccccattttatcttaaaaagttcagacaacactaaaacttattcgctgatgccatccgctcctcacccagtgaaagaggcatctccaggcttctcctggtccccgaagactgcttcagccacaccagctccactgaagaatgacgcaacagcctcatgcgggttttcagaaagaagcagcactccccactgtacgatgccttcgggaagactcagtggtgccaatgctgcagctgctgatggccctggcatttcacagcttggcgaagtggctcctctccccaccctgtctgctcctgtgatggagcccctcattaattctgagccttccactggtgtgactgagccgctaacgcctcatcagccaaaccaccagccctccttcctcacctctcctcaagaccttgcctcttctccaatggaagaagatgagcagcattctgaagcagatgagcctccatcagacgaacccctatctgatgaccccctgtcacctgctgaggagaaattgccccacattgatgagtattggtcagacagtgagcacatctttttggatgcaaatattggtggggtggccatcgcacctgctcacggctcggttttgattgagtgtgcccggcgagagctgcacgctaccactcctgttgagcaccccaaccgtaatcatccaacccgcctctcccttgtcttttaccagcacaaaaacctaaataagccccaacatggttttgaactaaacaagattaagtttgaggctaaagaagctaagaataagaaaatgaaggcctcagagcaaaaagaccaggcagctaatgaaggtccagaacagtcctctgaagtaaatgaattgaaccaaattccttctcataaagcattaacattaacccatgacaatgttgtcaccgtgtccccttatgctctcacacacgttgcggggccctataaccattgggtcaaccccaagaagaagaggaaggtgggccgcggaatggacaagaagtactccattgggctcgccatcggcacaaacagcgtcggctgggccgtcattacggacgagtacaaggtgccgagcaaaaaattcaaagttctgggcaataccgatcgccacagcataaagaagaacctcattggcgccctcctgttcgactccggggaaaccgccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatgccatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtgtag

SEQ ID NO: 90 - protein, 2152 aa, Artificial Sequence
  Synthetic polypeptide
MDYKDHDGDYKDHDIDYKDDDDKHVKRPAATKKAGQAKKKKLDVLPTCSCLDRVIQKDKGPYYTHLGAGPSVAAVREIMENRYGQKGNAIRIEIVVYTGKEGKSSHGCPIAKWVLRRSSDEEKVLCLVRQRTGHHCPTAVMVVLIMVWDGIPLPMADRLYTELTENLKSYNGHPTDRRCTLNENRTCTCQGIDPETCGASFSFGCSWSMYFNGCKFGRSPSPRRFRIDPSSPLHEKNLEDNLQSLATRLAPIYKQYAPVAYQNQVEYENVARECRLGSKEGRPFSGVTACLDFCAHPHRDIHNMNNGSTVVCTLTREDNRSLGVIPQDEQLHVLPLYKLSDTDEFGSKEGMEAKIKSGAIEVLAPRRKKRTCFTQPVPRSGKKRAAMMTEVLAHKIRAVEKKPIPRIKRKNNSTTTNNSKPSSLPTLGSNTETVQPEVKSETEPHFILKSSDNTKTYSLMPSAPHPVKEASPGFSWSPKTASATPAPLKNDATASCGFSERSSTPHCTMPSGRLSGANAAAADGPGISQLGEVAPLPTLSAPVMEPLINSEPSTGVTEPLTPHQPNHQPSFLTSPQDLASSPMEEDEQHSEADEPPSDEPLSDDPLSPAEEKLPHIDEYWSDSEHIFLDANIGGVAIAPAHGSVLIECARRELHATTPVEHPNRNHPTRLSLVFYQHKNLNKPQHGFELNKIKFEAKEAKNKKMKASEQKDQAANEGPEQSSEVNELNQIPSHKALTLTHDNVVTVSPYALTHVAGPYNHWVNPKKKRKVGRGMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKV

SEQ ID NO: 91 - DNA, 20 bp, Artificial Sequence
  Artificial polynucleotide
acccacccagcaaccaccag

SEQ ID NO: 92 - DNA, 20 bp, Artificial Sequence
  Artificial polynucleotide
gacaatgacaagggttgtgg

SEQ ID NO: 93 - DNA, 20 bp, Artificial Sequence
  Artificial polynucleotide
ggttttgagcaagccctcct

SEQ ID NO: 94 - DNA, 20 bp, Artificial Sequence
  Artificial polynucleotide
tcgttgtgacactactgagt

SEQ ID NO: 95 - DNA, 20 bp, Artificial Sequence
  Artificial polynucleotide
ctgccacgctgtgactcaga

SEQ ID NO: 96 - DNA, 20 bp, Artificial Sequence
  Artificial polynucleotide
aaaatcccaaaggcttcaga

SEQ ID NO: 97 - DNA, 20 bp, Artificial Sequence
  Artificial polynucleotide
aaagttataaatcacgttaa

SEQ ID NO: 98 - DNA, 20 bp, Artificial Sequence
  Artificial polynucleotide
aaatgcacttacccacgtat

SEQ ID NO: 99 - DNA, 20 bp, Artificial Sequence
  Artificial polynucleotide
aaatgttgtcatccatatcc

SEQ ID NO: 100 - DNA, 20 bp, Artificial Sequence
  Artificial polynucleotide
aacctgaggttagcagcaag

SEQ ID NO: 101 - DNA, 20 bp, Artificial Sequence
  Artificial polynucleotide
aactatatgctacctggata

SEQ ID NO: 102 - DNA, 20 bp, Artificial Sequence
  Artificial polynucleotide
aactcaaatgacctctatct

SEQ ID NO: 103 - DNA, 20 bp, Artificial Sequence
  Artificial polynucleotide
aactttttctttgcctgtac

SEQ ID NO: 104 - DNA, 20 bp, Artificial Sequence
  Artificial polynucleotide
aagcaagaatgaatattacc

SEQ ID NO: 105 - DNA, 20 bp, Artificial Sequence
  Artificial polynucleotide
aatcaatgatagctggagtg

SEQ ID NO: 106 - DNA, 20 bp, Artificial Sequence
  Artificial polynucleotide
aatgcctggctgtcattcag

SEQ ID NO: 107 - DNA, 20 bp, Artificial Sequence
  Artificial polynucleotide
aattcagagatcaggtcaag

SEQ ID NO: 108 - DNA, 20 bp, Artificial Sequence
  Artificial polynucleotide
acacagtctgtactcactct

SEQ ID NO: 109 - DNA, 20 bp, Artificial Sequence
  Artificial polynucleotide
acactacacctcaacatatc

SEQ ID NO: 110 - DNA, 20 bp, Artificial Sequence
  Artificial polynucleotide
acagaccttgaaacttcaag

SEQ ID NO: 111 - DNA, 20 bp, Artificial Sequence
  Artificial polynucleotide
acatgctagtagaccctgat

SEQ ID NO: 112 - DNA, 20 bp, Artificial Sequence
  Artificial polynucleotide
acatttcggcaatgacacac

SEQ ID NO: 113 - DNA, 20 bp, Artificial Sequence
  Artificial polynucleotide
accaactcctacccttacct

SEQ ID NO: 114 - DNA, 20 bp, Artificial Sequence
  Artificial polynucleotide
accaatcccttacctggagg

SEQ ID NO: 115 - DNA, 20 bp, Artificial Sequence
  Artificial polynucleotide
acccacccagcaaccaccag

SEQ ID NO: 116 - DNA, 20 bp, Artificial Sequence
  Artificial polynucleotide
acccctatgaatcagtaacc

SEQ ID NO: 117 - DNA, 20 bp, Artificial Sequence
  Artificial polynucleotide
accgcacataggaagacctg

SEQ ID NO: 118 - DNA, 20 bp, Artificial Sequence
  Artificial polynucleotide
accgggtgatgcaagcaaca

SEQ ID NO: 119 - DNA, 20 bp, Artificial Sequence
  Artificial polynucleotide
acggtgcagcagtagaggac

SEQ ID NO: 120 - DNA, 20 bp, Artificial Sequence
  Artificial polynucleotide
acgtctcgccccactgcaag

SEQ ID NO: 121 - DNA, 20 bp, Artificial Sequence
  Artificial polynucleotide
acgtggggggaccagtgcat

SEQ ID NO: 122 - DNA, 20 bp, Artificial Sequence
  Artificial polynucleotide
acgttaacggccgggtgagg

SEQ ID NO: 123 - DNA, 20 bp, Artificial Sequence
  Artificial polynucleotide
actagtaatgggtactattt

SEQ ID NO: 124 - DNA, 20 bp, Artificial Sequence
  Artificial polynucleotide
actcttgccaagagtaacct

SEQ ID NO: 125 - DNA, 20 bp, Artificial Sequence
  Artificial polynucleotide
actgaagacctagagaatta

SEQ ID NO: 126 - DNA, 20 bp, Artificial Sequence
  Artificial polynucleotide
actgcaccctaaagagggta

SEQ ID NO: 127 - DNA, 20 bp, Artificial Sequence
  Artificial polynucleotide
actgctgcaccgtgggtaag

SEQ ID NO: 128 - DNA, 20 bp, Artificial Sequence
  Artificial polynucleotide
actgtctctacatttagaat

SEQ ID NO: 129 - DNA, 20 bp, Artificial Sequence
  Artificial polynucleotide
agaaacatgacgaaagtgtc

SEQ ID NO: 130 - DNA, 20 bp, Artificial Sequence
  Artificial polynucleotide
agaaggagggaatcctaatg

SEQ ID NO: 131 - DNA, 20 bp, Artificial Sequence
  Artificial polynucleotide
agacataccagttatgatag

SEQ ID NO: 132 - DNA, 20 bp, Artificial Sequence
  Artificial polynucleotide
agagcggtggctgggtggcc

SEQ ID NO: 133 - DNA, 20 bp, Artificial Sequence
  Artificial polynucleotide
agatgtcaacactcctcatt

SEQ ID NO: 134 - DNA, 20 bp, Artificial Sequence
  Artificial polynucleotide
agcatgcggggtaaccgcag

SEQ ID NO: 135 - DNA, 20 bp, Artificial Sequence
  Artificial polynucleotide
agccagctagcatccagaaa

SEQ ID NO: 136 - DNA, 20 bp, Artificial Sequence
  Artificial polynucleotide
agccatccaaagagacaaca

SEQ ID NO: 137 - DNA, 20 bp, Artificial Sequence
  Artificial polynucleotide
agctagcctgaagcaggtgg

SEQ ID NO: 138 - DNA, 20 bp, Artificial Sequence
  Artificial polynucleotide
aggattaggcttaaaaggac

SEQ ID NO: 139 - DNA, 20 bp, Artificial Sequence
  Artificial polynucleotide
aggcttgggctgtcattcag

SEQ ID NO: 140 - DNA, 20 bp, Artificial Sequence
  Artificial polynucleotide
agggcgcaatggttcatgga

SEQ ID NO: 141 - DNA, 20 bp, Artificial Sequence
  Artificial polynucleotide
agggcgctgataaacaaagg

SEQ ID NO: 142 - DNA, 20 bp, Artificial Sequence
  Artificial polynucleotide
agggctccaaaaagtaaacc

SEQ ID NO: 143 - DNA, 20 bp, Artificial Sequence
  Artificial polynucleotide
agggtgattgcagttccgtg

SEQ ID NO: 144 - DNA, 20 bp, Artificial Sequence
  Artificial polynucleotide
agtcactgcactgcgcaaat

SEQ ID NO: 145 - DNA, 20 bp, Artificial Sequence
  Artificial polynucleotide
agtcattacctgagaaggca

SEQ ID NO: 146 - DNA, 20 bp, Artificial Sequence
  Artificial polynucleotide
agtccacactgaggttacat

SEQ ID NO: 147 - DNA, 20 bp, Artificial Sequence
  Artificial polynucleotide
agtcctctactgctgcaccg

SEQ ID NO: 148 - DNA, 20 bp, Artificial Sequence
  Artificial polynucleotide
agtgtatcttctgagatcaa

SEQ ID NO: 149 - DNA, 20 bp, Artificial Sequence
  Artificial polynucleotide
atactacttatatgccaatg

SEQ ID NO: 150 - DNA, 20 bp, Artificial Sequence
  Artificial polynucleotide
atactcactatagtaaaaat

SEQ ID NO: 151 - DNA, 20 bp, Artificial Sequence
  Artificial polynucleotide
atagtaaactctcagaaatg

SEQ ID NO: 152 - DNA, 20 bp, Artificial Sequence
  Artificial polynucleotide
atagtgtatctctacccata

SEQ ID NO: 153 - DNA, 20 bp, Artificial Sequence
  Artificial polynucleotide
atatggctttaaccacctct

SEQ ID NO: 154 - DNA, 20 bp, Artificial Sequence
  Artificial polynucleotide
atcacgttaacggccgggtg

SEQ ID NO: 155 - DNA, 20 bp, Artificial Sequence
  Artificial polynucleotide
atcccaaaggcttcagaagg

SEQ ID NO: 156 - DNA, 20 bp, Artificial Sequence
  Artificial polynucleotide
atctgccatattaggatccc

SEQ ID NO: 157 - DNA, 20 bp, Artificial Sequence
  Artificial polynucleotide
atcttccttacatctttggg

SEQ ID NO: 158 - DNA, 20 bp, Artificial Sequence
  Artificial polynucleotide
atcttgaaggagctgcacaa

SEQ ID NO: 159 - DNA, 20 bp, Artificial Sequence
  Artificial polynucleotide
atgatctatccattccgtat

SEQ ID NO: 160 - DNA, 20 bp, Artificial Sequence
  Artificial polynucleotide
atgtggactaaatatgtatt

SEQ ID NO: 161 - DNA, 20 bp, Artificial Sequence
  Artificial polynucleotide
atgtttgtaatagaaagtgt

SEQ ID NO: 162 - DNA, 20 bp, Artificial Sequence
  Artificial polynucleotide
attaccaacccttcttctac

SEQ ID NO: 163 - DNA, 20 bp, Artificial Sequence
  Artificial polynucleotide
attgtatgacacatacactt

SEQ ID NO: 164 - DNA, 20 bp, Artificial Sequence
  Artificial polynucleotide
atttggactgctaacaccat

SEQ ID NO: 165 - DNA, 20 bp, Artificial Sequence
  Artificial polynucleotide
caagaacacttaacagcttc

SEQ ID NO: 166 - DNA, 20 bp, Artificial Sequence
  Artificial polynucleotide
caagaacctggcatatacga

SEQ ID NO: 167 - DNA, 20 bp, Artificial Sequence
  Artificial polynucleotide
caatcatctactggtaagca

SEQ ID NO: 168 - DNA, 20 bp, Artificial Sequence
  Artificial polynucleotide
caatcccttacctggaggtg

SEQ ID NO: 169 - DNA, 20 bp, Artificial Sequence
  Artificial polynucleotide
cacacacaatgcataggaca

SEQ ID NO: 170 - DNA, 20 bp, Artificial Sequence
  Artificial polynucleotide
cacccagcaaccaccagagg

SEQ ID NO: 171 - DNA, 20 bp, Artificial Sequence
  Artificial polynucleotide
caccctgcatcccaactact

SEQ ID NO: 172 - DNA, 20 bp, Artificial Sequence
  Artificial polynucleotide
caccggaatgacctgggggg

SEQ ID NO: 173 - DNA, 20 bp, Artificial Sequence
  Artificial polynucleotide
cacctttgtattggtcatct

SEQ ID NO: 174 - DNA, 20 bp, Artificial Sequence
  Artificial polynucleotide
cactacctgaacaccaccac

SEQ ID NO: 175 - DNA, 20 bp, Artificial Sequence
  Artificial polynucleotide
cagaagcttcaaagagtaga

SEQ ID NO: 176 - DNA, 20 bp, Artificial Sequence
  Artificial polynucleotide
cagagggcgctgataaacaa

SEQ ID NO: 177 - DNA, 20 bp, Artificial Sequence
  Artificial polynucleotide
caggtcattccggtgaggga

SEQ ID NO: 178 - DNA, 20 bp, Artificial Sequence
  Artificial polynucleotide
cagtagacatacttatatag

SEQ ID NO: 179 - DNA, 20 bp, Artificial Sequence
  Artificial polynucleotide
cagtccttctgcgtttgact

SEQ ID NO: 180 - DNA, 20 bp, Artificial Sequence
  Artificial polynucleotide
cagtgggtattggcggcggt

SEQ ID NO: 181 - DNA, 20 bp, Artificial Sequence
  Artificial polynucleotide
catactagcttcctcaccaa

SEQ ID NO: 182 - DNA, 20 bp, Artificial Sequence
  Artificial polynucleotide
cattcctcaaacagctccag

SEQ ID NO: 183 - DNA, 20 bp, Artificial Sequence
  Artificial polynucleotide
ccaatcacccacctttctcc

SEQ ID NO: 184 - DNA, 20 bp, Artificial Sequence
  Artificial polynucleotide
ccaccgcagacacccgcaat

SEQ ID NO: 185 - DNA, 20 bp, Artificial Sequence
  Artificial polynucleotide
ccactacacacacaatgcat

SEQ ID NO: 186 - DNA, 20 bp, Artificial Sequence
  Artificial polynucleotide
ccagtgagctagcctgaagc

SEQ ID NO: 187 - DNA, 20 bp, Artificial Sequence
  Artificial polynucleotide
ccatcccacacaggcggagg

SEQ ID NO: 188 - DNA, 20 bp, Artificial Sequence
  Artificial polynucleotide
cccactctaccacacaggtt

SEQ ID NO: 189 - DNA, 20 bp, Artificial Sequence
  Artificial polynucleotide
cccatctcaccatgcaagag

SEQ ID NO: 190 - DNA, 20 bp, Artificial Sequence
  Artificial polynucleotide
cccccacctaagatctaagt

SEQ ID NO: 191 - DNA, 20 bp, Artificial Sequence
  Artificial polynucleotide
cccccaggtcattccggtga

SEQ ID NO: 192 - DNA, 20 bp, Artificial Sequence
  Artificial polynucleotide
ccccccaggtcattccggtg

SEQ ID NO: 193 - DNA, 20 bp, Artificial Sequence
  Artificial polynucleotide
cccctaatcccgaatagcat

SEQ ID NO: 194 - DNA, 20 bp, Artificial Sequence
  Artificial polynucleotide
cccctctactgcgccacaac

SEQ ID NO: 195 - DNA, 20 bp, Artificial Sequence
  Artificial polynucleotide
cccggaggcggcatgcctat

SEQ ID NO: 196 - DNA, 20 bp, Artificial Sequence
  Artificial polynucleotide
ccggacagcgacaggccccg

SEQ ID NO: 197 - DNA, 20 bp, Artificial Sequence
  Artificial polynucleotide
cctaaaaaatagctaaacag

SEQ ID NO: 198 - DNA, 20 bp, Artificial Sequence
  Artificial polynucleotide
cctaatttacactcaccctc

SEQ ID NO: 199 - DNA, 20 bp, Artificial Sequence
  Artificial polynucleotide
cctacgataggtagaaaccc

SEQ ID NO: 200 - DNA, 20 bp, Artificial Sequence
  Artificial polynucleotide
cctacttagatcttaggtgg

SEQ ID NO: 201 - DNA, 20 bp, Artificial Sequence
  Artificial polynucleotide
cctcaccggaatgacctggg

SEQ ID NO: 202 - DNA, 20 bp, Artificial Sequence
  Artificial polynucleotide
cctccctcaccggaatgacc

SEQ ID NO: 203 - DNA, 20 bp, Artificial Sequence
  Artificial polynucleotide
cctgagagtccatcatctgc

SEQ ID NO: 204 - DNA, 20 bp, Artificial Sequence
  Artificial polynucleotide
cctgagggtgagtgtaaatt

SEQ ID NO: 205 - DNA, 20 bp, Artificial Sequence
  Artificial polynucleotide
ccttgacattctctgtatgg

SEQ ID NO: 206 - DNA, 20 bp, Artificial Sequence
  Artificial polynucleotide
cgacatagccttccaagacc

SEQ ID NO: 207 - DNA, 20 bp, Artificial Sequence
  Artificial polynucleotide
cgcctaaaatccctatgcac

SEQ ID NO: 208 - DNA, 20 bp, Artificial Sequence
  Artificial polynucleotide
cgcctaccagtcaactcccc

SEQ ID NO: 209 - DNA, 20 bp, Artificial Sequence
  Artificial polynucleotide
cgcgacaccacagttggggg

SEQ ID NO: 210 - DNA, 20 bp, Artificial Sequence
  Artificial polynucleotide
cggcagttgctcaatatgaa

SEQ ID NO: 211 - DNA, 20 bp, Artificial Sequence
  Artificial polynucleotide
cgtgaagtgatcggtattta

SEQ ID NO: 212 - DNA, 20 bp, Artificial Sequence
  Artificial polynucleotide
ctaaagcgagggttcctaaa

SEQ ID NO: 213 - DNA, 20 bp, Artificial Sequence
  Artificial polynucleotide
ctaactacttcagttaccct

SEQ ID NO: 214 - DNA, 20 bp, Artificial Sequence
  Artificial polynucleotide
ctacctcagaggagtgtcaa

SEQ ID NO: 215 - DNA, 20 bp, Artificial Sequence
  Artificial polynucleotide
ctacctgctgaaatcagtga

SEQ ID NO: 216 - DNA, 20 bp, Artificial Sequence
  Artificial polynucleotide
ctactgcgccacaaccggaa

SEQ ID NO: 217 - DNA, 20 bp, Artificial Sequence
  Artificial polynucleotide
ctactgctgcaccgtgggta

SEQ ID NO: 218 - DNA, 20 bp, Artificial Sequence
  Artificial polynucleotide
ctattggcgtccctcgagca

SEQ ID NO: 219 - DNA, 20 bp, Artificial Sequence
  Artificial polynucleotide
ctcataaaatggctgctcaa

SEQ ID NO: 220 - DNA, 20 bp, Artificial Sequence
  Artificial polynucleotide
ctcctacttagatcttaggt

SEQ ID NO: 221 - DNA, 20 bp, Artificial Sequence
  Artificial polynucleotide
ctctacacagagattgtaca

SEQ ID NO: 222 - DNA, 20 bp, Artificial Sequence
  Artificial polynucleotide
ctctcattgcaacagtgctg

SEQ ID NO: 223 - DNA, 20 bp, Artificial Sequence
  Artificial polynucleotide
ctgagctcagagccttctgt

SEQ ID NO: 224 - DNA, 20 bp, Artificial Sequence
  Artificial polynucleotide
ctggcgcgcatgctcaggcg

SEQ ID NO: 225 - DNA, 20 bp, Artificial Sequence
  Artificial polynucleotide
cttaatgattatctctgggg

SEQ ID NO: 226 - DNA, 20 bp, Artificial Sequence
  Artificial polynucleotide
cttaccactgcaccctaaag

SEQ ID NO: 227 - DNA, 20 bp, Artificial Sequence
  Artificial polynucleotide
cttactcaagggaaaagctt

SEQ ID NO: 228 - DNA, 20 bp, Artificial Sequence
  Artificial polynucleotide
cttatgatttggtaggaagt

SEQ ID NO: 229 - DNA, 20 bp, Artificial Sequence
  Artificial polynucleotide
cttgcagtggggcgagacgt

SEQ ID NO: 230 - DNA, 20 bp, Artificial Sequence
  Artificial polynucleotide
cttgttggcatgtaaggatt

SEQ ID NO: 231 - DNA, 20 bp, Artificial Sequence
  Artificial polynucleotide
gaaacatgacgaaagtgtca

SEQ ID NO: 232 - DNA, 20 bp, Artificial Sequence
  Artificial polynucleotide
gaaaggtctgaggtcaactg

SEQ ID NO: 233 - DNA, 20 bp, Artificial Sequence
  Artificial polynucleotide
gaacagtgataggcaaagag

SEQ ID NO: 234 - DNA, 20 bp, Artificial Sequence
  Artificial polynucleotide
gaacatttgactaccagacc

SEQ ID NO: 235 - DNA, 20 bp, Artificial Sequence
  Artificial polynucleotide
gaacctgaggttagcagcaa

SEQ ID NO: 236 - DNA, 20 bp, Artificial Sequence
  Artificial polynucleotide
gaacttccagggctatttaa

SEQ ID NO: 237 - DNA, 20 bp, Artificial Sequence
  Artificial polynucleotide
gaattgttgggagacctatc

SEQ ID NO: 238 - DNA, 20 bp, Artificial Sequence
  Artificial polynucleotide
gacctcctacagcagttaaa

SEQ ID NO: 239 - DNA, 20 bp, Artificial Sequence
  Artificial polynucleotide
gactttggcggcggtaggca

SEQ ID NO: 240 - DNA, 20 bp, Artificial Sequence
  Artificial polynucleotide
gatctgccatattaggatcc

SEQ ID NO: 241 - DNA, 20 bp, Artificial Sequence
  Artificial polynucleotide
gatgtacaacctaaccctca

SEQ ID NO: 242 - DNA, 20 bp, Artificial Sequence
  Artificial polynucleotide
gattcaaagaggaaatacat

SEQ ID NO: 243 - DNA, 20 bp, Artificial Sequence
  Artificial polynucleotide
gattcctcgctactccaata

SEQ ID NO: 244 - DNA, 20 bp, Artificial Sequence
  Artificial polynucleotide
gcaaaccagatgaccatcgc

SEQ ID NO: 245 - DNA, 20 bp, Artificial Sequence
  Artificial polynucleotide
gcaaggtagtaaacaagtgc

SEQ ID NO: 246 - DNA, 20 bp, Artificial Sequence
  Artificial polynucleotide
gcaatgagaatccatatcca

SEQ ID NO: 247 - DNA, 20 bp, Artificial Sequence
  Artificial polynucleotide
gcagatctaagccgctgcag

SEQ ID NO: 248 - DNA, 20 bp, Artificial Sequence
  Artificial polynucleotide
gcaggacagtccttcaccac

SEQ ID NO: 249 - DNA, 20 bp, Artificial Sequence
  Artificial polynucleotide
gcagggtacgtggggcgact

SEQ ID NO: 250 - DNA, 20 bp, Artificial Sequence
  Artificial polynucleotide
gcctctgaacattccggatc

SEQ ID NO: 251 - DNA, 20 bp, Artificial Sequence
  Artificial polynucleotide
gcgaaggtacgtgaagtgat

SEQ ID NO: 252 - DNA, 20 bp, Artificial Sequence
  Artificial polynucleotide
gcgacagtgggtattggcgg

SEQ ID NO: 253 - DNA, 20 bp, Artificial Sequence
  Artificial polynucleotide
gcgcacaatacaaccaagag

SEQ ID NO: 254 - DNA, 20 bp, Artificial Sequence
  Artificial polynucleotide
gcgcagttgtaccatgaatg

SEQ ID NO: 255 - DNA, 20 bp, Artificial Sequence
  Artificial polynucleotide
gcggctctacagggaggaag

SEQ ID NO: 256 - DNA, 20 bp, Artificial Sequence
  Artificial polynucleotide
gctacaatgaaactttggag

SEQ ID NO: 257 - DNA, 20 bp, Artificial Sequence
  Artificial polynucleotide
gctcatcacaaatccagccc

SEQ ID NO: 258 - DNA, 20 bp, Artificial Sequence
  Artificial polynucleotide
gctcatgaaactttcatata

SEQ ID NO: 259 - DNA, 20 bp, Artificial Sequence
  Artificial polynucleotide
gctcctcagacagatgcgtc

SEQ ID NO: 260 - DNA, 20 bp, Artificial Sequence
  Artificial polynucleotide
gctggcgcgcatgctcaggc

SEQ ID NO: 261 - DNA, 20 bp, Artificial Sequence
  Artificial polynucleotide
gctgttctgtagttcgtctc

SEQ ID NO: 262 - DNA, 20 bp, Artificial Sequence
  Artificial polynucleotide
gcttctgaagacctagggga

SEQ ID NO: 263 - DNA, 20 bp, Artificial Sequence
  Artificial polynucleotide
gcttgtttgccgcagtgcag

SEQ ID NO: 264 - DNA, 20 bp, Artificial Sequence
  Artificial polynucleotide
ggaaaataaaacagcatgta

SEQ ID NO: 265 - DNA, 20 bp, Artificial Sequence
  Artificial polynucleotide
ggaattcgtcgcagtgaccg

SEQ ID NO: 266 - DNA, 20 bp, Artificial Sequence
  Artificial polynucleotide
ggacctcacgctccctttgc

SEQ ID NO: 267 - DNA, 20 bp, Artificial Sequence
  Artificial polynucleotide
ggactatccagagattaaga

SEQ ID NO: 268 - DNA, 20 bp, Artificial Sequence
  Artificial polynucleotide
ggcaaagagaggataccaaa

SEQ ID NO: 269 - DNA, 20 bp, Artificial Sequence
  Artificial polynucleotide
ggcggagtgccccgtcctgg

SEQ ID NO: 270 - DNA, 20 bp, Artificial Sequence
  Artificial polynucleotide
ggctatgtcgaaataacctg

SEQ ID NO: 271 - DNA, 20 bp, Artificial Sequence
  Artificial polynucleotide
ggctctcctacttagatctt

SEQ ID NO: 272 - DNA, 20 bp, Artificial Sequence
  Artificial polynucleotide
ggctggcgcgcatgctcagg

SEQ ID NO: 273 - DNA, 20 bp, Artificial Sequence
  Artificial polynucleotide
gggaagttgactctgatcac

SEQ ID NO: 274 - DNA, 20 bp, Artificial Sequence
  Artificial polynucleotide
ggggttgctctgccgacggg

SEQ ID NO: 275 - DNA, 20 bp, Artificial Sequence
  Artificial polynucleotide
gggtggctgccaagcttgct

SEQ ID NO: 276 - DNA, 20 bp, Artificial Sequence
  Artificial polynucleotide
gggtggtactgagaaactaa

SEQ ID NO: 277 - DNA, 20 bp, Artificial Sequence
  Artificial polynucleotide
gggttgctctgccgacgggt

SEQ ID NO: 278 - DNA, 20 bp, Artificial Sequence
  Artificial polynucleotide
ggtatcctgtccgctcgcat

SEQ ID NO: 279 - DNA, 20 bp, Artificial Sequence
  Artificial polynucleotide
gtaggaagtaggagatgaat

SEQ ID NO: 280 - DNA, 20 bp, Artificial Sequence
  Artificial polynucleotide
gtataatagtgaccactgcg

SEQ ID NO: 281 - DNA, 20 bp, Artificial Sequence
  Artificial polynucleotide
gtatatattgaaagtataaa

SEQ ID NO: 282 - DNA, 20 bp, Artificial Sequence
  Artificial polynucleotide
gtatcagaggtaaacatacc

SEQ ID NO: 283 - DNA, 20 bp, Artificial Sequence
  Artificial polynucleotide
gtatcctgtccgctcgcatt

SEQ ID NO: 284 - DNA, 20 bp, Artificial Sequence
  Artificial polynucleotide
gtcacagtgtgcggcaccag

SEQ ID NO: 285 - DNA, 20 bp, Artificial Sequence
  Artificial polynucleotide
gtcatttcatccggcctcat

SEQ ID NO: 286 - DNA, 20 bp, Artificial Sequence
  Artificial polynucleotide
gtcccccaactcctcggaca

SEQ ID NO: 287 - DNA, 20 bp, Artificial Sequence
  Artificial polynucleotide
gtcctctactgctgcaccgt

SEQ ID NO: 288 - DNA, 20 bp, Artificial Sequence
  Artificial polynucleotide
gtctagtgaggcacactgac

SEQ ID NO: 289 - DNA, 20 bp, Artificial Sequence
  Artificial polynucleotide
gtcttgtaaagatacagcaa

SEQ ID NO: 290 - DNA, 20 bp, Artificial Sequence
  Artificial polynucleotide
gtgacacagaactgaaataa

SEQ ID NO: 291 - DNA, 20 bp, Artificial Sequence
  Artificial polynucleotide
gtgacgcaacacagaccccc

SEQ ID NO: 292 - DNA, 20 bp, Artificial Sequence
  Artificial polynucleotide
gtgagctagcctgaagcagg

SEQ ID NO: 293 - DNA, 20 bp, Artificial Sequence
  Artificial polynucleotide
gtgtataaaggattcaagaa

SEQ ID NO: 294 - DNA, 20 bp, Artificial Sequence
  Artificial polynucleotide
gtgttcaccaatcccttacc

SEQ ID NO: 295 - DNA, 20 bp, Artificial Sequence
  Artificial polynucleotide
gtgttttctgttggggtgat

SEQ ID NO: 296 - DNA, 20 bp, Artificial Sequence
  Artificial polynucleotide
gttagagtactacttcttct

SEQ ID NO: 297 - DNA, 20 bp, Artificial Sequence
  Artificial polynucleotide
taactcaaatgacctctatc

SEQ ID NO: 298 - DNA, 20 bp, Artificial Sequence
  Artificial polynucleotide
taagatgacagagccctcac

SEQ ID NO: 299 - DNA, 20 bp, Artificial Sequence
  Artificial polynucleotide
tacaggccctaaatcaatcc

SEQ ID NO: 300 - DNA, 20 bp, Artificial Sequence
  Artificial polynucleotide
tacccacggtgcagcagtag

SEQ ID NO: 301 - DNA, 20 bp, Artificial Sequence
  Artificial polynucleotide
tactgctgcaccgtgggtaa

SEQ ID NO: 302 - DNA, 20 bp, Artificial Sequence
  Artificial polynucleotide
tagaggtgagtgggagcata

SEQ ID NO: 303 - DNA, 20 bp, Artificial Sequence
  Artificial polynucleotide
tagatgaactaaagaacctg

SEQ ID NO: 304 - DNA, 20 bp, Artificial Sequence
  Artificial polynucleotide
tagcatggttgactatctgt

SEQ ID NO: 305 - DNA, 20 bp, Artificial Sequence
  Artificial polynucleotide
tagtgttaggaagatcaaat

SEQ ID NO: 306 - DNA, 20 bp, Artificial Sequence
  Artificial polynucleotide
tatagaagctaacatgggaa

SEQ ID NO: 307 - DNA, 20 bp, Artificial Sequence
  Artificial polynucleotide
tatccgggctccgggtctct

SEQ ID NO: 308 - DNA, 20 bp, Artificial Sequence
  Artificial polynucleotide
tatccttaacctgtactgat

SEQ ID NO: 309 - DNA, 20 bp, Artificial Sequence
  Artificial polynucleotide
tatggtgagcagttccccaa

SEQ ID NO: 310 - DNA, 20 bp, Artificial Sequence
  Artificial polynucleotide
tattggcgtccctcgagcag

SEQ ID NO: 311 - DNA, 20 bp, Artificial Sequence
  Artificial polynucleotide
tatacacactcctagtataa

SEQ ID NO: 312 - DNA, 20 bp, Artificial Sequence
  Artificial polynucleotide
tcaactaagtggtcaaaaaa

SEQ ID NO: 313 - DNA, 20 bp, Artificial Sequence
  Artificial polynucleotide
tcaagaacctggcatatacg

SEQ ID NO: 314 - DNA, 20 bp, Artificial Sequence
  Artificial polynucleotide
tcacataaaaactacctcag

SEQ ID NO: 315 - DNA, 20 bp, Artificial Sequence
  Artificial polynucleotide
tcacctctaatgcctggtgg

SEQ ID NO: 316 - DNA, 20 bp, Artificial Sequence
  Artificial polynucleotide
tcacgtaatggggacacatt

SEQ ID NO: 317 - DNA, 20 bp, Artificial Sequence
  Artificial polynucleotide
tcactactgtaaaacaagct

SEQ ID NO: 318 - DNA, 20 bp, Artificial Sequence
  Artificial polynucleotide
tcactcttggttgctcagtg

SEQ ID NO: 319 - DNA, 20 bp, Artificial Sequence
  Artificial polynucleotide
tcatactagcttcctcacca

SEQ ID NO: 320 - DNA, 20 bp, Artificial Sequence
  Artificial polynucleotide
tccatgttcaattcaggttg

SEQ ID NO: 321 - DNA, 20 bp, Artificial Sequence
  Artificial polynucleotide
tcccaatactacttatgatt

SEQ ID NO: 322 - DNA, 20 bp, Artificial Sequence
  Artificial polynucleotide
tcccactcacctctaatgcc

SEQ ID NO: 323 - DNA, 20 bp, Artificial Sequence
  Artificial polynucleotide
tccctcaccggaatgacctg

SEQ ID NO: 324 - DNA, 20 bp, Artificial Sequence
  Artificial polynucleotide
tcctacttagatcttaggtg

SEQ ID NO: 325 - DNA, 20 bp, Artificial Sequence
  Artificial polynucleotide
tcgttgtgacactactgagt

SEQ ID NO: 326 - DNA, 20 bp, Artificial Sequence
  Artificial polynucleotide
tctacacagagattgtacaa

SEQ ID NO: 327 - DNA, 20 bp, Artificial Sequence
  Artificial polynucleotide
tctatcataactggtatgtc

SEQ ID NO: 328 - DNA, 20 bp, Artificial Sequence
  Artificial polynucleotide
tctcctacttagatcttagg

SEQ ID NO: 329 - DNA, 20 bp, Artificial Sequence
  Artificial polynucleotide
tctcgccccactgcaagcgg

SEQ ID NO: 330 - DNA, 20 bp, Artificial Sequence
  Artificial polynucleotide
tctgacctaattcagagatc

SEQ ID NO: 331 - DNA, 20 bp, Artificial Sequence
  Artificial polynucleotide
tcttggtggacgcattcctg

SEQ ID NO: 332 - DNA, 20 bp, Artificial Sequence
  Artificial polynucleotide
tgaaaaggtacgattttcca

SEQ ID NO: 333 - DNA, 20 bp, Artificial Sequence
  Artificial polynucleotide
tgaagtgatcggtatttagg

SEQ ID NO: 334 - DNA, 20 bp, Artificial Sequence
  Artificial polynucleotide
tgagctcagagccttctgtg

SEQ ID NO: 335 - DNA, 20 bp, Artificial Sequence
  Artificial polynucleotide
tgcatagggattttaggcgg

SEQ ID NO: 336 - DNA, 20 bp, Artificial Sequence
  Artificial polynucleotide
tgccttagacaacaacataa

SEQ ID NO: 337 - DNA, 20 bp, Artificial Sequence
  Artificial polynucleotide
tgctgtatcagccaggtgaa

SEQ ID NO: 338 - DNA, 20 bp, Artificial Sequence
  Artificial polynucleotide
tggaaaacacattctaggac

SEQ ID NO: 339 - DNA, 20 bp, Artificial Sequence
  Artificial polynucleotide
tggccagaatgtccaccgtg

SEQ ID NO: 340 - DNA, 20 bp, Artificial Sequence
  Artificial polynucleotide
tggctgccaagcttgctagg

SEQ ID NO: 341 - DNA, 20 bp, Artificial Sequence
  Artificial polynucleotide
tggggaattggcacaccagc

SEQ ID NO: 342 - DNA, 20 bp, Artificial Sequence
  Artificial polynucleotide
tgtagcatgcttttagagtt

SEQ ID NO: 343 - DNA, 20 bp, Artificial Sequence
  Artificial polynucleotide
tgtcgcgacaggtcctattg

SEQ ID NO: 344 - DNA, 20 bp, Artificial Sequence
  Artificial polynucleotide
tgtgaatttgtaaccaccca

SEQ ID NO: 345 - DNA, 20 bp, Artificial Sequence
  Artificial polynucleotide
tgttggggctccagcaatac

SEQ ID NO: 346 - DNA, 20 bp, Artificial Sequence
  Artificial polynucleotide
ttaaactctcctatacggaa

SEQ ID NO: 347 - DNA, 20 bp, Artificial Sequence
  Artificial polynucleotide
ttacactgaaagtaataatc

SEQ ID NO: 348 - DNA, 20 bp, Artificial Sequence
  Artificial polynucleotide
ttcaacatatcctacaactg

SEQ ID NO: 349 - DNA, 20 bp, Artificial Sequence
  Artificial polynucleotide
ttcaaccagagatgtttcat

SEQ ID NO: 350 - DNA, 20 bp, Artificial Sequence
  Artificial polynucleotide
ttcaccaatcccttacctgg

SEQ ID NO: 351 - DNA, 20 bp, Artificial Sequence
  Artificial polynucleotide
ttccggttgtggcgcagtag

SEQ ID NO: 352 - DNA, 20 bp, Artificial Sequence
  Artificial polynucleotide
ttccgtgtggcgagggtacg

SEQ ID NO: 353 - DNA, 20 bp, Artificial Sequence
  Artificial polynucleotide
ttctacaggaatactagtaa

SEQ ID NO: 354 - DNA, 20 bp, Artificial Sequence
  Artificial polynucleotide
ttgctgcattagctctaacc

SEQ ID NO: 355 - DNA, 20 bp, Artificial Sequence
  Artificial polynucleotide
ttggactgctaacaccatag

SEQ ID NO: 356 - DNA, 20 bp, Artificial Sequence
  Artificial polynucleotide
tttcagaggcatcaactaag

SEQ ID NO: 357 - DNA, 20 bp, Artificial Sequence
  Artificial polynucleotide
tttcatatagggttggaacc

SEQ ID NO: 358 - DNA, 20 bp, Artificial Sequence
  Artificial polynucleotide
tttgcctgtactctactaga

SEQ ID NO: 359 - DNA, 20 bp, Artificial Sequence
  Artificial polynucleotide
aaaatgaggtgctctttact

SEQ ID NO: 360 - DNA, 20 bp, Artificial Sequence
  Artificial polynucleotide
aaacccctgatccccctaca

SEQ ID NO: 361 - DNA, 20 bp, Artificial Sequence
  Artificial polynucleotide
aaacggctgagagaccacag

SEQ ID NO: 362 - DNA, 20 bp, Artificial Sequence
  Artificial polynucleotide
aaagacaatgacaagggttg

SEQ ID NO: 363 - DNA, 20 bp, Artificial Sequence
  Artificial polynucleotide
aacaaatggaagacaggagg

SEQ ID NO: 364 - DNA, 20 bp, Artificial Sequence
  Artificial polynucleotide
aacaggtcctagtataagct

SEQ ID NO: 365 - DNA, 20 bp, Artificial Sequence
  Artificial polynucleotide
aagactcccttgagcccggg

SEQ ID NO: 366 - DNA, 20 bp, Artificial Sequence
  Artificial polynucleotide
aagttcgcggggaatgaaca

SEQ ID NO: 367 - DNA, 20 bp, Artificial Sequence
  Artificial polynucleotide
aatatcggcaaccctctctt

SEQ ID NO: 368 - DNA, 20 bp, Artificial Sequence
  Artificial polynucleotide
aatccgtgtaaccttgaatt

SEQ ID NO: 369 - DNA, 20 bp, Artificial Sequence
  Artificial polynucleotide
aatgacaagggttgtggagg

SEQ ID NO: 370 - DNA, 20 bp, Artificial Sequence
  Artificial polynucleotide
aatgtttccagagtagttca

SEQ ID NO: 371 - DNA, 20 bp, Artificial Sequence
  Artificial polynucleotide
acatctagggatgtgagtta

SEQ ID NO: 372 - DNA, 20 bp, Artificial Sequence
  Artificial polynucleotide
acagagtctactatactctt

SEQ ID NO: 373 - DNA, 20 bp, Artificial Sequence
  Artificial polynucleotide
accaatgggaaaagggtaca

SEQ ID NO: 374 - DNA, 20 bp, Artificial Sequence
  Artificial polynucleotide
accctcccttcttcgataat

SEQ ID NO: 375 - DNA, 20 bp, Artificial Sequence
  Artificial polynucleotide
acctgtgcccactgacccac

SEQ ID NO: 376 - DNA, 20 bp, Artificial Sequence
  Artificial polynucleotide
actacactagaaaccaccta

SEQ ID NO: 377 - DNA, 20 bp, Artificial Sequence
  Artificial polynucleotide
agactctgtggggtatgatg

SEQ ID NO: 378 - DNA, 20 bp, Artificial Sequence
  Artificial polynucleotide
agactgttggtcggaagtgt

SEQ ID NO: 379 - DNA, 20 bp, Artificial Sequence
  Artificial polynucleotide
agagcctgagtccaaaggca

SEQ ID NO: 380 - DNA, 20 bp, Artificial Sequence
  Artificial polynucleotide
agaggtatgaaacacatcta

SEQ ID NO: 381 - DNA, 20 bp, Artificial Sequence
  Artificial polynucleotide
agataactgtaaggactgtg

SEQ ID NO: 382 - DNA, 20 bp, Artificial Sequence
  Artificial polynucleotide
aggtaatgcatcctgaggac

SEQ ID NO: 383 - DNA, 20 bp, Artificial Sequence
  Artificial polynucleotide
agtctcagtcccaagcagct

SEQ ID NO: 384 - DNA, 20 bp, Artificial Sequence
  Artificial polynucleotide
agtgaaaggataaccagaat

SEQ ID NO: 385 - DNA, 20 bp, Artificial Sequence
  Artificial polynucleotide
agtgaccgacacacccacct

SEQ ID NO: 386 - DNA, 20 bp, Artificial Sequence
  Artificial polynucleotide
agttcgcggggaatgaacaa

SEQ ID NO: 387 - DNA, 20 bp, Artificial Sequence
  Artificial polynucleotide
atacccaagcttgaactccc

SEQ ID NO: 388 - DNA, 20 bp, Artificial Sequence
  Artificial polynucleotide
atcacgaacctcctgatctc

SEQ ID NO: 389 - DNA, 20 bp, Artificial Sequence
  Artificial polynucleotide
atgaagtcccacggagtcaa

SEQ ID NO: 390 - DNA, 20 bp, Artificial Sequence
  Artificial polynucleotide
atgttaaacagagaagaggc

SEQ ID NO: 391 - DNA, 20 bp, Artificial Sequence
  Artificial polynucleotide
attgcaggccctgctcccga

SEQ ID NO: 392 - DNA, 20 bp, Artificial Sequence
  Artificial polynucleotide
caaattaactataatctcaa

SEQ ID NO: 393 - DNA, 20 bp, Artificial Sequence
  Artificial polynucleotide
cacacctacaataccagctg

SEQ ID NO: 394 - DNA, 20 bp, Artificial Sequence
  Artificial polynucleotide
cacagccaatggcaccatct

SEQ ID NO: 395 - DNA, 20 bp, Artificial Sequence
  Artificial polynucleotide
caccacaaacactgccttcg

SEQ ID NO: 396 - DNA, 20 bp, Artificial Sequence
  Artificial polynucleotide
caggtagacaacaccacgcc

SEQ ID NO: 397 - DNA, 20 bp, Artificial Sequence
  Artificial polynucleotide
caggtgcgtgccctcacacc

SEQ ID NO: 398 - DNA, 20 bp, Artificial Sequence
  Artificial polynucleotide
catgccaaaaagagggaggt

SEQ ID NO: 399 - DNA, 20 bp, Artificial Sequence
  Artificial polynucleotide
catgtccgggcatgcaacaa

SEQ ID NO: 400 - DNA, 20 bp, Artificial Sequence
  Artificial polynucleotide
cattgaggggaatgttctgg

SEQ ID NO: 401 - DNA, 20 bp, Artificial Sequence
  Artificial polynucleotide
ccacaagcttagcggtccag

SEQ ID NO: 402 - DNA, 20 bp, Artificial Sequence
  Artificial polynucleotide
ccacaatgagatgccacttg

SEQ ID NO: 403 - DNA, 20 bp, Artificial Sequence
  Artificial polynucleotide
cccacttaggtgccagggaa

SEQ ID NO: 404 - DNA, 20 bp, Artificial Sequence
  Artificial polynucleotide
ccccaagtggcatctcattg

SEQ ID NO: 405 - DNA, 20 bp, Artificial Sequence
  Artificial polynucleotide
cccctccatcatgcctttgt

SEQ ID NO: 406 - DNA, 20 bp, Artificial Sequence
  Artificial polynucleotide
cccttccacttcagcctccc

SEQ ID NO: 407 - DNA, 20 bp, Artificial Sequence
  Artificial polynucleotide
ccgtacccccataccaaaat

SEQ ID NO: 408 - DNA, 20 bp, Artificial Sequence
  Artificial polynucleotide
cctcagaatctatagactgt

SEQ ID NO: 409 - DNA, 20 bp, Artificial Sequence
  Artificial polynucleotide
cctgctactgtggcatgtcc

SEQ ID NO: 410 - DNA, 20 bp, Artificial Sequence
  Artificial polynucleotide
ctaaagactcccttgagccc

SEQ ID NO: 411 - DNA, 20 bp, Artificial Sequence
  Artificial polynucleotide
ctagaaaccacctaagggta

SEQ ID NO: 412 - DNA, 20 bp, Artificial Sequence
  Artificial polynucleotide
ctcaagcactgactgagctt

SEQ ID NO: 413 - DNA, 20 bp, Artificial Sequence
  Artificial polynucleotide
ctgcattgaggggaatgttc

SEQ ID NO: 414 - DNA, 20 bp, Artificial Sequence
  Artificial polynucleotide
cttgaaataatggccaggtg

SEQ ID NO: 415 - DNA, 20 bp, Artificial Sequence
  Artificial polynucleotide
gaaacccctgatccccctac

SEQ ID NO: 416 - DNA, 20 bp, Artificial Sequence
  Artificial polynucleotide
gacaatgacaagggttgtgg

SEQ ID NO: 417 - DNA, 20 bp, Artificial Sequence
  Artificial polynucleotide
gacgttgtgaggctgagact

SEQ ID NO: 418 - DNA, 20 bp, Artificial Sequence
  Artificial polynucleotide
gagaaaggcatgcatgctat

SEQ ID NO: 419 - DNA, 20 bp, Artificial Sequence
  Artificial polynucleotide
gagatagggttttcaccacg

SEQ ID NO: 420 - DNA, 20 bp, Artificial Sequence
  Artificial polynucleotide
gagcaatcctgcagcaacaa

SEQ ID NO: 421 - DNA, 20 bp, Artificial Sequence
  Artificial polynucleotide
gaggtgtgcaacaccacatt

SEQ ID NO: 422 - DNA, 20 bp, Artificial Sequence
  Artificial polynucleotide
gagtgttgggagagaatttg

SEQ ID NO: 423 - DNA, 20 bp, Artificial Sequence
  Artificial polynucleotide
gagttggtgtctgcattgag

SEQ ID NO: 424 - DNA, 20 bp, Artificial Sequence
  Artificial polynucleotide
gatcagtgctacattatggt

SEQ ID NO: 425 - DNA, 20 bp, Artificial Sequence
  Artificial polynucleotide
gcaacctattcctaagggat

SEQ ID NO: 426 - DNA, 20 bp, Artificial Sequence
  Artificial polynucleotide
gcactttgggaaagaaacca

SEQ ID NO: 427 - DNA, 20 bp, Artificial Sequence
  Artificial polynucleotide
gcctgtactaacaggcactt

SEQ ID NO: 428 - DNA, 20 bp, Artificial Sequence
  Artificial polynucleotide
gcgaggctgtgggggttgaa

SEQ ID NO: 429 - DNA, 20 bp, Artificial Sequence
  Artificial polynucleotide
gctattcaatcctagttatg

SEQ ID NO: 430 - DNA, 20 bp, Artificial Sequence
  Artificial polynucleotide
ggactagggattagagtaac

SEQ ID NO: 431 - DNA, 20 bp, Artificial Sequence
  Artificial polynucleotide
ggaggcagaggtcgcggtga

SEQ ID NO: 432 - DNA, 20 bp, Artificial Sequence
  Artificial polynucleotide
ggagggatgcactgatacgg

SEQ ID NO: 433 - DNA, 20 bp, Artificial Sequence
  Artificial polynucleotide
ggagttggtgtctgcattga

SEQ ID NO: 434 - DNA, 20 bp, Artificial Sequence
  Artificial polynucleotide
ggattcagtcaaggtgtcag

SEQ ID NO: 435 - DNA, 20 bp, Artificial Sequence
  Artificial polynucleotide
gggaaagaaaccaaggcggg

SEQ ID NO: 436 - DNA, 20 bp, Artificial Sequence
  Artificial polynucleotide
gggtagggggtcggaaagtg

SEQ ID NO: 437 - DNA, 20 bp, Artificial Sequence
  Artificial polynucleotide
ggtgatatacgctaatgtgc

SEQ ID NO: 438 - DNA, 20 bp, Artificial Sequence
  Artificial polynucleotide
ggttttgagcaagccctcct

SEQ ID NO: 439 - DNA, 20 bp, Artificial Sequence
  Artificial polynucleotide
gtaggtcatatggtaacttc

SEQ ID NO: 440 - DNA, 20 bp, Artificial Sequence
  Artificial polynucleotide
gtcctccaaacacatacaag

SEQ ID NO: 441 - DNA, 20 bp, Artificial Sequence
  Artificial polynucleotide
gtgaaacagcacagccacag

SEQ ID NO: 442 - DNA, 20 bp, Artificial Sequence
  Artificial polynucleotide
gtgataccactgcatgtcat

SEQ ID NO: 443 - DNA, 20 bp, Artificial Sequence
  Artificial polynucleotide
gtgggacttcatcgccccca

SEQ ID NO: 444 - DNA, 20 bp, Artificial Sequence
  Artificial polynucleotide
gttttgctccatcacccagg

SEQ ID NO: 445 - DNA, 20 bp, Artificial Sequence
  Artificial polynucleotide
tacaatgttaaacagagaag

SEQ ID NO: 446 - DNA, 20 bp, Artificial Sequence
  Artificial polynucleotide
tacctcaagcctgtactaac

SEQ ID NO: 447 - DNA, 20 bp, Artificial Sequence
  Artificial polynucleotide
tactgatcagtgctacatta

SEQ ID NO: 448 - DNA, 20 bp, Artificial Sequence
  Artificial polynucleotide
tagcaacctatgacatgcag

SEQ ID NO: 449 - DNA, 20 bp, Artificial Sequence
  Artificial polynucleotide
taggatcacacctgagttct

SEQ ID NO: 450 - DNA, 20 bp, Artificial Sequence
  Artificial polynucleotide
tcaaaagagcctgagtccaa

SEQ ID NO: 451 - DNA, 20 bp, Artificial Sequence
  Artificial polynucleotide
tcctacaaaggcatgatgga

SEQ ID NO: 452 - DNA, 20 bp, Artificial Sequence
  Artificial polynucleotide
tgatcagtgctacattatgg

SEQ ID NO: 453 - DNA, 20 bp, Artificial Sequence
  Artificial polynucleotide
tgctgactgtcatgcgttgt

SEQ ID NO: 454 - DNA, 20 bp, Artificial Sequence
  Artificial polynucleotide
tggcagtcaagataactgta

SEQ ID NO: 455 - DNA, 20 bp, Artificial Sequence
  Artificial polynucleotide
tgggtagggggtcggaaagt

SEQ ID NO: 456 - DNA, 20 bp, Artificial Sequence
  Artificial polynucleotide
tggtattagtcaagatagtg

SEQ ID NO: 457 - DNA, 20 bp, Artificial Sequence
  Artificial polynucleotide
tgtacctatcccttaggaat

SEQ ID NO: 458 - DNA, 20 bp, Artificial Sequence
  Artificial polynucleotide
tgttaaacagagaagaggct

SEQ ID NO: 459 - DNA, 20 bp, Artificial Sequence
  Artificial polynucleotide
ttcctacaaaggcatgatgg

SEQ ID NO: 460 - DNA, 20 bp, Artificial Sequence
  Artificial polynucleotide
ttgagtgttcaatctccctt

SEQ ID NO: 461 - DNA, 20 bp, Artificial Sequence
  Artificial polynucleotide
ttgggagagaatttgaggca

SEQ ID NO: 462 - DNA, 20 bp, Artificial Sequence
  Artificial polynucleotide
tttaggtctaagatccacct

SEQ ID NO: 463 - DNA, 20 bp, Artificial Sequence
  Artificial polynucleotide
tttgggaaagaaaccaaggc

SEQ ID NO: 464 - DNA, 20 bp, Artificial Sequence
  Artificial polynucleotide
acaccaatctctttagtgct

SEQ ID NO: 465 - DNA, 20 bp, Artificial Sequence
  Artificial polynucleotide
actacagaggcacgcatcca

SEQ ID NO: 466 - DNA, 20 bp, Artificial Sequence
  Artificial polynucleotide
agtatataggtggccttaca

SEQ ID NO: 467 - DNA, 20 bp, Artificial Sequence
  Artificial polynucleotide
atcatgtgcagggcttttac

SEQ ID NO: 468 - DNA, 20 bp, Artificial Sequence
  Artificial polynucleotide
caattgttaagcagaatctc

SEQ ID NO: 469 - DNA, 20 bp, Artificial Sequence
  Artificial polynucleotide
cacaatgctagcaggagttg

SEQ ID NO: 470 - DNA, 20 bp, Artificial Sequence
  Artificial polynucleotide
cactcaacaatttctatcct

SEQ ID NO: 471 - DNA, 20 bp, Artificial Sequence
  Artificial polynucleotide
caggtaaagtactttggttg

SEQ ID NO: 472 - DNA, 20 bp, Artificial Sequence
  Artificial polynucleotide
caggtaccaagaggcagact

SEQ ID NO: 473 - DNA, 20 bp, Artificial Sequence
  Artificial polynucleotide
cagtccagacttcttggcgt

SEQ ID NO: 474 - DNA, 20 bp, Artificial Sequence
  Artificial polynucleotide
ccaagggactggcggagccc

SEQ ID NO: 475 - DNA, 20 bp, Artificial Sequence
  Artificial polynucleotide
cccgggcgccgaggaccctg

SEQ ID NO: 476 - DNA, 20 bp, Artificial Sequence
  Artificial polynucleotide
cgaagaaattaacagaagag

SEQ ID NO: 477 - DNA, 20 bp, Artificial Sequence
  Artificial polynucleotide
cggcattctggggatgaatc

SEQ ID NO: 478 - DNA, 20 bp, Artificial Sequence
  Artificial polynucleotide
cggccgcggcgcaagacggg

SEQ ID NO: 479 - DNA, 20 bp, Artificial Sequence
  Artificial polynucleotide
ctgccacgctgtgactcaga

SEQ ID NO: 480 - DNA, 20 bp, Artificial Sequence
  Artificial polynucleotide
ctgtgactcagacggcattc

SEQ ID NO: 481 - DNA, 20 bp, Artificial Sequence
  Artificial polynucleotide
gaactgtatccaccctcagc

SEQ ID NO: 482 - DNA, 20 bp, Artificial Sequence
  Artificial polynucleotide
gaagtagttccagagcagta

SEQ ID NO: 483 - DNA, 20 bp, Artificial Sequence
  Artificial polynucleotide
gatgtgatctttgggccctt

SEQ ID NO: 484 - DNA, 20 bp, Artificial Sequence
  Artificial polynucleotide
gcacaaccacttaaaccccc

SEQ ID NO: 485 - DNA, 20 bp, Artificial Sequence
  Artificial polynucleotide
gcagtatgggaagaaatcac

SEQ ID NO: 486 - DNA, 20 bp, Artificial Sequence
  Artificial polynucleotide
gccttgatatgtaatgagca

SEQ ID NO: 487 - DNA, 20 bp, Artificial Sequence
  Artificial polynucleotide
ggataaaatacttttggaag

SEQ ID NO: 488 - DNA, 20 bp, Artificial Sequence
  Artificial polynucleotide
ggcagggcgccccgggaact

SEQ ID NO: 489 - DNA, 20 bp, Artificial Sequence
  Artificial polynucleotide
gtgacattcacaaccactcc

SEQ ID NO: 490 - DNA, 20 bp, Artificial Sequence
  Artificial polynucleotide
gtgactcagacggcattctg

SEQ ID NO: 491 - DNA, 20 bp, Artificial Sequence
  Artificial polynucleotide
gttccataaccaattcaaac

SEQ ID NO: 492 - DNA, 20 bp, Artificial Sequence
  Artificial polynucleotide
gttgggatatggttgccatt

SEQ ID NO: 493 - DNA, 20 bp, Artificial Sequence
  Artificial polynucleotide
taccgtatgaagggatccac

SEQ ID NO: 494 - DNA, 20 bp, Artificial Sequence
  Artificial polynucleotide
tacctgtgtagtgcacatgg

SEQ ID NO: 495 - DNA, 20 bp, Artificial Sequence
  Artificial polynucleotide
tactaccccacggtttgtag

SEQ ID NO: 496 - DNA, 20 bp, Artificial Sequence
  Artificial polynucleotide
tagtaacatactccgcgcat

SEQ ID NO: 497 - DNA, 20 bp, Artificial Sequence
  Artificial polynucleotide
tcactcatgccaaaatctaa

SEQ ID NO: 498 - DNA, 20 bp, Artificial Sequence
  Artificial polynucleotide
tcatgacccgggggtttaag

SEQ ID NO: 499 - DNA, 20 bp, Artificial Sequence
  Artificial polynucleotide
tccatatattcctgcagggt

SEQ ID NO: 500 - DNA, 20 bp, Artificial Sequence
  Artificial polynucleotide
tcgagcctgcgctccctccg

SEQ ID NO: 501 - DNA, 20 bp, Artificial Sequence
  Artificial polynucleotide
tcggcgcgcctgcacagcgc

SEQ ID NO: 502 - DNA, 20 bp, Artificial Sequence
  Artificial polynucleotide
tctagcagaaacgcaagttc

SEQ ID NO: 503 - DNA, 20 bp, Artificial Sequence
  Artificial polynucleotide
tctggactatggctctacag

SEQ ID NO: 504 - DNA, 20 bp, Artificial Sequence
  Artificial polynucleotide
tgtgactcagacggcattct

SEQ ID NO: 505 - DNA, 20 bp, Artificial Sequence
  Artificial polynucleotide
ttacctttaactcatgtaac

SEQ ID NO: 506 - DNA, 20 bp, Artificial Sequence
  Artificial polynucleotide
ttgtcacttggtgataccac

SEQ ID NO: 507 - DNA, 20 bp, Artificial Sequence
  Artificial polynucleotide
acactctctagcacagtata

SEQ ID NO: 508 - DNA, 20 bp, Artificial Sequence
  Artificial polynucleotide
atacatctctgccttttaag

SEQ ID NO: 509 - DNA, 20 bp, Artificial Sequence
  Artificial polynucleotide
atattatattcatgtagtgc

SEQ ID NO: 510 - DNA, 20 bp, Artificial Sequence
  Artificial polynucleotide
cagctgaaggtacatcacat

SEQ ID NO: 511 - DNA, 20 bp, Artificial Sequence
  Artificial polynucleotide
cttgtttttcattatgaaac

SEQ ID NO: 512 - DNA, 20 bp, Artificial Sequence
  Artificial polynucleotide
gagataatacttaatcgctg

SEQ ID NO: 513 - DNA, 20 bp, Artificial Sequence
  Artificial polynucleotide
gagcacattttctatgctct

SEQ ID NO: 514 - DNA, 20 bp, Artificial Sequence
  Artificial polynucleotide
tactgattttttctgcttgg

SEQ ID NO: 515 - DNA, 20 bp, Artificial Sequence
  Artificial polynucleotide
tggggcactgcagcacatgt

SEQ ID NO: 516 - DNA, 20 bp, Artificial Sequence
  Artificial polynucleotide
tttcggtacttaacagatgt

SEQ ID NO: 517 - DNA, 20 bp, Artificial Sequence
  Artificial polynucleotide
gcctacagggtatctctcct

SEQ ID NO: 518 - DNA, 20 bp, Artificial Sequence
  Artificial polynucleotide
atcatacaggagtgcctaca

SEQ ID NO: 519 - DNA, 20 bp, Artificial Sequence
  Artificial polynucleotide
tgagctcagagccttctgtg

SEQ ID NO: 520 - DNA, 20 bp, Artificial Sequence
  Artificial polynucleotide
ccatagctgaggcctttatt

SEQ ID NO: 521 - DNA, 20 bp, Artificial Sequence
  Artificial polynucleotide
aaaaccaggcaaccataatg

SEQ ID NO: 522 - DNA, 20 bp, Artificial Sequence
  Artificial polynucleotide
tcctgcgccgacctcgaatg

SEQ ID NO: 523 - DNA, 20 bp, Artificial Sequence
  Artificial polynucleotide
cagcaggaggaccgcattcg

SEQ ID NO: 524 - DNA, 20 bp, Artificial Sequence
  Artificial polynucleotide
gagccttctgtgtggtttgc

SEQ ID NO: 525 - DNA, 20 bp, Artificial Sequence
  Artificial polynucleotide
agccttctgtgtggtttgcg

SEQ ID NO: 526 - DNA, 20 bp, Artificial Sequence
  Artificial polynucleotide
gccttctgtgtggtttgcgg

SEQ ID NO: 527 - DNA, 20 bp, Artificial Sequence
  Artificial polynucleotide
gcccccgcaaaccacacaga

SEQ ID NO: 528 - DNA, 20 bp, Artificial Sequence
  Artificial polynucleotide
aagcaatatgaaatgttacc

SEQ ID NO: 529 - DNA, 20 bp, Artificial Sequence
  Artificial polynucleotide
tatgacgacactcatttgca

SEQ ID NO: 530 - DNA, 20 bp, Artificial Sequence
  Artificial polynucleotide
gttgggctacctttatcctt

SEQ ID NO: 531 - DNA, 20 bp, Artificial Sequence
  Artificial polynucleotide
gccatcttgcaacaatccca

SEQ ID NO: 532 - DNA, 20 bp, Artificial Sequence
  Artificial polynucleotide
gttagtctatgcatgggtat

SEQ ID NO: 533 - DNA, 20 bp, Artificial Sequence
  Artificial polynucleotide
tgatagagcagcgtggccca

SEQ ID NO: 534 - DNA, 20 bp, Artificial Sequence
  Artificial polynucleotide
gctaagcagtggactaagga

SEQ ID NO: 535 - DNA, 20 bp, Artificial Sequence
  Artificial polynucleotide
tcagatgttaaaggccaatg

SEQ ID NO: 536 - DNA, 20 bp, Artificial Sequence
  Artificial polynucleotide
gctgcccttgggagcccaga

SEQ ID NO: 537 - DNA, 20 bp, Artificial Sequence
  Artificial polynucleotide
ggcaagtttcaggcctggac

SEQ ID NO: 538 - DNA, 20 bp, Artificial Sequence
  Artificial polynucleotide
gcaagtttcaggcctggact

SEQ ID NO: 539 - DNA, 20 bp, Artificial Sequence
  Artificial polynucleotide
agtttcaggcctggactggg

SEQ ID NO: 540 - DNA, 20 bp, Artificial Sequence
  Artificial polynucleotide
gctttccctgccctgtttgc

SEQ ID NO: 541 - DNA, 20 bp, Artificial Sequence
  Artificial polynucleotide
accatgcagggactgggcat

SEQ ID NO: 542 - DNA, 20 bp, Artificial Sequence
  Artificial polynucleotide
ccatgcagggactgggcatg

SEQ ID NO: 543 - DNA, 20 bp, Artificial Sequence
  Artificial polynucleotide
gcatcactttctgagctgcc

SEQ ID NO: 544 - DNA, 20 bp, Artificial Sequence
  Artificial polynucleotide
ggcagctcagaaagtgatgc

SEQ ID NO: 545 - DNA, 20 bp, Artificial Sequence
  Artificial polynucleotide
gatgcaggggccgctctctg

SEQ ID NO: 546 - DNA, 20 bp, Artificial Sequence
  Artificial polynucleotide
tcagaagacctcatggattg

SEQ ID NO: 547 - DNA, 20 bp, Artificial Sequence
  Artificial polynucleotide
ggaccattagcattgccagg

SEQ ID NO: 548 - DNA, 20 bp, Artificial Sequence
  Artificial polynucleotide
gaccattagcattgccagga

SEQ ID NO: 549 - DNA, 20 bp, Artificial Sequence
  Artificial polynucleotide
accattagcattgccaggag

SEQ ID NO: 550 - DNA, 20 bp, Artificial Sequence
  Artificial polynucleotide
atgtggtgcatctcgatgag

SEQ ID NO: 551 - DNA, 20 bp, Artificial Sequence
  Artificial polynucleotide
cctcatcccctaccaccctt

SEQ ID NO: 552 - DNA, 20 bp, Artificial Sequence
  Artificial polynucleotide
ctagggcctaagggtggtag

SEQ ID NO: 553 - DNA, 20 bp, Artificial Sequence
  Artificial polynucleotide
actagggcctaagggtggta

SEQ ID NO: 554 - DNA, 20 bp, Artificial Sequence
  Artificial polynucleotide
tgagtactagggcctaaggg

SEQ ID NO: 555 - DNA, 20 bp, Artificial Sequence
  Artificial polynucleotide
atagtcaatgaaatctccca

SEQ ID NO: 556 - DNA, 20 bp, Artificial Sequence
  Artificial polynucleotide
agagaaagacgagatggcac

SEQ ID NO: 557 - DNA, 20 bp, Artificial Sequence
  Artificial polynucleotide
ataaagggcctgagcagggg

SEQ ID NO: 558 - DNA, 20 bp, Artificial Sequence
  Artificial polynucleotide
ctaatttcctcattgaaggt

SEQ ID NO: 559 - DNA, 20 bp, Artificial Sequence
  Artificial polynucleotide
aaaaaggctgcccgctgcgg

SEQ ID NO: 560 - DNA, 20 bp, Artificial Sequence
  Artificial polynucleotide
atttagaagacgatgatgag

SEQ ID NO: 561 - DNA, 20 bp, Artificial Sequence
  Artificial polynucleotide
tctgtattaataatgctgcc

SEQ ID NO: 562 - DNA, 20 bp, Artificial Sequence
  Artificial polynucleotide
tttagacgctgatgtagctt

SEQ ID NO: 563 - DNA, 20 bp, Artificial Sequence
  Artificial polynucleotide
agtccctaatatggctagtc

SEQ ID NO: 564 - DNA, 20 bp, Artificial Sequence
  Artificial polynucleotide
cagccaggttactgattcat

SEQ ID NO: 565 - DNA, 20 bp, Artificial Sequence
  Artificial polynucleotide
acccctatgaatcagtaacc

SEQ ID NO: 566 - DNA, 20 bp, Artificial Sequence
  Artificial polynucleotide
actagtattcctgtagaaga

SEQ ID NO: 567 - DNA, 20 bp, Artificial Sequence
  Artificial polynucleotide
gtaaggctgatttgggcaaa

SEQ ID NO: 568 - DNA, 20 bp, Artificial Sequence
  Artificial polynucleotide
gaacctgaggttagcagcaa

SEQ ID NO: 569 - DNA, 20 bp, Artificial Sequence
  Artificial polynucleotide
ttatgcaagcatgtgttttt

SEQ ID NO: 570 - DNA, 20 bp, Artificial Sequence
  Artificial polynucleotide
caacagaattttgtcaaaat

SEQ ID NO: 571 - DNA, 20 bp, Artificial Sequence
  Artificial polynucleotide
ggaccagtaaaataattatt

SEQ ID NO: 572 - DNA, 20 bp, Artificial Sequence
  Artificial polynucleotide
gcatccttgcatagaggacc

SEQ ID NO: 573 - DNA, 20 bp, Artificial Sequence
  Artificial polynucleotide
gatgacaggttggccattgg

SEQ ID NO: 574 - DNA, 20 bp, Artificial Sequence
  Artificial polynucleotide
cattgacgtgggttgcacca

SEQ ID NO: 575 - DNA, 20 bp, Artificial Sequence
  Artificial polynucleotide
gtacaaattgacgacacacc

SEQ ID NO: 576 - DNA, 20 bp, Artificial Sequence
  Artificial polynucleotide
ggactctatttgtgaggcca

SEQ ID NO: 577 - DNA, 20 bp, Artificial Sequence
  Artificial polynucleotide
acgtgcagcatatgtggcgt

SEQ ID NO: 578 - DNA, 20 bp, Artificial Sequence
  Artificial polynucleotide
cagtgtttcatatctagact

SEQ ID NO: 579 - DNA, 20 bp, Artificial Sequence
  Artificial polynucleotide
agtcggcctgccctgaaggg

SEQ ID NO: 580 - DNA, 20 bp, Artificial Sequence
  Artificial polynucleotide
acgctcctggcagagggcta

SEQ ID NO: 581 - RNA, 20 nt, Artificial Sequence
  Synthetic polynucleotide
acccacccagcaaccaccag

SEQ ID NO: 582 - RNA, 20 nt, Artificial Sequence
  Synthetic polynucleotide
gacaaugacaaggguugugg

SEQ ID NO: 583 - RNA, 20 nt, Artificial Sequence
  Synthetic polynucleotide
ugcuuguuugccgcagugca

SEQ ID NO: 584 - RNA, 20 nt, Artificial Sequence
  Synthetic polynucleotide
gguuuugagcaagcccuccu

SEQ ID NO: 585 - RNA, 20 nt, Artificial Sequence
  Synthetic polynucleotide
acauccucuauucugaucau

SEQ ID NO: 586 - RNA, 20 nt, Artificial Sequence
  Synthetic polynucleotide
ucguugugacacuacugagu

SEQ ID NO: 587 - RNA, 20 nt, Artificial Sequence
  Synthetic polynucleotide
cugccacgcugugacucaga

SEQ ID NO: 588 - RNA, 20 nt, Artificial Sequence
  Synthetic polynucleotide
ggaaccagucagaacaggug

SEQ ID NO: 589 - RNA, 20 nt, Artificial Sequence
  Synthetic polynucleotide
caguucagggcaugagauaa

SEQ ID NO: 590 - RNA, 20 nt, Artificial Sequence
  Synthetic polynucleotide
aaaaucccaaaggcuucaga

SEQ ID NO: 591 - RNA, 20 nt, Artificial Sequence
  Synthetic polynucleotide
aaagcaugcgcuacaauaac

SEQ ID NO: 592 - RNA, 20 nt, Artificial Sequence
  Synthetic polynucleotide
aaaguuauaaaucacguuaa

SEQ ID NO: 593 - RNA, 20 nt, Artificial Sequence
  Synthetic polynucleotide
aaaugcacuuacccacguau

SEQ ID NO: 594 - RNA, 20 nt, Artificial Sequence
  Synthetic polynucleotide
aaauguugucauccauaucc

SEQ ID NO: 595 - RNA, 20 nt, Artificial Sequence
  Synthetic polynucleotide
aaccugagguuagcagcaag

SEQ ID NO: 596 - RNA, 20 nt, Artificial Sequence
  Synthetic polynucleotide
aacuauaugcuaccuggaua

SEQ ID NO: 597 - RNA, 20 nt, Artificial Sequence
  Synthetic polynucleotide
aacucaaaugaccucuaucu

SEQ ID NO: 598 - RNA, 20 nt, Artificial Sequence
  Synthetic polynucleotide
aacuuuuucuuugccuguac

SEQ ID NO: 599 - RNA, 20 nt, Artificial Sequence
  Synthetic polynucleotide
aagcaagaaugaauauuacc

SEQ ID NO: 600 - RNA, 20 nt, Artificial Sequence
  Synthetic polynucleotide
aaucaaugauagcuggagug

SEQ ID NO: 601 - RNA, 20 nt, Artificial Sequence
  Synthetic polynucleotide
aaugccuggcugucauucag

SEQ ID NO: 602 - RNA, 20 nt, Artificial Sequence
  Synthetic polynucleotide
aauucagagaucaggucaag

SEQ ID NO: 603 - RNA, 20 nt, Artificial Sequence
  Synthetic polynucleotide
acacagucuguacucacucu

SEQ ID NO: 604 - RNA, 20 nt, Artificial Sequence
  Synthetic polynucleotide
acacuacaccucaacauauc

SEQ ID NO: 605 - RNA, 20 nt, Artificial Sequence
  Synthetic polynucleotide
acagaccuugaaacuucaag

SEQ ID NO: 606 - RNA, 20 nt, Artificial Sequence
  Synthetic polynucleotide
acagaugcgucaggcaucuc

SEQ ID NO: 607 - RNA, 20 nt, Artificial Sequence
  Synthetic polynucleotide
acaugcuaguagacccugau

SEQ ID NO: 608 - RNA, 20 nt, Artificial Sequence
  Synthetic polynucleotide
acauuucggcaaugacacac

SEQ ID NO: 609 - RNA, 20 nt, Artificial Sequence
  Synthetic polynucleotide
accaacuccuacccuuaccu

SEQ ID NO: 610 - RNA, 20 nt, Artificial Sequence
  Synthetic polynucleotide
accaaucccuuaccuggagg

SEQ ID NO: 611 - RNA, 20 nt, Artificial Sequence
  Synthetic polynucleotide
acccacccagcaaccaccag

SEQ ID NO: 612 - RNA, 20 nt, Artificial Sequence
  Synthetic polynucleotide
accccuaugaaucaguaacc

SEQ ID NO: 613 - RNA, 20 nt, Artificial Sequence
  Synthetic polynucleotide
accgcacauaggaagaccug

SEQ ID NO: 614 - RNA, 20 nt, Artificial Sequence
  Synthetic polynucleotide
accgggugaugcaagcaaca

SEQ ID NO: 615 - RNA, 20 nt, Artificial Sequence
  Synthetic polynucleotide
acggugcagcaguagaggac

SEQ ID NO: 616 - RNA, 20 nt, Artificial Sequence
  Synthetic polynucleotide
acgucucgccccacugcaag

SEQ ID NO: 617 - RNA, 20 nt, Artificial Sequence
  Synthetic polynucleotide
acguggggggaccagugcau

SEQ ID NO: 618 - RNA, 20 nt, Artificial Sequence
  Synthetic polynucleotide
acguuaacggccgggugagg

SEQ ID NO: 619 - RNA, 20 nt, Artificial Sequence
  Synthetic polynucleotide
acuaguaauggguacuauuu

SEQ ID NO: 620 - RNA, 20 nt, Artificial Sequence
  Synthetic polynucleotide
acucuugccaagaguaaccu

SEQ ID NO: 621 - RNA, 20 nt, Artificial Sequence
  Synthetic polynucleotide
acugaagaccuagagaauua

SEQ ID NO: 622 - RNA, 20 nt, Artificial Sequence
  Synthetic polynucleotide
acugcacccuaaagagggua

SEQ ID NO: 623 - RNA, 20 nt, Artificial Sequence
  Synthetic polynucleotide
acugcugcaccguggguaag

SEQ ID NO: 624 - RNA, 20 nt, Artificial Sequence
  Synthetic polynucleotide
acugucucuacauuuagaau

SEQ ID NO: 625 - RNA, 20 nt, Artificial Sequence
  Synthetic polynucleotide
agaaacaugacgaaaguguc

SEQ ID NO: 626 - RNA, 20 nt, Artificial Sequence
  Synthetic polynucleotide
agaaggagggaauccuaaug

SEQ ID NO: 627 - RNA, 20 nt, Artificial Sequence
  Synthetic polynucleotide
agacauaccaguuaugauag

SEQ ID NO: 628 - RNA, 20 nt, Artificial Sequence
  Synthetic polynucleotide
agagcgguggcuggguggcc

SEQ ID NO: 629 - RNA, 20 nt, Artificial Sequence
  Synthetic polynucleotide
agaugucaacacuccucauu

SEQ ID NO: 630 - RNA, 20 nt, Artificial Sequence
  Synthetic polynucleotide
agcaugcgggguaaccgcag

SEQ ID NO: 631 - RNA, 20 nt, Artificial Sequence
  Synthetic polynucleotide
agccagcuagcauccagaaa

SEQ ID NO: 632 - RNA, 20 nt, Artificial Sequence
  Synthetic polynucleotide
agccauccaaagagacaaca

SEQ ID NO: 633 - RNA, 20 nt, Artificial Sequence
  Synthetic polynucleotide
agcuagccugaagcaggugg

SEQ ID NO: 634 - RNA, 20 nt, Artificial Sequence
  Synthetic polynucleotide
aggagcggucagugacgcga

SEQ ID NO: 635 - RNA, 20 nt, Artificial Sequence
  Synthetic polynucleotide
aggauuaggcuuaaaaggac

SEQ ID NO: 636 - RNA, 20 nt, Artificial Sequence
  Synthetic polynucleotide
aggcuugggcugucauucag

SEQ ID NO: 637 - RNA, 20 nt, Artificial Sequence
  Synthetic polynucleotide
agggcgcaaugguucaugga

SEQ ID NO: 638 - RNA, 20 nt, Artificial Sequence
  Synthetic polynucleotide
agggcgcugauaaacaaagg

SEQ ID NO: 639 - RNA, 20 nt, Artificial Sequence
  Synthetic polynucleotide
agggcuccaaaaaguaaacc

SEQ ID NO: 640 - RNA, 20 nt, Artificial Sequence
  Synthetic polynucleotide
agggugauugcaguuccgug

SEQ ID NO: 641 - RNA, 20 nt, Artificial Sequence
  Synthetic polynucleotide
agucacugcacugcgcaaau

SEQ ID NO: 642 - RNA, 20 nt, Artificial Sequence
  Synthetic polynucleotide
agucauuaccugagaaggca

SEQ ID NO: 643 - RNA, 20 nt, Artificial Sequence
  Synthetic polynucleotide
aguccacacugagguuacau

SEQ ID NO: 644 - RNA, 20 nt, Artificial Sequence
  Synthetic polynucleotide
aguccucuacugcugcaccg

SEQ ID NO: 645 - RNA, 20 nt, Artificial Sequence
  Synthetic polynucleotide
aguguaucuucugagaucaa

SEQ ID NO: 646 - RNA, 20 nt, Artificial Sequence
  Synthetic polynucleotide
auacuacuuauaugccaaug

SEQ ID NO: 647 - RNA, 20 nt, Artificial Sequence
  Synthetic polynucleotide
auacucacuauaguaaaaau

SEQ ID NO: 648 - RNA, 20 nt, Artificial Sequence
  Synthetic polynucleotide
auaguaaacucucagaaaug

SEQ ID NO: 649 - RNA, 20 nt, Artificial Sequence
  Synthetic polynucleotide
auaguguaucucuacccaua

SEQ ID NO: 650 - RNA, 20 nt, Artificial Sequence
  Synthetic polynucleotide
auauggcuuuaaccaccucu

SEQ ID NO: 651 - RNA, 20 nt, Artificial Sequence
  Synthetic polynucleotide
aucacguuaacggccgggug

SEQ ID NO: 652 - RNA, 20 nt, Artificial Sequence
  Synthetic polynucleotide
aucccaaaggcuucagaagg

SEQ ID NO: 653 - RNA, 20 nt, Artificial Sequence
  Synthetic polynucleotide
aucugccauauuaggauccc

SEQ ID NO: 654 - RNA, 20 nt, Artificial Sequence
  Synthetic polynucleotide
aucuuccuuacaucuuuggg

SEQ ID NO: 655 - RNA, 20 nt, Artificial Sequence
  Synthetic polynucleotide
aucuugaaggagcugcacaa

SEQ ID NO: 656 - RNA, 20 nt, Artificial Sequence
  Synthetic polynucleotide
augaucuauccauuccguau

SEQ ID NO: 657 - RNA, 20 nt, Artificial Sequence
  Synthetic polynucleotide
augccugacgcaucugucug

SEQ ID NO: 658 - RNA, 20 nt, Artificial Sequence
  Synthetic polynucleotide
auguggacuaaauauguauu

SEQ ID NO: 659 - RNA, 20 nt, Artificial Sequence
  Synthetic polynucleotide
auguuuguaauagaaagugu

SEQ ID NO: 660 - RNA, 20 nt, Artificial Sequence
  Synthetic polynucleotide
auuaccaacccuucuucuac

SEQ ID NO: 661 - RNA, 20 nt, Artificial Sequence
  Synthetic polynucleotide
auuguaugacacauacacuu

SEQ ID NO: 662 - RNA, 20 nt, Artificial Sequence
  Synthetic polynucleotide
auuuggacugcuaacaccau

SEQ ID NO: 663 - RNA, 20 nt, Artificial Sequence
  Synthetic polynucleotide
caagaacacuuaacagcuuc

SEQ ID NO: 664 - RNA, 20 nt, Artificial Sequence
  Synthetic polynucleotide
caagaaccuggcauauacga

SEQ ID NO: 665 - RNA, 20 nt, Artificial Sequence
  Synthetic polynucleotide
caaucaucuacugguaagca

SEQ ID NO: 666 - RNA, 20 nt, Artificial Sequence
  Synthetic polynucleotide
caaucccuuaccuggaggug

SEQ ID NO: 667 - RNA, 20 nt, Artificial Sequence
  Synthetic polynucleotide
cacacacaaugcauaggaca

SEQ ID NO: 668 - RNA, 20 nt, Artificial Sequence
  Synthetic polynucleotide
cacccagcaaccaccagagg

SEQ ID NO: 669 - RNA, 20 nt, Artificial Sequence
  Synthetic polynucleotide
cacccugcaucccaacuacu

SEQ ID NO: 670 - RNA, 20 nt, Artificial Sequence
  Synthetic polynucleotide
caccggaaugaccugggggg

SEQ ID NO: 671 - RNA, 20 nt, Artificial Sequence
  Synthetic polynucleotide
caccuuuguauuggucaucu

SEQ ID NO: 672 - RNA, 20 nt, Artificial Sequence
  Synthetic polynucleotide
cacuaccugaacaccaccac

SEQ ID NO: 673 - RNA, 20 nt, Artificial Sequence
  Synthetic polynucleotide
cagaagcuucaaagaguaga

SEQ ID NO: 674 - RNA, 20 nt, Artificial Sequence
  Synthetic polynucleotide
cagagggcgcugauaaacaa

SEQ ID NO: 675 - RNA, 20 nt, Artificial Sequence
  Synthetic polynucleotide
caggucauuccggugaggga

SEQ ID NO: 676 - RNA, 20 nt, Artificial Sequence
  Synthetic polynucleotide
caguagacauacuuauauag

SEQ ID NO: 677 - RNA, 20 nt, Artificial Sequence
  Synthetic polynucleotide
caguccuucugcguuugacu

SEQ ID NO: 678 - RNA, 20 nt, Artificial Sequence
  Synthetic polynucleotide
caguggguauuggcggcggu

SEQ ID NO: 679 - RNA, 20 nt, Artificial Sequence
  Synthetic polynucleotide
cauaagcaaccugggaucaa

SEQ ID NO: 680 - RNA, 20 nt, Artificial Sequence
  Synthetic polynucleotide
cauacuagcuuccucaccaa

SEQ ID NO: 681 - RNA, 20 nt, Artificial Sequence
  Synthetic polynucleotide
cauuccucaaacagcuccag

SEQ ID NO: 682 - RNA, 20 nt, Artificial Sequence
  Synthetic polynucleotide
ccaaucacccaccuuucucc

SEQ ID NO: 683 - RNA, 20 nt, Artificial Sequence
  Synthetic polynucleotide
ccaccgcagacacccgcaau

SEQ ID NO: 684 - RNA, 20 nt, Artificial Sequence
  Synthetic polynucleotide
ccacuacacacacaaugcau

SEQ ID NO: 685 - RNA, 20 nt, Artificial Sequence
  Synthetic polynucleotide
ccaggucauuccggugaggg

SEQ ID NO: 686 - RNA, 20 nt, Artificial Sequence
  Synthetic polynucleotide
ccagugagcuagccugaagc

SEQ ID NO: 687 - RNA, 20 nt, Artificial Sequence
  Synthetic polynucleotide
ccaucccacacaggcggagg

SEQ ID NO: 688 - RNA, 20 nt, Artificial Sequence
  Synthetic polynucleotide
cccacucuaccacacagguu

SEQ ID NO: 689 - RNA, 20 nt, Artificial Sequence
  Synthetic polynucleotide
cccaucucaccaugcaagag

SEQ ID NO: 690 - RNA, 20 nt, Artificial Sequence
  Synthetic polynucleotide
cccccaccuaagaucuaagu

SEQ ID NO: 691 - RNA, 20 nt, Artificial Sequence
  Synthetic polynucleotide
cccccaggucauuccgguga

SEQ ID NO: 692 - RNA, 20 nt, Artificial Sequence
  Synthetic polynucleotide
ccccccaggucauuccggug

SEQ ID NO: 693 - RNA, 20 nt, Artificial Sequence
  Synthetic polynucleotide
ccccuaaucccgaauagcau

SEQ ID NO: 694 - RNA, 20 nt, Artificial Sequence
  Synthetic polynucleotide
ccccucuacugcgccacaac

SEQ ID NO: 695 - RNA, 20 nt, Artificial Sequence
  Synthetic polynucleotide
cccggaggcggcaugccuau

SEQ ID NO: 696 - RNA, 20 nt, Artificial Sequence
  Synthetic polynucleotide
cccucaccggaaugaccugg

SEQ ID NO: 697 - RNA, 20 nt, Artificial Sequence
  Synthetic polynucleotide
cccucaggucuuccuaugug

SEQ ID NO: 698 - RNA, 20 nt, Artificial Sequence
  Synthetic polynucleotide
ccgcacauaggaagaccuga

SEQ ID NO: 699 - RNA, 20 nt, Artificial Sequence
  Synthetic polynucleotide
ccggacagcgacaggccccg

SEQ ID NO: 700 - RNA, 20 nt, Artificial Sequence
  Synthetic polynucleotide
ccuaaaaaauagcuaaacag

SEQ ID NO: 701 - RNA, 20 nt, Artificial Sequence
  Synthetic polynucleotide
ccuaauuuacacucacccuc

SEQ ID NO: 702 - RNA, 20 nt, Artificial Sequence
  Synthetic polynucleotide
ccuacgauagguagaaaccc

SEQ ID NO: 703 - RNA, 20 nt, Artificial Sequence
  Synthetic polynucleotide
ccuacuuagaucuuaggugg

SEQ ID NO: 704 - RNA, 20 nt, Artificial Sequence
  Synthetic polynucleotide
ccuauugcgggugucugcgg

SEQ ID NO: 705 - RNA, 20 nt, Artificial Sequence
  Synthetic polynucleotide
ccucaccggaaugaccuggg

SEQ ID NO: 706 - RNA, 20 nt, Artificial Sequence
  Synthetic polynucleotide
ccucccucaccggaaugacc

SEQ ID NO: 707 - RNA, 20 nt, Artificial Sequence
  Synthetic polynucleotide
ccugagaguccaucaucugc

SEQ ID NO: 708 - RNA, 20 nt, Artificial Sequence
  Synthetic polynucleotide
ccugagggugaguguaaauu

SEQ ID NO: 709 - RNA, 20 nt, Artificial Sequence
  Synthetic polynucleotide
ccugucgcgacaccacaguu

SEQ ID NO: 710 - RNA, 20 nt, Artificial Sequence
  Synthetic polynucleotide
ccuugacauucucuguaugg

SEQ ID NO: 711 - RNA, 20 nt, Artificial Sequence
  Synthetic polynucleotide
cgacauagccuuccaagacc

SEQ ID NO: 712 - RNA, 20 nt, Artificial Sequence
  Synthetic polynucleotide
cgagcggacaggauaccauc

SEQ ID NO: 713 - RNA, 20 nt, Artificial Sequence
  Synthetic polynucleotide
cgccuaaaaucccuaugcac

SEQ ID NO: 714 - RNA, 20 nt, Artificial Sequence
  Synthetic polynucleotide
cgccuaccagucaacucccc

SEQ ID NO: 715 - RNA, 20 nt, Artificial Sequence
  Synthetic polynucleotide
cgcgacaccacaguuggggg

SEQ ID NO: 716 - RNA, 20 nt, Artificial Sequence
  Synthetic polynucleotide
cggcaguugcucaauaugaa

SEQ ID NO: 717 - RNA, 20 nt, Artificial Sequence
  Synthetic polynucleotide
cggucagugacgcgauggag

SEQ ID NO: 718 - RNA, 20 nt, Artificial Sequence
  Synthetic polynucleotide
cgugaagugaucgguauuua

SEQ ID NO: 719 - RNA, 20 nt, Artificial Sequence
  Synthetic polynucleotide
cguggggggaccagugcaua

SEQ ID NO: 720 - RNA, 20 nt, Artificial Sequence
  Synthetic polynucleotide
cguguggcgaggguacgugg

SEQ ID NO: 721 - RNA, 20 nt, Artificial Sequence
  Synthetic polynucleotide
cuaaagcgaggguuccuaaa

SEQ ID NO: 722 - RNA, 20 nt, Artificial Sequence
  Synthetic polynucleotide
cuaacuacuucaguuacccu

SEQ ID NO: 723 - RNA, 20 nt, Artificial Sequence
  Synthetic polynucleotide
cuaccucagaggagugucaa

SEQ ID NO: 724 - RNA, 20 nt, Artificial Sequence
  Synthetic polynucleotide
cuaccugcugaaaucaguga

SEQ ID NO: 725 - RNA, 20 nt, Artificial Sequence
  Synthetic polynucleotide
cuacugcgccacaaccggaa

SEQ ID NO: 726 - RNA, 20 nt, Artificial Sequence
  Synthetic polynucleotide
cuacugcugcaccgugggua

SEQ ID NO: 727 - RNA, 20 nt, Artificial Sequence
  Synthetic polynucleotide
cuauuggcgucccucgagca

SEQ ID NO: 728 - RNA, 20 nt, Artificial Sequence
  Synthetic polynucleotide
cucauaaaauggcugcucaa

SEQ ID NO: 729 - RNA, 20 nt, Artificial Sequence
  Synthetic polynucleotide
cuccuacuuagaucuuaggu

SEQ ID NO: 730 - RNA, 20 nt, Artificial Sequence
  Synthetic polynucleotide
cucuacacagagauuguaca

SEQ ID NO: 731 - RNA, 20 nt, Artificial Sequence
  Synthetic polynucleotide
cucucauugcaacagugcug

SEQ ID NO: 732 - RNA, 20 nt, Artificial Sequence
  Synthetic polynucleotide
cugagcucagagccuucugu

SEQ ID NO: 733 - RNA, 20 nt, Artificial Sequence
  Synthetic polynucleotide
cuggcgcgcaugcucaggcg

SEQ ID NO: 734 - RNA, 20 nt, Artificial Sequence
  Synthetic polynucleotide
cugucgcgacaccacaguug

SEQ ID NO: 735 - RNA, 20 nt, Artificial Sequence
  Synthetic polynucleotide
cuuaaugauuaucucugggg

SEQ ID NO: 736 - RNA, 20 nt, Artificial Sequence
  Synthetic polynucleotide
cuuaccacugcacccuaaag

SEQ ID NO: 737 - RNA, 20 nt, Artificial Sequence
  Synthetic polynucleotide
cuuacucaagggaaaagcuu

SEQ ID NO: 738 - RNA, 20 nt, Artificial Sequence
  Synthetic polynucleotide
cuuaugauuugguaggaagu

SEQ ID NO: 739 - RNA, 20 nt, Artificial Sequence
  Synthetic polynucleotide
cuugcaguggggcgagacgu

SEQ ID NO: 740 - RNA, 20 nt, Artificial Sequence
  Synthetic polynucleotide
cuuguuggcauguaaggauu

SEQ ID NO: 741 - RNA, 20 nt, Artificial Sequence
  Synthetic polynucleotide
gaaacaugacgaaaguguca

SEQ ID NO: 742 - RNA, 20 nt, Artificial Sequence
  Synthetic polynucleotide
gaaaggucugaggucaacug

SEQ ID NO: 743 - RNA, 20 nt, Artificial Sequence
  Synthetic polynucleotide
gaacagugauaggcaaagag

SEQ ID NO: 744 - RNA, 20 nt, Artificial Sequence
  Synthetic polynucleotide
gaacauuugacuaccagacc

SEQ ID NO: 745 - RNA, 20 nt, Artificial Sequence
  Synthetic polynucleotide
gaaccugagguuagcagcaa

SEQ ID NO: 746 - RNA, 20 nt, Artificial Sequence
  Synthetic polynucleotide
gaacuuccagggcuauuuaa

SEQ ID NO: 747 - RNA, 20 nt, Artificial Sequence
  Synthetic polynucleotide
gaauuguugggagaccuauc

SEQ ID NO: 748 - RNA, 20 nt, Artificial Sequence
  Synthetic polynucleotide
gaccuccuacagcaguuaaa

SEQ ID NO: 749 - RNA, 20 nt, Artificial Sequence
  Synthetic polynucleotide
gacuuuggcggcgguaggca

SEQ ID NO: 750 - RNA, 20 nt, Artificial Sequence
  Synthetic polynucleotide
gagcggacaggauaccaucg

SEQ ID NO: 751 - RNA, 20 nt, Artificial Sequence
  Synthetic polynucleotide
gaucugccauauuaggaucc

SEQ ID NO: 752 - RNA, 20 nt, Artificial Sequence
  Synthetic polynucleotide
gaugcgucaggcaucuccgg

SEQ ID NO: 753 - RNA, 20 nt, Artificial Sequence
  Synthetic polynucleotide
gauguacaaccuaacccuca

SEQ ID NO: 754 - RNA, 20 nt, Artificial Sequence
  Synthetic polynucleotide
gauucaaagaggaaauacau

SEQ ID NO: 755 - RNA, 20 nt, Artificial Sequence
  Synthetic polynucleotide
gauuccucgcuacuccaaua

SEQ ID NO: 756 - RNA, 20 nt, Artificial Sequence
  Synthetic polynucleotide
gcaaaccagaugaccaucgc

SEQ ID NO: 757 - RNA, 20 nt, Artificial Sequence
  Synthetic polynucleotide
gcaagguaguaaacaagugc

SEQ ID NO: 758 - RNA, 20 nt, Artificial Sequence
  Synthetic polynucleotide
gcaaugagaauccauaucca

SEQ ID NO: 759 - RNA, 20 nt, Artificial Sequence
  Synthetic polynucleotide
gcacgccugcgcggccgcag

SEQ ID NO: 760 - RNA, 20 nt, Artificial Sequence
  Synthetic polynucleotide
gcagaucuaagccgcugcag

SEQ ID NO: 761 - RNA, 20 nt, Artificial Sequence
  Synthetic polynucleotide
gcagcgagucuggcgcagag

SEQ ID NO: 762 - RNA, 20 nt, Artificial Sequence
  Synthetic polynucleotide
gcaggacaguccuucaccac

SEQ ID NO: 763 - RNA, 20 nt, Artificial Sequence
  Synthetic polynucleotide
gcaggcuggcgcgcaugcuc

SEQ ID NO: 764 - RNA, 20 nt, Artificial Sequence
  Synthetic polynucleotide
gcaggguacguggggcgacu

SEQ ID NO: 765 - RNA, 20 nt, Artificial Sequence
  Synthetic polynucleotide
gccucugaacauuccggauc

SEQ ID NO: 766 - RNA, 20 nt, Artificial Sequence
  Synthetic polynucleotide
gcgaagguacgugaagugau

SEQ ID NO: 767 - RNA, 20 nt, Artificial Sequence
  Synthetic polynucleotide
gcgacaguggguauuggcgg

SEQ ID NO: 768 - RNA, 20 nt, Artificial Sequence
  Synthetic polynucleotide
gcgcacaauacaaccaagag

SEQ ID NO: 769 - RNA, 20 nt, Artificial Sequence
  Synthetic polynucleotide
gcgcaguuguaccaugaaug

SEQ ID NO: 770 - RNA, 20 nt, Artificial Sequence
  Synthetic polynucleotide
gcgccccaaugcgagcggac

SEQ ID NO: 771 - RNA, 20 nt, Artificial Sequence
  Synthetic polynucleotide
gcggcgacaguggguauugg

SEQ ID NO: 772 - RNA, 20 nt, Artificial Sequence
  Synthetic polynucleotide
gcggcucuacagggaggaag

SEQ ID NO: 773 - RNA, 20 nt, Artificial Sequence
  Synthetic polynucleotide
gcuacaaugaaacuuuggag

SEQ ID NO: 774 - RNA, 20 nt, Artificial Sequence
  Synthetic polynucleotide
gcucaucacaaauccagccc

SEQ ID NO: 775 - RNA, 20 nt, Artificial Sequence
  Synthetic polynucleotide
gcucaugaaacuuucauaua

SEQ ID NO: 776 - RNA, 20 nt, Artificial Sequence
  Synthetic polynucleotide
gcuccucagacagaugcguc

SEQ ID NO: 777 - RNA, 20 nt, Artificial Sequence
  Synthetic polynucleotide
gcuggcgcgcaugcucaggc

SEQ ID NO: 778 - RNA, 20 nt, Artificial Sequence
  Synthetic polynucleotide
gcuguucuguaguucgucuc

SEQ ID NO: 779 - RNA, 20 nt, Artificial Sequence
  Synthetic polynucleotide
gcuucugaagaccuagggga

SEQ ID NO: 780 - RNA, 20 nt, Artificial Sequence
  Synthetic polynucleotide
gcuuguuugccgcagugcag

SEQ ID NO: 781 - RNA, 20 nt, Artificial Sequence
  Synthetic polynucleotide
ggaaaauaaaacagcaugua

SEQ ID NO: 782 - RNA, 20 nt, Artificial Sequence
  Synthetic polynucleotide
ggaauucgucgcagugaccg

SEQ ID NO: 783 - RNA, 20 nt, Artificial Sequence
  Synthetic polynucleotide
ggaccucacgcucccuuugc

SEQ ID NO: 784 - RNA, 20 nt, Artificial Sequence
  Synthetic polynucleotide
ggacuauccagagauuaaga

SEQ ID NO: 785 - RNA, 20 nt, Artificial Sequence
  Synthetic polynucleotide
ggcaaacaagcacgccugcg

SEQ ID NO: 786 - RNA, 20 nt, Artificial Sequence
  Synthetic polynucleotide
ggcaaagagaggauaccaaa

SEQ ID NO: 787 - RNA, 20 nt, Artificial Sequence
  Synthetic polynucleotide
ggcggagugccccguccugg

SEQ ID NO: 788 - RNA, 20 nt, Artificial Sequence
  Synthetic polynucleotide
ggcuaugucgaaauaaccug

SEQ ID NO: 789 - RNA, 20 nt, Artificial Sequence
  Synthetic polynucleotide
ggcucuccuacuuagaucuu

SEQ ID NO: 790 - RNA, 20 nt, Artificial Sequence
  Synthetic polynucleotide
ggcuggcgcgcaugcucagg

SEQ ID NO: 791 - RNA, 20 nt, Artificial Sequence
  Synthetic polynucleotide
gggaaguugacucugaucac

SEQ ID NO: 792 - RNA, 20 nt, Artificial Sequence
  Synthetic polynucleotide
gggacgcgccccaaugcgag

SEQ ID NO: 793 - RNA, 20 nt, Artificial Sequence
  Synthetic polynucleotide
gggguugcucugccgacggg

SEQ ID NO: 794 - RNA, 20 nt, Artificial Sequence
  Synthetic polynucleotide
ggguggcugccaagcuugcu

SEQ ID NO: 795 - RNA, 20 nt, Artificial Sequence
  Synthetic polynucleotide
gggugguacugagaaacuaa

SEQ ID NO: 796 - RNA, 20 nt, Artificial Sequence
  Synthetic polynucleotide
ggguugcucugccgacgggu

SEQ ID NO: 797 - RNA, 20 nt, Artificial Sequence
  Synthetic polynucleotide
gguauccuguccgcucgcau

SEQ ID NO: 798 - RNA, 20 nt, Artificial Sequence
  Synthetic polynucleotide
ggucagugacgcgauggagc

SEQ ID NO: 799 - RNA, 20 nt, Artificial Sequence
  Synthetic polynucleotide
guaggaaguaggagaugaau

SEQ ID NO: 800 - RNA, 20 nt, Artificial Sequence
  Synthetic polynucleotide
guauaauagugaccacugcg

SEQ ID NO: 801 - RNA, 20 nt, Artificial Sequence
  Synthetic polynucleotide
guauauauugaaaguauaaa

SEQ ID NO: 802 - RNA, 20 nt, Artificial Sequence
  Synthetic polynucleotide
guaucagagguaaacauacc

SEQ ID NO: 803 - RNA, 20 nt, Artificial Sequence
  Synthetic polynucleotide
guauccuguccgcucgcauu

SEQ ID NO: 804 - RNA, 20 nt, Artificial Sequence
  Synthetic polynucleotide
gucacagugugcggcaccag

SEQ ID NO: 805 - RNA, 20 nt, Artificial Sequence
  Synthetic polynucleotide
gucauuucauccggccucau

SEQ ID NO: 806 - RNA, 20 nt, Artificial Sequence
  Synthetic polynucleotide
gucccccaacuccucggaca

SEQ ID NO: 807 - RNA, 20 nt, Artificial Sequence
  Synthetic polynucleotide
guccucuacugcugcaccgu

SEQ ID NO: 808 - RNA, 20 nt, Artificial Sequence
  Synthetic polynucleotide
gucgcgacagguccuauugc

SEQ ID NO: 809 - RNA, 20 nt, Artificial Sequence
  Synthetic polynucleotide
gucuagugaggcacacugac

SEQ ID NO: 810 - RNA, 20 nt, Artificial Sequence
  Synthetic polynucleotide
gucuuguaaagauacagcaa

SEQ ID NO: 811 - RNA, 20 nt, Artificial Sequence
  Synthetic polynucleotide
gugacacagaacugaaauaa

SEQ ID NO: 812 - RNA, 20 nt, Artificial Sequence
  Synthetic polynucleotide
gugacgcaacacagaccccc

SEQ ID NO: 813 - RNA, 20 nt, Artificial Sequence
  Synthetic polynucleotide
gugacgcgauggagcgggca

SEQ ID NO: 814 - RNA, 20 nt, Artificial Sequence
  Synthetic polynucleotide
gugagcuagccugaagcagg

SEQ ID NO: 815 - RNA, 20 nt, Artificial Sequence
  Synthetic polynucleotide
guguauaaaggauucaagaa

SEQ ID NO: 816 - RNA, 20 nt, Artificial Sequence
  Synthetic polynucleotide
guguucaccaaucccuuacc

SEQ ID NO: 817 - RNA, 20 nt, Artificial Sequence
  Synthetic polynucleotide
guguuuucuguuggggugau

SEQ ID NO: 818 - RNA, 20 nt, Artificial Sequence
  Synthetic polynucleotide
guuagaguacuacuucuucu

SEQ ID NO: 819 - RNA, 20 nt, Artificial Sequence
  Synthetic polynucleotide
guugugccguucugccccga

SEQ ID NO: 820 - RNA, 20 nt, Artificial Sequence
  Synthetic polynucleotide
uaacucaaaugaccucuauc

SEQ ID NO: 821 - RNA, 20 nt, Artificial Sequence
  Synthetic polynucleotide
uaagaugacagagcccucac

SEQ ID NO: 822 - RNA, 20 nt, Artificial Sequence
  Synthetic polynucleotide
uacaggcccuaaaucaaucc

SEQ ID NO: 823 - RNA, 20 nt, Artificial Sequence
  Synthetic polynucleotide
uacccacggugcagcaguag

SEQ ID NO: 824 - RNA, 20 nt, Artificial Sequence
  Synthetic polynucleotide
uacugcugcaccguggguaa

SEQ ID NO: 825 - RNA, 20 nt, Artificial Sequence
  Synthetic polynucleotide
uagaggugagugggagcaua

SEQ ID NO: 826 - RNA, 20 nt, Artificial Sequence
  Synthetic polynucleotide
uagaugaacuaaagaaccug

SEQ ID NO: 827 - RNA, 20 nt, Artificial Sequence
  Synthetic polynucleotide
uagcaugguugacuaucugu

SEQ ID NO: 828 - RNA, 20 nt, Artificial Sequence
  Synthetic polynucleotide
uaguguuaggaagaucaaau

SEQ ID NO: 829 - RNA, 20 nt, Artificial Sequence
  Synthetic polynucleotide
uauagaagcuaacaugggaa

SEQ ID NO: 830 - RNA, 20 nt, Artificial Sequence
  Synthetic polynucleotide
uauccgggcuccgggucucu

SEQ ID NO: 831 - RNA, 20 nt, Artificial Sequence
  Synthetic polynucleotide
uauccuuaaccuguacugau

SEQ ID NO: 832 - RNA, 20 nt, Artificial Sequence
  Synthetic polynucleotide
uauggugagcaguuccccaa

SEQ ID NO: 833 - RNA, 20 nt, Artificial Sequence
  Synthetic polynucleotide
uauuggcgucccucgagcag

SEQ ID NO: 834 - RNA, 20 nt, Artificial Sequence
  Synthetic polynucleotide
uauacacacuccuaguauaa

SEQ ID NO: 835 - RNA, 20 nt, Artificial Sequence
  Synthetic polynucleotide
ucaacuaaguggucaaaaaa

SEQ ID NO: 836 - RNA, 20 nt, Artificial Sequence
  Synthetic polynucleotide
ucaagaaccuggcauauacg

SEQ ID NO: 837 - RNA, 20 nt, Artificial Sequence
  Synthetic polynucleotide
ucacauaaaaacuaccucag

SEQ ID NO: 838 - RNA, 20 nt, Artificial Sequence
  Synthetic polynucleotide
ucaccucuaaugccuggugg

SEQ ID NO: 839 - RNA, 20 nt, Artificial Sequence
  Synthetic polynucleotide
ucacguaauggggacacauu

SEQ ID NO: 840 - RNA, 20 nt, Artificial Sequence
  Synthetic polynucleotide
ucacuacuguaaaacaagcu

SEQ ID NO: 841 - RNA, 20 nt, Artificial Sequence
  Synthetic polynucleotide
ucacucuugguugcucagug

SEQ ID NO: 842 - RNA, 20 nt, Artificial Sequence
  Synthetic polynucleotide
ucauacuagcuuccucacca

SEQ ID NO: 843 - RNA, 20 nt, Artificial Sequence
  Synthetic polynucleotide
uccauguucaauucagguug

SEQ ID NO: 844 - RNA, 20 nt, Artificial Sequence
  Synthetic polynucleotide
ucccaauacuacuuaugauu

SEQ ID NO: 845 - RNA, 20 nt, Artificial Sequence
  Synthetic polynucleotide
ucccacucaccucuaaugcc

SEQ ID NO: 846 - RNA, 20 nt, Artificial Sequence
  Synthetic polynucleotide
ucccucaccggaaugaccug

SEQ ID NO: 847 - RNA, 20 nt, Artificial Sequence
  Synthetic polynucleotide
uccguguggcgaggguacgu

SEQ ID NO: 848 - RNA, 20 nt, Artificial Sequence
  Synthetic polynucleotide
uccuacuuagaucuuaggug

SEQ ID NO: 849 - RNA, 20 nt, Artificial Sequence
  Synthetic polynucleotide
ucguugugacacuacugagu

SEQ ID NO: 850 - RNA, 20 nt, Artificial Sequence
  Synthetic polynucleotide
ucuacacagagauuguacaa

SEQ ID NO: 851 - RNA, 20 nt, Artificial Sequence
  Synthetic polynucleotide
ucuaucauaacugguauguc

SEQ ID NO: 852 - RNA, 20 nt, Artificial Sequence
  Synthetic polynucleotide
ucuccuacuuagaucuuagg

SEQ ID NO: 853 - RNA, 20 nt, Artificial Sequence
  Synthetic polynucleotide
ucucgccccacugcaagcgg

SEQ ID NO: 854 - RNA, 20 nt, Artificial Sequence
  Synthetic polynucleotide
ucugaccuaauucagagauc

SEQ ID NO: 855 - RNA, 20 nt, Artificial Sequence
  Synthetic polynucleotide
ucuugguggacgcauuccug

SEQ ID NO: 856 - RNA, 20 nt, Artificial Sequence
  Synthetic polynucleotide
ugaaaagguacgauuuucca

SEQ ID NO: 857 - RNA, 20 nt, Artificial Sequence
  Synthetic polynucleotide
ugaagugaucgguauuuagg

SEQ ID NO: 858 - RNA, 20 nt, Artificial Sequence
  Synthetic polynucleotide
ugaccgaggcgaggaggcua

SEQ ID NO: 859 - RNA, 20 nt, Artificial Sequence
  Synthetic polynucleotide
ugacgcaucugucugaggag

SEQ ID NO: 860 - RNA, 20 nt, Artificial Sequence
  Synthetic polynucleotide
ugagcucagagccuucugug

SEQ ID NO: 861 - RNA, 20 nt, Artificial Sequence
  Synthetic polynucleotide
ugcauagggauuuuaggcgg

SEQ ID NO: 862 - RNA, 20 nt, Artificial Sequence
  Synthetic polynucleotide
ugccgcugcugcagcgaguc

SEQ ID NO: 863 - RNA, 20 nt, Artificial Sequence
  Synthetic polynucleotide
ugccuuagacaacaacauaa

SEQ ID NO: 864 - RNA, 20 nt, Artificial Sequence
  Synthetic polynucleotide
ugcuguaucagccaggugaa

SEQ ID NO: 865 - RNA, 20 nt, Artificial Sequence
  Synthetic polynucleotide
uggaaaacacauucuaggac

SEQ ID NO: 866 - RNA, 20 nt, Artificial Sequence
  Synthetic polynucleotide
uggccagaauguccaccgug

SEQ ID NO: 867 - RNA, 20 nt, Artificial Sequence
  Synthetic polynucleotide
uggcugccaagcuugcuagg

SEQ ID NO: 868 - RNA, 20 nt, Artificial Sequence
  Synthetic polynucleotide
uggggaauuggcacaccagc

SEQ ID NO: 869 - RNA, 20 nt, Artificial Sequence
  Synthetic polynucleotide
uguagcaugcuuuuagaguu

SEQ ID NO: 870 - RNA, 20 nt, Artificial Sequence
  Synthetic polynucleotide
ugucgcgacaccacaguugg

SEQ ID NO: 871 - RNA, 20 nt, Artificial Sequence
  Synthetic polynucleotide
ugucgcgacagguccuauug

SEQ ID NO: 872 - RNA, 20 nt, Artificial Sequence
  Synthetic polynucleotide
ugugaauuuguaaccaccca

SEQ ID NO: 873 - RNA, 20 nt, Artificial Sequence
  Synthetic polynucleotide
uguuggggcuccagcaauac

SEQ ID NO: 874 - RNA, 20 nt, Artificial Sequence
  Synthetic polynucleotide
uuaaacucuccuauacggaa

SEQ ID NO: 875 - RNA, 20 nt, Artificial Sequence
  Synthetic polynucleotide
uuacacugaaaguaauaauc

SEQ ID NO: 876 - RNA, 20 nt, Artificial Sequence
  Synthetic polynucleotide
uuaggcggagguagguauau

SEQ ID NO: 877 - RNA, 20 nt, Artificial Sequence
  Synthetic polynucleotide
uucaacauauccuacaacug

SEQ ID NO: 878 - RNA, 20 nt, Artificial Sequence
  Synthetic polynucleotide
uucaaccagagauguuucau

SEQ ID NO: 879 - RNA, 20 nt, Artificial Sequence
  Synthetic polynucleotide
uucaccaaucccuuaccugg

SEQ ID NO: 880 - RNA, 20 nt, Artificial Sequence
  Synthetic polynucleotide
uuccgguuguggcgcaguag

SEQ ID NO: 881 - RNA, 20 nt, Artificial Sequence
  Synthetic polynucleotide
uuccguguggcgaggguacg

SEQ ID NO: 882 - RNA, 20 nt, Artificial Sequence
  Synthetic polynucleotide
uucuacaggaauacuaguaa

SEQ ID NO: 883 - RNA, 20 nt, Artificial Sequence
  Synthetic polynucleotide
uugcugcauuagcucuaacc

SEQ ID NO: 884 - RNA, 20 nt, Artificial Sequence
  Synthetic polynucleotide
uuggacugcuaacaccauag

SEQ ID NO: 885 - RNA, 20 nt, Artificial Sequence
  Synthetic polynucleotide
uuucagaggcaucaacuaag

SEQ ID NO: 886 - RNA, 20 nt, Artificial Sequence
  Synthetic polynucleotide
uuucauauaggguuggaacc

SEQ ID NO: 887 - RNA, 20 nt, Artificial Sequence
  Synthetic polynucleotide
uuugccuguacucuacuaga

SEQ ID NO: 888 - RNA, 20 nt, Artificial Sequence
  Synthetic polynucleotide
aaaaugaggugcucuuuacu

SEQ ID NO: 889 - RNA, 20 nt, Artificial Sequence
  Synthetic polynucleotide
aaaccccugaucccccuaca

SEQ ID NO: 890 - RNA, 20 nt, Artificial Sequence
  Synthetic polynucleotide
aaacggcugagagaccacag

SEQ ID NO: 891 - RNA, 20 nt, Artificial Sequence
  Synthetic polynucleotide
aaagacaaugacaaggguug

SEQ ID NO: 892 - RNA, 20 nt, Artificial Sequence
  Synthetic polynucleotide
aacaaauggaagacaggagg

SEQ ID NO: 893 - RNA, 20 nt, Artificial Sequence
  Synthetic polynucleotide
aacagguccuaguauaagcu

SEQ ID NO: 894 - RNA, 20 nt, Artificial Sequence
  Synthetic polynucleotide
aagacucccuugagcccggg

SEQ ID NO: 895 - RNA, 20 nt, Artificial Sequence
  Synthetic polynucleotide
aaguucgcggggaaugaaca

SEQ ID NO: 896 - RNA, 20 nt, Artificial Sequence
  Synthetic polynucleotide
aauaucggcaacccucucuu

SEQ ID NO: 897 - RNA, 20 nt, Artificial Sequence
  Synthetic polynucleotide
aauccguguaaccuugaauu

SEQ ID NO: 898 - RNA, 20 nt, Artificial Sequence
  Synthetic polynucleotide
aaugacaaggguuguggagg

SEQ ID NO: 899 - RNA, 20 nt, Artificial Sequence
  Synthetic polynucleotide
aauguuuccagaguaguuca

SEQ ID NO: 900 - RNA, 20 nt, Artificial Sequence
  Synthetic polynucleotide
acaucuagggaugugaguua

SEQ ID NO: 901 - RNA, 20 nt, Artificial Sequence
  Synthetic polynucleotide
acagagucuacuauacucuu

SEQ ID NO: 902 - RNA, 20 nt, Artificial Sequence
  Synthetic polynucleotide
accaaugggaaaaggguaca

SEQ ID NO: 903 - RNA, 20 nt, Artificial Sequence
  Synthetic polynucleotide
acccucccuucuucgauaau

SEQ ID NO: 904 - RNA, 20 nt, Artificial Sequence
  Synthetic polynucleotide
accugugcccacugacccac

SEQ ID NO: 905 - RNA, 20 nt, Artificial Sequence
  Synthetic polynucleotide
acuacacuagaaaccaccua

SEQ ID NO: 906 - RNA, 20 nt, Artificial Sequence
  Synthetic polynucleotide
agacucugugggguaugaug

SEQ ID NO: 907 - RNA, 20 nt, Artificial Sequence
  Synthetic polynucleotide
agacuguuggucggaagugu

SEQ ID NO: 908 - RNA, 20 nt, Artificial Sequence
  Synthetic polynucleotide
agagccugaguccaaaggca

SEQ ID NO: 909 - RNA, 20 nt, Artificial Sequence
  Synthetic polynucleotide
agagguaugaaacacaucua

SEQ ID NO: 910 - RNA, 20 nt, Artificial Sequence
  Synthetic polynucleotide
agauaacuguaaggacugug

SEQ ID NO: 911 - RNA, 20 nt, Artificial Sequence
  Synthetic polynucleotide
agguaaugcauccugaggac

SEQ ID NO: 912 - RNA, 20 nt, Artificial Sequence
  Synthetic polynucleotide
agucucagucccaagcagcu

SEQ ID NO: 913 - RNA, 20 nt, Artificial Sequence
  Synthetic polynucleotide
agugaaaggauaaccagaau

SEQ ID NO: 914 - RNA, 20 nt, Artificial Sequence
  Synthetic polynucleotide
agugaccgacacacccaccu

SEQ ID NO: 915 - RNA, 20 nt, Artificial Sequence
  Synthetic polynucleotide
aguucgcggggaaugaacaa

SEQ ID NO: 916 - RNA, 20 nt, Artificial Sequence
  Synthetic polynucleotide
auacccaagcuugaacuccc

SEQ ID NO: 917 - RNA, 20 nt, Artificial Sequence
  Synthetic polynucleotide
aucacgaaccuccugaucuc

SEQ ID NO: 918 - RNA, 20 nt, Artificial Sequence
  Synthetic polynucleotide
augaagucccacggagucaa

SEQ ID NO: 919 - RNA, 20 nt, Artificial Sequence
  Synthetic polynucleotide
auguuaaacagagaagaggc

SEQ ID NO: 920 - RNA, 20 nt, Artificial Sequence
  Synthetic polynucleotide
auugcaggcccugcucccga

SEQ ID NO: 921 - RNA, 20 nt, Artificial Sequence
  Synthetic polynucleotide
caaauuaacuauaaucucaa

SEQ ID NO: 922 - RNA, 20 nt, Artificial Sequence
  Synthetic polynucleotide
cacaccuacaauaccagcug

SEQ ID NO: 923 - RNA, 20 nt, Artificial Sequence
  Synthetic polynucleotide
cacagccaauggcaccaucu

SEQ ID NO: 924 - RNA, 20 nt, Artificial Sequence
  Synthetic polynucleotide
caccacaaacacugccuucg

SEQ ID NO: 925 - RNA, 20 nt, Artificial Sequence
  Synthetic polynucleotide
cagguagacaacaccacgcc

SEQ ID NO: 926 - RNA, 20 nt, Artificial Sequence
  Synthetic polynucleotide
caggugcgugcccucacacc

SEQ ID NO: 927 - RNA, 20 nt, Artificial Sequence
  Synthetic polynucleotide
caugccaaaaagagggaggu

SEQ ID NO: 928 - RNA, 20 nt, Artificial Sequence
  Synthetic polynucleotide
cauguccgggcaugcaacaa

SEQ ID NO: 929 - RNA, 20 nt, Artificial Sequence
  Synthetic polynucleotide
cauugaggggaauguucugg

SEQ ID NO: 930 - RNA, 20 nt, Artificial Sequence
  Synthetic polynucleotide
ccacaagcuuagcgguccag

SEQ ID NO: 931 - RNA, 20 nt, Artificial Sequence
  Synthetic polynucleotide
ccacaaugagaugccacuug

SEQ ID NO: 932 - RNA, 20 nt, Artificial Sequence
  Synthetic polynucleotide
cccacuuaggugccagggaa

SEQ ID NO: 933 - RNA, 20 nt, Artificial Sequence
  Synthetic polynucleotide
ccccaaguggcaucucauug

SEQ ID NO: 934 - RNA, 20 nt, Artificial Sequence
  Synthetic polynucleotide
ccccuccaucaugccuuugu

SEQ ID NO: 935 - RNA, 20 nt, Artificial Sequence
  Synthetic polynucleotide
cccuuccacuucagccuccc

SEQ ID NO: 936 - RNA, 20 nt, Artificial Sequence
  Synthetic polynucleotide
ccguacccccauaccaaaau

SEQ ID NO: 937 - RNA, 20 nt, Artificial Sequence
  Synthetic polynucleotide
ccucagaaucuauagacugu

SEQ ID NO: 938 - RNA, 20 nt, Artificial Sequence
  Synthetic polynucleotide
ccugcuacuguggcaugucc

SEQ ID NO: 939 - RNA, 20 nt, Artificial Sequence
  Synthetic polynucleotide
cuaaagacucccuugagccc

SEQ ID NO: 940 - RNA, 20 nt, Artificial Sequence
  Synthetic polynucleotide
cuagaaaccaccuaagggua

SEQ ID NO: 941 - RNA, 20 nt, Artificial Sequence
  Synthetic polynucleotide
cucaaaucaaucucccugua

SEQ ID NO: 942 - RNA, 20 nt, Artificial Sequence
  Synthetic polynucleotide
cucaagcacugacugagcuu

SEQ ID NO: 943 - RNA, 20 nt, Artificial Sequence
  Synthetic polynucleotide
cugcaauucguuccuuccag

SEQ ID NO: 944 - RNA, 20 nt, Artificial Sequence
  Synthetic polynucleotide
cugcauugaggggaauguuc

SEQ ID NO: 945 - RNA, 20 nt, Artificial Sequence
  Synthetic polynucleotide
cuugaaauaauggccaggug

SEQ ID NO: 946 - RNA, 20 nt, Artificial Sequence
  Synthetic polynucleotide
gaaaccccugaucccccuac

SEQ ID NO: 947 - RNA, 20 nt, Artificial Sequence
  Synthetic polynucleotide
gacaaugacaaggguugugg

SEQ ID NO: 948 - RNA, 20 nt, Artificial Sequence
  Synthetic polynucleotide
gacguugugaggcugagacu

SEQ ID NO: 949 - RNA, 20 nt, Artificial Sequence
  Synthetic polynucleotide
gagaaaggcaugcaugcuau

SEQ ID NO: 950 - RNA, 20 nt, Artificial Sequence
  Synthetic polynucleotide
gagauaggguuuucaccacg

SEQ ID NO: 951 - RNA, 20 nt, Artificial Sequence
  Synthetic polynucleotide
gagcaauccugcagcaacaa

SEQ ID NO: 952 - RNA, 20 nt, Artificial Sequence
  Synthetic polynucleotide
gaggugugcaacaccacauu

SEQ ID NO: 953 - RNA, 20 nt, Artificial Sequence
  Synthetic polynucleotide
gaguguugggagagaauuug

SEQ ID NO: 954 - RNA, 20 nt, Artificial Sequence
  Synthetic polynucleotide
gaguuggugucugcauugag

SEQ ID NO: 955 - RNA, 20 nt, Artificial Sequence
  Synthetic polynucleotide
gaucagugcuacauuauggu

SEQ ID NO: 956 - RNA, 20 nt, Artificial Sequence
  Synthetic polynucleotide
gcaaccuauuccuaagggau

SEQ ID NO: 957 - RNA, 20 nt, Artificial Sequence
  Synthetic polynucleotide
gcacuuugggaaagaaacca

SEQ ID NO: 958 - RNA, 20 nt, Artificial Sequence
  Synthetic polynucleotide
gccuguacuaacaggcacuu

SEQ ID NO: 959 - RNA, 20 nt, Artificial Sequence
  Synthetic polynucleotide
gcgaggcuguggggguugaa

SEQ ID NO: 960 - RNA, 20 nt, Artificial Sequence
  Synthetic polynucleotide
gcuauucaauccuaguuaug

SEQ ID NO: 961 - RNA, 20 nt, Artificial Sequence
  Synthetic polynucleotide
ggacuagggauuagaguaac

SEQ ID NO: 962 - RNA, 20 nt, Artificial Sequence
  Synthetic polynucleotide
ggaggcagaggucgcgguga

SEQ ID NO: 963 - RNA, 20 nt, Artificial Sequence
  Synthetic polynucleotide
ggagggaugcacugauacgg

SEQ ID NO: 964 - RNA, 20 nt, Artificial Sequence
  Synthetic polynucleotide
ggaguuggugucugcauuga

SEQ ID NO: 965 - RNA, 20 nt, Artificial Sequence
  Synthetic polynucleotide
ggauucagucaaggugucag

SEQ ID NO: 966 - RNA, 20 nt, Artificial Sequence
  Synthetic polynucleotide
gggaaagaaaccaaggcggg

SEQ ID NO: 967 - RNA, 20 nt, Artificial Sequence
  Synthetic polynucleotide
ggguagggggucggaaagug

SEQ ID NO: 968 - RNA, 20 nt, Artificial Sequence
  Synthetic polynucleotide
ggugauauacgcuaaugugc

SEQ ID NO: 969 - RNA, 20 nt, Artificial Sequence
  Synthetic polynucleotide
gguuuugagcaagcccuccu

SEQ ID NO: 970 - RNA, 20 nt, Artificial Sequence
  Synthetic polynucleotide
guaggucauaugguaacuuc

SEQ ID NO: 971 - RNA, 20 nt, Artificial Sequence
  Synthetic polynucleotide
guccuccaaacacauacaag

SEQ ID NO: 972 - RNA, 20 nt, Artificial Sequence
  Synthetic polynucleotide
gugaaacagcacagccacag

SEQ ID NO: 973 - RNA, 20 nt, Artificial Sequence
  Synthetic polynucleotide
gugaguggagguugacuaaa

SEQ ID NO: 974 - RNA, 20 nt, Artificial Sequence
  Synthetic polynucleotide
gugauaccacugcaugucau

SEQ ID NO: 975 - RNA, 20 nt, Artificial Sequence
  Synthetic polynucleotide
gugggacuucaucgccccca

SEQ ID NO: 976 - RNA, 20 nt, Artificial Sequence
  Synthetic polynucleotide
guuuugcuccaucacccagg

SEQ ID NO: 977 - RNA, 20 nt, Artificial Sequence
  Synthetic polynucleotide
uacaauguuaaacagagaag

SEQ ID NO: 978 - RNA, 20 nt, Artificial Sequence
  Synthetic polynucleotide
uaccucaagccuguacuaac

SEQ ID NO: 979 - RNA, 20 nt, Artificial Sequence
  Synthetic polynucleotide
uacugaucagugcuacauua

SEQ ID NO: 980 - RNA, 20 nt, Artificial Sequence
  Synthetic polynucleotide
uagcaaccuaugacaugcag

SEQ ID NO: 981 - RNA, 20 nt, Artificial Sequence
  Synthetic polynucleotide
uaggaucacaccugaguucu

SEQ ID NO: 982 - RNA, 20 nt, Artificial Sequence
  Synthetic polynucleotide
ucaaaagagccugaguccaa

SEQ ID NO: 983 - RNA, 20 nt, Artificial Sequence
  Synthetic polynucleotide
uccuacaaaggcaugaugga

SEQ ID NO: 984 - RNA, 20 nt, Artificial Sequence
  Synthetic polynucleotide
ugaucagugcuacauuaugg

SEQ ID NO: 985 - RNA, 20 nt, Artificial Sequence
  Synthetic polynucleotide
ugcugacugucaugcguugu

SEQ ID NO: 986 - RNA, 20 nt, Artificial Sequence
  Synthetic polynucleotide
uggcagucaagauaacugua

SEQ ID NO: 987 - RNA, 20 nt, Artificial Sequence
  Synthetic polynucleotide
uggguagggggucggaaagu

SEQ ID NO: 988 - RNA, 20 nt, Artificial Sequence
  Synthetic polynucleotide
ugguauuagucaagauagug

SEQ ID NO: 989 - RNA, 20 nt, Artificial Sequence
  Synthetic polynucleotide
uguaccuaucccuuaggaau

SEQ ID NO: 990 - RNA, 20 nt, Artificial Sequence
  Synthetic polynucleotide
uguuaaacagagaagaggcu

SEQ ID NO: 991 - RNA, 20 nt, Artificial Sequence
  Synthetic polynucleotide
uuccuacaaaggcaugaugg

SEQ ID NO: 992 - RNA, 20 nt, Artificial Sequence
  Synthetic polynucleotide
uugaguguucaaucucccuu

SEQ ID NO: 993 - RNA, 20 nt, Artificial Sequence
  Synthetic polynucleotide
uugggagagaauuugaggca

SEQ ID NO: 994 - RNA, 20 nt, Artificial Sequence
  Synthetic polynucleotide
uuuaggucuaagauccaccu

SEQ ID NO: 995 - RNA, 20 nt, Artificial Sequence
  Synthetic polynucleotide
uuugggaaagaaaccaaggc

SEQ ID NO: 996 - RNA, 20 nt, Artificial Sequence
  Synthetic polynucleotide
acaccaaucucuuuagugcu

SEQ ID NO: 997 - RNA, 20 nt, Artificial Sequence
  Synthetic polynucleotide
acuacagaggcacgcaucca

SEQ ID NO: 998 - RNA, 20 nt, Artificial Sequence
  Synthetic polynucleotide
aguauauagguggccuuaca

SEQ ID NO: 999 - RNA, 20 nt, Artificial Sequence
  Synthetic polynucleotide
aucaugugcagggcuuuuac

SEQ ID NO: 1000 - RNA, 20 nt, Artificial Sequence
  Synthetic polynucleotide
caauuguuaagcagaaucuc

SEQ ID NO: 1001 - RNA, 20 nt, Artificial Sequence
  Synthetic polynucleotide
cacaaugcuagcaggaguug

SEQ ID NO: 1002 - RNA, 20 nt, Artificial Sequence
  Synthetic polynucleotide
cacucaacaauuucuauccu

SEQ ID NO: 1003 - RNA, 20 nt, Artificial Sequence
  Synthetic polynucleotide
cagguaaaguacuuugguug

SEQ ID NO: 1004 - RNA, 20 nt, Artificial Sequence
  Synthetic polynucleotide
cagguaccaagaggcagacu

SEQ ID NO: 1005 - RNA, 20 nt, Artificial Sequence
  Synthetic polynucleotide
caguccagacuucuuggcgu

SEQ ID NO: 1006 - RNA, 20 nt, Artificial Sequence
  Synthetic polynucleotide
ccaagggacuggcggagccc

SEQ ID NO: 1007 - RNA, 20 nt, Artificial Sequence
  Synthetic polynucleotide
cccgggcgccgaggacccug

SEQ ID NO: 1008 - RNA, 20 nt, Artificial Sequence
  Synthetic polynucleotide
cgaagaaauuaacagaagag

SEQ ID NO: 1009 - RNA, 20 nt, Artificial Sequence
  Synthetic polynucleotide
cggcauucuggggaugaauc

SEQ ID NO: 1010 - RNA, 20 nt, Artificial Sequence
  Synthetic polynucleotide
cggccgcggcgcaagacggg

SEQ ID NO: 1011 - RNA, 20 nt, Artificial Sequence
  Synthetic polynucleotide
cugccacgcugugacucaga

SEQ ID NO: 1012 - RNA, 20 nt, Artificial Sequence
  Synthetic polynucleotide
cugugacucagacggcauuc

SEQ ID NO: 1013 - RNA, 20 nt, Artificial Sequence
  Synthetic polynucleotide
gaacuguauccacccucagc

SEQ ID NO: 1014 - RNA, 20 nt, Artificial Sequence
  Synthetic polynucleotide
gaaguaguuccagagcagua

SEQ ID NO: 1015 - RNA, 20 nt, Artificial Sequence
  Synthetic polynucleotide
gaugugaucuuugggcccuu

SEQ ID NO: 1016 - RNA, 20 nt, Artificial Sequence
  Synthetic polynucleotide
gcacaaccacuuaaaccccc

SEQ ID NO: 1017 - RNA, 20 nt, Artificial Sequence
  Synthetic polynucleotide
gcaguaugggaagaaaucac

SEQ ID NO: 1018 - RNA, 20 nt, Artificial Sequence
  Synthetic polynucleotide
gccuugauauguaaugagca

SEQ ID NO: 1019 - RNA, 20 nt, Artificial Sequence
  Synthetic polynucleotide
ggauaaaauacuuuuggaag

SEQ ID NO: 1020 - RNA, 20 nt, Artificial Sequence
  Synthetic polynucleotide
ggcagggcgccccgggaacu

SEQ ID NO: 1021 - RNA, 20 nt, Artificial Sequence
  Synthetic polynucleotide
gugacauucacaaccacucc

SEQ ID NO: 1022 - RNA, 20 nt, Artificial Sequence
  Synthetic polynucleotide
gugacucagacggcauucug

SEQ ID NO: 1023 - RNA, 20 nt, Artificial Sequence
  Synthetic polynucleotide
guuccauaaccaauucaaac

SEQ ID NO: 1024 - RNA, 20 nt, Artificial Sequence
  Synthetic polynucleotide
guugggauaugguugccauu

SEQ ID NO: 1025 - RNA, 20 nt, Artificial Sequence
  Synthetic polynucleotide
uaccguaugaagggauccac

SEQ ID NO: 1026 - RNA, 20 nt, Artificial Sequence
  Synthetic polynucleotide
uaccuguguagugcacaugg

SEQ ID NO: 1027 - RNA, 20 nt, Artificial Sequence
  Synthetic polynucleotide
uacuaccccacgguuuguag

SEQ ID NO: 1028 - RNA, 20 nt, Artificial Sequence
  Synthetic polynucleotide
uaguaacauacuccgcgcau

SEQ ID NO: 1029 - RNA, 20 nt, Artificial Sequence
  Synthetic polynucleotide
ucacucaugccaaaaucuaa

SEQ ID NO: 1030 - RNA, 20 nt, Artificial Sequence
  Synthetic polynucleotide
ucaugacccggggguuuaag

SEQ ID NO: 1031 - RNA, 20 nt, Artificial Sequence
  Synthetic polynucleotide
uccauauauuccugcagggu

SEQ ID NO: 1032 - RNA, 20 nt, Artificial Sequence
  Synthetic polynucleotide
ucgagccugcgcucccuccg

SEQ ID NO: 1033 - RNA, 20 nt, Artificial Sequence
  Synthetic polynucleotide
ucggcgcgccugcacagcgc

SEQ ID NO: 1034 - RNA, 20 nt, Artificial Sequence
  Synthetic polynucleotide
ucuagcagaaacgcaaguuc

SEQ ID NO: 1035 - RNA, 20 nt, Artificial Sequence
  Synthetic polynucleotide
ucuggacuauggcucuacag

SEQ ID NO: 1036 - RNA, 20 nt, Artificial Sequence
  Synthetic polynucleotide
ugugacucagacggcauucu

SEQ ID NO: 1037 - RNA, 20 nt, Artificial Sequence
  Synthetic polynucleotide
uuaccuuuaacucauguaac

SEQ ID NO: 1038 - RNA, 20 nt, Artificial Sequence
  Synthetic polynucleotide
uugucacuuggugauaccac

SEQ ID NO: 1039 - RNA, 20 nt, Artificial Sequence
  Synthetic polynucleotide
acacucucuagcacaguaua

SEQ ID NO: 1040 - RNA, 20 nt, Artificial Sequence
  Synthetic polynucleotide
auacaucucugccuuuuaag

SEQ ID NO: 1041 - RNA, 20 nt, Artificial Sequence
  Synthetic polynucleotide
auauuauauucauguagugc

SEQ ID NO: 1042 - RNA, 20 nt, Artificial Sequence
  Synthetic polynucleotide
cagcugaagguacaucacau

SEQ ID NO: 1043 - RNA, 20 nt, Artificial Sequence
  Synthetic polynucleotide
cuuguuuuucauuaugaaac

SEQ ID NO: 1044 - RNA, 20 nt, Artificial Sequence
  Synthetic polynucleotide
gagauaauacuuaaucgcug

SEQ ID NO: 1045 - RNA, 20 nt, Artificial Sequence
  Synthetic polynucleotide
gagcacauuuucuaugcucu

SEQ ID NO: 1046 - RNA, 20 nt, Artificial Sequence
  Synthetic polynucleotide
uacugauuuuuucugcuugg

SEQ ID NO: 1047 - RNA, 20 nt, Artificial Sequence
  Synthetic polynucleotide
uggggcacugcagcacaugu

SEQ ID NO: 1048 - RNA, 20 nt, Artificial Sequence
  Synthetic polynucleotide
uuucgguacuuaacagaugu

SEQ ID NO: 1049 - RNA, 20 nt, Artificial Sequence
  Synthetic polynucleotide
gccuacaggguaucucuccu

SEQ ID NO: 1050 - RNA, 20 nt, Artificial Sequence
  Synthetic polynucleotide
aucauacaggagugccuaca

SEQ ID NO: 1051 - RNA, 20 nt, Artificial Sequence
  Synthetic polynucleotide
ggucggcuucaggagggaag

SEQ ID NO: 1052 - RNA, 20 nt, Artificial Sequence
  Synthetic polynucleotide
cuaagcaguggacuaaggau

SEQ ID NO: 1053 - RNA, 20 nt, Artificial Sequence
  Synthetic polynucleotide
guuucaggccuggacugggu

SEQ ID NO: 1054 - RNA, 20 nt, Artificial Sequence
  Synthetic polynucleotide
gcagcucagaaagugaugca

SEQ ID NO: 1055 - RNA, 20 nt, Artificial Sequence
  Synthetic polynucleotide
acaguucagggcaugagaua

SEQ ID NO: 1056 - RNA, 20 nt, Artificial Sequence
  Synthetic polynucleotide
gggcaugagauaagggcagu

SEQ ID NO: 1057 - RNA, 20 nt, Artificial Sequence
  Synthetic polynucleotide
gcgcacugcagcgcagacca

SEQ ID NO: 1058 - RNA, 20 nt, Artificial Sequence
  Synthetic polynucleotide
gccagguuacugauucauag

SEQ ID NO: 1059 - RNA, 20 nt, Artificial Sequence
  Synthetic polynucleotide
guggauaggugcguucaagg

SEQ ID NO: 1060 - RNA, 20 nt, Artificial Sequence
  Synthetic polynucleotide
ugagcucagagccuucugug

SEQ ID NO: 1061 - RNA, 20 nt, Artificial Sequence
  Synthetic polynucleotide
ccauagcugaggccuuuauu

SEQ ID NO: 1062 - RNA, 20 nt, Artificial Sequence
  Synthetic polynucleotide
aaaaccaggcaaccauaaug

SEQ ID NO: 1063 - RNA, 20 nt, Artificial Sequence
  Synthetic polynucleotide
uccugcgccgaccucgaaug

SEQ ID NO: 1064 - RNA, 20 nt, Artificial Sequence
  Synthetic polynucleotide
cagcaggaggaccgcauucg

SEQ ID NO: 1065 - RNA, 20 nt, Artificial Sequence
  Synthetic polynucleotide
gagccuucugugugguuugc

SEQ ID NO: 1066 - RNA, 20 nt, Artificial Sequence
  Synthetic polynucleotide
agccuucugugugguuugcg

SEQ ID NO: 1067 - RNA, 20 nt, Artificial Sequence
  Synthetic polynucleotide
gccuucugugugguuugcgg

SEQ ID NO: 1068 - RNA, 20 nt, Artificial Sequence
  Synthetic polynucleotide
gcccccgcaaaccacacaga

SEQ ID NO: 1069 - RNA, 20 nt, Artificial Sequence
  Synthetic polynucleotide
aagcaauaugaaauguuacc

SEQ ID NO: 1070 - RNA, 20 nt, Artificial Sequence
  Synthetic polynucleotide
uaugacgacacucauuugca

SEQ ID NO: 1071 - RNA, 20 nt, Artificial Sequence
  Synthetic polynucleotide
guugggcuaccuuuauccuu

SEQ ID NO: 1072 - RNA, 20 nt, Artificial Sequence
  Synthetic polynucleotide
gccaucuugcaacaauccca

SEQ ID NO: 1073 - RNA, 20 nt, Artificial Sequence
  Synthetic polynucleotide
guuagucuaugcauggguau

SEQ ID NO: 1074 - RNA, 20 nt, Artificial Sequence
  Synthetic polynucleotide
ugauagagcagcguggccca

SEQ ID NO: 1075 - RNA, 20 nt, Artificial Sequence
  Synthetic polynucleotide
gcuaagcaguggacuaagga

SEQ ID NO: 1076 - RNA, 20 nt, Artificial Sequence
  Synthetic polynucleotide
ucagauguuaaaggccaaug

SEQ ID NO: 1077 - RNA, 20 nt, Artificial Sequence
  Synthetic polynucleotide
gcugcccuugggagcccaga

SEQ ID NO: 1078 - RNA, 20 nt, Artificial Sequence
  Synthetic polynucleotide
ggcaaguuucaggccuggac

SEQ ID NO: 1079 - RNA, 20 nt, Artificial Sequence
  Synthetic polynucleotide
gcaaguuucaggccuggacu

SEQ ID NO: 1080 - RNA, 20 nt, Artificial Sequence
  Synthetic polynucleotide
aguuucaggccuggacuggg

SEQ ID NO: 1081 - RNA, 20 nt, Artificial Sequence
  Synthetic polynucleotide
gcuuucccugcccuguuugc

SEQ ID NO: 1082 - RNA, 20 nt, Artificial Sequence
  Synthetic polynucleotide
accaugcagggacugggcau

SEQ ID NO: 1083 - RNA, 20 nt, Artificial Sequence
  Synthetic polynucleotide
ccaugcagggacugggcaug

SEQ ID NO: 1084 - RNA, 20 nt, Artificial Sequence
  Synthetic polynucleotide
gcaucacuuucugagcugcc

SEQ ID NO: 1085 - RNA, 20 nt, Artificial Sequence
  Synthetic polynucleotide
ggcagcucagaaagugaugc

SEQ ID NO: 1086 - RNA, 20 nt, Artificial Sequence
  Synthetic polynucleotide
gaugcaggggccgcucucug

SEQ ID NO: 1087 - RNA, 20 nt, Artificial Sequence
  Synthetic polynucleotide
ucagaagaccucauggauug

SEQ ID NO: 1088 - RNA, 20 nt, Artificial Sequence
  Synthetic polynucleotide
ggaccauuagcauugccagg

SEQ ID NO: 1089 - RNA, 20 nt, Artificial Sequence
  Synthetic polynucleotide
gaccauuagcauugccagga

SEQ ID NO: 1090 - RNA, 20 nt, Artificial Sequence
  Synthetic polynucleotide
accauuagcauugccaggag

SEQ ID NO: 1091 - RNA, 20 nt, Artificial Sequence
  Synthetic polynucleotide
auguggugcaucucgaugag

SEQ ID NO: 1092 - RNA, 20 nt, Artificial Sequence
  Synthetic polynucleotide
ccucauccccuaccacccuu

SEQ ID NO: 1093 - RNA, 20 nt, Artificial Sequence
  Synthetic polynucleotide
cuagggccuaagggugguag

SEQ ID NO: 1094 - RNA, 20 nt, Artificial Sequence
  Synthetic polynucleotide
acuagggccuaaggguggua

SEQ ID NO: 1095 - RNA, 20 nt, Artificial Sequence
  Synthetic polynucleotide
ugaguacuagggccuaaggg

SEQ ID NO: 1096 - RNA, 20 nt, Artificial Sequence
  Synthetic polynucleotide
auagucaaugaaaucuccca

SEQ ID NO: 1097 - RNA, 20 nt, Artificial Sequence
  Synthetic polynucleotide
agagaaagacgagauggcac

SEQ ID NO: 1098 - RNA, 20 nt, Artificial Sequence
  Synthetic polynucleotide
auaaagggccugagcagggg

SEQ ID NO: 1099 - RNA, 20 nt, Artificial Sequence
  Synthetic polynucleotide
cuaauuuccucauugaaggu

SEQ ID NO: 1100 - RNA, 20 nt, Artificial Sequence
  Synthetic polynucleotide
aaaaaggcugcccgcugcgg

SEQ ID NO: 1101 - RNA, 20 nt, Artificial Sequence
  Synthetic polynucleotide
auuuagaagacgaugaugag

SEQ ID NO: 1102 - RNA, 20 nt, Artificial Sequence
  Synthetic polynucleotide
ucuguauuaauaaugcugcc

SEQ ID NO: 1103 - RNA, 20 nt, Artificial Sequence
  Synthetic polynucleotide
uuuagacgcugauguagcuu

SEQ ID NO: 1104 - RNA, 20 nt, Artificial Sequence
  Synthetic polynucleotide
agucccuaauauggcuaguc

SEQ ID NO: 1105 - RNA, 20 nt, Artificial Sequence
  Synthetic polynucleotide
cagccagguuacugauucau

SEQ ID NO: 1106 - RNA, 20 nt, Artificial Sequence
  Synthetic polynucleotide
accccuaugaaucaguaacc

SEQ ID NO: 1107 - RNA, 20 nt, Artificial Sequence
  Synthetic polynucleotide
acuaguauuccuguagaaga

SEQ ID NO: 1108 - RNA, 20 nt, Artificial Sequence
  Synthetic polynucleotide
guaaggcugauuugggcaaa

SEQ ID NO: 1109 - RNA, 20 nt, Artificial Sequence
  Synthetic polynucleotide
gaaccugagguuagcagcaa

SEQ ID NO: 1110 - RNA, 20 nt, Artificial Sequence
  Synthetic polynucleotide
uuaugcaagcauguguuuuu

SEQ ID NO: 1111 - RNA, 20 nt, Artificial Sequence
  Synthetic polynucleotide
caacagaauuuugucaaaau

SEQ ID NO: 1112 - RNA, 20 nt, Artificial Sequence
  Synthetic polynucleotide
ggaccaguaaaauaauuauu

SEQ ID NO: 1113 - RNA, 20 nt, Artificial Sequence
  Synthetic polynucleotide
gcauccuugcauagaggacc

SEQ ID NO: 1114 - RNA, 20 nt, Artificial Sequence
  Synthetic polynucleotide
gaugacagguuggccauugg

SEQ ID NO: 1115 - RNA, 20 nt, Artificial Sequence
  Synthetic polynucleotide
cauugacguggguugcacca

SEQ ID NO: 1116 - RNA, 20 nt, Artificial Sequence
  Synthetic polynucleotide
guacaaauugacgacacacc

SEQ ID NO: 1117 - RNA, 20 nt, Artificial Sequence
  Synthetic polynucleotide
ggacucuauuugugaggcca

SEQ ID NO: 1118 - RNA, 20 nt, Artificial Sequence
  Synthetic polynucleotide
acgugcagcauauguggcgu

SEQ ID NO: 1119 - RNA, 20 nt, Artificial Sequence
  Synthetic polynucleotide
caguguuucauaucuagacu

SEQ ID NO: 1120 - RNA, 20 nt, Artificial Sequence
  Synthetic polynucleotide
agucggccugcccugaaggg

SEQ ID NO: 1121 - RNA, 20 nt, Artificial Sequence
  Synthetic polynucleotide
acgcuccuggcagagggcua

SEQ ID NO: 1122 - RNA, 20 nt, Artificial Sequence
  Synthetic polynucleotide
cuacuuuagauucuauugua

SEQ ID NO: 1123 - DNA, 6136 bp, Artificial Sequence
  Synthetic polynucleotide
acattttactagtttattttgttgttcaaaattttctactcttggctattggaaggtcttttaattggctactatatcattttcgtatacacactcctagtataatggcatgaatccgtttatgaggctaaagccctcaaggcctaattaccttttaacagccccaccttttaatactgctacagtggcagttagtttccactaactgtaggggacaaaaattcaaaccatagcaagaacataccaaaaccatttttaaaaacaataatgcatttatttctgcaaaaaatggccataaagggccacctttatatgtgatcatgtagatcaccctctatatgataaggaagacaaatggagaacgaattagatagagaactgttgtgaattaaagtattaggtttggaatacaactttttgggttgattgctcatatttcttagaacagaaattctcagtgtacaactttaactctgaagaaagctacccatccaatgaagaagtagcaaagtgactgtttgccatttctatcttccttctcaatagatttattgaaacatagcatatacatcacctctccacatttgttgtacttttgttatttctagttcattcttgaggtttctctttaacacagcgactattaattctagcccagttggccaggagctgtggctcacacctgtaatcccagcacttgaggttgaggtgggtggaatgcttgagctcaggagttcaagacctttgggcaacatggtgaaaccccatctctactaaaaacacaaatttagctgggtgtggtgatgggcacctgcagtcccatcaccatactcgggaggctgaggcaggagaatcacttgaacttgggaagcagaggttgcagcgagccgagattgtgccactgcactccagcctgggtgacagagcgagactccgtctcaaaaaaaaaaaaaaaattctagtgctgctcaagaagcctctagaagatttgttcatggatgtttacattgtgcctttcagggtacacttttttaaatcttattttacagccaaatacataattatctgatttgtcagtggttttattctccattgtgaaatgtaaagtaatgatgcaacctataattgatgtgaataatttattttgcaattactatttatgtcagtatcagcaaatactacattcatttgtatagatgatattaatctacaggttttatggattattagttcatttcatcctcacagcatctcaagaggtcggcactagttttactcccaatcacctagtgaatttgatctaaagccctacaagaaaggaatggatgaaacttgattttgactgagtctatctttgaagtctttacttacatcacgtgttttagaatatctttttttgtatggaaaacacattctaggaccggattaagaaatgttactctagggccaggcgcagtggctcatgcctgtaataccagcattttgggaggttgaggcaggcagatcacgtgaggtcagaagtttgagaccagcctggccaatgtggcaaaataccgtctctactaaaaatacaaaaaaattatccaggcatgctggcgggcacctgtaatcccagctactcaggaagctgaggcaggagaattgcttgaactcaggaggaggaggttgcagtgagccaagattgcaccattgcactccagcctgggcaacagagcgagaatctgtctcaaaaaaaaaaaaaaaaaaaaaaaggtattctaccccggaagtcatgttcccaaattagatgtaaatcctgtgttaaattgatttaatatttaggtctaagatccacctaggaatgattttatgtgtaatgtgaggttatagatcagattttatttttccctgtggatatatttcttgaaatatttatagaaaaattcacatttccccaattaatcttataaattatttgtgtatatatgcttgtgtctgatttgctctctcctttttttttccattggtttatttctctgtgaaaatatcacactattttagtaagacacttcatttaacacagaattttctctctagggccttctcttagccaagtgtgctattatataaagaagaaagagtgaaaacctctatgccttgaacctacaccaatattctcagttgtcccttgaataaagagcagaatttagaactcactattactgatgacacttaggtctttaatgttcttgtgaatcttagacaacccccacaacatcattccctgctttatttgcttagtccattcttggtggattttatgagatgtcaacactcctcattaggattccctccttctgaagcctttgggattttctacccacccagcaaccaccagaggtggcagaaacccctgcagacatccttcttgcgcaagcgcagttgtaccatgaatgcggtcgtcctgctgcagaccacgcccaccaagggcggggccacatccactgcagctgagctcagagccttctgtggggtctgcagtggcaaggccagccgccccctgtcttccctctggagctgtttgaggaatgcagttcttttgcataagaaaggcttttctctccagtcattctgcttgctgatcaagaacacttaacagcttcaggctttcctggtaatattcattcttgcttttgtgtttttgttttaaggtccaaatatctttaatacttgtatgcagttttcttttttataatttcacataaacatcatctaaaaccaacttcgcatgttcgcattctgtattaacaactttcgtttttgtatttaaaaatacattcataatcactttttcaaaatatcttgtcagttattttcatcttttccatgatcattctttaccagtgcaatggaaatattacttttattaggaaaagcagatgtcacgcggtttttagtgttgtcgagaaatttcctatgaaacatctctggttgaaaaggagaggaacaaaattttatgtgaggcacaagatcacatatgctcagaatcgcatgctttaataaaggctgtgattatttgcattatattgtttagcttgttctaattgtaaaaggtagacaaagaattaatttgtgtagacagaacattgcgactcccattctaaatgtagagacagtcactgcactgcgcaaataggaatgtcgtcatgttgtttgcttttctccttaccccaaaactgtagacattggctagagtctttgatttttttcccaacttgcttttgtaattgtcggcatgatgtttgcacagataaccaatgatatagatatatatttttttcttactatttgatgaatgattaatattttgggttttttttatgttttcagtgaatcaaaaatatcattcagaaaatgttgtcatccatatccaggtagcatatagttttataatctgatcttgtaataaggaaattacagtccttattgattttctgttattagttagcattttttaagttttcatgcttgtgagtatttgggaattttttttttactctgtcaataatttaacacctttgtattggtcatctgggtcgatgtctattgtttaggctcaacctttgacactcctctgaggtagtttttatgtgaatttgtaaccacccaagggattcaccttgccagctgtctagacagagccgatttatcaagacaagggaactataatagagaagcaagaattcacccagagccggctgtgcaggagaccatagttttattattactcaaatcaatctccctgtagggggatcaggggtttctaagaataatttggtgggtagggggtcggaaagtggggagtgctgattggtcatgttggagatgaaatcatagggagtggaagctgtcctcttgcgctgagtcagttccccggttggggccataagatcagatgagtcagtttattgatctgggtggtatcagctgatccatcgggagcagggcctgcaatagatctcaagcactgactgagcttaggtttcacaatagtgatgttatccccaggagcagtttgaggcagtttagagtcctgcagtctccagctggatgaatcctaaaccgtaatttttaatcttgtagctgatttgttattcctacaaaggcatgatggaggggggttgttttgggaaagggctgttactgtcttcatttaaaagctaaactataaaccgaattcctcctgaagttcgcggggaatgaacaagggcagcttggaggttagaagcaagatggagtcagttaggtcagatctctttcactgtaataattttctcagttatgattttttgcaaagatggtttcaaattgatggcttagtgtcctgggtagtgaaaatggaattcctcaacctgaattgaacatggatttcctcctgaagaaacaatttagggctccaaaaagtaaaccaggagcctggcacagtggtcacacctgtaatcccagcactttgggaggctaaggcgggtggatcataaggccaggagtttgatactagcctggacaacatggtgaaaccctgtctacttaaaaaaaaaaaatacaaaaattagccgggcatgctgatatgcacctgtaatcccagctactcgggaggctgaggcaggagaattgcttgaacttgggaggctgaggttgcagtgagtcaacatcgagccactgcactccagtctgggtgacagagaaagactctgtggggtatgatgaggcttctcttcaaatagcctgatcaatcgtttattctttaattaatagtaacccccacccccttttcctttttctcttttttctttctgcctttgttgcatgcccggacatgccacagtagcaggcttatcagtaccagctcacattcctttccttatttggaaaggagactagctctctagctcattgcagacaccccttccccttttcccctctctcccttatgtgcccaccttatctaaagaaagttcaattgtctagccaaccaggattagttcagactgtgcgacctgacctccaccaatgggaaaagggtacaggggcaggacttggggcaggaataaagactcttgtgcccctttgttctggtgtgctttcatggcgactggccaaggagaagcacccctctgcgcagaagtaaaattgctttgctaagaatcctttgtttgagtgttcaatctcccttaggattttgagcattattcccaaccattcaatccaaaaaaaaaagtagaccaggattgatttagggcctgtattataaattgtacaaatgccaacaagattgtattccactagaggaacgcattctctagtgaaggaagcattcctgcaggatgagtccctgagactcactagcaaatttcatacaagtaatgaaatcctcagtctaatggaataaaaacaattccaggggcatttgttaaagatgtataagatgtgagaaaccaacagactaacagcattgatactttatggatatttttattccaaattaatagatgtgttacaggaaaagggtcctgatccagaccccaagagagggttgccgatattcagggccagtctgcagagtaaagtgaaagcaagcttactaagaaagtaaagcagaaaaagaatggttactccatagacagagcagccctgtgggctgctggattcccattttcatggttatttattgattatatgctaaaccaagggtggattatccatgcctcccctttttagaccatataaagtaactttctgatgttgccatggcatttgtaaactgtcgtggtgctggagggatgtagcactgaggacgaccagaggtcactctcgtcaccatcatggttttggtggattttagccggcctctttactgcagcctgttttatcagcaagatctttatgacctgtatcttgtgccagtctcctgatgtgacttaaaatgccttaaccatctgggaatgcagcccagtaggtctcatcctcattttacccagccctattcaagatggagttgctctggttcacatgcctctgacagatggacatttgtttgccaagcaatgctgcaatcaggggctctggatttcagtcctccatacagagaatgtcaagg

SEQ ID NO: 1124 - DNA, 5319 bp, Artificial Sequence
  Synthetic polynucleotide
ctgacaccttgactgaatccctgtgaaatatttcacctgaaaatccagctaacctgtgcccactgacccacaggaatggagataatatatttacattgttgtaaactgctaaattgactggatgtggtggctcacacctgtaatcccagcatcttgggaaaccaaggtggggggaatcacttgaggccaggagtttgagactagcctgagcaacgtagcaagaccctgtctctacaaaattaaaaaattagccgagtgtggtggtgtgtgcttgtagtcctagctattctggaggttgaatgggaagatttctttagcccaggagttcgtggttacagtgagctatgatcacaccactgcactccagaatggatgacaacgtgagaccttgtgatatggtttggctgtgtctccacccaaatctcaccttgaattataatcccatgtgtcaagggtggggccaggtggagataattgaaacatggggggtggtttcccctatactgttctcttggtcatgaataagtctcattagatctgatgcttttataaatgggagttcccctgcacaagcgctcttgcctgccaccatgtaacatgtgcctttgcttctcctttgctttcctccatgatggtgagggcttcctagccatgtggaactgtgagtccatgaaacctctttctttataaattacccagtctcagatatgtctttattagcaggctgagaatcaactaatacaccctgtattaatgtattaatacatgaataaaaaaagttgctaaatatgtagaactacactctgtgtcttccctttagtgttaggaagatcaaataggcactacaagagcattttcttggtaaatggtatctaaattccctcttatcaatggcaattactcatttggactgctaacaccataggggcaaaaatcctgacatgtatgaatcttgtgggagaagcacttggaaaaaaaacaaaaacaaaaacaaaaaacagagacaaacatttctgagcaaaaatgcaagctttttaaaaaaaatagtttgttttgatcagttcacatcatgtaggtcatttcatccggcctcattggcatataagtagtatatattgagctaggcgtggtggcttacgcctgtaatcgcagcactttgggaggccgaggcaggcggatcacgaggtgaagagatcgagaccatcctggccaacatggtaaaaccccgtctctactaaaaatacaaaaattagctgggcatggtggcgggctcctgtagtcccagctactcgggagagtgaggcaggagaatcacttgaacccgggaggcagaggttgcaatgagcgaagatcgcaccactgcaccccatcctggcgacagagtgagatctgtctaaaaaaaaaaaaagtagtatatattgaaagtataaaggggtgaaccactttgaattgcgtatgacaaaagaagctcagagttcaggtgaaaaatgtgttattacaaactcaactatcaatttaatctgtctttaatgtatgagtcaaaattaagtgttttctgttggggtgatcggacccaacaccaggccgtgggggcgatgaagtcccacggagtcaaaggaatgagaaaagacaagttaagagagaaactgggaccaggggccaatgctagtatggaggctgcgaaggccccaagctctgggaccccatgctatttattggtgatcaaacaaagaaacaggtggcgaggctgtgggggttgaaaggaagtggtgtatcaagcaaatgagctacagctgtgatggtttagcattttctttgaaacatatggctacttgagataatgggagtgctagaagcaaggagccagcaagtctagacacattccaaaggccacaaggggttttagaccctggaccccagacacgttccaagactcttttacattatgtcagacatccaggccctgcctcaaattctctcccaacactcagcttttctcccaacactttctattacaaacatcaacggaaaaatcaatgatctgtttaaaatattgtaaataccatcactatgtttgaagatatgttttaattttttattattcctttaattaaaagaactgtatgccaattaataagctgttgccttttagatcaaaattaatcattcattgcctgctctgtgaaaattgagtcagatacccttaaaaatatattgtgttgcaatagtaagttttttcagtaattgaaggtggttggacattgtaaaaagagattttcctttctgattccactgtggtctctcagccgtttcccatagcatgcatgcctttctccagcactatatttctacagtatgcatgagcaatcctgcagcaacaatggcttttctagcatcaggctcctacaacgcatgacagtcagcagcactagtggtcagccacttcctatatcccctacccttaggtggtttctagtgtagtgcttccaataatatacctacctgtgaacacctttccctggcacctaagtgggaagatttctggaaagttccactggtgccgcacactgtgactattctgtgtagcctctaacaagctctggattgcaatccacttatagtaaataaatgtccacttcaaacattccctcctcaaattcttgtgttttgtctgcctcctgattggaccctcactgaaagaattaatgctggcattgttccaagagatatttccacagagatgaatctgggaggttgtgttggtcattcctttatgtttgggcacattccacagctccttgtcaatgtgcaatgggatactagcaacctatgacatgcagtggtatcacaattaatcaagcaatgacctgtggttgattgtggcaaagtgccaactgaaacacgtgccttgggagcccgagtgactgctgcactgaatgttatggcagtcaagataactgtaaggactgtggggtggaatggattcagttgagggtccttgagtgattacgaacagaaaataacaagcttaggttccttaattctcagttcaagccacggtttgaaaacctgagaacttccatggcgactataaaggaaattcatttgttgtagctgcagggctgatattgttaaatatcaaaaccaagaatgagatcctgaaacttggaatgggtcaggaaacttggaatgggggaatctgtttagactcagttgaaactgaccatctggaaacagaaagtcaggctgagcctttcttaccagtttaagtagcttgtctctattgtttgaagaaaatatctttcccttgcacaaaagctgggttagataccattaaaggggatgcccatttttcttaaagtcccctacaaatcatcactttttgctagtagcatgactagaccccataactaggattgaatagcaacctattcctaagggataggtacacactatgacctgtaggaaacagctacatgcaaaaggaactgtgacatatattggaagaaacctggggaatgttggaagagcattctaaggccgttatactaaggaggacaaaatataacagtagattgggttaattaatctcagtgctattactggggattcaagatttattgtgctgcaggcatgtggctcacacctataatcccagcactttggatgccaaggcaggaggataagatgagtccataaatttaagaccggtttgggcaacataaagagaccttgtatctacaaaaaataaataaataaataatccagtgagagtggtgcacctgtagtcccagctacttgggggctgagatcagaggatcacttgagccctggaggttgagggtgcagtgagctgtgatcatgcaactgcagtccagcctgggcaacagagccagaccctgtctcaaaaaattaacaaaaaaagatttattttgtagttctcctggtagagatctttcatctccttggttagatgtattccttggtattgccgtcttaggtagaggaagagggctcaaacatcttgcttatgtgataactttgccaaagacacaatgtacaatttgttttaaaggccagtgagggatgtaccagtaatcagaatgttttggcaaacagaagactttggtggtagctaactgatcacagggtccctagaatgaaatagatgagaagcatacttagagtattgtttgatctctatatcaagaaaagttccgggtctggtagtcaaatgttcaacagatgcctacctagctttcaaccctaagtcaattcacagttgcagagtgataccacacggaccatgaaaagaacacttgtttgtatgactgagacaaaaaagaatgtcatcgtgttctaccttacctgggaatatgctcaattggtgacaaaattgttgccactctgtgcatgaccacaaaagtaccgcaggtattgattttgaagttacacataaactttagtgagtgggcatatttgcaaatatgtaacctgagaataatgaagattaactatctttaaacaacataaggagtcaacatcagtttctgaagtaaaataatgactaagttaaattttcaagggcaaattcagattccaagtgcacagaacataggaagaaaaggacatgcaaatcagaaaaattcacaagtgtaatgaggcgagaagggacatggcgcaccaattacaaaggaatcagaatgatctgttcatagagttgtaaatggggttgtggaggaagaaatgaggatgaacagataggcaagacaagttcttttccttctatgtaaataagagaaactgagaaattgtaacgtaagacaagatctaatattatttttggacttcagaatggtattgcataaatgcttaattgttcctgatttcgggaggggaaaatggaaatagcatttccgttgcatttttgtgaacaaaatttgagtgatcatgttctttgttgcatgtggtagatggagaggtgaatgaaagattcagctttcttcctgtacttgaggcatctgtgaattgggaattcagacaaacagataagcaactaattctggatataaaatttgtgatttaggctatgcattctactggggaatttcagatgtataggaatgtagttgaaatttaagtggtcagggaatgctatctagaggaagaaaattatgactagagacaagaataaatagtagtaacttaaaaaaatatatatcagggaagaaaaaatggatttttgttccaggaaagaaactaagtatgaacagatgatgaactatttcaggaactgtgtctgcaattcgttccttccag

SEQ ID NO: 1125 - DNA, 801 bp, Artificial Sequence
  Synthetic polynucleotide
gttttgctccatcacccaggcggagtgcagtggtgccatcacagcttactgcagtctccacctctcaggctcaagtgattctcctacctcagcctcccgagtagctgggaccagaggtgtgcaacaccacatttggcttttttattttttattttgtagagacgagtcttgctatgttgcccaggctggtctcgaactcctggattcaagcgatctgcctgcctcagcctcccaaaggattgggattacaggcttgagcccctgtgcccggcccctatgttttccttttttttttttttaaatttgaaacggagtcctgctctgtcgcccaggctggagtgcagtggtgcaatctccctcaccgcgacctctgcctcccgggctcaagggagtctttagtctcagtcccaagcagctgggactgcaggcgcccgccaccatgcctggctaatttttgtattttttagtagagacagagttttaccatgttggtcaggctgatcacgaacctcctgatctcaggtgatccacctgtctcggcctcccaaagtgctgatattacaggcgtgtgaaccaccgtgcctggccgttttcatttctttgtgatctgcttaggagtagaattgtaggtcatatggtaacttcagggttaacatttgtaagcattgccaaactcttccactgtggctgtgctgtttcacatcccaatcagcagtgttagagggttccaatttctccacctcctccacaacccttgtcattgtctttttaattctagccccaagtggcatctcattgtgg

SEQ ID NO: 1126 - DNA, 3197 bp, Artificial Sequence
  Synthetic polynucleotide
gtgatcagagtcaacttcccatgttattttatgtgaatatgtatcaatgaactattagaaattgcaatgtttttgtaggtcaaaacaattcaatattgacaattttatatgattcagcctaatacatcattttttccatcagccatattgcatgttattatttatatatacgtttatttagttagcctatctgttctctgtgaggaacattacacagtttctgagattttgttacgatagaagtcagcattgataagcctctgcgcacctgttttaatgtaggatcattgggaactgagcagtagcttacagggcttgggaggggatttaaccatttagtcaacctccactcacatcaccctcccttcttcgataatgggcaattgagaaagggcaaatgtgtgtaaaaactatgcagatgcagtttcaggattattttcttcttttctgcagtgtcttttcccaagctacatcagcgtctaaattttggagttactgatgcttccgttatttctgcagatagaatattatgtcgtaaataatgtttacttgtccattagtattatataaaagtgcctgtttctccccactccagctatcattgattattggtaaaaattttaatgtatccaattctaataggcaaagaaaggctctcctacttagatcttaggtgggggtctaagttcatatttttgttttacaaaaattgaatagccagtgtttttgagttgaatgatctatccattccgtataggagagtttaattatattgtgattttgggggagataattttcacaatttaccccctcaaaatgtagaaagtagaaaaagggtagaaaaatcgcaccaaatttgaacaatgtagcatgcttttagagttagggatattttcttttggaaggatttcaagtctggcgtatttgcattgattgtggttatggcgcatttaacttttccaaaccagcttttttgtaccgcacataggaagacctgagggtgagtgtaaattaggattgtttggccagtggtgtgtgcaaaaatttattgtagaaccaagaattatttctttagtattttacacattttaaaaaacaggtagacatgtccattgatcccaggttgcttatggtttctagaggccccctctcattgcaacagtgctgtggggccctaggggtccagtagccccctccccccaggtcattccggtgagggagggagctgggacccctgcactgcggcaaacaagcacgcctgcgcggccgcagaggcaggctggcgcgcatgctcaggcggggatgtgtgcgaagcctgccgctgctgcagcgagtctggcgcagagtggagcggccgccggagatgcctgacgcatctgtctgaggagcggtcagtgacgcgatggagcgggcaaggtcagctgtgccggtggcttctctcaagagacagcctggggagcggccacttttattcatcagatattccaagtttttaggacttggagtactgaataaacggaatttgggccctaaagtcctttgttctggagaaccagatccggaatgttcagaggcttgctgttgtgccgttctgccccgatggtatcctgtccgctcgcattggggcgcgtcccccatccgcccccaactgtggtgtcgcgacaggtcctattgcgggtgtctgcggtgggaagggcggtggtgactgggagcatgcggggtaaccgcagtgggcaagggacatgggtggaggtgggtacatcagggtgattgcagttccgtgtggcgagggtacgtggggggaccagtgcatagggattttaggcggaggtaggtatattggagtgattgtggcggggcgaaggtacgtgaagtgatcggtatttagggggtgttgagcgcaggtaggtgtataatagtgaccactgcgtggtggagcagggtacgtggggcgactggggggggaattcgtcgcagtgaccgaggcgaggaggctatggcagtggaccagggggatgagtcggtgtgacagtggtgggggttgtgttggtggtcgcggcgcggggagagtccgtaggaagtggctgggaggtagggggaaggcggcgacagtgggtattggcggcggtgggcattggcagcgggtaggcatggcggtggtggactttggcggcggtaggcatggcggcggtgggcatggcatggaggcggtgggcatggcggccgcggggcctgtcgctgtccggagtgactaagggacgctgaatgattgctgtgtagaggagggggcatcagaaggggcagtagcacagtccgcagcctttaattttcctgtgacctgcgatggtcatctggtttgcctactgtggtggtggtgcttttttattaaaactgcgcaatgcctacactgccgcaggggctgcagaaatgcgtggaatccttgcgtaccctttaggaaccctcgctttagaggctatggcataagaagactggaatgatgtgcagaacttgcatttacagatcttttgactgataggccaggtgatgcctgggggttacttttttttcattgtttctctctgttgggaaccaggtcttggaaggctatgtcgaaataacctgggggtctgtgttgcgtcactgccattgtgcagctccttcaagatggccgccgctgcagcggcttagatctgcgcaagcgcttcagcgggggggtggccgcttcctccctgtagagccgccagtggggagggggcaggtggtgtgtgttcagcttctgctgtttcggagtttcagccgtaccctctttagggtgcagtggtaaggagaggaagccggccaaggtgggcggctgccccctccccaaagtgctgcttgggaaaggatgcaggttgcgcagacgcagcagaggtgacagtcgcctccgcttgcagtggggcgagacgtggggcaggggagcgagggcttacgcagcttttgcctggaaggaatggcagccctccccttcacctggctgatacagcagctgttcctttccggttgtggcgcagtagaggggggaggagggagatgggttggcgcaggctccgcctagcaagcttggcagccaccccttacccacggtgcagcagtagaggactggaggggaatgtggctagaggccaggcatttgtaccacctccgcctgtgtgggatgg

SEQ ID NO: 1127 - DNA, 20571 bp, Artificial Sequence
  Synthetic polynucleotide
gggatcctaatatggcagatccctttggaaaggcctcttgaagtttcaaggtctgtgtcctactcagtgaattggctctgtgctagggctttttggaggcagggagtggcagcccttcctgtgatgcagaggaaggaagcttggttgagagacccttatgttgttgtctaaggcagggtactaactgatttggatttattcacagaccttgccttatcttgatttcactattctatctgaccagtgtgtcattgccgaaatgttttccacatttatagttactctgtatctttatgtttcccttaccctgttccttttcctaataaacatctttgttttacaaaaaattttgtttataaggtgtcaaggatgccaagataacctcctttgtttatcagcgccctctgtttagctattttttaggaagaatcatttaagcaggttgtgagtactgccctcaattacttggacaatgaagttttaaaaatcttatttagacctgtcactggtcctgtctcctttttttttttttttgtggcccagggccagaaagcttgagtgagaattgttactacttaaggtagcctctctccatttctggatgctagctggctcaaagttttttttttttttttttttttttttttttttttaaatagaccagggtctccctatgttgtccaggctgaactcgaactgctagactcaagctatatcctctccctcctcagtctctctctggccactgtggctggcccaaattctctttttgttaattctcctcagatttgttttggaggcaatgaggtttcttatattcatggggtacagattagcagcctatcattaaccttatttttttgtttttaggagacagggtctggctatgttgcccaggttggagtgcagtgatgcagttatagctccctgtaacctcaaactcctggatcaatctagactcctgcctcagcctcccaagtgcctgttagtacaggcttgaggtactgtgcccagctaactttttatttttttgtagagaggggtctccttatgttgcccagactggtctcgaactcctggactcaagcagtgcccctgcctaggcctcccaaagtgctggcattgcagccatgagccgctatacctggccaactttgtttatttttattcttgagatcttagttaaattttatggaaaatttaggccgtaataaaattgggattccattgctttttgcttttcaatatatttccggattattactttcagtgtaagtgatctgagggcaaacatttaacaccctttctatatgatgattttttcatgatgtcctcagtttcataaaatgttggtttctgtgttggggatgcagttagataaatgcatgtgtgtgtgtatctagaacattcaataaagtagtgttttcaaaatcttatagatgacattttatatgtagattaaaaataagtagatttaagccaggtgcggtggctcatgtctatagtcccagcacttttggaggccaaggcagaaggatcactggagcctgggagttcaagcttgggtattgagaccccgtctctacatttaaaaaaaggtaaattcagaaagttgaaataatagaatattttggtttgatggctatttcctcagttgacctcagacctttcatattgagcaactgccgaatgggttctttttgttttttggtaagacagtgtttgcagttgtacactattagattaatcatggtttagttgatagtttctttaattgtattatattaaggtactacattattagctgtatttgtgtgcccaccataatgtagcactgatcagtactaaattcctttttatggctgaaagacattcgtttggatatatcatattttcttcattcgtaagttgatggacatttgagttgtttccactctttggctattatgaataatgctactaggaacacgtataatacgtttaatacaaagaattagtttcacaaggttttacttagccctcgtatatgccaggttcttgaaattttgtctttttggaagtatgagagtagtggtagctgggaaggctgcaggtataatagaaagaacatgaactgtctgggcatggtggctcatgcctgtaatcccagcactttgggaggccgaggtgggtagatcacctgagggcaggagctcgacatcagcctggccaacatggtgaaaccctgtctctactataaatacaaaaattagctgggtgtggtggtgcatgcctgtaatcccagctactttggaggctgagtttgggtcttgtaatattacttcatctagcagtagagttagtgtcatatgttactttcatgattaacatttttaggaagttggcaaagtctatatataacactttatgtttccaccagcagtttccccacatcctttacaaatattgtggtgtcagatggtggcatttcagtgggggttttttcattttgaggcagggtctggctctgtcacccaggctggtgtgccagtggcatgatctcaggtcactgcaacctctgcctcctaggctcaagcgccctctcaccttagcctcctgagtagctggtaccacaggcatgcaccaccacacccagctaatttttgtatttttttgtatagatgttgcccaagctggtctcctgagctcaagtgatcgctggcctcagccttccatgaaccattgcgccctgcctcattgggattttgatttgcattttttagtgactaatgttcagcatcatttcatatagattagccacttgtatgtgtttggaggactgtctattcaagtgttttacccatcgtagcaatagaatttgcccttttattgttgagttgtgagagttccttacatattctggataccaaacttgaaaaaatacttttttgaggtgtgagtgacatacaaaagctgtagatagttaatgttaaaagttgatatgctaagagataagtacatgcctttgaaatcattcttatcaaaattagtggcataaatgtgtctgtcacctgaaaaagtttctttgcctctccctaccttcttttttttcattgtttggattttctttttgtttgttttggatactagaaacttagctgtatgttaagcaaatattttttcctattctggttatcctttcactttctttagtgttttcggaactttattttttgatgaagtccactttattttttatactgcttgtaattcaggtgttatatttaatagaccattgtgtaatcaaaagtaataaacattatcagtgtataaaattttaccatttagaaatcaaatatgtgtgtatggcttgattagaacatttaaactcttgaaagaataaaacagctgtgcgttattggtaagctattttaccaatctaaaagtctaacatgacttaaaaacatctagggttcagagaattattaatagcttttggcatgtatctttgaattttgcttttgagatattacttgtgttactttagtgaaaggtcctgctgacctttctgatttatcatctttaaaggttcataggtggtttctgcagtccctaatatggctagtctggtgtgggatttcatgttctgttcccttgagcagccattttatgaggggttctgcagacgtaacatggcgggttttttgtttgtttgtttgtttttgctatgtggatagtttttagcttgagcagctcttctattgctttgcaaatcctaaagaaagcaccatttgcatgtttttttaaagtagatattttataatttaaactatgttctattccactatataagtatgtctactgtatcctaaaaatattttttgaaaccattttggaaatcaggtttactgtttttggggggaaggtgagtagggtagtggtggtatgtatgtatctacatctgcataagtgggaattttatatgtacgtgactacaaatagaggtagatatattattttaatttcattttctgattttttttaaaaccagattttctgatgtcatgggcttctaaaaggagaaaaaagtggttatagcatagattatgactgtttgaaggttaaagatcttgtaataattttaccttgttttaattagatttaaaaatccattatattaaatgtagttctaaatataaccttgacaaatagttatttcatagattgatgcagtctaccaaacaaatgcctctcttttctgtttcagggatcgcttacacctgagacgaactacagaacagcacgtaccagaggtggaagtccaagtcaaacgcagaaggactgcctcactgagcaaccaagagtgagtacagactgtgttgggaacaaatgcaagtcagaatctcctttcagattagaacaaaatatcatgcaatgaggggattaaaatgaacataattgaagaagcagacacatctaatacagaagatgtagtaaaaaagcaatgaaattcttaaccatattatcctaaaataattcaccctcttaaagattttagtataattttcttttttcctatacgtgggtaagtgcattttatctttataaaattttgtaattacccttcaacataagcatacttaaatttttctgcattattaaatcactttgtatgcttgcaaaatgtactgtgcgctgaagtgattaatttacatattcattcttgctctgtgatatttaggtgtttgtttttggttagtgcatttattctgttaaaatttaaaatacatttgtgttttttatcctgttttaagtatatatatgtggaatgtatatataggatgtatacacagacacactcagacacatattttaatttagatttctagaagggaaattttaattaaaaataggtgaacattttaaatgacctaatacatatttagtccacattgaaactttggcattttgtcattgccattaaaattttgatggcattaaaatttgatgccattaaaattttgatcagtaggtagcatttttttcttagctacaattgttttttttaattataagtattaaaaattcatgaagatgattctttttgtaaaacagttttgcataaaaagtaagtctcattttaaagcaactaccaacttaactggccttttcctccccagagataatcattaagtaaatagccatcagaccttgtcctatgcattgtgtgtgtagtggggagagaggaagggaggacatgggacacatttgaagtttgtgtgcatacgacaccgtgctgtctgtattttgacaatatgtcttggtgatcttttcatatcagtacacataaatctaccttattttacaaagtgtgcgtggtgtgataacattattattgtaatcgttagcttgctgctggatatttagtttgtttgcattgtttggctattaacaatgaaaagaacagtcactttgaaaacgcagtgtaggccaggcatggtggcccatgcctgtaatcccagcactttgggaggctaagccaggcggatcacaaggtcaggagttcgagaccagcctggccaacatggtgaaaccccgtctctcctaaaaatacaaaaattaaccgagtgtggtggcacgtgcctgtaatcccagctacttgggaggctgaggcaggagaattgtttgagcccaggagacggaggttgcaatgagctaagatggcaccactgcactctagcctgggtgacagagcaagactccatctcaggaaaaaaaaaaaaagaaaatgcagtgtatgaatactaatttattttatttttttcaaattaaaggtttttcctcatagtaaaaactgagtagtagtttgtgtgtcttttgttatttcctcattttatatgtgaaaagttgagctggatgtggtggctcatgcctctaatcccagcatgttgggaggctgaggtggatcacttgagcccaggaggtggagaccagcctgggcaacatagagagacctcctctctacagaaaaataaattagccaggcgtggtgttgtctacctgtagtctcagctacctgggaggctgaagtggaagggtcacttgatcccaggaggttgaggctgcagtgagccatgtttgtgccactgtactccagtctaggtgacagaatgagatctagtctcaaaaagaaaaaaaaagttgtaaaacatctgttagcaagttttaaattatggtgagggtaaagattttttttttattttttcctatttattttgccgatttttactatagtgagtatatttaagtacattttcattttttaaaattgctttggaagacgtattttatgtacgttttgtttgtatattggtaaatattttgaaagtattttgtgtaatataatttgtcctttatatataataggttattcgatcattcattttatttgatatgaaatctcttttttcatctcttcattctttttgagacggagttttgctcttgttgcccaggatggagtgcaacctccgcctaccaagttcaagtgattctcctgcctcagtctcccaagtaactgggattataggtgtgtgccaccatgcctggctaattttgtatttttttagtagagacagggtttctccatgttggtcaggctgggctcgaactcccgacctcagatgatccgcccgccttggtttctttcccaaagtgctaggattacaggtgtgagccactgcgcccagcctctcccttcattcttttgcagctctgacgtttttttaaagtgccattactattccctgagtttctagaggtagttcagagatggtggtggaggtagggctgaggtttaggactcttaggtttaggactgttaagtgggtgaaggttctatcttgctatatttttgttacagtttttttcctatgtaacctcagtgtggactaaataacaggtagtctcatcctcatcttgttgtgggatgacgttagccttctaggcatcttgtccatatttatggaaacattttgatggttttgaagaagaaataacttgagtttttcttatctgcactgcatatatatgtacaaatactgcagggtcttgagttgggggaatgatctgagtaaagtgtagagttagggaaatagtataatttttcccccttgtgcgttttcattttgagagtaattggattatggaatgggtaagcaggattggttaacactgcctcattgtccatgtgtttaggattctcaggattggtatacttagaatgttatactagaagtgttcaatatttacccattcatctcctacttcctaccaaatcataagtagtattgggaggcagtcttcctggaggagttgtgtggttattagccctaaagatcaatgaaggaggctgggtgcggtggctcacgcctgtaatcccagcactttgggaggcgaaggtgggcagatcacctgaggtcaggagttcaagaccagcctggccaacatggtgaaacctcatctctagaaaaataaaaaaatcagccgggtgtggtagtgtgcacctgtaatcccagctactcaggaggctgaggtggaagaatcgcttgaacctgggaggcaaaggttgcagtgagccgagatggtgccattggctgtgcctgggtgacatagcaagactccatctcaaaataaagtaaaaataaaaagttcactgatttattttaaattttttattctgtctttcagtgtatttgtcactaaattagctcttattctttggggctggatgatgtatcgtgagttcgaagggaaattggaaattggagctggagagttgtcttaatatattttatgctaagcttcacatgtcttttcaaattaatattctgcactctatgaattaaattctgtgtaggctgcctcattttctcagtctttaatcttctattgggttacaggttagatctgagggggacattgcaatagagaattatcttacgtttcaagataacattatatatatttttttttactgaaacactttttagacctttatgaataataatcagaaacttagttgtcatttgtaaacagaggttagagtggagaatgcccaaattaagatttatttattcagtgattagactatgaattagagtataaagtagactatgaattagagtgtgaagatttagaggtatgtgcagtgcaattctgactacttggagttagtgcaaatttcacagttgtctagatgccctccagaacattcccctcaatgcagacaccaactccaagctccagggttggcagggcacccacacttccgaccaacagtctatagattctgaggtcctactattttctcagactttataattgactaaggttttataaatgactagtagatctcaagaaagtgctatacttactagttttttttataaaggatacaaatgaacagatgcatagagcaaaatctgggcaggtcccacacaaatcttccctgtcctcaggatgcattacctgcacattagcgtatatcaccaatcaggaagctcaccagaactccagatgtcgatgatttttgttggggattcattatgttgacaagattgaatcactggacatgtcactgaattcagtcagcaactctcctgccctcccaggagggcttgctcaaaaccccaaccctttagtctttgtttgatcttttgtggcctggttagtcaccatcttgagacatctcattaccaagaactcaggtgtgatcctagggacccaccttgagtaacaaacacatttctttcactgggtaaattccaaggatgtataggatgtataggttacttcccaggaaccaggagcaaaggccagttaaattctttattatacagaagcaattttaaaatgttactgaaaaaccatagaaaaaagtccaatttccaaatccttacatgccaacaagggcagcattgttggtgatcccccagctcctcacctcaaggcaagctgtcatttcctgtatgtcttccttctcatcatcagatttcacagccaggttactgattcataggggtatgtcgagggcttttgtctggaattccctgcttaccagtagatgattggtttacatattttaagagcccttaaatagccctggaagttctatatttcaggtagtgacttgtcaggaggatcaatatgaaggacggcctaaacatctccctgcaaatgctttagcagcatctccctcccctcacccctggggagaaaggattgttcacacaggataatttgaaagtgagtgactatatatggtactgatggtgtttcttcaaggctgccagctcttgtgtttttagttcttttggaaatatttctagacttggtaagtatcttaagtatccactagtcccttgtacaatctctgtgtagagattcctgttttgtgttataatttctcataagatagtgtcatcctctgaccctaaagaaagatcttcttccctgggcgtggtggctcacgcttgtaatcccagcactttgggaggccaaagtgggtggatcacctgaggtcatgagttccagatcagcctggccaacatggtgaaaccccatctctactaaaaatacaaaaaaattagccgagagtggtggcaggcacctgtagtctcagctactcgggaggctgacactggagaatggcgtgaacccaggaggcggagcttgcagtgagcccagatcgtgccactgcactccagcctgggcgacagagtgagactctgtcttaaaaaaaaaaaaaaaaattagctgggtgtggtggtaggtgcctgtaatcccagctacttgggaggctgaggcaggagaatcacttgaaccccgaaggcagtgtttgtggtgagcagaaattgcttcattgcactccagcctgggcaacagaatgagaccctgtctggaaaaaaaaaaaaaaaaaaaaggaatatcttcttaaatgtaagggtacctagttttctttcatatggtttcctataaagacaaatgtatttttatcatttatatatattgtgctcttgttgtaggtgtcagttgtacccgaggcgttctcagcagcagcaagtacctgtggtggatttccaggctgaactgaggcaggcattcttagctgagacaccaagaggtggttaaagccatattggagtagcgaggaatctgattccaagcaaaaaccaggttagagctaatgcagcaatgatcaagaataaagaatcattaaagaatggggtgttgggggttctcttaattatcagtgacacattaatttttttccttagagcttcatacaataaacacattgcagaaagttttagaaagttttgcaggaccttaaattttctgccctgacactttcgtcatgtttctgtattccagttattgtagcgcatgcttttcaggtagttttcttttgcgttttttttaattatataaaaatatttcactataaatggctcttgaataccctgttactgaatgtttataatttccttttgtgggaggtgtcatttttttctgccagtagtttgaacaaagctttagtagatagctttgtataaaacttagaataattttttcaaatatgcagatgtgaaaataaattgaatcaaggaatatgaacttaaggtacatttttctaaattatattgcaaagtattaaattagtattcattgctttcgaaaaataaagggccagttacaacatactttcacagcattattttaattcaaatatggtttatgtcagtgaattataaatacacagcacataattaacaatgagtattagagaatctaaatactttctcatgcttatttactaattcctttttgttttgtctttttctcacttttctgttttaaccaatgtatttcctctttgaatcagcagacaatgtaataaaaatattattcataatttttcaatgtgatgtagatttatttagttctttttatgttgctttcattttttaggaaaaagttattacaaagtcgattttgatttgttttcttgtttttcctctgtgatttttctttttttctactttaagttttagaatgccctcatttctgagagtttactatttactttttacaaagtaagagcagagaggaattgggtttactgataaattccttctagcctttttttaagtctgtttttgaagtattatccttcaacttgaatcagttaggcttgtttatggccaccattttgagttattaattaatttatttatttagagatgaagtttcactctgttgcccaggctggagtgcagtggtatggtttcggctcactgcaacctctgcctcccaggttcaactgattttcctgcctcagcctcccaagtatctgggattataggcacccaccaccacacctggctaatttttgtatttttggtagagacggggtttcaccatgttctccaggctggtctcaaactcctgaactcaggtgatccacctgccttggcctctcaaagtgctgggattacaggcgtgacctaccgcgcccggcccattttgagtttaaatcaaaggagacagaaattttctgagaaatcctcctttcttgaatcctttatacacaacattttctagcaattgcataagcaaattggaatctaatcattgtccaaagcttttcccttgagtaagggaatttttcattgaataggtgtgcacaactgattttttactttttaggaattttttagcttctcagttcagttcacagatttttgtaaatttttaaatacaacatttaagtcatacattatagtctgcaagtaggaccatctaattttatgatacttaattatatcttattctagtctcaactgctagttctacctaaatagtacccattactagtattcctgtagaagaagggttggtaatatatctggaccactacctgtgtctataaatgaaattttattggaacacaactcagttcatttattatgttatctttggccacattggtgctacggtggtgcagctttgagtagttgtgacagattgaatgacaggcaaagcctaaaatacttagtatcttgctatttaaaagaaaataattggctgagttttattttagaggagcagattgaaagaagaaaacaccaaagtgggggccagtcaatatagtcataatgtcatagaaggtgataaagaatagattatagaagtatatacgaagtatacattacagcgtagacaatcagatgaatttcttggggaagggatcagtctaagagtcatgttaaagtctgctgtggaggaagatgggtagtctttaacaacatttaaacacttcgtacccgttttcattaaaaatgtaaggcacacgatggccaggaaaaataagaaaataaactattaatgtggccgggtgtggtggctcacgcctgtcatcctagcacttggggaggctgaggcaggtggatcacctgaggtcagaagttcgagaccaacctggccacatggcgaaaccccatctgttctaaaaatacaaatattagccgggtgtagtgggggacacctgtaatcccagctactcgggaggctgaggcaggagaatttcttgaaccctaggggcagaggttgcattgagcagagattgtgccactgcgcttcagcctgcgtgacagagcaagacttctatctcaaaaaaaaattgtgttatgaatattctgtttttctttggtacctaaatatttaccagggtacatatccaactggatacctacctagtagttgggatgcagggtgtaatattagtatgacttttaaacgtttttgttacattgttttctaaacacatcatgagaagtgctgatggccactaaaaattagtagaccattagtttctcagtaccaccctttgtattctggtgtttttttgtttttgttaaataaacctttccaataaaggaaatttgttttcttgtgtttttaattggaattattgtgtattaatattttgcatttagttttcttttgtcacttgtttttgcatactcttcgatggaggatctctatggagaccctctataattttaaattttaacagatcaatactgatattccatggtgtgtctcatttgctatgtgtgccttggccaaccctagtcatcttattttactatttgctatgtgtgctttggccaaccctagtcatcttattttacttttgaattaaagtagatttacatttttatgtagtttaatcatttgaaagtctacagtttcctaaagtttgaaaacttgtgcttctgtgagggataccagttcgttaatttgcttatgtgccctttgatctcagaagatacactatgactgctcatccatccttgcagtgtgatgttgctactctcgtcgcctttctcatgaagttttgttttcttgtgctgtaaagataaatttgaccactctgcagtgatttattttcttctttcacttacaaggtcatgactacattgtatcacatttagggatgagagagaactgtttctaaattccaaatctgtctcttaagggaatccttttaactgctgtaggaggtcaactcctattgtgagtaaaacatatatctcttctagctatacaggttattattatattatgatatatatataaatataaaataataatctgtccctaattctctaggtcttcagttctttgggccataactcacatccctagatgtgtttcatacctctggaccgctaagcttgtgggagttatttatatcctattgctcaaggtcattgccaaggtctgatttttcacaaaaaaattccaacctctggcataaatgggttaaaaacggctttagtgatgtagtgctgataaacataaaattcacccaatttaagtgcatgaagcaatgatttaagtaaatttacagaactgtgcaatcattgccatgaactactctggaaacattttcatcaccacagtaagatctgtcttgctcattcacagtcatgatccttgttgtctctttggatggctgaaggtcccaagtgttgcagtgctataaaaatggatattgctggctgggcgcagtggctcatgcctgtaatcctagcacttggggaggctgaggccggcaggtcacctgaagtcaggagttcgagactagcctggccaacgtagcgaaattccttctctgctaaaaatacaaaaattagccgggtgtggtggcgggcgcctgtaatcctagctactcaggaggctgaggcaggagaaccacttgaacccgggaagtggaggctgcagtgagccgagatcactccattgcactccagcctgggcaacagagtgagactctgtctcaaaaaaaaaaaaaaaaggatattgctaatgagtctcatataccatgttttcttggatttagactgctttataaagaatcataagataatattcaaaacttctaagttatagtccttaaactcttactcatatttaggacagaaactacatttaatttttgtggaggtctttgtttgcttatttgaaaagttcttttgagatctttataggttttatttaatacattttttaatgtacagacatcagattcatggccactgaatgacagccaggcattagagtattctttttttttttttttttttgagatggagtttcactcttgctgcccaggctggagtgcaatggtgcgatctccgctcaaaaagatacttttgaatccttttcaaaagtaatttgtgtttcattttttaattagtatgataagcacttcaagaccctttacaattctggctagatttggttgccatatttaatagcattttataaataatggagaaaagggaaacattatgcatgcttgattacggtcatgagctgcattaacaattccgttcaacaacagactgcatatgtcagagcagtcccttaagattacaaaaccatgttttcattgtgccttctctctcttccccaccacccccaaccctgagatggagtcttgctctgttgcccaggctggagtacagtggtgccatctcagctcactgcaaactctgcctcctggtttcaagcgattctcctgcctcagcctcctgagtagctgggattacaggcgcctgccaccacacccagctaatgtttgtatttttagtagagatagggttttcgccatgttggccaggctggtcttgaattcttgacctcaggtgatccgcccacctcggtttcccaaagtgttgggattatgggcgtgagccaccacacccagcctcttctctgtttaacattgtattgtgagtgttcatagtatgcaggatagtaacatgatatacaggtttgtagcctaggatctaccatattgcctaagagtatagtagactctgtgtatcctctaggtttgtgtaagtacactgttcatactgtgatgaaatcaccaaatgcatttctcctaatgtgtccccattacgtgacttatttatttattttgagatgaagttttgctcttgtccctccttgttgcccaggctggagtgcagtggcgcgatctcagctcactgcaacctctgcctcccaggttcaagcgattctcctacctcagcctctcaagtagctgggattacaggcacccaccaccatgcctggctaatttttttgtatttctagcagagacgggatttcaccatgttggccaggctggtctcgaactcttgacctcaggtgatccacctgccttggcctcccctggtgctgggattacaggcgtgagccacctcacccggccgttaacgtgatttataactttgtccttcattacttgatctatgaaagttgtttatgtgagtctatatatatatattccatagtcatgtgttctcttttgacatgggaagtcttaggcaggtttataacacacttggctcttagtggggaaagattacctgccatcatcttgaagcgaatttaggggaagaaagaatattgcaaatgacaacataggtatttcagaaaaaaggaaaaatctgaatgaaagagtaattgagatgttgatacagagatgacttcatattaattctgaactagagagagagacactaccttaaataaggatggaaagaaaaatactaaatttgggaaaatgacgcttagtttttgaaacatttaggaattactaggtaaaataaaattcaatgactttcatcagtcctaagtgtgtcaaatgtgagagttaagaatgagggactagggaaatgtggcagtcccttggtgaggaagctagtatgagaggaaggatgtttttgaacgtgtctgtcatagtgattttcatgcaaaattgtctgcctgttttaaaacatggtagattgcagtgcagctttaacatgctttcctctgcaggctccatctactctttgaagcttctgcccagcttgcattgtttctaggagaacctgcgtcatacctttatctatagccttcccctaggtcttcagaagcatcaagttttaactgtggacattggatttggtggaacagcaatcatggtaagctgtatgataaggctgagggttgaaatgtgctgtaagggccgaaagaaatagtttgccagcatgtgcagtgatcttgggttctgaatgttagaaataaggatacatccatggatatggattctcattgcattgagtatcagctgaagatgagctgatttttttatttttatttttttcagacggggtcttgctctgtctcccagtctggagtgcagtggtgcggtcttggctcactgcaaccctgggttcaagagattctcgtgcctcagcctctttattagaaacagggtttcactgtgttggccaggctggtctcaggctcctgacctctggtgatccacccacctcggcctcccaaagtgctgggattacaggcatgagatgagccactgtgcctggccatgagaaatgtttcatgatgtgagaaaatacaggagtttttggtcatgattccaccagtggtgaaggactgtcctgcagatgatggactctcaggtcagattacaatagaaataaagagggctttgaaaatggaagggttttggcattaacagtacagagaaaagcagtctggagagagagaacaccctacatcattgtggtttggtttacttaggggagtggacagtttagagcatgcatcgtcacagagaaggataatcacctgtaaggaggtgaaaattggttcttgagatgtgaaaaaatacttcataatcctttgtggtcctccaaagagccatactaaatatggaagaatgttggtgaaaaaggagagaatatttgtttagttaaggaaaccaggcttagattatgtaagggtggagaagaaacaggagaagattagaagactagggtgttggcaagctgaacatgactcttgtcttactgcttctagactgttggcaagagtagcaagatgctgcagcacattgactatagaatgagatgtatcctgcaagatggccgaatcttcattggcacctttaaggcttttgacaagcatatgaatttgatcctctgtgattgtgatgagttcagaaagatcaagtaaggctgatttgggcaaatgggggttggacacagaggcagtgggaagggaaggccagagctggagtaagggatttccgagggtaactgaagtagttagggaaactatggtagagccagtctattgtttaacctcttgacctgatctctgaattaggtcagagtctgcatctgaactcttttaagtttgtaaaacaatcatggatttattactgggagtaaaagtatatgctatggtcagaatactcgtatttatttttaaaattgagaaaaatgctatattaattaattggttggcttctgcagcagtcttagaccaaatcttggttgaaatggagaagaacagttgttcttcatggttgaggagttgggatatggttgccatttggtatcctctctttgcctatcactgttcccattatttcagttctgtgtcacctgtcattatgatttaattatttaagtaattttttaaagaaatttccattttaatgtggaaacccaggtttcaattttgtttttcttaaaaggtatttcacaaaagaaattagtcttgttgtagtgtaacatcaaaggaacattctgtaaaagactaaagttagattagaaaatagcttcactatttacccttgtatattaactgtgtttacagatattttttggcttgtttttcagtgataactcagtaaatgtttagcatcagttgtcttaattgatacttgaggttgtataaatattttgatgagtgagtgatcactaaaatttatctcactaaaatttaattctgatttgtaggccaaagaatgcgaagcaaccagagcgtgaagaaaagcgggttttgggtctggtgttgctgcgtggggagaacttggtatccatgactgtggaggggccaccccccaaagatgtaaggaagatgtagggcaggacagaactttaatttgcagggacatcatattatgtgagatgtctgaaatcagggtagagcagacacagttcaacatggattgtcaaataaaatgtgccaggcacttaatatcaattgttggttgcctatgctattcgggattaggggatttttggtgaattattctatgtgctgggcattacctaggtaagggtaggagttggtgattcatgtttgcttggtatgtttataaaagaggtgttttcacaatttgacaaataatgtgcattttatacacaagatacctccatggtatacttgcttgtttgttcatttggacacagaactaatatgagataggtgtcatgggaaaatggtggagagaagtgatctctgatgaataagaatactgagaacttgtaaattgtttgattttaggctatgaattttcttgtttcagactggcattgctcgggtaccacttgctggagctgctggaggccctggggttggtagggcagctggtagaggagtaccagctggtgtgccaattccccaggcccctgctggattggcaggccctgtccgaggagttgggggaccatcccagcaggtgaggaaccagcagagggttttatattattgggagaatatgactaagccggaggccgaggagatttaaaaacctaagtgtatgtgtcatacaatgtaaacagcatatggtttaggaggaaccaaaacacagatatatgggaggaagggaataaaactagctgcttaaaatggtcattgggtgattaaagttacccaggttactcttggcaagagtaacttgccactagtggtgagtgaacagaatgcatggatataccacagttgaaaagaccaaagggctttttggatgtcagtgtactaagattagtttttaaaatatgggaataggccgggtgcagtggcttacgcctgtaatccagcactttgggaggccgaggtgggcagatcacttgaggtcaggagttcgacaccagcccggcccacgtggtgaaaaccctatctctactaaaaatacaaaaatcagctgtgtgtggtggtgggtgcctgtaatcccagctaccaggcagggagacaggagaatcgcttgaacccaggagtgggaggctgcagtgagctgagattgcgccattacactccagcctgggtgacaagagtgaaactgtctcaaaaaaaaacatgggaataatgagagaagtacattgcaacagttgtttgtaactaattggtcatttttctcatttattttctgtgtttgaataatgtgaaggtttgcatcgctttgactgtttcccgccctgccttctcaggtaatgactccacagggaagaggcactgtagcagctgctgctgttgctgcgactgccagtattgctggagccccaacacagtacccaccaggacggggcactccgcccccacccgtcggcagagcaaccccacctccaggtaagggattggtgaacacgaagacgaacttgaatctctgatgagagatagcttactgatttaagacacagcctgagagcctaagaatttggggaatatgcttccttcttctagatactggtttttaatgaaagacatagaagagtaattgtcatatagaagttatttgactctatcattgttgtctggcccatttctttagggattatttgggctaaattctaacttttctaagccattttatgaggcctttatttctaccatttttcactgtaggcattatggctcctccacctggtatgagaccacccatgggcccaccaattgggcttccccctgctcgagggacgccaataggcatgccgcctccgggaatgagaccccctccaccaggcattagaggtgagtgggagcataggggtttgatggttcagccaggcccctgaatatgtgtatcctctttttctcaatgtttctatttcctttccaggtccacctcccccaggaatgcgtccaccaaga

SEQ ID NO: 1128 - DNA, 740 bp, Artificial Sequence
  Synthetic polynucleotide
tcatgacccgggggtttaagtggttgtgctgcttcacacatggcctcctgcttctcatgttgctatgtcacattatctgaagggaagtggggatgggatgaaaggccttgtaaggccacctatatactgaaggaagttgtagtgaatcacagtccatgctcattacatatcaaggcatttgaaatagaagttactctttttcttgggatgttgacctcagttgatcaggttttacagtgtcctatgatcagaatagaggatgtgatactggaataatcattttctaccattgagtaagtatttatttataaattactaaatgagtggaagatcctgggtactgggaggcaggaggttgcatatttgagttgataagttgtactactgataataaagataactgacacactaaaacatacttggtacactgaaatacaaattacggttatgtccagtggtatcaccaagtgacaaactgggagctaaatagttcagaaatgccctagtctctaggtccagtttgaattggttatggaacaaagaagccatttataaaagggaagagatttttaggttttgcagctaaggtttatagtaggcagcgagggaatgattcatagtgattgtagcagttggtttaccaagcactaaagagattggtgttaactccataatactgatagactgattggaattacattttttctcttgaatttaccaggagtggttgtgaatgtcac

SEQ ID NO: 1129 - DNA, 10379 bp, Artificial Sequence
  Synthetic polynucleotide
caaccaaagtactttacctggatctttcaagacagagcttctcagttcagacaaattgactgatgataagacaaacacttttcctctacaaaccgtggggtagtatgtagagagacctgtgatttcttcccatactgctctggaactacttctaattatacttttttctttgtttgtttttgaaggggaagcatttctccttgcattggtttgctggtatggatggcccttagattttggcatgagtgattatgcagcctatttgaagaaaacctgttttcaggacctttttaattaaatatggcctgttacatgagttaaaggtaagttataagagctctagattctgcaagatgcttgcttcttgtagttattttgcttggtgataatacattttcttttgtctggagcttcctggactttttgagaatcctgattgtgatgatataataggagaaaacttgggatatagatagtataattgagtgtggtgaatcttttgttatggaatgctagtctaagcttctgtgtgtttccactagggacacacatcaacatatcagtcttaaaacattcttggtagttaatgcttgatgttacttaaaaatcatcctacattcttgcattaaactacatacacgtggctgggcgcggtggctcatgcctgtaatcccagcactttgggaggccgaggcaggcagatcccgaggtcagaagatcgagaccatcctggctaacacggtgaaacctcgtctctacaaaaaattcaaaaaattagccgggcgtggtggcaggcacctgtagtcccagctactcgggagcctgaggcaggagaatggtgtgaacctggtggagcttgcagtgagccgagatcgcgccactgcactccagcctgggcgacagagtaaaactctgtctcaaaaaaaaagaaaaaaaaaaaaaaaagaaaatacatacatgtctcatctaaaacatggttggtcaacaactttctgatttcctgatacacattttattaaccggctccttaaatatatccatgttgcatatcatgtttgacatgctttcattgtagcttatagaataatttcaaatagaattttgggataaaaattatgctaccttaaaataggttatgaagtatttgaggtattctctttttatatgataggtaaacttggcttggaattaaaaagattttgaaagttgcttagggagctccctgcttttgtgaactcattattccttaattatgatgtcaaatttcagaagtttgttattaacataccggtattttgctttttatagattcttgtaatagaatattacaagtagaaactgatttacatagataattatgtgaaatagtgctgcttgactaggattttcatgatctcatttcaaagattagcaaaaacaccctttacctggtatgtttacctctgatactaccttcactgatttcagcaggtagtcagttataatccatggattgaagttttgatttatttatttatttatttgagacagagtctcactctgttgcgcaagctggagtacagtggtgtgatcttggctcactttagcctctgccttccgggctgaagcgattctcctgcctcagcctcctgagtagctgggattacaggtgtgggccaccacacctggctaatttttgtatttttggtagagacagggtttcaccatgttggccaggttggtctcaaactcctgacctcaggtgatccgcccaactcagcctcccgaagtgctgggattacaggtgtgagccaccgcaccccgccatgggttgaagtttttaaatgcatgttttccggtagttaatacattgcttgattctgtcttataattgcatttcagttctcttcatcttgtccatacacataataatgcttagtatataataaagcaaggtatattttcatgggatttttgtagtgggagaatgacatctgagcctgaaataggatatttgtgttgtaactttttcagtgtcatacgataccattttttgaccacttagttgatgcctctgaaaggaagagagaatcagatcgctaaaatttgatgtgtcgtcagtgtggatcctactcagtagtgtcacaacgaggctgaaaatcatattaagtgcttttaggacagaaacgtaaactgctcaggtaaaataaccttataaagagaaataagcatatattgcagaggccttggctaggttcatgatgacacaggaccttgtctgaacataatgatttcaaaatttgagcttaaaaatgacactctgaaatccagtcagtgtgcctcactagacttttcgatttcaagattttctgcagaaaatgttttgaaaactttgaatacttaaaaatggcaggtgtagtattgcactttgctagttgctcagatacccttttttatttgtatagatattctgagttcctttttttttctacatgttgtacgttgtcgaaagctaaaaggaaacttatccttggatcacggaaggcagaggcatttggtgagatggaaacaaggatgtgtaaaaatgagacgaccacctctcggattaaaaaaaaaaagtgccagagttctagggttctaagtgatgtccaggaaggaggaggaataatatttatggagcatatattatggaacacagtgagtatagtacctgcctttaaatgaatactgttggttttttaggacagttgcttttttttcttttttcttcagctgtgtgcagttgattaacttgtacagagcctatcacacaatagatgtttaagaaatattaagtgaatgaatgaggcagcattgctaatttttgtatagtgagacagtatctcacagtccaggctggagttcagtggcattaacataactcactgcagccttgaacacctgagctcaaacgatcctttcaccttatcctccagagtagctgggactacagtcgcgtgtcaacatgcctggctaattttagttttctaatttttttagagttgggatctcactatgttgcttagactggtcttgaactcctggcctcatgccatcctcttgcctcagctggtattgtaggtgtgagccactgtgccaggtactaattttttttatgttagaaaacaagtttttaggtattttatagttacttccttatgacaatatctcatcagaatctaggcctatctgatatctgactccatacgtttataccgtgtagtcagccattcccagatagaggtcatttgagttacttagtgtttaccagcacttgtttactaccttgcgatgatatttacagtgtatttttgggtatggttttggggttatttggttgttgttaaaatgcctgtaatcccggcactttgggaggttgaggcgggtggatcacctgaggtcaggagttcgagcccagcctggccaacatggcgaaaccctacctctactaaaaatacaaaaattagcctggcgtggtggcaggcacctgtaatcccaactacgcaggaggctaaggcaggagaattgcttgaacccgggaggcggaggttgcagtgagccgagatcgcgccaatggcactccattcagcttgagtgacagaggtgagactcccatctcaaggtaaaaaaaaaaaatagaaatcagcaactgctatttgagtttacaggagtgagaagtcctctcttgcatggtgagatggggaacatttaattcaatagaaatatttgagaccaggtatggtggctcaagcctgtaatcccagcactttgggaggctgagttgggtggatcacctgacatcaggagtttgagactagcctggccaacatagcaataccctgtctcaaagatacaaaaattacccaagcttggtggtgtgtgtctgtaatcccagctactcaggaggctgaggcaggagaatcacttgaacctgggcggcaaaggttgcagtgagccgagaccatgccactgcactccagcctgggtgacagagtgagactccgtctggaaaaaaaaagaaaaaaatgagatataaagttcaaaggatgtatttattaagccaatataaagtaaagaggtggggaatgttgtagaagtatatgttgataaagggatcaggtggttgtacaataagaagggggttgcagacatggagaaacagttcttctagctcttccactttcagtatcatctgtgtgggtcactggaatcctttttcacaatgcctgcttgcccatgagttttaagaactagtacagtgtagaaatgattagtaggaaaaacttggctgggttaatttgctttgctgagccgattcaacttaaaaagcgaggtccatactaagtgcccaagaatcaagaaggaggctcacatttgaacttgatgtttagttaatgtgaatcattttctgttgaactaatgagaatacagtgaccttgagggttaggttgtacatctgaattgttcgggctgtttcctgtcttttggatgttttgtttgtatagatattttcattttatgttacatcatttgttctttggtagcaaaaattgagttgaaatgatacagttcctgaaatctatgatttcatgtttcggagatatgtatcaggaataatagtgtgggttctcatgagaaaatctggattgcattactgtgctactttggtttcttgtcagagaagttagtagtttagacctccttcagaatcctttctttggactttgtgtttcttacgttgtgccagccttgattttatttgtctgtcctctgtctttctataccatttgtttacacttaactctagaattttttgatgcatgttttcagtgtactctagtcctaaaattggtttacatttaattattggggagaatagcaagttgtcgatagtatctgtggtggcattagtgtatttctgtttcagttctttggacagtttgctagtggaaatagtcattgtatttagctctgtgagacccgactggcagattgctgtctctctctctctctctctctctctctgtgtgtgtgtgtgtgtgtgtgtgtgtgtgtgtgtgtgtatgcgcgcccgcgtttgtttccttcatgtagattgctgtgtgtgtctttttcttcataattccccttaattctgtgtgctttgaatgacactgttgttgttactgttgttttatatccattgtcacataaaactcttatcatttattcttccaggccagggactgtccttatgggtagagatacactatctgcaggaatttatttttttacgtatttttactagttgatgggaaagacattgggaaagacctgggctggatttgtgatgagctgtgtttactgagcatgatgaagtaaagctcaacgtgattactctgaagtccagccttactgtcagtctaaggactcaggatacaaatactcaaaacataaattcttagactctaaatttcacatggtttttcctatcagtacaggttaaggataatggcgtgttgtaggtttttccagtgcaatacaaggaagttggttctagatcctttagtacagttaatgttttgtagagtgtgttctatgttcactgcattggtatgtctcatctttggcatgatcttggctcaccgcaacctccgcctcttgcattcaagcacttcttgtctcagcctccagagtagctgggattacaagcatgtgccaacatgcccggctaatttttgtatgcttttgtggagacaggctttgccatgtttgccaggttggtctctaagtgatccacccgcctcagccccacaaagtgctgggattacatgcgtgagccaccgtacccagccacacctttgcttatttctactatgcatttcaagtgatcatttcaagagaataagtacttaggagctcctatgctctttttttttttaaccctgttgcttgcatcacccggttccaaccctatatgaaagtttcatgagcatgtttctgtcactgaggcagcttctccatttggcatataattgagaaattgctagtttgtggctgagacattggtttgagaggagacgtaatttatatatttagcagaatgcttaagttacatttccagagattaatttaattctacttttaattaagttaaccctttgctgtatctttacaagacgtaggtggagatttaattttgactcttagattcagttaaggtgatgaaattggcattatatagtttttgatgctgagccccacggagattaaaagatttgctaaacaccatgcaatttttgaatcacatctctaatctcacattattctgtactctattttttgcttgtatgattgatgggtctttcattatctgtgattgacattctatgagtaggtgcttttgctttgcctataagtcgttattatgaaggaggaatggtgaataagaaggtaatttagaaaagcctatattaaatataccatgaacattgaatatagcaagatcttattctctagttgttatcttagttgataaattctgtatgtgttatgtgtttgtgtatacatatgtacatattaatgatgtgggtgttgatactgtgatcacatgacaaatgactggtttatagtgaaaagctgttggggttcagagaaatgaacattcttctaaaagtgaggtagataagttattttatagttcttgcctttgagtctctgttcttctggtagaaatgttagtgtatttataacctctcagatttgtttggttctcttagtgttcttgatccaggcagttagcgatatgttggatgaattgagaaaaatgagtttttcctttcctactaactgaattagattagaaataagcagttaagtgagaaaaatagggaattagtgccagatatgttgaggtgtagtgttgacagggggtcttttaccttcagatgtttggtgcaacttagagaatgcagtggtagttgctggcccagcccatccaaacaacctcatttgtggggtgatacttcacagatgacttaaaataaatcccctgaaaataagaatctctggaagagatgacacgtgtatgtgcgtgtgtgcacgtgtgtgcgtgtgtgcacgtgtgtgtgtgtgtgtgttttggtggagtggtaggaggagggttggcttaatgatgagaatcattatttcttgaattggatgacactttccattcctgcaaagggagcgtgaggtccagccttgctaattcatgatcaaagtaatttgctgagagtagtggaaggaatgagttagcagaagatggcaaattgcattaatgagtagtaggaggaaaacaaataagcaaattttaatgtggttggaacatgttcctgagttgtatgaagggagacaggtaaattgagagtaatgttatgattgtttcatttgttggaaataaggaaaacttgcatggtttgaattgtggaacaataggtggtgtatgttaggagtcagaattatcatttaacatgtaagtttctgttttacttcagataaagaatgtcagctacctttcccatgttagcttctataccttttcttgaggtgaccataaataggtaatattgtctttcactgagattgttttaattagcgaagtgtgctgattaaatgtcttttcttgataaattattagaatgaataaaggatggtatttctagatagccttggttagtcagtgatttgtttaaatttagtgtggataagaatcacctgagggctttttaaacacagtttactgggtcctgaccttagtgtttctggcttattaggtataagtatgagaagttgatttttatcaagtttctagatgctacagatctaggaaccatacttagagtgctattatatgtcttttaatgtacactgatatggaaaaagcaatgatacatcatgttatcgcatattttaaaatgtgatcatgaccagggaatcttgaaagttgtcttatgtaataatgtaaatcaatgtagaataccatatatttttttcttgcaaggttattattaataggaatgactatttgtagccaatgtgttcaaatgcatgggaggacgtaaacacttcataatttcctggaaatacagacatatatcacttaaaataatacatttctaatataaatttaaattatctacgtgatatgaggaaacgagcttatttttgataattttcctagcagtgaatcaactatactcatttgaatctaaattaaattaaatttgagtaaattcagccatttctaaattcagtccatataaattcactttgggtaaaagaagacaaaattcctgaaaacagtagattaaaaaaataacattttaagagcaggtatgatctcaatgaatatagtgtggtaatttctgcctaatacctacccttttggaggaaggaaaggtaatcctgtttgtgtttaatgttaattactgaactctttataacttgttttatttatttattgtttgattttaagcatctgcttctggtggacttacatatgttgggtttgggaaatttcaacatacaggttggcattccagtggctatagttgtttcttgaaatttagtactgttttccaatgatgattctctttgggaataggctgttctcttttacttaaaaactgtaaaattagccacttcagttgagactcctccaggtttggaagtggtgaagaatatggatatattcagatcaaatcagtcattctctgtgatacagcaactgaacaagaaacccagacttcaaacaagcagacttctttgggttggactgaataaaatgagaagaaattacagaggatttgaaaatgtatatggcctaggaattattgaaattattttggagggtggcaacaaatttgggacattgtactttctgttttaatgtcaattgtataatgtgtaactctagtgagcatggctggataccgtggcttcacattttaggtagactgggtgttttatctactgagtgaattacagccttcggcaatgatgtcaaggtagcaacaaaacccaaaaaatccatcctttagaaattgttttaagtcacaatgatgataccttctttaaaagctttgacttactgaaataatgcttttcatttgtccaagatgagtacatagcagaatttggaaatatggttccaaaaggctaaatagactgacttttttgatatttttcccttatagggagaaagaaacattaacggtttatcagatgccttgactgtactttttactgctttgtcttcaaggcctagtgtaataattaacatctagtatgtgtttgatggatagccaatttttgcttcattggtatgttgttaccacagtcattggtagagtcaatatatgaatgaagaaagtataacaaatttgccctctagtagagtacaggcaaagtatggccgtcagtttcaccctgattttttttcctccattcttttttagggtttaggtcaagaacaagggcatttcattgtaccacatagctgagagtagggaattgggacatactgtcttgctttaaattaaagttgtctgtttcatcaagtagagaggttcctaagatattgccagatttctgactcggggtttgttaaaaatagaaaaggtagttccttaaggttgtattttattggatgcctccacctgcttcaggctagctcactggagaataaatattccaggttaaaaaaaaaaaagcatgtgttcacgtgaacttgtcctttgagcaaacctgttggaaatcaagaggtcattgctgttggagaggataagagggtgtacttgttggtgtgttaagagtagttcacaacataaaaataaatgtaaatgcatctattatatttgggttggcaaaatatattggttatttactctgttgattcctgtagtgtacataaatatgatctacaactctgtgaacaaaaaggcttgaaaaattgttctgaaaatggacaaatcatgaacagataattgaacaagaaaataagcagaaagtaaatttgtgaaatatttcaaactcatttacattcagaaacatctttttaagatcactgtgatacattcgactgtcaattctgaagagacgtataaaggaaatgccagctaaggagcaaggaaacagctttcattcatgggctaaattgcagcttaaaaaagatggaccaggattatttcctttagaaacttactgtacaaaagatactgttcaccccattagagaactgatttgaagttactcttccctgtgagggctctgtcatcttaactgtattcacatactttcaactgttccccttgctgctaacctcaggttctttagttcatcta

SEQ ID NO: 1130 - DNA, 463 bp, Artificial Sequence
  Synthetic polynucleotide
ctgtagagccatagtccagatctactctatttgtgtgctccttacaacatcagaacagcaactctcaatccggattcatccccagaatgccgtctgagtcacagcgtggcaggtgcataaagaattatctactgaattgagaaacatttcaaatatataaaacttgaaatattttgggtagtctgtacaaactcaagttgacaagccaaccctgcaggaatatatggatagaaggggaaggataacatgttgaattcagctacatgcctcatcagctagaaaaccttggaagaaagaaagatagattgatacttgcctgaacttcatcaaccaataatattttttttcttgctatggagagtccactcgcctttggaagatttaccagtaaaagccctgcacatgatatacttgtgtgcacatgcaataaggacatcattccccaactcctgctagcattgtg

SEQ ID NO: 1131 - DNA, 853 bp, Artificial Sequence
  Synthetic polynucleotide
tgatagagcagcgtggcccaggggaggggctaagcagtggactaaggatggggaggctggagaagaggctgaggggagtcacatgttgtcctgagatgagaaagggaaaaaaagaaacatgttcatcttcaagtgcaaactctcttcagattgaaagaaagtttttagtttcttgggaagcagaaaagaatataggtgaggagtttgagagattcattaatttctttgtgaaattcaacataaaggaagatattctgtgttgctctttgttcactatatgtgtggggctcacagggaagcaatatagacaaagtttccaaattctcagatgttaaaggccaatggggattcagaaaacgcaacagaaactaaactgcaagtggaagagctgcccttgggagcccagagggcaagtttcaggcctggactgggtgggcaagggaggcctccaggaggaggagctttggaggagattgtggagagtgaggagcagtgagtccagcaaacagggcagggaaagcgaggaggaaacagtgtgtctgtggcacaggccagggcatgagaagagggtggtgggttggaaggaccactgggatgagtaatacatgaggaggcaggagaaggaagggcagaccatgcagggactgggcatgggggtgagggtgccgggcagctcagaaagtgatgcaggggccgctctctgaggactgcattctgtggggagggatttgaatgtctcagttcttttatgaaatgttatccccaatccatgaggtcttctgattcaggaaccagtcagaacaggtgagggtggccccagtccactgtgcaggaccattagcattgccaggag

SEQ ID NO: 1132 - DNA, 358 bp, Artificial Sequence
  Synthetic polynucleotide
cattatggttgcctggtttttggaacctggggtactttgtactcctccagcaacagccagaggtggcagagagctgtcagtcatccctgggtgcctcctgcgcaagcgcagttgtcctcctgcgccgacctcgaatgcggtcctcctgctgcagaccacgcccaccaagggctggccgcagccactgtagctgagctcagagccttctgtgtggtttgcgggggcaaggtcagctgccccctgccctctctctggggctatttgaggaatgcggcctttttgcataagaaaggcttttctctacagtaactgtggtcgctgatcaatgacaccttcctggtaacatttcatattgctt

SEQ ID NO: 1133 - DNA, 769 bp, Artificial Sequence
  Synthetic polynucleotide
gttgggctacctttatccttgggattgttgcaagatggccgtgtcatgtaaagggctgtgtcttttagcatcatgaataagatgaaagagttgaaggccaacttcattcaaatgtcttctcgatgtgtccttccctttttctcctggactgctctgtgatccatgtctctgtagaaaatggctggcataagtcaatttctgactgggagctgtcatgattactgtctcccactgttaacactgtacttggaggtcttgatggaattgggcaaacctgttctttcagaccctcagtttcatctcaacacaaaactctccaaaagtccttgctgctccacagcagggaactctcttgaagcaataacatgaggaaatagtgggcttgcatggctgcctcacattcctcaggtatcactgtgctcattgcctgatgtactgtgtcttgccaaccatcattccacgtaatttacccattcttggtttttacattggttaaggcatgtacaaaccctgttagtttttctgcagtatttggattcataccttctgaaattacttgacgattactgtgtctctggttatttgcttctagttggcaatacaaaggaacaatgtgaatgaaactcataaagaaaaacctatgatttaatgggcgtgtgtaacactagagggaaggtcagagttagtctatgcatgggtataggtaggagtctcagtgggtgtcagccattctgtgtcaacaaatcaaatatttaaggtggatctaaaa

SEQ ID NO: 1134 - DNA, 359 bp, Artificial Sequence
  Synthetic polynucleotide
cctcatcccctaccacccttaggccctagtactcaccattctactttgtatttatataaatgagcttttttagattccgcttacacatgggttcatgtggtatttgtctttctcttcccggcatatttcatttaacatattgtcatgcagatacattcattctgcctccaatgacagtaattctccctttttaaggccgataatatcctgttttgcatatacactgcattctttttatccattaatctgttgatgggcacttaggttgatcccatatttggctattgtgactaattctggaatgaatgtgggagtgcagatatctatccaacacaccggatagtcaatgaaatctccca

SEQ ID NO: 1135 - DNA, 109 bp, Artificial Sequence
  Synthetic polynucleotide
acagttcagggcatgagataagggcagtgggttggaaggatcgctgggatgaataatatatgaggaggcaggagcaggaaggacagaccataaagggcctgagcagggg

SEQ ID NO: 1136 - DNA, 1144 bp, Artificial Sequence
  Synthetic polynucleotide
ccgcacataggaagacctgagggtgagtgtaaattaggattgtttggccagtggtgtgtgcaaaaatttattgtagaaccaagaattatttctttagtattttacacattttaaaaaacaggtagacatgtccattgatcccaggttgcttatggtttctagaggccccctctcattgcaacagtgctgtggggccctaggggtccagtagccccctccccccaggtcattccggtgagggagggagctgggacccctgcactgcggcaaacaagcacgcctgcgcggccgcagaggcaggctggcgcgcatgctcaggcggggatgtgtgcgaagcctgccgctgctgcagcgagtctggcgcagagtggagcggccgccggagatgcctgacgcatctgtctgaggagcggtcagtgacgcgatggagcgggcaaggtcagctgtgccggtggcttctctcaagagacagcctggggagcggccacttttattcatcagatattccaagtttttaggacttggagtactgaataaacggaatttgggccctaaagtcctttgttctggagaaccagatccggaatgttcagaggcttgctgttgtgccgttctgccccgatggtatcctgtccgctcgcattggggcgcgtcccccatccgcccccaactgtggtgtcgcgacaggtcctattgcgggtgtctgcggtgggaagggcggtggtgactgggagcatgcggggtaaccgcagtgggcaagggacatgggtggaggtgggtacatcagggtgattgcagttccgtgtggcgagggtacgtggggggaccagtgcatagggattttaggcggaggtaggtatattggagtgattgtggcggggcgaaggtacgtgaagtgatcggtatttagggggtgttgagcgcaggtaggtgtataatagtgaccactgcgtggtggagcagggtacgtggggcgactggggggggaattcgtcgcagtgaccgaggcgaggaggctatggcagtggaccagggggatgagtcggtgtgacagtggtgggggttgtgttggtggtcgcggcgcggggagagtccgtaggaagtggctgggaggtagggggaaggcggcgacagtgggtattgg

SEQ ID NO: 1137 - DNA, 169 bp, Artificial Sequence
  Synthetic polynucleotide
gttattgtagcgcatgcttttcaggtagttttcttttgcgttttttttaattatataaaaatatttcactataaatggctcttgaataccctgttactgaatgtttataatttccttttgtgggaggtgtcatttttttctgccagtagtttgaacaaagctttagtag

SEQ ID NO: 1138 - DNA, 2154 bp, Artificial Sequence
  Synthetic polynucleotide
ctgcccacctgcagctgtcttgatcgagttatacaaaaagacaaaggcccatattatacacaccttggggcaggaccaagtgttgctgctgtcagggaaatcatggagaataggtatggtcaaaaaggaaacgcaataaggatagaaatagtagtgtacaccggtaaagaagggaaaagctctcatgggtgtccaattgctaagtgggttttaagaagaagcagtgatgaagaaaaagttctttgtttggtccggcagcgtacaggccaccactgtccaactgctgtgatggtggtgctcatcatggtgtgggatggcatccctcttccaatggccgaccggctatacacagagctcacagagaatctaaagtcatacaatgggcaccctaccgacagaagatgcaccctcaatgaaaatcgtacctgtacatgtcaaggaattgatccagagacttgtggagcttcattctcttttggctgttcatggagtatgtactttaatggctgtaagtttggtagaagcccaagccccagaagatttagaattgatccaagctctcccttacatgaaaaaaaccttgaagataacttacagagtttggctacacgattagctccaatttataagcagtatgctccagtagcttaccaaaatcaggtggaatatgaaaatgttgcccgagaatgtcggcttggcagcaaggaaggtcgacccttctctggggtcactgcttgcctggacttctgtgctcatccccacagggacattcacaacatgaataatggaagcactgtggtttgtaccttaactcgagaagataaccgctctttgggtgttattcctcaagatgagcagctccatgtgctacctctttataagctttcagacacagatgagtttggctccaaggaaggaatggaagccaagatcaaatctggggccatcgaggtcctggcaccccgccgcaaaaaaagaacgtgtttcactcagcctgttccccgttctggaaagaagagggctgcgatgatgacagaggttcttgcacataagataagggcagtggaaaagaaacctattccccgaatcaagcggaagaataactcaacaacaacaaacaacagtaagccttcgtcactgccaaccttagggagtaacactgagaccgtgcaacctgaagtaaaaagtgaaaccgaaccccattttatcttaaaaagttcagacaacactaaaacttattcgctgatgccatccgctcctcacccagtgaaagaggcatctccaggcttctcctggtccccgaagactgcttcagccacaccagctccactgaagaatgacgcaacagcctcatgcgggttttcagaaagaagcagcactccccactgtacgatgccttcgggaagactcagtggtgccaatgctgcagctgctgatggccctggcatttcacagcttggcgaagtggctcctctccccaccctgtctgctcctgtgatggagcccctcattaattctgagccttccactggtgtgactgagccgctaacgcctcatcagccaaaccaccagccctccttcctcacctctcctcaagaccttgcctcttctccaatggaagaagatgagcagcattctgaagcagatgagcctccatcagacgaacccctatctgatgaccccctgtcacctgctgaggagaaattgccccacattgatgagtattggtcagacagtgagcacatctttttggatgcaaatattggtggggtggccatcgcacctgctcacggctcggttttgattgagtgtgcccggcgagagctgcacgctaccactcctgttgagcaccccaaccgtaatcatccaacccgcctctcccttgtcttttaccagcacaaaaacctaaataagccccaacatggttttgaactaaacaagattaagtttgaggctaaagaagctaagaataagaaaatgaaggcctcagagcaaaaagaccaggcagctaatgaaggtccagaacagtcctctgaagtaaatgaattgaaccaaattccttctcataaagcattaacattaacccatgacaatgttgtcaccgtgtccccttatgctctcacacacgttgcggggccctataaccattgggtc

SEQ ID NO: 1139 - protein, 718 aa, Artificial Sequence
  Synthetic polypeptide
LPTCSCLDRVIQKDKGPYYTHLGAGPSVAAVREIMENRYGQKGNAIRIEIVVYTGKEGKSSHGCPIAKWVLRRSSDEEKVLCLVRQRTGHHCPTAVMVVLIMVWDGIPLPMADRLYTELTENLKSYNGHPTDRRCTLNENRTCTCQGIDPETCGASFSFGCSWSMYFNGCKFGRSPSPRRFRIDPSSPLHEKNLEDNLQSLATRLAPIYKQYAPVAYQNQVEYENVARECRLGSKEGRPFSGVTACLDFCAHPHRDIHNMNNGSTVVCTLTREDNRSLGVIPQDEQLHVLPLYKLSDTDEFGSKEGMEAKIKSGAIEVLAPRRKKRTCFTQPVPRSGKKRAAMMTEVLAHKIRAVEKKPIPRIKRKNNSTTTNNSKPSSLPTLGSNTETVQPEVKSETEPHFILKSSDNTKTYSLMPSAPHPVKEASPGFSWSPKTASATPAPLKNDATASCGFSERSSTPHCTMPSGRLSGANAAAADGPGISQLGEVAPLPTLSAPVMEPLINSEPSTGVTEPLTPHQPNHQPSFLTSPQDLASSPMEEDEQHSEADEPPSDEPLSDDPLSPAEEKLPHIDEYWSDSEHIFLDANIGGVAIAPAHGSVLIECARRELHATTPVEHPNRNHPTRLSLVFYQHKNLNKPQHGFELNKIKFEAKEAKNKKMKASEQKDQAANEGPEQSSEVNELNQIPSHKALTLTHDNVVTVSPYALTHVAGPYNHWV

SEQ ID NO: 1140 - DNA, 86 bp, Artificial Sequence
  Synthetic polynucleotide
gtttaagagctatgctggaaacagcatagcaagtttaaataaggctagtccgttatcaacttgaaaaagtggcaccgagtcggtgc

SEQ ID NO: 1141 - RNA, 86 nt, Artificial Sequence
  Synthetic polynucleotide
guuuaagagcuaugcuggaaacagcauagcaaguuuaaauaaggcuaguccguuaucaacuugaaaaaguggcaccgagucggugc

SEQ ID NO: 1142 - RNA, 106 nt, Artificial sequence
  Synthetic polynucleotide
ggaaccagucagaacaggugguuuaagagcuaugcuggaaacagcauagcaaguuuaaauaaggcuaguccguuaucaacuugaaaaaguggcaccgagucggugc